SEQUENCE LISTING (25 sequences)

SEQ ID NO: 1 - DNA, 26699 bp, Pseudomonas aeruginosa
  misc_feature 1..26699: note = Description of sequence: polynucleotide, name: PAKAF R1 PYOCIN
  source 1..26699: mol_type = genomic DNA
tgatcaaagccatcgatgaaatgctcaagctctgggccgaggaaatgcacgcgccaggcagcaacggaggcggctacgccggcggcaacctgatcgccatgctgatcgccagcaagggcgaggtggtccgcggccaccgtggcagccgggtgatcctcgaccgtgtggcggaggtcgatcgcctggtaaatcgcctacccgaggaactgaagaacgtggtggtggagcactatctcaatcgcgacagcttccccgagcagaagtaccgccactgcggttgcagccgcaacaccttctatctgcgcctgcatgtggcgcaccagggtatccaggacggcctgctgcggcgggtggcctgagttcctggaccggataccgtgtcccccctggaattttccaaccggccttcgagccggtttttttatgcctgtcgcccatggaggcggtgagtcccatgcacagcagcggaccctgcacggtagaggcggattcgggctggcgtagccatccttttcgtcagtcgatgccgggctttccacatcggaagtgggtatgcgcttgcccggatgcctggcgaaggttcccagggcggtatatcaccctcatatcgtggtgcgtctccctcctattgcagtggattgtcgagaaggcattgccgggctgggaatcgggcggtaaaaagtagtcattcttgtaaaggtgcgtccccagggaggcactcgtgaagcaccggaacccggccctggcgccgggttttttattgcccgggaaaacgttcgtgcggacgtgcccggcgctgcctgccaaggagggacccatgggcaacgaaccgcagacactgacggaaatgccgctctgggtactgatcctgctcgccgcgctgggcggcgtcagcggcgagatgtggcgtgccgacaaggccggtctcggcggctgggccttgttgcggcgcctggcgctgcgctccggcgcgtcgattgtctgcggcgtggcggtgatgctgctggcgttggcttgcggcgccgcgctgctgttcgccgcggcgctgggcagcctgaccgccgcggccggcgcggagatcgcagtcggtctctacgaacgctgggccgcccggcgcctgggggtttgcgagctgcccgaggaacagtcggacgaccgtggcccgcattgaaactgatcgaaggagtcaaccatgcctgaacaggctgttacgctcgaggctctgtacgcggccatcgagcaggtactgcgtgagcgtctgccggaggcgcagttgatcggcttctggccaggcgtgccggaaaataccccggcggtttccctggagatagccgagctactgcccgagcgcgatcccggtaccggcgagagtgccctgctgtgccgcctgcaggcgcggataatggtgccacctggtgccgatcgccaggcggtatccattgcttgcggaatcgttcggacattgcgcgagcagacctggaacctgtctctggaaccggcgcgcttcgtacgctcggccgtcgacggcagtcgcgaggagctgaagagcctgcgtgtctggctggtcgagtggacgcagtcgctgcgtctcggagatccggagtgggcctgggaggaccagccgccgggcagcctgatgctgggcttcgacccgcagaccggccccggccatgagccggactacttcgctccggaggcgttggcatgagctatgtcagtgcggagcatgaccgcatgctcgccgcgatgatcctgccctgcgtggtggtcgccgtggacctggcagcggcgcgggtacgggtgcgctccggcgactggaccagcggctggctgcgctggcactccctggcagccggcaaggttcgccactggcgtgcgccgagcataggcgaacaggggatactgctcagcccgtcgggcggagtgtcaatgggcacctttattcccggtctgtacggcgatgcgggcacggcgccggacaacagcgccagcagtgagacctggcgtttcgacgacggcgcctcgttgagttacgactgggctgcgcatcgctaccgcgtcgagctgcccagcggcaccgtggaagtgagggtcggcgccagcgaggtgcgggtcagcgacggggcggtcagtctcaaggcgccgaagatcagcctggaaggaccggtggagatcgccgggacactgacggtcagcggagacatcctcggcggcggctcgatcatcgacaccgccggcaacagcaaccaccacacccattgaacatacgacggggctgccgaagggcggtccgtcgaacattcaaccggcccgcgcgagcgggccttttcgtttgcggagttcgccatgggcaacactcacagccattcggagccaggcgcggcctgtctctgccggagcgggagggcagggcgatgatcgggatggatcgccgtagcgggctgcccctgcccggcctggctcatcttaaacagtccgttgaggacatcctgaccaccccgttgggcagcaggcgcatgcgccccgagtacggcagcaagctgcggcggatggtcgacatgccggtgagcgaaggctggaaaagcgccgtgcaggccgaggtagcccgttccctggggcgctgggaaccgcgcatcgcattgtctgccgtgcgagtcgtcgcggtcgtcgatggccgcgtggatctgctcctgagcggcgtgttcgagggcgagaacatcaatatggaggtctcggcgtgatcatcgatctttcccagttgccggagccagaggttatcgaaaacctcgatttcgagacgatttaccaagagctgttgggcgacttccgcgaagccatggctggcgaatggacagcggaggtggagtccgatccggttctcaaacttctgcaactggcggcctatcgagaactgctgctgcgggcgcggatcaacgatgcggcgcgggcggtgatgctggcatatgccagcggtgccgatctcgaccagatcggtgccggcttcaatgtgcagcgtttgctgatcaggcccgcacagcccgaggcggtaccgccggtggaggcgcagtacgagagcgacaagtcgctgcgcaatcgcatccagctcgcgttcgagcagctatccgtcgcaggaccgcggaacgcctatatagcccatgcactgggcgcggatggaagggtggcggatgcctctgcgaccagtccggcgccctgcgaagtgctgatcagcgtgctcggggtggaaggcaacgggcaggcaccggaagcggtgttgcaggcagtgcgcctggcgctgaacgcggaggacgtgcgtcctgtcgcggatcgggtaacggtgcgctcggcaggaatcgttccctatcaggtcaaggcgcagctctacctgtttcccggccccgaggccgagctgatccgtgccgccgccgaggcttcgctgcgcgactacatttccgcccagcgccgcctgggccgcgacatccggcgttcggccctgttcgccaccctgcatgtcgaaggcgtgcagcgcgtcgaactgcaggagcctgcggccgacgtggtcctggatgaaacccaggcggcctattgcacggggtacgcgatcaccttgggaggcgtcgatgagtagccgcctgctgccgccaaacaggagttctctggaacgctctctgggtgatgtattgcctgccgagctgccggtgccgcttcgtgagcttaacgatccggcacgctgtgaggcggccttgctgccctacctggcctggacgcgctcggtggaccgctgggacccggactggagcgacgaggccaagcgcaatgcggtagcgacgtccttcgtcctgcaccagcgcaaaggcacgctgaccgcgttgcgccaagtggtcgagccgatcggtgcgctgagcgaggtcaccgaatggtggcagcgaagcccgcttggcgtgccggggaccttcgagatcaccgtggacgtcagcgaccgtggcatcgacgaaggcaccgtactggagctggagcgcttgctcgatgacgtccgcccggtgagcagacacctgacccggctggacctgcgcattaccccggtaatccggtcccgtcacggactggccgtgaccgacggcgacaccctggaaatcttcccctggaaacagtgacatgacgaccaatactccgaaatacggtggcctgctcaccgacataggtgccgctgcgctggctgcggccagtgcagcaggcaagaaatggcagccgactcatatgctgatcggcgatgccggcggtgcgccgggcgacacgccggatccattgccttctgcggcgcagaagagcctgatcaaccaacgccatcgggctcagctgaatcggctgttcgtttccgacaagaacgccaataccttggttgccgaggtggtgctgccagttgaggtaggtggcttctggatccgcgagatcggcctgcaggatgccgacggcaagttcgtcgcggtatccaactgcccgcccagctacaaggctgcaatggaaagtggcagtgcgcggacccagaccattcgggtgaacatcgcgctctccggcctggagaatgtccagctgctgatcgacaacggcatcatctacgccactcaggactgggtgaaggaaaaggtcgctgccgatttcaagggccgcaagattctggctggcaatggcttggtcggtgggggcgatctttctgccgaccgcagcattggtctggcgccttccggcgtgacggcgggcagctatcgttcggtcacggtgaacgccaacggggtggtcacccagggcagcaatccgaccaccctggccggctatgcgatcggagatgcctataccaaggccgataccgatggaaaactggcgcagaaagcgaacaaggccaccaccctggccggctatggcattaccgatgcgctgcgagtcgatggcaacgccgtgtcatccagcaggctggccgcaccgcgtagcctggcagccagtggcgatgcctcctggtcggtgaccttcgacggcagtgccaatgtttctgcgccgctgagtctttccgctaccggtgtggcggcgggcagctatccgaaggtgaccgtggatacgaagggaagggtgactgctggaatggcgctggcggcgacggacattcccgggctggatgcttcgaagttggtcagcggggtgctggccgagcagcgtttgccggtattcgcgcgcgggttggctactgctgtctcgaacagtagcgatccgaacaccgcgaccgtgccgttgatgctgaccaatcatgcgaacggacctgttgccggacgatacttctacatccagtcgatgttctatccggatcagaacggcaatgcttcgcagattgcaacgagctacaacgctacatccgagatgtatgtacgggtgtcctacgcggccaaccctagcatccgggagtggttgccctggcagcgttgcgacattggaggttccttcacgaagacgactgacggatccattggaaatggcgtcaatataaacagcttcgtcaattccggatggtggttgcaatcgacatcggaatgggcggcgggtggagctaactatcccgtggggctggccggtttgctgattgtctaccgcgcacatgcagaccatatctatcagacctacgtaacactcaacggaagcacatattcgcgctgctgctatgcgggctcttggcgtccgtggcggcagaactgggacgatggaaacttcgatccggccagctacctgccaaaggcgggatttacctgggcggctttgccgggtaagccggcaactttcccgccctcagggcataaccacgataccagccagatcacctccggcatcttgcctctcgctcgtggtggccttggcgccaatacagcggccggagcacgcaacaacattggtgccggagtgccggccacggcgagccgggcgctcaatggttggtggaaggacaacgataccggtctgatcgtccagtggatgcaggtgaacgtaggagatcatcccggtggaataatcgatcgtaccttgacgttcccgatcgcgttccctagcgcctgtctgcatgtcgtaccgactgtcaaggaggtggggcgaccagcgacgtccgcgtcgaccgttacggtcgccgatgtcagcgtcagcaacacgggatgtgtgatcgtttcctccgagtactacggactggctcaaaactatggcatcagagtgatggccatcggctattgaggtgaaaatgatttttttccatgccgccacgggcggcttttattcgaaggaaattcatgggtcccgcatgcctctggaggatgaaatgcatcctctcgaagacgccgagtaccaggctcttcttcgtgcgcagagcgaagggaaacgaattgtcacggaccacaccggtcgccctatctgcgtcgatccaccggctccggccaaggacattctggtccaacgggaacgcatctggcgcgaccggcagttacagctcaccgacgggcctctcgctcggcatcgtgacgagcaggacctgggaaaaactacgactctgagccaagagcagcttcgtgagctaactctctatcgcgccgttcttcgcgactggcctattgccgcggagtttcccgacctgaacgcaaggcccgagccgcctgcctggctccaatcgctcatcaccccctgaaccccgccttgtgcggggtttttcattaatggagatctacctatgagtttcttccacggcgttacggtaaccaacgtcgatatcggtgcgcgcaccatcgcgctgccggccagttccgtcatcggcctctgcgatgtgttcacgccgggggcgcaggcaagcgccaagcccaacgtcccggtgctgctcaccagcaagaaggacgccgccgcggcgttcggcatcggctcgtctatctacctggcctgcgaggccatctataaccgtgcccaggccgtgatcgtggcggtgggcgtggaagccgcggagactcccgaggcccaggccagcgccgtcataggtggtatcagtgccgccggcgagcgcaccgggctgcaggctttgctcgacggcaagagccgcttcaatgcccagccacgtctactagttgcgccgggtcattcggcccagcaagcggtggccaccgccatggacgggctggccgagaaactgcgggccatcgccattctcgatggtcccaatagcaccgacgaggcggccgtcgcctacgccaagaacttcggcagcaagcgccttttcatggtcgacccgggcgttcaggtttgggacagcgccaccaatgccgcgcgcaacgccccggcttcggcctacgccgccggcctgttcgcctggaccgacgccgagtacggcttctggtcctcgccgtcgaacaaggagatcaagggcgtcaccggcaccagccgtccggtggagttcctcgacggcgatgagacctgtcgcgccaacctgctcaacaacgccaatatcgcgacgatcattcgcgacgatggctatcgcctgtggggcaaccgcaccttgtccagcgacagcaagtgggctttcgtcacccgtgttcggaccatggacctggtaatggatgcgatcctcgccgggcacaagtgggcggtggaccgcggtatcaccaagacctacgtgaaggatgtcaccgagggcctgcgcgccttcatgcgcgatctgaagaaccagggagcggtgatcaacttcgaggtctatgccgacccggacctgaacagcgccagccagctggcccagggcaaggtgtactggaacatccgcttcaccgatgtgccgcctgccgagaaccccaatttccgtgtcgaggtgaccgatcagtggctcaccgaagttctggatgtcgcctaaggagcgcccgtgatgattccgcaaaccctgaccaataccaatctgttcatcgacggcgtgagcttcgccggtgacgtgccatccctgaccctgcccaagctggctgtgaagaccgagcaataccgtgccggcggcatggatgcgccggtatccatcgacatgggcctggaggcgatggaagccaagttctccaccaacggtgcccgccgagaagcgctgaatttcttcggcctggccgaccagagcgctttcaatggcgtgttccgtggctccttcaagggccagaagggcgccagcgtgccagtggtggctaccctgcgcggcctgctcaaggaggtcgacccgggcgactggaaagccggcgagaaagccgagttcaagtacgccgttgcggtcagctactacaagctggaagtcgatggccgcgaggtctacgagatcgatccggtcaacggtgtccgcgcgatcaacggtgtcgaccagttggccggcatgcgcaacgacctcggcctgtaagaggagctccggacatgacccaagagaatcgactgccgggctggctgacgctggatgctgacgccgccctcgttcgtctctcgcgtccggcacagtgcaacggggtcagcgtcgacacgctgaccctgcgtgcacccaccgtgcgtgatatccgcctggccggcaaggtggccggcgacgatgccgaggagcgcgaactgcaactgttcgcctcgctggcgcaggtcagccgccaggacctggaggggctgaagctgagcgactaccagcggctgcaaggagcctactttcgcctggtgcaagacgacacggacgacaccttcgcgtatgcgtcaactggcgaggcgcctggccattgagctgggcttccaggccagcgaactggagcgcatgaccctgggcgacctgctctggtggctcgccgagggcgaggagtgagcgacggcgccgtccgccaggcgagcggcgcccgcgctacttcagtaggtccgcggaggcggacatcgataacagacgagacgaaccgtcatgagtaaagacatggacctggtggtctccatcggcggcatcgccgacccctccttgggcaaggcattcgagaccgtcaaggcacggctcgacagtctccaggaacgttcccgccaggcgtccagcctgagagatgtgctgggagacgccatacgcctggagcgagaactcgccgatatgcgcaaggtcggggaccgcggcgttgctgagcatgcccggcagcttggcgaacgccaggagcaactgaagcgactcggcatcgaggccagggtcgcgggcgatgcctacgctcgactgggcgagatgcagcgtggcctggatatgcaggtccgcggcctgcaacggctggagcaggccagccaggcaatgccattggctagcgcattttccggactggtcgtggaggccagcaagacggctgccggttatcaagcgcggttgcgcgacctggcgatccgcaacggcctggacgtcggccgggagccagccttggcatccctgatccaggacagtgccagccagagcggcctgggacgcacggcgacgctggacatgctggagcacttgaacgccaccggcatggggttcgccgccgcgcaaatgaatctgggactggcgggccgcttcggctttggccaagggattgcttcagccgaggttgcggggctggttcgagcgttgcaactggcccagggttcggactcgccagagcaattgtccgcctccctcgaccgcctggtcgtcctgggtaaaggcagagtcggcagtgaggccctggcgcgtcgcctgcctgccttgttgtcagcgctgggcaatgcgggagaggccacggccggcgatgtcggtgcactgggggccttgctggagattcaggcaaagaacaccacgccagacaaagcggacgtgcggatgaaggcctggctggagttcgtcggcagcggcagcttgaaacgcgcttatggccaggactacgaccgggacctggaagcgctgcgcaaggacggagcgagtctgctggaggccaacctggagctggctgcgcgctatcgggataagggcagcaagctcagcgccggcgtggcgtcgccggcgcttgaagcctatcgagcgtcgcgcggcgagttccagggcttgctcgaatcccagcagtcttccgtaggctcttcggagcgcgatgcgcagcgtcgcaaggggatgtcccaggagctgtggaaggcttccagcgacagttgggagagggcgcagaccgctttgggcagcgccctgaatccatatctggacaacctggccaaggggagcgcggtactcggcgagtcgactgcggaactgctcgaagcctatccgcggacgacggccggtcttaccgccgccgcaggtgcggtgttatccgggtatctcgcctacaagggagggcgcggcgctatcgacgtgttgcgtggcggtcggctcggtcggcgagggaccgctgccgtcggcgacctgatcgaacggggtgcaggccgggtatcgggtggtagcgaaatacagcgcgtgttcgttaccaactggccggtgccgggaggcgactctacgctggagtccgcgaggaggccagcacaacgaaagcgcggtcaaacaccacgcaggaagagaggcaagggcggtgggctgaaggcccgctcccttccatcccttggcttctcggctggaggaggcttgggggcgatggcaggaaagctgccgcgcctgtcgcgtttgcccatacgcaacgcgccgctgcaagtggcctcgtcgttgatcgatgttgctgaggtctactccagcgacctgtcggagagcgaaaagaccgttgcctatggcgaggcaggtggctccctggctggttccctggctggcgccgctctgggagcgagcatcggctcggtggtgccggtggtcggtacgctgatcggtggattggttggcggcgctatcggcgcctggggcggtagcgaactgggggggcgcctggggcgcagtctggctggcgatccgccggcggcctcggacaacaagccggcggtggccgtaccacaggccggacccgtcgcggctgcgcccaactggaccttcgcgccgcagatcaacctgacggtgcaaggcaacgtgcacgagccgcagcgcctggccgacgagttgctgccctacctgcaacgcatgcttgtcgacttcgccgacgagcggcagcggcgcagcctctacgacccggcgatggtttaaggagtccccatggcatatctggaacaattgcaggccggcctgaggtacctgggtcgcgccggcgagtccggacgcaagagtctggacaaggtggtcgctccggtgaacggcgcgatcagcgagatccgcggcgcagccgcggagctggagaacctgcccggcgtatcgccggaaatggctgcccggctgcagcgtgccatgcgcggcatcggccaggcgcagggcaaggtgaaccgcgtggtctccacctatgaccgggcgagccgggcgttgcttggtatcgacgaacgcctggatgcgctgaaggtgcaggtgaacagtgccgcgcaggcggtcggcaaggtcgctggcgacatcagtccgacgctggcgggggtgctgccgtcgtggctgctggcaccctcggcgacgcccccgagcgaggccgcagcgcccttgccgcacctgctggtactgcagccgctgaccgccaatgcccaaccgttctacttcaacctgaatactgccgccttcgacgccctgcagcgcaacagtgcctacaactggagcgggcaggtgcgcctgggtcggcggccggcgctgcagagcgtcggcatgggcgaggagagcatcctgctcaagggtgcggtattcccgctgcgtcgacaggtaggtaaccaggaaaaggtcgtcggtctggagcagctcgaagcgctgcgccggttggcggagcggcgtgagccgctgatcctgagcagcggctacggcgaggtgcagatgggcctctggtgtctggtgcggatcagcgagaaccagagcgccctgctgggcaacggcgctccccgcaaacaaaccttcgacctggagttcaagcgctatggcgacgacctgccgaaccgctgacggcgacatgctggacagcctctgctaccacgtctatggccatctgttgggctgcgttgaggcgaccctcgacgccaatcccgggctggccgatgagcagcagccattccgcgccggcttgctgatcagtttccctgacatgccggtggtcaatgtcgaacaggtgcgcctgtgggattgatcgaccgctcacccgcaaccccgccttggcggggtttttcttttctggagagatcaggtgcaaccgagtttccgtatcgttgccgacggcaccgacgtcacccagcggctgaatgaccgcctgctcaagctgaccctgctggacaagccgggcatggagtccgacagcctgaccttgaggctcgacgatcgcgatggacaggtggccttacccaggcgcggtgcggtgctggaggttcatctcggctatgccggcgagccactgatgcgcatgggacgctttaccgtggacaccttgcagtgggctggtccgccggaccgcctgaccgttactgccaaggccggcgacatgcgcggcagtggcaagacgatacgcagcggcggttgggagggcactaccctggctcaggtctgccgcgatgttggcgcacgcaacggctggcgcgtggagtgtccgttgcaggtggcgatcgcccgggtcgaccaggtcaacgagtccgactaccacttcgtcacccgtctggcgcgccagtacgactgcaccgccaagctggccgagggcatgctcatggtgctgccgcgacagagcgggcagagcgccacggggcgtcggatcgaacctttggtgttgggacgtgctgatgtcggcagcttcgacgttaccttcgacgaccgcagcctgatgagaacggtgaagacccgctaccaattgcccggcagcggcgaggtcaagagcgtcgagttgaagaacccgaaggcaccggctacggctacgggcgagcatgtcgaccggcatctctatgccagccgtggagaggccgagcaggcggcgaaggctcgcctggcgagcttcagccgctccagtgccagtgtgcgcctggagctgccagggcgtggcgacctgttcgccgagcgcagcctgctgctccaaggcttcaaggcgggaatcgacggcgagttcctgatcgactcggtggagcacacctacagctccagcggatggaccactgtcgtgcaatgcaacggcggccgaggcggcaaggggtgacgcctgagacccatcggagtgcaggaggatcgatgaaactgaccgagcagcaattgctgcgcatttttcccaacgcccgcctcgtcgcgggcgttttcgttgcggcgttgcaacgggccatggacgagagggagatcgacacaccggcgcggcgtgccgcgtttctcgcccaggtcggccacgaaagcagccagttgacccggctggtggagaacctcaactacagtgcccaaggcttggcggcgacctggccgagccgctatctcggccccgacgggcagcccaacgcgttggccttgcggctggcgcgcaatccgcaggcgattgccgacaacacctacgccacgcgcaacggcaatggcgacgaagcgtccggcgatggctggcgcttccgtgggcgtggcttgctacagatcaccgggcgtgccaactaccggttggtcggcgaggccctcggcgagccgctggaagccgagccctggcgcctggagcaacccgtgccggcggcccgcagcgccgcctggtggtgggccggtcacgggctcaacgagctggccgaccgcggcgagttcgctgccatcacccgccgcatcaacggcggcctgaatggccaggcggagcgcctggcgttgtggcagcgggccagggcggtgctgtcatgagccggctcgctctgcttctgctggccgtgttgctggtcctgctggccggcgccttgctcggcggcggcctggttgcccgccactatcgtccgcaactggaggaggccctgggccaactcactgccagccgcgtcgccagcggccagctcgaggctttgctcgatgagcagcagcgagcgctggcggcggtgcgggcgagcgccgagaggcgcgcgaaggacgccgagcaggcactcggcgaggccagggcgcaggccgcggagcagtatgccgcggccgtgcgtctgctccaggaacccgacattggtgtggactgccaggcggcaggtgcggcgatcgaccgggagctgggactatgactcgtctcctgctggggctttgtctgcttttcgcgggctgcgcagcctcgccgacgacacctcgccccgtgcgcgtcgaggttcccctcgcagtgccctgccgtgtacctgacgtgcgcccgccgagctgggccggcgccacgctgaaggccggcgattcgctgcaggccaaggttcgcgcattgctcgccgagcgccgccagcggcagggctacgaactcgaattgcaggcggcgttgcgtgcctgccgctgagacactgggcgcatatggacgtcgatggacgtgtgtccggcgcatattgccgtgtattgattccgcagcgttgtcgagccgggaatcggccggtacaacgtagtcatgcttgtacaggtgtgtccccccagggatgtcacctgcaacctcagagcccggcacgtgtgccgggctttttcgtttgcatccgacaacggctcgggacgtggaggctcctcgccgaccgcgtaccgcgccacggctgaccgtcctggggacggggccagacgacccgcctggtgcgggtcttttcatgtgcataaaggagagttttccatgtccatcctgactcaaggtacccagatctacgccctggttccgccggtatccggtaccggtgccgctaccgtcctggagatcgaaggcgtgacctcgttcaacccgggcggcaatccggccgaccagatcgaagacccgtgcctgagcgacacttcgcgcaagtacaagaaaggcctgcgcacccctggccaggcgaccctcggcatcaacgccgatccacgcctggccagtcacgttcggttgttccagctgtccgagaaagatggcgagacgagcgtcaagtgggccatcggctggtccgacggaatcgacgtaaagccgaccgtcagcaccgaaggcgacgatttcgtgttgccgccggcgcgcacctggttcaccttcgaaggctacgtcagcgacttccccttcgatttcgccagcaacacgctggtcgctacccaggccaccatccagcgttccggcgccggcaagtggacgccgaagtcggcttgaggagcgcgtagatgaatctcaacgaacttcgtgcggcgggcggtttcatcgagtcggcgctggtgcgcaaggagattagctggacccgcgttcccgccggcaggaagaaggcggtcagcgacaccttccaggtgttcgtccgacgcaacagttttggcgcggtggagcgcctgttctccgccgaaggcgaccagcagagccgcaacgcgcgctacctcgccgaatgcattcgcctgggcgagacgggagaggaaagcctgacctacgaacaggcctacgacctcgacccggcgctgggcttcctgctgctgcaggcggtcggtgaggtcaaccgggtcgaggacgcggaaaaaaactgacccccgccgacgaggtttggcacgagctggtgctgaacggcgtcggcgggtcgaccattgccgaggccaaggagcggctcagctacgccgagtaccgcgcctgggtcgcctacctgaacaagcgcggctcgctccatccgggacaccggctggagcttgcgctggctcggatcgctgcgttgctcgggcatgcgctgggagcggacgccgatcccgacgcgttccgtccgcatatggctctccagcccctttcgctgcaccaggcgatggatcaatgggcatgacaggccaccccgctgcggcggggtctttttctggaagacatgaatcatggccacgaataccgacggcagcctgacgctcgacctggtccttcgcagcgaagggtacagggccgggatggacaaggttggcaggatcaacgatcagaaaatgcacgccatggaggcgcgcgcggaaaaggctggcaaggttatcggcaaaagcctggacagttcggcgctgattgccagcagcgtgctggaccaggcgctggacatgctgggcaggaccagtcgccaggcgggtcaggccaagaagcctgtgcagagcgcccaggacaaggtactggccgagtggaagacccggcagaaggaactgggcgaagcctggaagagctatcgcgaaccactccaggatctgtccaagctcaacgaagcactactgaagaactcttccgacaagctcgacaaggcgctgctcaatctcagcgagaccggcaagctgtcgcttgccaacgtgggcaaggccgcctacgccgatgccgcgcgcctcgcctcgcggcagatgacgctgatgctgctggacgggctgtttggctgggtcgccagcgtcggtaccgagaagcccaaggtcgacgacaaggcgggcaagggacaggcgaaggccggcgacgacgagaaggaacagccgtcgctccagtcgcaggtcttcaagcagtggctgttgcagatgaacagtgtctggggcgcctaccgcgcgccgctgcaggatatctccgggatgaccgacgagctgttcaggaatgcgtcggagaagctcgagaagtcgctgttcaatttcgccactaccggaaagctgtccttgagcaacttcgccaagacggtgatcgacgatgtcgcccggatcgccgcgcggcagctttcaatgctcgccctggacggattgttcggctggatgaatggcaaggccggcatcaccgaggcgcaactggccagccagaagccctatacctcgctactggaaaaggcccgagcagatgcgggacaagcggctgcgggcgctcccgcggcccagggtgccgcgccaatgccggcagcggcgatggatgtcggcgcgatggtggccactgcttccgggcagaccggggacggttccaaggtatcggctggaggggcttcggcgagcgctggcaagccggtgggcagttgggtcgaacagatggacgcctcctgggcgagcctgcgcgaccaggcgcaggacgtctcgggaatgatggacatgctgtttaccaacgccttcaccaatatggagaacgccctgttcacctttgccaccacgggcaagctgtcgttcaaggatttcgccgactcggtgatccaggatatggcgcggatcgccgcgcggcaggcgacgctgcagatcatcggcggcatcgtcggtgcggtcagcgggttcttcggtagcggcgcaacggcgggctcgcggatttccgactataccggctcggacatggccaattgggtcagcaagcaacgcgccggaggcatgcctgggttcgccaggggcggtgccttcaacgatggcatccagagcgcgccggcgctgttcagcatggccggcggtcgtccggcgctgatcggcgagcgtgggccggaagccatcatgccgctgagtcgcggttccgatggcgtgctcggcgtgcgcgcgctcggcggcggcgaggggggcaacgtcttcaatttctccaccagcgtcagcctgggcggcggccgcgagggcgcggcgacggccagcggcgacgacggtacgggacagcagctggcgggaatgatcaacgatgccgcgcgcaacgtggtggcgcaggagctgcgccccggcggcctggtatggaggatggtgaatggctgatctggaacgctttacctgggacatctcgatcgattccgccggccaggcgaaccaactggtacgccaggtgcagtacggcggcggctacagccaggcgctcggcgacgggctgaacaacctcagcgagacctggcaggtttcgcgtaccggcgatctcgcgctgatcggcccgatccgcgatttcctcaagcgccacggcggctaccgctcgttcctctggaccttgcccactggcgaaccggtacgggtgcgcgcccagggctggcaattgcggccgcgcggcaacggcgtgttcaccctgaacaccaccttccagcaagtcttcaatccgtgaggtaaacatgaccatcacagccgatgaccaggccctcgagcctggggcgctggtgcgcctgttcgacctggattgcaccgggttcggcggcgagatgctgcgcttccacggccacctgcagcaggggccgatccactggcagggcaacgcctaccaagcttggccgctggaggcgcgcggcttcgagcagcgcggcgacggccgggccagttcgccgacccttagcgtgggcaacatcgacggcagcatcagcgcgctctgcctgttcttcgatggcctggtaggcgcgcgcctgaccgtgcgcgagacctatgcgcactacctggatgcggccaacttcgccgaaggcaacccgcaggccgacccctcccaggagcgcctgaacatctggttcctcgagcagaagaccgccgagaacagcgtccaggtgacctgggagctgtccgctccgccggatttccagggccagcagatcccggcgcgccagatcacctcgctgtgccactggtgcatcaccaacgagtaccgcgggcaggactgcaactacaccggcacggcgatgttcgatgccgacggcaatccggtggacgatccggcgctggaccgctgcggcggccgggtcagcgattgcaagctgcgcttcggcgcggacaacccgctgtcccacggcggcttcgccggcgccggcctggtcaggatgtgagcatggaactgagccgcagcctgcagcgggccatcgccgcacacgccgcccgcgagcatccgcgcgaatgctgcgggctgatcgttcgcggtgcgcgtcaacgccgctacgtggcctgtcgcaacgcagccggatcgcccagcgagcacttcgtgatcgatcaccaggactggtgcgccgccgaggaccagggcgaggtcctggccatcgtccacagccacccggacgttccggccacgccgagcatggccgatcgggtcagttgcgaactgcatggtctgccctgggtgatcctgtcctggccggaaggcgatgtcgcgcatctagcgccggagggctatcgggcgccgctgctcggccgcgagttcgcccacggcgtgctcgactgctggagcctctgtcgcgactggtaccgccgcgaggcaggtttggagcttccggactatccgcgccgcgacggttggtgggaaaccggcgagagcctctacgagcagcactatgcggcggccggattccggccggtgccgctggccggaatccgccgcggcgacatgctggtgatgcaggtcgggagggcgctgcacccgaaccacgcgggcatctacctgggcaatgactggcgtctggacagcgagccggtccaggcgcttggcggcgacggaccgttcctgctgcaccacctgtacggacggctgtcgacccgcgacgtgttcggcggaccctggatcgaacgcacgcgcctggtcttgcggcacacgcggatgccgcagtgaacgacatattcaagcgagccgtcggaatcggctcttcacgagaggaacaggtccatgagtgacaccctgagtcagggcctcaccaccatccgtctgtacggggttctgggcaagcgcttcggccgcatgcacggccggttgttggaaagcggcacggtacgcgaggcgatgagtgccctgaagcacaccatggagggattcgagacgttcatgcgcgaggcggaatcgaaagggctgaccttcgccgtgttccgtgggcgtaccaacctgtccggcgagcaactggacatgcgcggacgcgaggatatccgcatcgtgccgttggtgatcggaagcaagcagtccggattgtttcagacggtattaggagctgcactcattgccgtaggtgtcttcgcaacgagtcttacgctcgggacaagcacgtttctgatctctgccggcgccagcatgatgctcggcggcgtcatgcagatgctcagcccccaacccaagggcctgaagggccgagaggcccccgagaacgcccccagctatgccttcggcggcccggtcaacaccatcgcccagggccatccggtcggcgtgctctacggcaagcgccgcatcggcggcgcggtgatcagcgccggcatctatgccgaggaccggctgtagccggcaacgccgtaacaggcccgccatgcgcgggcgtttttttgcctgaaggaacgtcatgaacaagaccatcacgggccacaagggtggcagcaagaagccgcgccagccggtggagatgccggactcggtgcgctcgatcgcgcgggcgaagattctcctggcactgggcgaaggcgagttcgacggtggcgtcgacggccgttcgatctacctggacgatacgccgctgctggcggcggacggctcggtgaacttccctggagtgacctgggagttccgtccgggctcggtggaccaggaacacattgccggtgtgcccgccgtggaaaacgaactggcggtcggcgtcgagctcaagagtgacgcgccctgggtccgcgcggtgagcaacacccagctctcggcggtgcgcctgcgcctgtcctggccggccatccagcgccagcaggaaaacggtgacgtggtcggctaccacatcgactacgcgatcgacatcgccgtcgacggcggtgcctggcaggaagcgctgaaggcttcgctggacgacaagtccaccagccgctacgagcgctcccaccgtgtcgacctgccggaggcgcggagcggctggcaggtgcgcgtgcgccgcctgacgccgaaccagaacaacaaccgcatcgccgacaccatgcgggtcgaggcgatcaccgaggtgatcgacgccaagctgcgctacccgaacaccgcgctgctgttcgtcgagttcgatgccagccagttccagagcattccgcagatatcggtggaagcgcgcggccggcgggtgcgggtgccgagcaactacgatccgcagacccgtagctacagcggcacctgggacggctcgttcaagtcggcctggaccagcaacccggcctggcactggtacgacatcgtgttgcacaagcgcttcggcctcggtcggcggatcgacgcgagcatggtcgacaagtggtcgctgtaccgcatcgcccagtactgcgaccagtcggtgcccgacggcaagggcggccaggagccgcgcttcagctgcaacctgtacctgcagagtcgcgccgaagcctggaccgtgctgcgcgacctggcagcgatcttccgcggcatgtcctactggtccggcgcggaaatggtggcggtatccgacatgccggaggacgaggcctacaccttctcaccgtcgaacaccgtgcgtggcgacgacggcagccacttcaactacagcagcagccgccagcgcgatcgccacaccctggccctggtcaactacgacaatccgggcaacggttaccagagccaaccggtagcggtgaacaatgaccgcgcgcagcgccgctacggcatcagccagttggagatcaccgcgatcggttgcacctccgagggcgaggcgcagcggcgtggccagtgggcgctgctgaccgaggagctggagcaggacgcggtgaccttccgcaccggcatggatggccgtgggctggcgccggggaagatcatcgccgtagccgacccggtcaagtccggcaagcagatcggcggacgcctgagcgcggtggatggccgcgcgctgaccctcgaccgcgacgtcgaggcccgacccggcgatcgcctgctggtcaacctgccgaacggcaaggccgaggcgcgcagcgtccagtcggtggtaggccgcgtgctgagcgtgaccgccgcctattcggagacgcctcggccccaggggcagtgggcgctgcagagcaacagcctgaccacccagcgcttccgcatcatgagcatcacccggccggaggacaatcttttcgagatcaccgcgctgcaacacaacgcgagcaagttcgacgccatcgacaacggtgcgcgcatcgagctgccgccggtcaccagcattccgccgggcgtgcaggcgccgccgcagaacgtgcggatcaaggctttcaccaaggtcgaccaggggttggcggtgaccagcctgtcggcctcctgggatgccgcgccgaacgcggtggcctacgaggccgaatggcgcaaggactcgggcaactgggtgcgggtgccgcgaacctcggcgctcggtttcgacgtgccgggcatctatgccggtcgctacctggtgcgggtacgcgccttgaacgtgatggaggtcggttcggtctacgccagcagtgtggaaaccgctctcgagggcaagaccacaccgccgccggcgctggcctacctgcgctgcgtggccggcccctggcgcatcggcctggagtgggggtttccggccagcggcgcggcggacaccgcctacaccgagatccagcagtccgccacgcccggcggcagcgaggagaccgcacgggcgctgggcctgttcgcctacccaggcaatacccacctggtatcgccgataccggccggcgaacggctggcgttccgcggtcgcttgatcgaccgtagcggcaacgtcggcgcctggtcgaactgggtcaccggcaccagctccagcgacgccagcgaatacaaccagttgatcacccaggagtacgtcgagtcggcgctgggccagcaatttttctccgatatcgagcggatgcaggtggatatcgggggcttgcagaagcaggtcggcgacctcgccgacattctgctgtacgacccggccaagatctacgcgaagaacgacatggtgcgacaggggccgcggttgtaccaggcactgaaggctgtgccggcgaagacggcgccgccgaacgcggcctactggtccgatatcggccagtcgctggagaccgccaacgggctggcgcagcaggtggcgagccataccgctgaaatcagcgaactcgacggcaggatcgaagcagcggtatcgaatctggatgtgctgcaagctgccgcccgcggggagccggcgaccggagagaaggcggatgcgctgaagggctgggacaccattgctcgagccgccaccgaagtcaccgtgcgggcgaacgaggacgaagcgcaggcgaagcgggcgagcttgcttgaagcgcggaccgggaccgcggagggcaggatcgccaccgtcgagtcggtcgttgcgtcgaacaatgccgtaaccgtccagcgattggatcagctcaccggccaggttgcgagcaacgcctcggccatcagcaccgaacagaccgtccgcgccaacgcggacagcgcactggggcagcgggtggataccgtcagcgcgcgcaccgataccaacgaggcgaacatccagaccacatctcaagcggttacctcgctggatggcaacgtcaaggcgctctacagcgtgaagctccaggcgcatgccaacgggcagaagtacgccgctggctggcaactgggcttcgacagcggtacgagcgtgacgaccatggcgttccaggctgatcggttcctctggttcaacagttccagcgggcagaccgtggcgccggtctcgatcgtcggcggccagatgttcatcaacaacgcgatgattcaggatgggtcaatcaccaatgcgaagatcggcaacgtgatccagtcgaccgccctcggtgccaacggcgagccgctgtggaagttggataagggcggcgcgttcacaatgaacagcgccacgtctggcggcttcatgaggcagacagcggaggccaccaaggtctacgacgcgaatcttgtgctgcgggtacagatcgggaatctagacgtatgagctacggaatccgcctgagaaatgcggccggctccatcctgatggagctcaccggccaatcggcgcgcacggtctaccggcagtcgctcggcgccatcaccaacgggatgacggtgacggtgccgggttttgatcctgcgcgcggtgttgtgttcatcatcgcgagcggaaacgaattcggtgaagtgccccgatacacaatttccggaagcgtggtgacgttccactggaacggttcatccggaacaacttatgtactgcatgcggtgatgttctcatgagctacggagtattaattcgcggggatgctgggcaaacaataatcgacgatagtaatccgtgcattcatttcgctgcgtcgggaacttatggacatacgaccggcagagaaactgttattcaatatgcctttccaatacagtccccgtatgagccgtatgtcttcgtgcgcccaaatggtccgcatcaaatctatttgttcaggcatatcggcgccccggggaactggactggatttgcattctggcagacgatctatcgggacgtggaccctccaatctacggcggaaagtggaaagctggcgcggtcatgttgccgaaaaccggtgggtggggaatgcaggttttcgactcccagtcgcgtgtgatgttcgacagtaaccgggacatcgttcgctatgtcggtggcgcacaggtttggaataagtattcgtacaacccgagctggccaggcgggatggcactacaaacgtggtatctgccgttcacatatggagttgaggcctacttccaagtcagccatttcaatgtcaaagcattcataacgttagaagcgccgcgcataggttttcttgagaactcaatgagcttgatatttgtttcatcagttgtagagtttgaaactaaccatcagttcaattggccgcttattgtagtggcgtaaatatatctggaggactatatggcttggtattccacaggcacggttgctgtcacgctgaattcgccgacagtcaccggcactgggaccgcattttccgccaacgcccgggtcggcgatgcatttcgcggacccgatgggcgttggtacgaggtcacaaacgtcgccagttcgacggtgatctcgatcaaacccaactaccagggcagcacggccagcggccagccctatgcagtggcgccgatcctgggctacgacaaggacctatcagatcgtttcaaccagatcgcgatggactggggggcgacccttgcgggcataaagccgtgggccctgtccaataccggcacgcaagcgcaggcggacatgggaatgacggcggtggggcggggactcaacgccgcagcgactgctgagaatgccctgagctttattggtggcatgccgaagtcgatgtccaatctgcgggctgtcagcgacgccaataatgtcccgaacgaatgtgggttctacggtatcggagcgtcgccttgggcgaacttgccgccgggggtcgacggtatcaaccctatcggatccatgctctaccaccatccgtacgatgtgagcaccgccgtgcagatgctcattccgcgaacctctgacctcatgtacttccgccggaagctctccggcaactggagcgcatgggtgcggctactctcggataagcagcttgtaggtacggtatctgttgacgggtcgaatgttccgaacggtgcggtcatgcagcagaacgggaccacggctatcaacgtgggcaccagtctgcgtttcgccgacggcactcagattatctacgcgaagctccgtctggaattcagcgcggtagacatcttgacccgccagtacacgttccccatgagctttttcgaacccccgaatgtcaccgctactttaattcaaggtcagcagtcggatatcaatccattgcagttccagcaactcggtccggtattggtcgctgctactactgttagcgcgtgcaacgtacgagtcatgcgccctacttacgtatcgagtggctgggcttctgggaacttcatcgactgttctgtcaacgcggtagggagatggcgctaatgaagttcttgctaaaacccgacctgcaagtcggattgcctggccaagaacgggtaagctcggtttctgtaaacggattgcggttgactatcgacggcgtagagttcgatttttctccacttgcagtgggtgggtacttgcctccggaggcatacatcaacataacccccctgcaagaggtggaggttcgaagcgactttcttctcgtacgctacatccaccaagtgacagcagacattctgactgcttatcgcgccgagattgaaccaattttgatggaagttgacggacctgtggagctaccgaaatgaacattgactggacccaactgagaacccccgagcagcaggccgccgaacgcttgcaggctgagtacgatgccgcagccgcggcgcgggcaaatgcctaccgcctggagagtgacccgctcaagaccgaggctgaattcgatgcgatcaaggccggcaccgagccggactactctgactggatcgccaaggtagaagagatcaaggggcgatatccacttccttga

SEQ ID NO: 2 - DNA, 2106 bp, Pseudomonas aeruginosa
  misc_feature 1..2106: note = Description of sequence: polynucleotide, name: PAKAF R1 pyocin tail
  source 1..2106: mol_type = genomic DNA
atgacgaccaatactccgaaatacggtggcctgctcaccgacataggtgccgctgcgctggctgcggccagtgcagcaggcaagaaatggcagccgactcatatgctgatcggcgatgccggcggtgcgccgggcgacacgccggatccattgccttctgcggcgcagaagagcctgatcaaccaacgccatcgggctcagctgaatcggctgttcgtttccgacaagaacgccaataccttggttgccgaggtggtgctgccagttgaggtaggtggcttctggatccgcgagatcggcctgcaggatgccgacggcaagttcgtcgcggtatccaactgcccgcccagctacaaggctgcaatggaaagtggcagtgcgcggacccagaccattcgggtgaacatcgcgctctccggcctggagaatgtccagctgctgatcgacaacggcatcatctacgccactcaggactgggtgaaggaaaaggtcgctgccgatttcaagggccgcaagattctggctggcaatggcttggtcggtgggggcgatctttctgccgaccgcagcattggtctggcgccttccggcgtgacggcgggcagctatcgttcggtcacggtgaacgccaacggggtggtcacccagggcagcaatccgaccaccctggccggctatgcgatcggagatgcctataccaaggccgataccgatggaaaactggcgcagaaagcgaacaaggccaccaccctggccggctatggcattaccgatgcgctgcgagtcgatggcaacgccgtgtcatccagcaggctggccgcaccgcgtagcctggcagccagtggcgatgcctcctggtcggtgaccttcgacggcagtgccaatgtttctgcgccgctgagtctttccgctaccggtgtggcggcgggcagctatccgaaggtgaccgtggatacgaagggaagggtgactgctggaatggcgctggcggcgacggacattcccgggctggatgcttcgaagttggtcagcggggtgctggccgagcagcgtttgccggtattcgcgcgcgggttggctactgctgtctcgaacagtagcgatccgaacaccgcgaccgtgccgttgatgctgaccaatcatgcgaacggacctgttgccggacgatacttctacatccagtcgatgttctatccggatcagaacggcaatgcttcgcagattgcaacgagctacaacgctacatccgagatgtatgtacgggtgtcctacgcggccaaccctagcatccgggagtggttgccctggcagcgttgcgacattggaggttccttcacgaagacgactgacggatccattggaaatggcgtcaatataaacagcttcgtcaattccggatggtggttgcaatcgacatcggaatgggcggcgggtggagctaactatcccgtggggctggccggtttgctgattgtctaccgcgcacatgcagaccatatctatcagacctacgtaacactcaacggaagcacatattcgcgctgctgctatgcgggctcttggcgtccgtggcggcagaactgggacgatggaaacttcgatccggccagctacctgccaaaggcgggatttacctgggcggctttgccgggtaagccggcaactttcccgccctcagggcataaccacgataccagccagatcacctccggcatcttgcctctcgctcgtggtggccttggcgccaatacagcggccggagcacgcaacaacattggtgccggagtgccggccacggcgagccgggcgctcaatggttggtggaaggacaacgataccggtctgatcgtccagtggatgcaggtgaacgtaggagatcatcccggtggaataatcgatcgtaccttgacgttcccgatcgcgttccctagcgcctgtctgcatgtcgtaccgactgtcaaggaggtggggcgaccagcgacgtccgcgtcgaccgttacggtcgccgatgtcagcgtcagcaacacgggatgtgtgatcgtttcctccgagtactacggactggctcaaaactatggcatcagagtgatggccatcggctattga

SEQ ID NO: 3 - protein, 701 aa, Pseudomonas aeruginosa
  REGION 1..701: note = Description of sequence: peptide, name: PAKAF R1 pyocin tail fiber
MTTNTPKYGGLLTDIGAAALAAASAAGKKWQPTHMLIGDAGGAPGDTPDPLPSAAQKSLINQRHRAQLNRLFVSDKNANTLVAEVVLPVEVGGFWIREIGLQDADGKFVAVSNCPPSYKAAMESGSARTQTIRVNIALSGLENVQLLIDNGIIYATQDWVKEKVAADFKGRKILAGNGLVGGGDLSADRSIGLAPSGVTAGSYRSVTVNANGVVTQGSNPTTLAGYAIGDAYTKADTDGKLAQKANKATTLAGYGITDALRVDGNAVSSSRLAAPRSLAASGDASWSVTFDGSANVSAPLSLSATGVAAGSYPKVTVDTKGRVTAGMALAATDIPGLDASKLVSGVLAEQRLPVFARGLATAVSNSSDPNTATVPLMLTNHANGPVAGRYFYIQSMFYPDQNGNASQIATSYNATSEMYVRVSYAANPSIREWLPWQRCDIGGSFTKTTDGSIGNGVNINSFVNSGWWLQSTSEWAAGGANYPVGLAGLLIVYRAHADHIYQTYVTLNGSTYSRCCYAGSWRPWRQNWDDGNFDPASYLPKAGFTWAALPGKPATFPPSGHNHDTSQITSGILPLARGGLGANTAAGARNNIGAGVPATASRALNGWWKDNDTGLIVQWMQVNVGDHPGGIIDRTLTFPIAFPSACLHVVPTVKEVGRPATSASTVTVADVSVSNTGCVIVSSEYYGLAQNYGIRVMAIGY

SEQ ID NO: 4 - DNA, 16761 bp, Pseudomonas aeruginosa
  misc_feature 1..16761: note = Description of sequence: polynucleotide, name: PAO1 R2 Pyocin
  source 1..16761: mol_type = genomic DNA
tcaggatgcgatgctgtcagcgcccggctgtgctcgacgggcgcttccggcttggttcgcgcgctggtcgacgtaggcggcgaggtgatcgagggccacgaactgcatggccttgtgactatcgtcgagggttgtcaccggcagggcgatccgcccgcatgccagggccttgctgaagttttccttgttcaggttgcgaaaccagcgttcgcgaacctgctccagcggcaccaggacgtcgccgaagatgcggtagaccaattccacggtttcctggcgtggaatcggagtgctaggggcgatggatggctgcatggtattccctcctgcggctacacgtcgttgagggaaatatagctcaggttgttttcttgttcaatagctgaagttgtagagcgggcgagcgccaggcgctcaccgcaccagggacgggccgcgccggtggcgtacggtggaccaccagaagacccaaccgatcatgctgatctggcggctgcgcatgtcctccggcgagtactcctcgtccggatattcctcgcggttgaagctgcgcaggcgaatgccgccgccgggcaggcgatagacgaacttcacccgcagcatgccgtcatgttcgagggcgtagatctcgccatcggtgatatgggtggtggcggtgtccacgccgatggtggagccatccatgatcagcggttccatgctgttgccggtgagttgggcgcagatcgccgccgacggatcgacgcccgaggcacgcagcgtggcgtaggaaaaacgcagcttgcgcccctctatctcgcgcaccgcagtgcgtccggcgccggcggacatctccacttccttgtacagcggcagttccacttcgtcttcgtccagcggtgtgtcgctgtcccacggatgcagcggttccagcaccaatgggctgccatcggccacggacacggcactgcgcgcggctggagcgccttcgccggtctgcagccagaccggcgagacgcccagggccgccgcgatctcgatcagcttgcgggtgctctgggccttgcccgaggtcagcttgtgaatggtgttctgcgaaacccctgccgcttcggcgagggtttcctgcttcaggttgcgcatcgccatggcctgcttgaggcgagcggcgaagctgtcgggcgggatctgggtgctcttgtccatgctcggcaatctacagaccgatggattttctgtaaagagcctaggtgttgacgataaatagctttggttgtaatttctcttccgtcagaaagcggaaggggtgagttccacaacttcccacccgcctttcagagatttcacatgggtgcagggacgcacccggttgtcggcgaccgacgatgcaggtgccgactgcttgggtgccggggccatgggaaagcgctccggcacctggagggccggagcgccgtccatgcagacgctccgtcccgctcattcattctgtcgccgacctgcttcaggtacggcggcgtcgtacagggagtcattgccatgtcgcgagatacgcactcgccatttccgcgcagaggaacgcaggtcgccgaaccggcgccgcgcatctgtcatgttccatccattcggccgtcgggcgtcgggcacgccagggaggctttccatggctgaccttgccgatcacgccaacgaactggtcctggctcgcctcgacggcctcctggcggcgcgcccggcgctggccatccgcgagtccgcggaagactgcgaggactgcggcgagcccattccccaggcgcgccgccgggcggcaccgggctgcagtcgctgcatcgactgccaggaccgccacgagcgccgttgaaccgacctcatgccgagccctcgcgggcagtgaaaggagacacgaccgtgatcaaagccatcgatgaaatgctcaagctctgggccgaggaaatgcacgcgccaggcagcaacggaggcggctacgccggcggcaacctgatcgccatgctgatcgccagcaagggcgcggtggtccgcggccaccgtggcagccgggtgatcctcgaccgtgtggcggaggtcgatcgcctggtaaatcgcctacccgaggaactgaagaacgtggtggtggagcactatctcaatcgcgacagcttccccgagcagaagtaccgccactgcggttgcagccgcaacaccttttatctgcgcctgcatgtggcgcaccagggtatccaggacggcctgctgcggcgggtggcctgagttcctggaccggataccgtgtcccccctggaattttccaaccggccttcgagccggtttttttatgcctgtcgcccatggaggcggtgagtcccatgcacagcagcggaccctgcacgctagaggcgggttcgggctggcggagccgtccttttcgtcagtcgatgccgggcttttccatatcggaagtgggtacgcgcttgcccggatgcctggcgaaggttcccagggcggtatatcaccctcatatcgtggtgcgtctccctcctattgcagtggattgtcgagaaggcattgccgggctgggaatcgggcggtaaaaagtagtcattcttgtaaaggtgcgtccccagggaggcactcgtgaagcaccggaacccggccctggcgccgggttttttattgcccgggaaaacgttcgtgcggacgtgcccggcgctgcctgccaaggagggacccatgggcaacgaaccgcagacactgacggaaatgccgctctgggtactgatcctgctcgccgcgctgggcggcgtcagcggcgagatgtggcgtgccgacaaggccggtctcggcggctgggccttgttgcggcgcctggcgctgcgctccggcgcgtcgattgtctgcggcgtggcggtgatgctgctggcattggcttgcggcgccgcgctgctgttcgccgcggcgctgggcagcctgaccgccgcggccggcgcggagatcgcagtcggtctctacgaacgctgggccgcccggcgcctgggggtttgcgagctgcccgaggaacagtcggacgaccgtggcccgcattgaaactgatcgaaggagtcaaccatgcctgaacaggctgttacgctcgaggctctgtacgcggccatcgagcaggtactgcgtgagcgtctgccggaggcgcagttgatcggcttctggccaggcgtgccggaaaataccccggcggtttccctggaaatagccgagctactgcccgagcgcgatcccggtaccggcgagagtgccctgctgtgccgcctgcaggcgcggataatggtgccgcctggtgccgatcgccaggcggtatccattgcttgcggaatcgttcggacattgcgcgagcagacctggaacctgtctctgcagccggcgcgcttcgtacgctcggccgtcgacggcagtcgcgaggagctgaagagcctgcgtgtctggctggtcgagtggacgcagtcgctgcgtctcggagacccggagtgggcctgggaggaccagccgcccggcagcctgatgctgggcttcgacccgcagaccggccccggccatgagccggactacttcgctccggaggcgttggcatgagctatgtcagtgcggagcatgaccgcatgctcgccgcgatgatcctgccctgcgtggtggtcgccgtggacctggcagcggcgcgggtacgggtgcgctccggcgactggaccagcggctggctgcgctggcactccctggcagccggcaaggttcgccactggcgtgcgccgagcataggcgaacagggggtactgctcagcccgtcgggcggagtgtcaatgggcacctttattcccggtctgtacggcgatgcgggcacggcgccggacaacagcgccagcagtgagacctggcgtttcgacgacggcgcctcgttgagttacgactgggctgcgcatcgctaccgcgtcgagctgcccagcggcaccgtggaagtgagggtcggcgccagcgaggtgcgggtcagcgacggggcggtcagtctcaaggcgccgaagatcagcctggaaggaccggtggagatcgccgggacactgacggtcagcggagacatcctcggcggcggctcgatcatcgacaccgccggcaacagcaaccaccacacccattgaacatacgacggggctgccgaagggcggtccgtcgaacattcaaccggcccgcgcgagcgggccttttcgtttgcggagttcgccatgggcaacactcacagccattcggagccaggcgcggcctgtctctgccggagcgggagggcagggcgatgatcgggatggatcgccgtagcgggctacccctgtccggcctggctcatcttaaacagtccgtcgaggacatcctgaccaccccgttgggcagcaggcgcatgcgccccgagtacggcagcaagctgcggcggatggtcgacatgccggtgagcgaaggctggaaaagcgccgtgcaggccgaggtagcccgttccctggggcgctgggaaccgcgcatcggattgtctgccgtgcgagtcgtcgcggtcgtcgatggccgcgtggatctgctcctgagcggcgtgttcgagggcgagaacatcaatatggaggtctcggcgtgatcatcgatctttcccagttgccggagccagaggttatcgaaaacctcgatttcgagacgatttaccaagagctgttgggcgacttccgcgaagccatggctggcgaatggacagcggaggtggagtccgatccggttctcaagcttctgcaactggcggcctatcgagaactgctgctgcgggcgcggatcaacgatgcggcgcgggcggtgatgctggcatacgccagcggtgccgatctcgaccagatcggtgccggcttcaatgtgcagcgtttgctgatcaggcccgctcagcccgaggcggtaccgccggtggaggcgcaatacgagagcgacaagtcgctgcgcaatcgcatccagctcgcgttcgagcagctgtccgtcgcaggaccgcggaacgcctatatagcccatgcgctgggcgcggatggaagggtggcggatgcctctgcgaccagtccggcgccctgcgaagtgctgatcagcgtgctcggggtggaaggcaacgggcaggcaccggaagcggtgttgcaggcagtgcgcctggcgctgaacgcggaggacgtgcgtcctgtcgcggatcgggtaacggtgcgctcggcaggaatcgttccctatcaggtcaaggcgcagctctacctgtttcccggtcccgaggccgagctgatccgtgccgccgccgaggcttcgctgcgcgactacatttccgcccagcgccgcctgggccgcgacatccggcgttcggccctgttcgccaccctgcatgtcgaaggcgtgcaacgcgtcgaactgcaggagcctgcggccgacgtggtcctggatgaaacccaggcggcctattgcacggggtacgcgatcaccttgggaggcgtcgatgagtagccgactgctgccgccaaacaggagttctctggaacgctctctgggtgatgtattgcctgccgaactgccggtgccgcttcgtgagcttcacgatccggcacgctgtgaggcggccttgttgccctacctggcctggacgcgctcggtggaccgctgggacccggactggagcgacgaggccaagcgcaatgcggtagcgacgtccttcgtcctgcaccagcgcaaaggcacgctgactgcgttgcgccaagtggtcgagccgatcggtgcgctgagcgaggtcaccgaatggtggcagcgaagcccgaccggcgtgccggggaccttcgagatcaccgtggacgtcagcgaccgtggcatcgacgaaggcaccgtactggagctggagcgcttgctcgatgacgtccgcccggtgagcagacacctgacccggctggacctgcgcattaccccggtaatccggtcccgtcacggactagccgtgaccgacggcgacaccctggaaatcttcccctggaaacagtgacatgacgaccaatactccgaaatacggtggcctgctcaccgacataggtgccgctgcgctggctacggccagcgcagcaggcaagaaatggcagccgactcatatgctgatcggcgatgccggcggtgcgccgggcgacacgccggatccattgccttctgcggcgcagaagagcctgatcaaccaacgccatcgggcccagctgaatcggctgttcgtttccgacaagaacgccaataccttggttgccgaggtggtgctgccagttgaggtaggtggcttctggatccgcgagatcggcctgcaggatgccgacggcaagttcgtcgcggtatccaactgcccgcccagctacaaggctgcaatggaaagtggcagtgcgcggacccagaccattcgggtgaacatcgcgctctccggcctggagaatgtccagctgctgatcgacaacggcatcatctacgccactcaggactgggtgaaggaaaaggtcgctgccgatttcaagggccgcaagatcctggctggcaatggcctgctcggtgggggcgatctttctgccgaccgcagcattggcctggcgccttccggcgtgacggcgggcagctatcgttcggtcacggtgaacgccaacggggtggtcacccagggcagcaatccgaccaccctggccggctatgcgatcggagatgcctataccaaggccgataccgacggaaaactggcgcagaaagcgaacaaggccaccaccctggccggctatggcatcaccgatgcgctgcgagtcgatggcaacgccgtgtcatccagcaggctggccgcaccgcgcagcctggcagccagtggcgatgcctcctggtcggtgaccttcgacggcagtgccaatgtttctgcgccgctgagtctttccgctaccggtgtggcggcgggcagctatccgaaggtgaccgtggatacgaagggaagggtgactgctggaatggcgctggcggcgacggacattcccgggctggatgcgtcgaagctggtcagcggggtgctggccgagcagcgtttgccggtattcgcgcgcgggttggctactgctgtctcgaacagtagcgatccgaacaccgcgaccgtgccgttgatgctgaccaatcatgcgaacggacctgttgccggacgatacttctacatccagtcgatgttctatccggatcagaacggcaatgcttcgcagattgcaacgagctacaacgctacatccgagatgtatgtacgggtgtcctacgcggccaaccctagcatccgggagtggttgccctggcagcgctgcgacattggaggttccttcaccaaagaggccgatggtgaactgcctggaggcgtcaacctggattcgatggtgacctcagggtggtggagccagagttttactgcccaagctgccagtggagccaactaccctatagttcgggccggcctgcttcatgtgtacgccgcgagtagcaatttcatctatcagacgtatcaagcctacgatggtgagagtttctatttccggtgccggcattcaaatacctggtttccctggcgtcgcatgtggcatggcggagacttcaaccccagtgactatctgttgaagtcggggttctattggaatgcgttaccgggaaaacctgccacttttccaccatccgcacataaccatgacgtcggacagcttacttcgggcattctccccctggcacgtggcggcgtcggttcgaatacggcagcgggagcacgtagcactatcggagcaggggttcctgcgactgcttcccttggggcgagcggatggtggcgggacaatgacactggcctcattaggcaatgggggcaggtcacttgccccgccgatgccgatgcttcgattacgttcccgattcctttccctacgctatgcctcggcggatatgcgaatcagacgagtgctttccatccgggaacggatgccagtacaggtttccgtggagcgactaccactaccgcggtgattcgcaatggctactttgctcaggcggttctttcatgggaggcatttggacgatgaagggcgaatattatttctctccaagccaggtggcattctatccggcctccttgcgagaggtttatgaatacgcaggctgctggccagtcgatggcgagtgggtcagcgcagagctacatgaacaactgatgaacgaacaggcggcaggccgagcaatcagttccgacgtgaatgggaacccagtagcgatcgagcgccctccgctttcccgtcagcaacgtagcacccatgagcggagatggcgggatagtcagctgttggcgaccgacggcctagttgttcgccatcgagatcaattggaaaccggaaaggaaacgaccttactccctgtccaataccatgaactcatgtcgtacagagccagcttacgggattggccggaagagcctttatttcccgacagtggcggacgcccgtccgtaccagattggctcagacgttatgtcaccccctgaaccccgcctcgtgcggggtttttcattaatggagatctacctatgagtttcttccacggcgttacggtaaccaacgtcgatatcggtgcgcgcaccatcgcgctgccggccagctccgtcatcggcctctgcgatgtgttcacgccgggggcgcaggcaagcgccaagcccaacgttccggtgctgctcaccagcaagaaggacgccgccgcggcgttcggcatcggctcgtctatctacctggcctgcgaggccatctataaccgtgcccaggccgtgatcgtggcggtgggcgtggaaaccgcggagactcccgaggcccaggccagcgccgtcataggtggtatcagtgccgccggcgagcgcaccgggctgcaggctttgctcgacggcaagagtcgcttcaatgcccagccgcgtctactagttgcgccgggtcattcggcccagcaagcggtggccaccgccatggacgggctggccgagaaactgcgggccatcgccattctcgatggtcccaatagcaccgacgaggcggccgtcgcctacgccaagaacttcggcagcaagcgcctgttcatggtcgacccgggcgtgcaggtttgggacagcgccaccaatgccgcgcgcaacgccccggcttcggcctacgccgccggcctgttcgcctggaccgacgccgagtacggcttctggtcctcgccgtcgaacaaggagatcaagggcgtcaccggcaccagccgtccggtggagttcctcgacggcgatgagacctgtcgcgccaacctgctcaacaacgccaatatcgcgacgatcattcgcgacgatggctatcgcctgtggggcaaccgcaccttgtccagcgacagcaagtgggctttcgtcacccgtgttcggaccatggacctggtaatggatgcgatcctcgccgggcacaagtgggcggtggaccgcggcatcaccaagacctacgtgaaggatgtcaccgagggcctgcgcgccttcatgcgcgatctgaagaaccagggagcggtgatcaacttcgaggtctatgccgacccggacctgaacagcgccagccagctggcccagggcaaggtgtactggaacatccgcttcaccgatgtgccgcctgccgagaaccccaatttccgtgtcgaggtgaccgatcagtggctcaccgaagttctggatgtcgcctaaggagcgcccgtgatgattccgcaaaccctgaccaataccaacctgttcatcgacggcgtgagcttcgccggtgacgtgccatccctgaccctgcccaagctggctgtgaagaccgagcaataccgtgccggcggcatggatgcgccggtatccatcgacatgggcctggaggcgatggaggccaagttctccaccaacggtgcccgccgagaagcgctgaatttcttcggcctggccgaccagagcgctttcaatggcgtgttccgtggctccttcaagggccagaagggcgccagcgtgccagtggtggctaccctgcgcggcctgctcaaggaggtcgacccgggcgactggaaagccggcgagaaagccgagttcaagtacgccgttgcggtcagctactacaagctggaagtcgatggccgcgaggtctacgagatcgatccggtcaacggtgtccgcgcgatcaacggtgtcgaccagttggccggcatgcgcaacgacctcggcctgtaagaggagctccggacatgacccaagagaatcgactgccgggctggctgacgctggatgccgacgccgccctcgttcgtctctcgcgtccggcacagtgcaacggggtcagcgtcgacacgctgaccctgcgtgcacccaccgtgcgtgatatccgcctggccggcaaggtggccggcgacgatgccgaggagcgcgaactgcaactgttcgcctcgctggcgcaggtcagccgccaggacctggaggggctgaagctgagcgactaccagcggctgcaaggcgcctactttcgcctggtgcaagacgacacggacgacaccttcgcgtatgcgtcaactggcgaggcgcctggccattgagctgggcttccaggcctgcgaactggagcgcatgaccctgggcgacctgctctggtggctcgccgagggcgaggagtgagcgacggcgccgtccgccaggcgagcggcgcccgcgctacttcagtaggtccgcggaggcggacatcgataacagacgagacgaaccgtcatgagtaaagacatggacctggtggtctccatcggcggcatcgccgacccctccttgggcaaggcattcgagaccgtcaaggcacggctcgacagtctccaggaacgtgcccgccaggcttccagcctgagggatgtgctgggagacgccatacgcctggagcgagagctcgccgatatgcgcaaggtcggggatcgcggcgttgctgagcatgcccggcagcttggcgaacgccaggagcaactgaagcgactcggcatcgaggccagggccgcgggcgatgcctacgctcgactgggcgagatgcagcgtggcctggatatgcaggtccgcggcctgcaacggctggagcaggccagccaggcaatgccattggctagcgcattttccggactggtcgtggaagccagcaagacggctgccggttatcaagcgcggttgcgcgacctgtcgatccgcaacggcctggacgtcggccgggagccagccttggcatccctgatccaggacagcgccaaccagagcggcctgggacgcacggtgacgctggacatgctggagcacttgaacgccaccggcatggggttcgccgccgcgcaaatgaatctgggactggcgggccgcttcggctttggccaagggattgcttcagccgaggttgcggggctggttcgagcgttgcaactggcccagggttcggactcgccagagcaattgtccgccaccctcgaccgcctggtcgtcctgggtaaaggcagagtcggcagtgaggccctggcgcgtcgcctgcccgccttgttgtcagcgctgggcaatgcgggagaggccacggccggcgatgtcggtgcattgggtgccttgctggagattcaggcaaagaacaccacgccagacaaagcggacgtgcggatgaaggcctggctggagttcgtcggcagcggcagcttgaaacgcgcttatggccaggactacgaccgggacctggaagcgctgcgcaaggacggagcgagtctgctggaggccaacctggagctggctgcgcgctatcgggataagggcggcaagctcagcgccggcgtggcgtcgccggcgcttgaagcctatcgagcgtcgcgcggcgagttccagggcttgctcgaatcccagcagtcttccgtaggctcttcggagcgcgatgcgcagcgtcgcaaggggatgtcccaggagctgtggaaggcttccagcgacagttgggagagggcgcagaccgctttgggcagcgccctgaatccatatctggacaacctggccaaggggagcgcggtactcggcgagtcgactgcggagctgctcgaagcctatccgcggacgacggccggtcttaccgccgccgcaggtgcggtgttatccgggtatctcgcctacaagggagggcgcggcgctatcgacgtgctgcgtggcggtcggctcggtcggcgagggaccgctgccgtcggcgacctgatcgaacggggtgcaggccgggtatcgggtggtagcgaaatacagcgcgtgttcgttaccaactggccggtgccgggaggcgactctacgctggagtccgcgaggaggccagcacaacgaaagcgcggtcaaacaccacgcaggaagagaggcaagggcggtgggctgaaggcccgctcccttccatcccttggcttctcggctggaggaggcttgggggcgatggcaggaaagctgccgcgcctgtcgcgtttgcccatacgcaacgcgccgctgcaagtggcctcgtcgttgatcgatgttgctgaggtctactccagcgacctgtcggagagcgaaaagaccgttgcctatggcgaggcaggtggctccctggctggttccctggctggcgccgctctgggagcgagcatcggctcggtggtgccggtggtcggtacgctgatcggtggattggttggcggcgctatcggcgcctggggcggtagcgaactgggggggcgcctggggcgcagtctggctggcgatccgccggcggcctcggacaacaagccggcggtggccgtaccacaggccggacccgtcgcggctgcgcccaactggaccttcgcgccgcagatcaacctgacggtgcaaggcaacgtgcacgagccgcagcgcctggccgacgagttgctgccctacctgcaacgcatgcttgtcgacttcgccgacgagcggcagcggcgcagcctctacgacccggcgatggtttaaggagtccccatggcatatctggaacaattgcaggccggcctgaggtacctgggtcgcgccggcgagtccggacgcaagagtctggacaaggtggtcgctccggtgaacggcgcgatcagcgagatccgcggcgcagccgcggagctggagaacctgcccggcgtatcgccggaaatggctgcccggctgcagcgtgccatgcgcggcatcggccaggcgcagggcaaggtgaaccgcgtggtctccacctatgaccgggcgagccgggcgttgcttggtatcgacgaacgcctggatgcgctgaaggtgcaggtgaacagtgccgcgcaggcggtcggcaaggtcgctggcgacatcagtccgacgctggcgggggtgctgccgtcgtggctgctggcaccctcggcgacgcccccgagcgaggccgcagcgtccttgccgcacctgctggtactgcagccgctgaccgccaatgcccaaccgttctacttcaacctgaatactgccgccttcgacgccctgcagcgcaacagtgcctacaactggagcgggcaggtgcgcctgggtcggcggccggcgctgcagagcgtcggcatgggcgaggagagcatcctgctcaagggtgcggtattcccgctgcgtcgacaggtaggtaaccaggaaaaggtcgtcggtctggagcagctcgaagcgctgcgccggttggcggagcggcgtgagccgctgatcctgagcagcggctacggcgaggtgcagatgggcctctggtgtctggtgcggatcagcgagaaccagagcgccctactgggcaacggcgctccccgcaaacaaaccttcgacctggagttcaagcgctatggcgacgacctgccgaaccgctgacggcgacatgctggacagcctctgctaccacgtctatggccatctgttgggctgcgtcgaggcgaccctcgacgccaatcccgggctggccgatgagcagcagccattccgcgccggcttgctgatcagtttccctgacatgccggtggtcaatgtcgaacaggtgcgcctgtgggattgatcgaccgctcacccgcaaccccgccttggcggggtttttcttttctggagaaaccaggtgcaaccgagtttccgtatcgttgccgacggcaccgacgtcacccagcggctgaatgaccgcctgctcaagctgaccctgctggacaagccgggcatggagtccgacagcctgaccttgaggatcgacgatcgcgatggacaggtggccttacccaggcgcggtgcggtgctggaggttcatctcggctatgccggcgagccactgatgcgcatgggacgctttaccgtggacaccttgcagtgggctggtccgccggactgcctgaccgtcactgccaaggccggcgacatgcgcggcagtggcaagacgatacgcagcggaggttgggagggcactaccctggctcaggtctgccgcgatgttggcgcacgcaacggctggcgcgtggagtgtccgttgcaggtggcgatcgcccgggtcgaccaggtcaatgagtccgactaccacttcgtcacccgtctggcgcgccagtacgactgcaccgccaagctggccgagggcatgctcatggtgctgccgcgacagagcgggcagagcgccacggggcgtcggatcgaacctttggtgttgggacgtgctgacgtcggtagcttcgacgttaccttcgacgaccgcagcctgatgagaacggtgaagacccgctaccaattgcccggcagcggcgaggtcaagagcgtcgagttgaagaacccgaaggcaccggctacggctatgggcgagcatgtcgaccggcacctctataccagccgtggagaggccgagcaggcggcgaaggctcgcctggcgagcttcagccgctccagcgccagtgtgcgcctggaactgccagggcgtggcgacctgttcgccgagcgcagcctgctgctccaaggcttcaaggcgggaatcgacggcgagttcctgatcgactcggtggagcacacctacagctccagcggatggaccactgtcgtgcaatgcaacggcggccgaggcggcaaggggtgacgcctgaaacccatcggagtgcaggaggatcgatgaaactgaccgagcagcaattgctgcgcatttttcccaacgcccgcctcgtcgcgggcgttttcgttgtggcgttgcaacgggccatggacgagagggagatcgacacaccggcgcggcgtgccgcgtttctcgcccaggtcggccacgaaagcagccagttgacccggctggtggagaacctcaattacagcgcccaaggcttggcggcgacctggccgggtcgctatctcggccccgacgggcagcccaacgcgttggccttgcggctggcgcgcaatccgcaggcgattgccgacaacacctacgccacgcgcaacggcaatggcgacgaagcgtccggcgatggctggcgcttccgcgggcgtggcttgctacaaatcaccgggcgtgccaactaccggttggtcggcgaggccctcggcgagccgctggaagccgagccctggcgcctggagcagcccgtgccggcggcccgcagcgccgcctggtggtgggccggtcacgggctcaacgagctggccgaccgcggcgagttcgctgccatcacccgccgcattaacggcggcctgaatggccaggcggagcgcctggcgttgtggcagcgggccagggcggtgctgtcatgagccggctcgctctgctcctgccggccgtgttgttggtcctgctggccggcgccttgctcggcggcggcctggttgcccgccattatcgtccgcaactggaggaggccctgggacaactcactgccagccgcgtcgccagcggccagctcgaggctttgctcgatgagcagcagcgcgcgctggcggcggtgcgggcgagcgccgagaggcgcgcgaaggacgtcgagcaggcactcggcgaggccagggcgcaggccgcggagcagtatgccgcggccgtgcgtctgctccaagaacccgactttggcacggactgccaggcggcaggtgcggcgatcgaccgggagctgggactatgactcgtctcctgctggggctttgtctgcttttcgcgggctgcgcagcctcaccgacgacacctcgcccagtgcgcgtcgaggttcccctggcagtgccctgccgtgtacctgacgtgcgcccgccgagctgggccggcgccacgctgaaggccggcgattcgctgcaggccaaggttcgcgcattgctcgccgagcgccgccagcggcagggctacgaactcgaattgcaggcggcattgcgtgcctgccgctgagacactgggcgcatatggacgtcgatggacgtgtgtccggcgcatattgccgtgtattgattccgcagcgttgtcgagccgggaatcggtcggtacaacgtagtcatgcttgtacaggtgtgtccccccagggatgtcacctgcaacctcagagcccggccagtgtgccgggctttttcgtttgcatccgacaacggctcgggacgtggaggctcctcgccgaccgcgtaccgcgccacggctgaccgtcctggaggcggggccagacgacccgcctggtgcgggtcttttcatgtgcataaaggagagttttcca

SEQ ID NO: 5 - DNA, 2076 bp, Pseudomonas aeruginosa
  misc_feature 1..2076: note = Description of sequence: polynucleotide, name: PAO1 R2 Pyocin tail
  source 1..2076: mol_type = genomic DNA
atgacgaccaatactccgaaatacggtggcctgctcaccgacataggtgccgctgcgctggctacggccagcgcagcaggcaagaaatggcagccgactcatatgctgatcggcgatgccggcggtgcgccgggcgacacgccggatccattgccttctgcggcgcagaagagcctgatcaaccaacgccatcgggcccagctgaatcggctgttcgtttccgacaagaacgccaataccttggttgccgaggtggtgctgccagttgaggtaggtggcttctggatccgcgagatcggcctgcaggatgccgacggcaagttcgtcgcggtatccaactgcccgcccagctacaaggctgcaatggaaagtggcagtgcgcggacccagaccattcgggtgaacatcgcgctctccggcctggagaatgtccagctgctgatcgacaacggcatcatctacgccactcaggactgggtgaaggaaaaggtcgctgccgatttcaagggccgcaagatcctggctggcaatggcctgctcggtgggggcgatctttctgccgaccgcagcattggcctggcgccttccggcgtgacggcgggcagctatcgttcggtcacggtgaacgccaacggggtggtcacccagggcagcaatccgaccaccctggccggctatgcgatcggagatgcctataccaaggccgataccgacggaaaactggcgcagaaagcgaacaaggccaccaccctggccggctatggcatcaccgatgcgctgcgagtcgatggcaacgccgtgtcatccagcaggctggccgcaccgcgcagcctggcagccagtggcgatgcctcctggtcggtgaccttcgacggcagtgccaatgtttctgcgccgctgagtctttccgctaccggtgtggcggcgggcagctatccgaaggtgaccgtggatacgaagggaagggtgactgctggaatggcgctggcggcgacggacattcccgggctggatgcgtcgaagctggtcagcggggtgctggccgagcagcgtttgccggtattcgcgcgcgggttggctactgctgtctcgaacagtagcgatccgaacaccgcgaccgtgccgttgatgctgaccaatcatgcgaacggacctgttgccggacgatacttctacatccagtcgatgttctatccggatcagaacggcaatgcttcgcagattgcaacgagctacaacgctacatccgagatgtatgtacgggtgtcctacgcggccaaccctagcatccgggagtggttgccctggcagcgctgcgacattggaggttccttcaccaaagaggccgatggtgaactgcctggaggcgtcaacctggattcgatggtgacctcagggtggtggagccagagttttactgcccaagctgccagtggagccaactaccctatagttcgggccggcctgcttcatgtgtacgccgcgagtagcaatttcatctatcagacgtatcaagcctacgatggtgagagtttctatttccggtgccggcattcaaatacctggtttccctggcgtcgcatgtggcatggcggagacttcaaccccagtgactatctgttgaagtcggggttctattggaatgcgttaccgggaaaacctgccacttttccaccatccgcacataaccatgacgtcggacagcttacttcgggcattctccccctggcacgtggcggcgtcggttcgaatacggcagcgggagcacgtagcactatcggagcaggggttcctgcgactgcttcccttggggcgagcggatggtggcgggacaatgacactggcctcattaggcaatgggggcaggtcacttgccccgccgatgccgatgcttcgattacgttcccgattcctttccctacgctatgcctcggcggatatgcgaatcagacgagtgctttccatccgggaacggatgccagtacaggtttccgtggagcgactaccactaccgcggtgattcgcaatggctactttgctcaggcggttctttcatgggaggcatttggacgatga

SEQ ID NO: 6 - protein, 691 aa, Pseudomonas aeruginosa
  REGION 1..691: note = Description of sequence: peptide, name: PAO1 R2 pyocin tail fiber
MTTNTPKYGGLLTDIGAAALATASAAGKKWQPTHMLIGDAGGAPGDTPDPLPSAAQKSLINQRHRAQLNRLFVSDKNANTLVAEVVLPVEVGGFWIREIGLQDADGKFVAVSNCPPSYKAAMESGSARTQTIRVNIALSGLENVQLLIDNGIIYATQDWVKEKVAADFKGRKILAGNGLLGGGDLSADRSIGLAPSGVTAGSYRSVTVNANGVVTQGSNPTTLAGYAIGDAYTKADTDGKLAQKANKATTLAGYGITDALRVDGNAVSSSRLAAPRSLAASGDASWSVTFDGSANVSAPLSLSATGVAAGSYPKVTVDTKGRVTAGMALAATDIPGLDASKLVSGVLAEQRLPVFARGLATAVSNSSDPNTATVPLMLTNHANGPVAGRYFYIQSMFYPDQNGNASQIATSYNATSEMYVRVSYAANPSIREWLPWQRCDIGGSFTKEADGELPGGVNLDSMVTSGWWSQSFTAQAASGANYPIVRAGLLHVYAASSNFIYQTYQAYDGESFYFRCRHSNTWFPWRRMWHGGDFNPSDYLLKSGFYWNALPGKPATFPPSAHNHDVGQLTSGILPLARGGVGSNTAAGARSTIGAGVPATASLGASGWWRDNDTGLIRQWGQVTCPADADASITFPIPFPTLCLGGYANQTSAFHPGTDASTGFRGATTTTAVIRNGYFAQAVLSWEAFGR

SEQ ID NO: 7 - DNA, 2531 bp, Pseudomonas aeruginosa
  misc_feature 1..2531: note = Description of sequence: polynucleotide, name: R3 tail
  source 1..2531: mol_type = genomic DNA
atgacgactaatactccgaaatacggtggcctgctcaccgacataggtgccgctgcgctggctgcggccagcgcagcaggcaagaaatggcagccgactcatatgctgatcggcgatgccggcggtgcgccgggcgacacgctggatccattgccttctgcggcgcagaagagcctgatcaaccaacgccatcgggcccagctgaatcggctgttcgtttccgacaagaacgccaataccttggttgccgaggtggtgctgccagttgaggtaggtggcttctggatccgcgagatcggcctgcaggatgccgacggcaagttcgtcgcggtatccaactgcccgcccagctacaaggctgcgatggaaagtggcagtgcgcggacccagaccattcgggtgaacatcgcgctctccggcctggagaatgtccagctgctgatcgacaacggcatcatttacgccactcaggactgggtgaaggaaaaggtcgctgccgatttcaagggccgcaagatcctggctggcaatggcctgctcggtgggggggatctttctgccgaccgcagcattggcctggcgccttccggcgtgacggcgggcagctatcgttcggtcacggtgaacgccaacggggtggtcacccagggcagcaatccgaccaccctggccggctatgcgatcggagatgcctataccaaggccgataccgacggaaaactggcgcagaaagcgaacaaggccaccaccctggccggctatggcatcaccgatgcgctgcgagtcgatggcaacgccgtgtcatccagcaggctggccgcaccgcgcagcctggcagccagtggcgatgcctcctggtcggtgaccttcgacggcagtgccaatgtttctgcgccgctgagtctttccgctaccggtgtggcggcgggcagctatccgaaggtgaccgtggatacgaagggaagggtgrctgctggaatggcgctggcggcgacggacattcccgggctggatgcgtcgaagctggtcagcggggtgctggccgagcagcgtttgccggtattcgcgcgcgggttggctactgctgtctcgaacagtagcgatccgaacaccgcgaccgtgccgttgatgctgaccaatcatgcgaacggacctgttgccggacgatacttctacatccagtcgatgttctatccggatcagaacggcaatgcttcgcagattgcaacgagctacaacgctacatccgagatgtatgtacgggtgtcctacgctgccaaccctagcatccgggagtggttgccctggcagcgctgcgacattggcggttccttcaccaaagaggccgatggtgaactgcctggaggcgtcaacctggattcgatggtgacctcggggtggtggagccaaagttttactgcccaagctgccagtggagccaactaccctatagctcgggccggcttgcttcatgtgtacgccgcgagtagcaatttcatctatcagacgtatcaagcctacgacggtgagagtttctatttccggtgccggtattcaaatacctggcttccctggcgtcgtatgtggcatggcggagacttcaaccccagtgactatctgttgaagtcggggttctattggaatgcgctaccgggaaaacctgccacttttccaccatccgcacataaccatgacgtcggacagcttacttcgggcatcctccccctggcacgtggcggcgtcggttcgaatacggcagcgggagcacgtagcactatcggagcaggggttcctgcgactgcttcccttggggcgagcggatggtggcgggacaatgacactggcctcattaggcaatggggacaggtcacttgccccgccgatgccgatgcttcgattacgttcccgattcctttccctacgctatgcctcggcggatatgcgaatcagacgagtgctttccagccgggaacggatgccagtacaggtttccgtggagcgactaccactaccgcggtgattcgcaatggctactttgctcaggcggttctctcatgggaggcatttggacgatgaagggtgaatattatttctcgccaagccaggtggcattctatccggcctccttgcgagaggtttatgaacacgcaggctgctggccagtcgatggggagtgggtcagcgcagagctacatgaacaactgatgaacgaacaggcggcaggccgagcaatcagttccgacgtgaatgggaacccagtagcgatcgagcgccctccgctttcccgtcagcaacgtagcacccatgagcggagatggcgggatagtcagctgttggcgaccgacggcctagttgttcgccatcgagatcaattggaaaccggaaaggaaacgaccttactccctgtccaataccatgaactcatgtcgtacagagccagcttacgggattggccggaagagcctttatttcccgacagtggcggacgtccgtccgtaccagattggctcagacgttatgtcaccccctga

SEQ ID NO: 8 - protein, 691 aa, Pseudomonas aeruginosa
  REGION 1..691: note = Description of sequence: peptide, name: R3 pyocin tail fiber
MTTNTPKYGGLLTDIGAAALAAASAAGKKWQPTHMLIGDAGGAPGDTLDPLPSAAQKSLINQRHRAQLNRLFVSDKNANTLVAEVVLPVEVGGFWIREIGLQDADGKFVAVSNCPPSYKAAMESGSARTQTIRVNIALSGLENVQLLIDNGIIYATQDWVKEKVAADFKGRKILAGNGLLGGGDLSADRSIGLAPSGVTAGSYRSVTVNANGVVTQGSNPTTLAGYAIGDAYTKADTDGKLAQKANKATTLAGYGITDALRVDGNAVSSSRLAAPRSLAASGDASWSVTFDGSANVSAPLSLSATGVAAGSYPKVTVDTKGRVXAGMALAATDIPGLDASKLVSGVLAEQRLPVFARGLATAVSNSSDPNTATVPLMLTNHANGPVAGRYFYIQSMFYPDQNGNASQIATSYNATSEMYVRVSYAANPSIREWLPWQRCDIGGSFTKEADGELPGGVNLDSMVTSGWWSQSFTAQAASGANYPIARAGLLHVYAASSNFIYQTYQAYDGESFYFRCRYSNTWLPWRRMWHGGDFNPSDYLLKSGFYWNALPGKPATFPPSAHNHDVGQLTSGILPLARGGVGSNTAAGARSTIGAGVPATASLGASGWWRDNDTGLIRQWGQVTCPADADASITFPIPFPTLCLGGYANQTSAFQPGTDASTGFRGATTTTAVIRNGYFAQAVLSWEAFGR

SEQ ID NO: 9 - DNA, 2531 bp, Pseudomonas aeruginosa
  misc_feature 1..2531: note = Description of sequence: polynucleotide, name: R4 tail
  source 1..2531: mol_type = genomic DNA
atgacgaccaatactccgaaatacggtggcctgctcaccgacataggtgccgctgcgctggctgcggccagtgcagcaggcaagaaatggcagccgactcatatgctgatcggcgatgccggcggtgcgccgggcgacacgccggatccattgccttctgcggcgcagaagagcctgatcaaccaacgccatcgggctcagctgaatcggctgttcgtttccgacaagaacgccaataccttggttgccgaggtggtgctgccagttgaggtaggtggcttctggatccgcgagatcggcctgcaggatgccgacggcaagttcgtcgcggtatccaactgcccgcccagctacaaggctgcaatggaaagtggcagtgcgcggacccagaccattcgggtgaacatcgcactctccgggctggagaatgtccagctgctgatcgacaacggcattatctacgccactcaggactgggtgaaggaaaaggtcgctgccgatttcaagggccgcaagatcctggctggcaatggcctggtcggtgggggcgatctttctgccgaccgcagcattggcctggcgccttccggcgtgacggcgggcagctatcgctcggtcacggtgaacgccaacggggtggtcacccagggcagcaatccgaccaccctggccggctatgcgatcggagatgcctataccaaggccgataccgacggaaaactggcgcagaaagcgaacaaggccaccaccctggccggctatggcatcaccgatgcgctgcgagtcgatggcaacgccgtgtcatccagcaggctggccgcaccgcgcagcctggcagccagtggcgatgcctcctggtcggtgaccttcgacggcagtgccaatgtttctgcgccgctgagtctttccgctaccggtgtggcggcgggcagctatccgaaggtgaccgtggatacgaagggaagggtgactgctggaatggcgctggcggcgacggacattcccgggctggatgcgtcgaagctggtcagcggggtgctggccgagcagcgtttgccggtattcgcgcgcgggttggctactgctgtctcgaacagtagcgatccgaacaccgcgaccgtgccgttgatgctgaccaatcatgcgaacggacctgttgccgggcgatacttctacatccagtcgatgttctatccggatcagaacggcaatgcttcgcagattgcaacgagctacaacgctacatccgagatgtatgtacgggtgtcctatgcggccaaccctagcatccgggagtggttgccctggcagcgctgcgacattggaggttccttcaccaaagaggccgatggtgaactgcctggaggcgtcaacctggattcgatggtgacctcagggtggtggagccaaagttttactgcccaagctgccactggagccaactaccctatagttcgggccggcctgcttcatgtgtacgccgcgagtagcaatttcatctatcagacgtatcaagcctacgatggtgagagtttctatttccggtgccggcattcaaatacctggtttccctggcgtcgcatgtggcatggcggagacttcaaccccagtgactatctgttgaagtcggggttctattggaatgcgttaccgggaaaacctgccacttttccaccatccgcacataaccatgacgtcggacagcttacttcgggcattctccccctggcacgtggcggcgtcggttcgaatacggcagcgggagcacgtagcactatcggagcaggggttcctgcgactgcttcccttggggcgagcggatggtggcgggacaatgacactggcctcattaggcaatgggggcaggtcacttgccccgccgatgccgatgcttcgattacgttcccgattcctttccctacgctatgcctcggcggatatgcgaatcagacgagtgctttccatccgggaacggatgccagtacaggtttccgtggagcgactaccactaccgcggtgattcgcaatggctactttgctcaggcggttctttcatgggaggcatttggacgatgaagggcgaatattatttctctccaagccaggtggcattctatccgrcctccttgcgagaggtttatgaatacgcaggctgctggccagtcgatggcgagtgggtcagcgcagagctacatgaacaactgatgaacgaacaggcggcaggccgagcaatcagttccgacgtgaatgggaacccagtagcgatcgagcgccctccgctttcccgtcagcaacgtagcgcccatgagcggagatggcgggatagtcagctgttggcgaccgacggcctagttgttcgccatcgagatcaattggaaaccggaaaggaaacgaccttactccctgtccaataccatgaactcatgtcgtacagagccagcttacgggattggccggaagagcctttatttcccgacagtggcggacgtccgtccgtaccagattggctcagacgttatgtcaccccctga

SEQ ID NO: 10 - protein, 691 aa, Pseudomonas aeruginosa
  REGION 1..691: note = Description of sequence: peptide, name: R4 pyocin tail fiber
MTTNTPKYGGLLTDIGAAALAAASAAGKKWQPTHMLIGDAGGAPGDTPDPLPSAAQKSLINQRHRAQLNRLFVSDKNANTLVAEVVLPVEVGGFWIREIGLQDADGKFVAVSNCPPSYKAAMESGSARTQTIRVNIALSGLENVQLLIDNGIIYATQDWVKEKVAADFKGRKILAGNGLVGGGDLSADRSIGLAPSGVTAGSYRSVTVNANGVVTQGSNPTTLAGYAIGDAYTKADTDGKLAQKANKATTLAGYGITDALRVDGNAVSSSRLAAPRSLAASGDASWSVTFDGSANVSAPLSLSATGVAAGSYPKVTVDTKGRVTAGMALAATDIPGLDASKLVSGVLAEQRLPVFARGLATAVSNSSDPNTATVPLMLTNHANGPVAGRYFYIQSMFYPDQNGNASQIATSYNATSEMYVRVSYAANPSIREWLPWQRCDIGGSFTKEADGELPGGVNLDSMVTSGWWSQSFTAQAATGANYPIVRAGLLHVYAASSNFIYQTYQAYDGESFYFRCRHSNTWFPWRRMWHGGDFNPSDYLLKSGFYWNALPGKPATFPPSAHNHDVGQLTSGILPLARGGVGSNTAAGARSTIGAGVPATASLGASGWWRDNDTGLIRQWGQVTCPADADASITFPIPFPTLCLGGYANQTSAFHPGTDASTGFRGATTTTAVIRNGYFAQAVLSWEAFGR

SEQ ID NO: 11 - DNA, 14782 bp, Pseudomonas aeruginosa
  misc_feature 1..14782: note = Description of sequence: polynucleotide, name: R5 pyocin
  source 1..14782: mol_type = genomic DNA
caccgcaccagggacggggcgcgccggtggcgtacggtggaccaccagaagacccagccgatcatgctgatctggcggctgcgcatgtcctccggcgagtactcctcgtccgggtattcctcgcggttgaagctgcgcagccgaatgccgccgccgggcaggcgatagacgaacttcacccgcagcatgccgtcatgttcgagggcgtagatctcgccatcggtgatatgggtggtggcggtgtccacgccgatggtggagccatccatgatcagcggttccatgctgttgccggtgagttgggcgcagatcgccgccgacggatcgacgcccgaggcacgcagcgtggcgtaggaaaaacgcagcttgcgcccctctatctcgcgcaccgcagtgcgtccggcgccggcggacatctccacttccttgtacagcggcagttccacttcgtcttcgtccagcggtgtgtcgctgtcccacggatgcagcggttccagcaccaatgggctgccatcggccacggacacggcactgcgcgcggctggagtgccttcgccggtctgcagccagaccggcgaaacgcccagggccgccgcgatctcgatcagcttgcgggtgctctgggccttgcccgaggtcagcttgtgaatggtgttctgcgaaacccctgccgcttcggcgagggtttcctgcttcaggttgcgcatcgccatggcctgcttgaggcgagcggcgaagctgtcgggcgggatctgggtgctcttgtccatgctcgacaatctacagaccgatggattttctgtaaagagcctaggcgttgacgataaatagctttggttgtaatttctcttccgtcagaaagcggaaggggtgagttcctcaacttcccacccgcctttcagagatttcacatgggtgcagggacgcacccggttgtcggcgaccgacgatgcaggtgccgactgcttgggtgccggggccatgggaaagcgctccggcacctggagggccggagcgccgtccatgcagacgctccgtcccgctcattcattctgtcgccgacctgcttcaggtacggcggcgtcgtacagggagtcattgccatgtcgcgagatacgcactcgccatttccgcgcagaggaacgcaggtcgccgaaccggcgccgcgcatctgtcatgttccatccattcggccgtcgggcgtcgggcacgccagggaggctttccatggctgaccttgccgatcacgccaacgaactggtcctggctcgcctcgacggcctcctggcggcgcgcccggcgctggccatccgcgagtccgcggaagactgcgaggactgcggcgagcccattccccaggcgcgccgccgggcggcaccgggctgcagtcgctgcatcgactgccaggaccgccacgagcgccgttgaaccgacctcatgccgagccctcgcgggcagtgaaaggagacacgaccgtgatcaaagccatcgatgaaatgctcaagctctgggccgaggaaatgcacgcgccaggcagcaacggaggcggctacgccggcggcaacctgatcgccatgctgatcgccagcaagggcgaggtggtccgcggccaccgtggcagccgggtgatcctcgaccgtgtggcggaggtcgatcgcctggtaaatcgcctacccgaggaactgaagaacgtggtggtggagcactatctcaatcgcgacagcttccccgagcagaagtaccgccactgcggttgcagccgcaacaccttctatctgcgcctgcatgtggcgcaccagggtatccaggacggcctgctgcggcgggtggcctgagttccgggaccggataccgtgtcccccctggaattttccaaccggccttcgagccggtttttttatgcctgtcgcccatggaggcggcgagccccatgcacagcagcggaccctgcacgctagaggcgggttcgggctggcggagccgtccttttcgtcagtcgatgccgggcttttccatatcggaagtgggtacgcgcttgcccggatgcctggcgaaggttcccagggcggtatatcaccctcatatcgtggtgcgtctccctcctattgcagtggattgtcgagaaggcattgccgggctgggaatcgggcggtaaaaagtagtcattcttgtaaaggtgcgtccccagggaggcactcgtgaagcaccggaacccggccctggcgccgggttttttattgcccgggaaaacgttcgtgcggacgtgcccggcgctgcctgccaaggagggacccatgggcaacgaaccgcagacactgacggaaatgccgctctgggtactgatcctgctcgccgcgctgggcggcgtcagcggcgagatgtggcgtgccgacaaggccggtctcggcggctgggccttgttgcggcgcctggcgctgcgctccggcgcgtcgattgtctgcggcgtggcggtgatgctgctggcattggcttgcggcgccgcgctgctgttcgccgcggcgctgggcagcctgaccgccgcggccggcgcggagatcgcagtcggtctctacgaacgctgggccgcccggcgcctgggggtttgcgagctgcccgaggaacagtcggacgaccgtggcccgcattgaaactgatcgaaggagtcaaccatgcctgaacaggctgttacgctcgaggctctgtacgcggccatcgagcaggtactgcgtgagcgtctgccggaggcgcagttgatcggcttctggccaggcgtgccggaaaataccccggcggtttccctggaaatggccgagctactgcccgagcgcgatcccggtaccggcgagagtgccctgctgtgccgcctgcaggcgcggataatggtgccgcctggtgccgatcgccaggcggtatccattgcttgcggaatcgttcggacattgcgcgagcagacctggaacctgtctctgcagccggcgcgcttcgtacgctcggccgtcgacggcagtcgcgaggagctgaagagcctgcgtgtctggctggtcgagtggacgcagtcgctgcgtctcggagacccggagtgggcctgggaggaccagccgcccggcagcctgatgctgggcttcgacccgcagaccggccccggccatgagccggactacttcgctccggaggcgttggcatgagctatgtcagtgcggagcatgaccgcatgctcgccgcgatgatcctgccctgcgtggtggtcgccgtggacctggcagcggcgcgggtacgggtgcgctccggcgactggaccagcggctggctgcgctggcactccctggcagccggcaaggttcgccactggcgtgcgccgagcataggcgaacagggggtactgctcagtccgtcgggcggagtgtcaatgggtacctttattcccggtctgtacggcgatgcgggcacggcgccggacaacagcgccagcagtgagacctggcgtttcgacgacggcgcctcgttgagttacgactgggctgcgcatcgctaccgcgtcgagctgcccagcggcaccgtggaagtgagggtcggcgccagcgaggtgcgggtcagcgacggggcggtcagtctcaaggcgccgaagatcagcctggaaggaccggtggagatcgccgggacactgacggtcagcggagacatcctcggcggcggctcgatcatcgacaccgccggcaacagcaaccaccacacccattgaacatacgacggggctgccgaagggcggtccgtcgaacattcaaccggcccgcgcgagcgggccttttcgtttgcggagttcgccatgggcaacactcacagccattcggagccaggcgcggcctgtctctgccggagcgggagggcagggcgatgatcgggatggatcgccgtagcgggctgcccctgtccggcctggctcatcttaaacagtccgtcgaggacatcctgaccaccccgttgggcagcaggcgcatgcgccccgagtacggcagcaagctgcggcggatggtcgacatgccggtgagcgaaggctggaaaagcgccgtgcaggccgaggtagcccgttccctggggcgctgggaaccgcgcatcgcattgtctgccgtgcgagtcgtcgcagtcgtcgatggccgcgtggatctgctcctgagcggcgtgttcgagggcgagaacatcaatatggaggtctcggcgtgatcatcgatctttcccagttgccggagccagaggttatcgaaaacctcgatttcgagacgatttaccaggagctgttgggtgacttccgcgaagccatggctggcgaatggacagcggaggtggagtccgatccggttctcaagcttctgcaactggcggcctatcgagaactgctgctgcgggcgcggatcaacgatgcggcgcgggcggtgatgctggcatacgccagcggtgccgatctcgaccagatcggtgccggcttcaatgtgcagcgtttgctgatcaggcccgctcagcccgaggcggtaccgccggtggaggcgcaatacgagagcgacaagtcgctgcgcaatcgcatccagctcgcgttcgagcagctatccgtcgcaggaccgcggaacgcctatatagcccatgcgctgggcgcggatggaagggtggcggatgcctctgcgaccagtccggcgccctgcgaagtgctgatcagcgtgctcggggtggaaggcaacgggcaggcaccggaagcggtgttgcaggcagtgcgcctggcgctgaacgcggaggacgtgcgtcctgtcgcggatcgggtaacggtgcgctcggcaggaatcgttccctatcaggtcaaggcgcagctctacctgtttcccggtcccgaggcggaactgatccgtgccgccgccgaggcttcgctacgcgactacatttccgcccagcgccgcctgggccgcgacatccggcgttcggccctgttcgccaccctgcatgtcgaaggcgtgcagcgcgtcgaactgcaggagcctgcggccgacgtggtcctggatgaaacccaggcggcctattgcacggggtacgcgatcaccttgggaggcgtcgatgagtagccgactgctgccgccaaacaggagttcactggaacgctctctgggtgatgtattgcctgccgaactgccggtgccgcttcgtgagcttaacgatccggcacgctgtgaggcggccttgttgccctacctggcctggacgcgctcggtggaccgctgggacccggactggagcgacgaggccaagcgcaatgcggtagcgacgtccttcgtcctgcaccagcgcaaaggcacgctgaccgcgttgcgccaagtggtcgagccgatcggtgcgctgagcgaggtcaccgaatggtggcagcgaagcccgaccggcgtgccggggaccttcgagatcaccgtggacgtcagcgaccgtggcatcgacgaaggcaccgtactggagctggagcgcttgctcgatgacgtccgcccggtgagccgacacctgacccgcctggacctgcgcatcaccccggtaatccggtcccgtcacggactggccgtgaccgacggcgacaccctggaaatcttcccctggaaacagtgacatgacgaccaatactccgaaatacggtggcctgctcaccgacataggtgccgctgcgctggctgcggccagcgcagcaggcaagaaatggcagccgactcatatgctgatcggcgatgctggcggcgcaccgggtgctactccggatccgatacccgcagccacgcagacgaaactgatcaaccagcgctaccgggcgcagctcaatcgcttgttcgtgtcggacaagaacatcaatactctggttgccgaggtggtgctaccagttgaggtaggtggcttctggatccgcgagatcggcctgcaggatgccgacggcaagttcgtcgcggtatccaactgcccgcccagctacaaggcagcaatggaaagtggcagtgcgcggacccagaccattcgggtgaacatcgcgctctccggcctggagaatgtccagttgctgatcgacaacggcatcatctacgccactcaggactgggtgaaggaaaaggtcgctgccgatttcaagggccgcaagatcctggctggcaatggcctggtcggtgggggcgatctttctgccgaccgcagcattggcctggcgccttccggcgtgacggcgggcagctatcgctcggtcacggtgaacgccaacggggtggtcacccagggcagcaatccgagcaccctggccggctatgcgatcggagatgcctataccaaggccgataccgacggaaaactggcgcagaaagcgaacaaggccaccaccctggccggctatggcatcaccgatgcgctgcgagtcgatggcaacgccgtgtcatccagcaggctggccgcaccgcgtagcctggcagccagtggcgatgcctcctggtcggtgaccttcgacggcagtgccaatgtttctgcgccgctgagtctttccgctaccggtgtggcagcgggcagctatccgaaggtgaccgtggatacgaagggaagggtgactgctggaatggcgctggcggcgacggacattcccgggctggatgcttcgaagttggtcagcggggtgctggccgagcagcgtttgccggtattcgcgcgcgggttggctactgctgtctcgaccactagcgatccgaacaccgcgaccgtgccgttgatgctgaccaatcatgcgaacggacctgttgccggacggtacttctacatccagtcgatgttctatccggatcagaacggcaatgcttcgcagattgcaacgagctacaacgctacatccgagatgtatgtacgggtgtcctacgcggccaaccctagcgcccgggactggctgccctggaagcgctgcgacataggtggttcgttcagcaaggaggcggacggggccttgggcggtgcggtcaatctcaactcgctgatcacgtcgggatggtggtaccagacggccaatgcacaggccgaaagtggggcgaactacccggtcccccgggccggcttgcttcaagtgcataatgcaggcaccaatttcatctaccagacataccaggtttatgacggtgaagggttctattttcgttgccgctacaccaacacctggtatccatggcggcgtgtatggcatggagcggacttcaatccgaacgactatctgctgaaaagtggctttacgtgggccgccctgccaggaaagccagcgacctttccgcctactggccacaaccacgacgccgcccagatcacgtcgggcatcctgcctctggcgcgcggcggtcttggttcgaatacggcagcgggggcgcgcaacaatatcggggctggggttcccgcgacggcgaaccgatcgctgaatggctggtggaaggataacgatacaggactcatcgtgcagtggatgacagtgagtgtcggcgatcatccgggtggaatcgtcaaccgctcgctgacctttccgatcgcgtttcccactacctgcctgcacgtggtgccgagcgtcaaggaactggggcggccggcgacgtcggcatcgaccgtcaccctcgcagatgtcagcgtcagcaccacggggtgtgtgattgtcgctaccgagtatcacggtgcggtccagaactatgcgatcaggcttgtggccattggctgttgaggtgacccatgatttttttccatgccgccactggcggtttctattcgaaggatgtccacggcgaccgcatgcccatcgacgcgcggatgtatcccctcgaggaagctgaatacctggcgctgttggtagcccagagcgagggcaagcagatcgtcgccgacgccgccggccgtccgttctgtatcgacccgccggccccggcagaagaggtcctggcccaccgtgagcgcatctggcgcgatcgccagttgacgcttactgatggccccatcgcccgtcaccgggatgagctcgacctgggcaagattacgaccctgaaccaggcgcaactgctcgaactcacgctgtaccgcgccagccttcgagactggccggcatccgcggcgtttcctgatttaggcgcaagacccgagccgccgctgtggctcgaaccgctcatcaccccctgaaccccgccccgtgcggggtttttcattaatggagatctacctatgagtttcttccacggcgttacggtaaccaacgtcgatatcggtgcgcgcaccatcgcgctgccggccagctccgtcatcggcctctgcgatgtgttcacgccgggggcgcaggcaagcgccaagcccaacgttccggtgctgctcaccagcaagaaggacgccgccgcggcgttcggcatcggctcgtctatctacctggcctgcgaggccatctataaccgtgcccaggccgtgatcgtggcggtgggcgtggaagccgcggagactcccgaggcccaggccagcgccgtcataggtggtgtcagcgccgccggcgagcgcaccggacttcaggctctgctagacggcaagagccgcttcaatgctcagccgcgtctactagttgcgccgggtcattcggcccagcaagcggtggccaccgccatggacgggctggccgagaaactgcgggccatcgccattctcgatggccccaatagcaccgacgaggcggccgtcgcctacgccaagaacttcggcagcaagcgcctgttcatggtcgacccgggcgtgcaggtttgggacagcgccaccaatgccgcacgcaaggccccggcttcggcctacgccgccggcctgttcgcctggaccgacgccgagtacggcttctggtcgtcgccgtcgaacaaggagatcaagggcatcaccggcaccagccgtccggtggagttcctcgacggtgatgagacctgtcgcgccaacctgctcaacaacgccaatatcgccacgatcattcgcgacgatggctatcgcctgtggggcaatcgcaccttgtccagcgacagcaagtgggctttcgtcacccgcgttcggaccatggacctggtgatggatgcgatcctcgccgggcacaagtgggcggtggaccgcggcatcaccaagacctacgtgaaggatgtcaccgagggcctgcgcgccttcatgcgcgatctgaagaaccagggcgcggtgatcaacttcgaggtctatgccgacccagacctgaacagcgccagccagctggcccagggcaaggtgtactggaacatccgcttcaccgatgtgccgcctgccgagaaccccaatttccgtgtcgaggtgaccgatcagtggctcaccgaagttctggatgtcgcctaaggagcgcccgtgatgattccgcaaaccctgaccaataccaacctgttcatcgacggcgtgagcttcgccggtgacgtgccatccctgaccctgcccaagctggctgtgaagaccgagcaataccgtgccggcggcatggatgcgccggtatccatcgacatgggcctggaggcgatggaggccaagttctccaccaacggtgcccgccgagaagcactgaatttcttcggcctggccgaccagagcgctttcaatggcgtgttccgcggctccttcaagggccagaagggcgccagcgtgccagtggtggctaccctgcgcggcctgctcaaggaagtcgacccgggcgactggaaggccggcgagaaagccgagttcaagtacgccgttgcggtcagctactacaagctggaagtcgatggccgcgaggtctacgagatcgatccggtcaacggtgtccgcgcgatcaacggtgtcgaccagttggccggcatgcgcaacgacctcggcctgtaagaggagctccggacatgacccaagagaatcgactgccgggctggctgacgctggatgctgacgccgccctcgttcgtctctcgcgtccggcacagtgcaacggggtcagcgtcgacacgctgaccctgcgtgcacccaccgtgcgtgatatccgcctggccggcaaggtggccggcgacgatgccgaggagcgcgaactgcaactattcgcctcgctggcgcaggtcagccgccaggacctggaggggctgaagctgagcgactaccagcggctgcaaggcgcctactttcgcctggtgcaagacgacacggacgacaccttcgcgtatgcgtcaactggcgaggcgcctggccattgagctgggcttccaggccagcgaactggagcgcatgaccctgggcgacctgctctggtggctcgccgagggcgaggagtgagcgacggcgccgtccgccaggcgagcggcgcccgcgctacttcagtaggtccgcggaggcggacatcgataacagacgagacgaaccgtcatgagtaaagacatggacctggtggtctccatcggcggcatcgccgacccctccttgggcaaggcattcgagaccgtcaaggcacggctcgacagtctccaggaacgttcccgccaggcgtccagcctgagggatgtgctgggagacgccatacgcctggagcgagaactcgccgatatgcgcaaggtcggggaccgcggcgttgctgagcatgcccggcagcttggcgaacgccaggagcaactgaagcgactcggcatcgaggccagggccgcgggcgatgcctacgctcgactgggcgagatgcagcgtggcctggatatgcaggtccgcggcctgcaacggctggagcaggccagccaggcaatgccattggctagcgcattttccggactggtcgtggaagccagcaagacggctgccggttatcaagcgcggttgcgcgacctggcgatccgcaacggcctggacgtcggccgggagccagccttggcatccctgatccaggacagtgccagccagagcggcctgggacgcacggcgacgctggacatgctggagcacttgaacgccaccggcatggggttcgccgccgcgcaaatgaatctgggactggcgggccgcttcggctttggccaagggattgcttcagccgaggttgcggggctggttcgagcgttgcaactggcccagggttcggactcgccagagcaattgtccgcctccctcgaccgcctggtcgtcctgggtaaaggcagagtcggcagtgaggccctggcgcgtcgcctgcctgccttgttgtcagcgctgggcaatgcgggagaggccacggccggcgatgtcggtgcactgggggccttgctggagattcaggcaaagaacaccacgccagacaaagcggacgtgcggatgaaggcctggctggagttcgtcggcagcggcagcttgaaacgcgcttatggccaggactacgaccgggacctggaagcgctgcgcaaggacggagcgagtctgctggaggccaacctggagctggctgcgcgctatcgggataagggcggcaagctcagcgccggcgtggcgtcgccggcgcttgaagcctatcgagcgtcgcgcggcgagttccagggcttgctcgaatcccagcagtcttccgtaggctcttcggagcgcgatgcgcagcgtcgcaaggggatgtcccaggagctgtggaaggcttccagcgacagttgggagagggcgcagaccgctttgggcagcgccctgaatccatatctggacaacctggccaaggggagcgcggtactcggcgagtcgactgcggagctgctcgaagcctatccgcggacgacggccggtcttaccgccgccgcaggtgcggtgttatccgggtatctcgcctacaagggagggcgcggcgctatcgacgtgctgcgtggcggtcggctcggtcggcgagggaccgctgccgtcggcgacctgatcgaacggggtgcaggccgggtatcgggtggtagcgaaatacagcgcgtgttcgttaccaactggccggtgccgggaggcgactctacgctggagtccgcgaggaggccagcacaacgaaagcgcggtcaaacaccacgcaggaagagaggcaagggcggtgggctgaaggcccgctcccttccatcccttggcttctcggctggaggaggcttgggggcgatggcaggaaagctgccgcgcctgtcgcgtttgcccatacgcaacgcgccgctgcaagtggcctcgtcgttgatcgatgttgctgaggtctactccagcgacctgtcggagagcgaaaagaccgttgcctatggcgaggcaggtggctccttggctggttccctggctggcgccgctctgggagcgagcatcggctcggtggtgccggtggtcggtacgctgatcggtggattggttggcggcgctatcggcgcctggggcggtagcgaactgggggggcgcctggggcgcagtctggctggcgatccgccggcggcctcggacaacaagccggcggtggccgtaccacaggccggacccgtcgcggctgcgcccaactggaccttcgcgccgcagatcaacctgacggtgcaaggcaacgtgcacgagccgcagcgcctggccgacgagttgctgccctacctgcaacgcatgcttgtcgacttcgccgacgagcggcagcggcgcagcctctacgacccggcgatggtttaaggagtccccatggcctatctggaacaattgcaggccggcctgaggtacctgggtcgcgccggcgagtccggacgcaagagtctggacaaggtggtcgctccggtgaacggcgcgatcagcgagatccgcggcgcagccgcggagctggagaacctgcccggcgtatcgccggaaatggctgcccggctgcagcgtgccatgcgcggcatcggccaggcgcagggcaaggtgaaccgcgtggtctccacctatgaccgggcgagccgggcgttgcttggtatcgacgaacgcctggatgcgctgaaggtgcaggtgaacagtgccgcgcaggcggtcggcaaggtcgctggcgacatcagtccgacgctggcgggggtgctgccgtcgtggctgctggcaccctcggcgacgcccccgagcgaggccgcagcgcccttgccgcacctgctggtactgcagccgctgaccgccaatgcccaaccgttctacttcaacctgaatactgccgccttcgacgccctgcagcgcaacagtgcctacaactggagcgggcaggtgcgcctgggtcggcggccggcgctgcagagcgtcggcatgggcgaggagagcatcctgctcaagggtgcggtattcccgctgcgtcgacaggtaggtaaccaggaaaaggtcgtcggtctggagcagctcgaaacgctgcgccggttggcggagcggcgtgagccgctgatcctgagcagcggctacggcgaggtgcagatgggcctctggtgtctggtgcggatcagcgagaaccagagcgccctactgggcaacggcgctccccgcaaacaaaccttcgacctggagttcaagcgctatggcgacgacctgccgaaccgctgacggcgacatgctggacagcctctgctaccacgtctatggccatctgttgggctgcgtcgaggcgaccctcgacgccaatcccgggctggccgatgagcagcagccattccgcgccggcttgctgatcagtttccctgacatgccggtggtcaatgtcgaacaggtgcgcctgtgggattgatcgaccgctcacccgcaaccccgccttggcggggtttttcttttctggagaaaccaggtgcaaccgagtttccgtatcgttgccgacggcaccgacgtcacccagcggctgaatgatcgcctgctcaagctgaccctgctggacaagccgggcatggagtccgacagcctgaccttgaggatcgacgatcgcgatggacaggtggccttacccaggcgcggtgcggtgctggaggttcatctcggctatgccggcgagccactgatgcgcatgggacgctttaccgtggacaccttgcagtgggctggtccgccggactgcctgaccgtcactgccaaggccggcgacatgcgcggcagtggcaagacgatacgcagcggaggttgggagggcactaccctggctcaggtctgccgcgatgttggcgcacgcaacggctggcgcgtggagtgtccgttgcaggtggcgatcgcccgggtcgaccaggtcaatgagtccgactaccacttcgtcacccgtctggcgcgccagtacgactgcaccgccaagctggccgagggcatgctcatggtgctgccgcgacagagcgggcagagcgccacggggcgtcggatcgaacctttggtgttgggacgtgctgacgtcggtagcttcgacgttaccttcgacgaccgcagcctgatgagaacggtgaagacccgctaccaattgcccggcagcggcgaggtcaagagcgtcgagttgaagaacccgaaggcaccggctacggctatgggcgagcatgtcgaccggcacctctataccagccgtggagaggccgagcaggcggcgaaggctcgcctggcgagcttcagccgctccagcgccagtgtgcgcctgggactgccagggcgtggcgacctgttcgccgagcgcagcctgctgctccaaggcttcaaggcgggaatcgacggcgagttcctgatcgactcggtggagcacacctacagctccagcggatggaccactgtcgtgcaatgcaacggcggccgaggcggcaaggggtga

SEQ ID NO: 12 - DNA, 2109 bp, Pseudomonas aeruginosa
  misc_feature 1..2109: note = Description of sequence: polynucleotide, name: R5 tail
  source 1..2109: mol_type = genomic DNA
atgacgaccaatactccgaaatacggtggcctgctcaccgacataggtgccgctgcgctggctgcggccagcgcagcaggcaagaaatggcagccgactcatatgctgatcggcgatgctggcggcgcaccgggtgctactccggatccgatacccgcagccacgcagacgaaactgatcaaccagcgctaccgggcgcagctcaatcgcttgttcgtgtcggacaagaacatcaatactctggttgccgaggtggtgctaccagttgaggtaggtggcttctggatccgcgagatcggcctgcaggatgccgacggcaagttcgtcgcggtatccaactgcccgcccagctacaaggcagcaatggaaagtggcagtgcgcggacccagaccattcgggtgaacatcgcgctctccggcctggagaatgtccagttgctgatcgacaacggcatcatctacgccactcaggactgggtgaaggaaaaggtcgctgccgatttcaagggccgcaagatcctggctggcaatggcctggtcggtgggggcgatctttctgccgaccgcagcattggcctggcgccttccggcgtgacggcgggcagctatcgctcggtcacggtgaacgccaacggggtggtcacccagggcagcaatccgagcaccctggccggctatgcgatcggagatgcctataccaaggccgataccgacggaaaactggcgcagaaagcgaacaaggccaccaccctggccggctatggcatcaccgatgcgctgcgagtcgatggcaacgccgtgtcatccagcaggctggccgcaccgcgtagcctggcagccagtggcgatgcctcctggtcggtgaccttcgacggcagtgccaatgtttctgcgccgctgagtctttccgctaccggtgtggcagcgggcagctatccgaaggtgaccgtggatacgaagggaagggtgactgctggaatggcgctggcggcgacggacattcccgggctggatgcttcgaagttggtcagcggggtgctggccgagcagcgtttgccggtattcgcgcgcgggttggctactgctgtctcgaccactagcgatccgaacaccgcgaccgtgccgttgatgctgaccaatcatgcgaacggacctgttgccggacggtacttctacatccagtcgatgttctatccggatcagaacggcaatgcttcgcagattgcaacgagctacaacgctacatccgagatgtatgtacgggtgtcctacgcggccaaccctagcgcccgggactggctgccctggaagcgctgcgacataggtggttcgttcagcaaggaggcggacggggccttgggcggtgcggtcaatctcaactcgctgatcacgtcgggatggtggtaccagacggccaatgcacaggccgaaagtggggcgaactacccggtcccccgggccggcttgcttcaagtgcataatgcaggcaccaatttcatctaccagacataccaggtttatgacggtgaagggttctattttcgttgccgctacaccaacacctggtatccatggcggcgtgtatggcatggagcggacttcaatccgaacgactatctgctgaaaagtggctttacgtgggccgccctgccaggaaagccagcgacctttccgcctactggccacaaccacgacgccgcccagatcacgtcgggcatcctgcctctggcgcgcggcggtcttggttcgaatacggcagcgggggcgcgcaacaatatcggggctggggttcccgcgacggcgaaccgatcgctgaatggctggtggaaggataacgatacaggactcatcgtgcagtggatgacagtgagtgtcggcgatcatccgggtggaatcgtcaaccgctcgctgacctttccgatcgcgtttcccactacctgcctgcacgtggtgccgagcgtcaaggaactggggcggccggcgacgtcggcatcgaccgtcaccctcgcagatgtcagcgtcagcaccacggggtgtgtgattgtcgctaccgagtatcacggtgcggtccagaactatgcgatcaggcttgtggccattggctgttga

SEQ ID NO: 13 - protein, 702 aa, Pseudomonas aeruginosa
  REGION 1..702: note = Description of sequence: peptide, name: R5 pyocin tail fiber
MTTNTPKYGGLLTDIGAAALAAASAAGKKWQPTHMLIGDAGGAPGATPDPIPAATQTKLINQRYRAQLNRLFVSDKNINTLVAEVVLPVEVGGFWIREIGLQDADGKFVAVSNCPPSYKAAMESGSARTQTIRVNIALSGLENVQLLIDNGIIYATQDWVKEKVAADFKGRKILAGNGLVGGGDLSADRSIGLAPSGVTAGSYRSVTVNANGVVTQGSNPSTLAGYAIGDAYTKADTDGKLAQKANKATTLAGYGITDALRVDGNAVSSSRLAAPRSLAASGDASWSVTFDGSANVSAPLSLSATGVAAGSYPKVTVDTKGRVTAGMALAATDIPGLDASKLVSGVLAEQRLPVFARGLATAVSTTSDPNTATVPLMLTNHANGPVAGRYFYIQSMFYPDQNGNASQIATSYNATSEMYVRVSYAANPSARDWLPWKRCDIGGSFSKEADGALGGAVNLNSLITSGWWYQTANAQAESGANYPVPRAGLLQVHNAGTNFIYQTYQVYDGEGFYFRCRYTNTWYPWRRVWHGADFNPNDYLLKSGFTWAALPGKPATFPPTGHNHDAAQITSGILPLARGGLGSNTAAGARNNIGAGVPATANRSLNGWWKDNDTGLIVQWMTVSVGDHPGGIVNRSLTFPIAFPTTCLHVVPSVKELGRPATSASTVTLADVSVSTTGCVIVATEYHGAVQNYAIRLVAIGC

SEQ ID NO: 14 - DNA, 840 bp, Pseudomonas aeruginosa
  misc_feature 1..840: note = Description of sequence: polynucleotide, name: galU
  source 1..840: mol_type = genomic DNA
atgatcaagaaatgtcttttcccggccgccggttacggcacccgtttcctccccgccaccaaggccatgcccaaggaaatgctgccggtggtgaacaagccgctgatccagtacgcggtggaggaggcgctggaagccggcctttccgagatcggcatcgtcaccggccgcggcaagcgttcgctggaagaccacttcgacatcagctacgagctggaacaccagatccgcaacaccgacaaggaaaagtacctggtcggcatccgtcggctgatcgacgagtgcaccttcgcctacacccgccaggtggagatgaagggcctcggccacgccatcctcaccggtcgtccgctgatcggcgacgagccgttcgccgtggtcctggccgacgacctgtgcctgaacctcgaaggcgacagcgtgctgaagcagatggtcaagctgtacaaccagttccgctgctccatcgtggcgatccaggaagtgccgccggaagagaccaacaagtacggcgtgatcgccggcgagatgatccgcgacgatatcttccgggtgaacaccatggtcgagaagccgaagccggaagaggcgccgtcgaacctggcgatcatcggccgctacatcctgaccccggacatcttcgacctgatcgagcagactgaaccgggcaagggcggcgagatccagatcaccgatgccctgatgaagcaggcccaggacggctgcgtgctggcctacaagttcaagggcaagcgtttcgactgcggcagcgccgagggttacatcgaggcgaccaacttctgctacgaaaacctctacaagaccggcaaggctcactga

SEQ ID NO: 15 - DNA, 1299 bp, Pseudomonas aeruginosa
  misc_feature 1..1299: note = Description of sequence: polynucleotide, name: hmgA
  source 1..1299: mol_type = genomic DNA
atgaacctcgactccactgccctcgcctatcaatcgggcttcggcaacgaattcagcagcgaagcgctccccggcgccctgccggtcggccagaactccccgcagaaagcgccctacggcctgtacgccgaactgctctccggcaccgccttcaccatggctcgcagcgaggcccggcgcacctggctgtaccgcatcacgccgtcggccaagcatccgccgttccgccgcctggaacgacagatcgccggtgccgaactggatgcgccgactcccaaccgcctgcgctgggacccgctggcactgcccgagcagcccaccgacttcctcgacggcctgctgcgcatggccgccaacgcgcccggcgacaagcccgccggcgtgagcatctaccagtacctggccaaccgctcgatggagcgttgcttctacgacgccgatggcgaactgctgctggtcccgcaattgggccgtctgcgcctgtgcaccgaactcggcgcgctgcaagtcgaaccgctggagatcgcggtgatcccgcgcgggatgaagttccgcgtcgagctgctcgacggcgaggcacgcggctatatcgccgagaaccacggcgcgccgctgcgcctgcccgacctcggcccgatcggcagcaatggcctggccaatccgcgcgacttcctgaccccggtggcgcgctacgaggacagccgccagccgctgcaactggtgcagaaatacctcggcgagctgtgggcctgcgagcttgaccactcgccgctggacgtggtcgcctggcacggcaacaacgtgccctacaagtacgacctgcgccgcttcaacaccatcggcacggtcagcttcgaccacccggacccgtcgatcttcaccgtgctgacctcccccaccagcgtccccggcctggccaacatcgacttcgtgatcttcccgccgcgctggatggtggccgagaacaccttccgtccgccatggttccaccgcaacctgatgaacgaattcatgggcctgatccagggcgcctatgacgccaaggccggcggcttcgtgcccggcggcgcctcgctgcacagttgcatgagcgcccacggcccggacgcggaaagctgcgacaaggccatcgccgccgacctcaagccgcacaggatcgaccagaccatggccttcatgttcgagaccagccaggtcctccggccgagccgtgccgccctcgagacgccggccctgcagaatgactacgatgcctgctgggcgtcgctcgtatccaccttcaacccgcaacggagataa

SEQ ID NO: 16 - DNA, 1191 bp, Pseudomonas aeruginosa
  misc_feature 1..1191: note = Description of sequence: polynucleotide, name: MexX
  source 1..1191: mol_type = genomic DNA
atgcacatccaatggaccggctcgctgcgcgggctgctggcggccctggtcgccctattcctgctgggctgcgaagaagcagcggacgccgggaagactgcggaggcccccgccgaggtcggcgtgatcgtcgccaggccggcgcctatcggcatcaccagcgagctgcccggacgcctggaagcgtaccgccaggctgaagtgcgggcgcgcgtcgccggcatcgtcacccgtcgcctgtacgaggaaggccaggacgtccgcgccggcaccgtgctgttccagatcgaccctgcgcccttgaaggcggccctggacatcagccgcggcgccctggcccgggccgaggccagccacgcggcggcggccgacaagctcaagcgctacgccgacctgatcaaggaccgcgccatcagcgaacgcgagtacaccgaagcgcagaccgacgcgcgccaggccctggcgcagatcgcctcggccaaggccgaactggagcaggcccgcctgcgcctgggctacgccacggtcaccgcgccgatcgacggccgcgcgcggcgtgcgctggtcaccgaaggcgcgctggtcggcgaggactcgccgacaccgctgacccgcgtcgagcagatcgatccgatctacgtgaacttctcccagccggccggcgaagtcgccgccatgcagcgggcgatccgcgaaggccaggtgaagggtgtcgccgacaaggacatcgccgtgcgcctggtcctggccgacggcagcgagtacccgctggccggcgagctgctgttctccgacctggcggtcgaccccggcaccgacaccatcgccatgcgtgccctgttccgcaatccgcatcgcgaattgctgcccggcggctacgtgcaggtgcgcctgcagcgcgcggtgaacccgcaggcgatcaccgtcccgcgcgacgcgctgatccgtaccgcccagtccgccgtggtcaaggtggtcaacccaaagggcttggtggaagacgtggaggtccgcgccgacaccctgcagggccgcgactggatcatcagccgcgggctcaagggcggcgagtgggtgatcgtcgagaacgccgcccagcatgccgccggctccagcgtccaggcggtggtccgccagccggccagcgccgacgccccctcaccgctggccgcctcgccggcgggccagtga

SEQ ID NO: 17 - DNA, 3138 bp, Pseudomonas aeruginosa
  misc_feature 1..3138: note = Description of sequence: polynucleotide, name: MexY
  source 1..3138: mol_type = genomic DNA
atggctcgtttcttcattgaccggccggtcttcgcctgggtgatctccctgctgatcgtgctcgccggggtcctggcgatccgcttcctgccggtcgcccagtacccggacatcgcgccgccggtggtcaacgtcagcgccagctatcccggcgcctcggccaaggtggtcgaggaagcggtgaccgcgatcatcgagcgcgagatgaacggcgcgcccggcctgctctacaccaaggccaccagcagcaccggccaggcctcgctgaccctgaccttccgccagggcgtgaacgccgacctcgccgcggtggaagtgcagaaccgcctgaagatcgtcgagtcgcgcctgcccgaatcggtgcggcgcgacggcatctacgtggagaaggcggcggacagcatccagctgatcgttacccttacctcctccagcggccgctacgacgccatggagctgggcgagatcgcctcgtccaacgtgttgcaggcgctgcgccgggtggagggcgtgggcaaggtcgagacctggggcgccgagtacgccatgcgcatctggcccgacccggccaagctgacctcgatgaacctcagcgccagcgacctggtcaacgccgtgcgccggcacaacgcccgcctcaccgtgggcgacatcggcaacctcggggtccccgactcggcgccgatcagcgccacggtgaaggtcgacgacaccctggtgacgcccgagcagttcggcgaaattccgctgcgcatccgcgccgacggcggcgcgatccgcctgcgcgacgtggcccgcgtcgagttcggccagagcgagtacggcttcgtctcgcgggtcaaccaaatgaccgccaccggcctggcggtgaagatggcgcccggctccaacgcggtggccaccgccaagcgcatccgcgccaccctcgacgagctgtcgcgctacttcccggagggcgtgagctacaacatcccctatgacacctcggcgttcgtcgagatctcgatcaggaaggtggtcagcaccctgctcgaggcgatgctgctggtgttcgccgtgatgtacctgttcatgcagaacttccgcgccaccctgatcccgacactggtggtgccggtggccctgctgggcaccttcacggtgatgctcggcctgggcttctcgatcaacgtgctgaccatgttcggcatggtcctggcgatcggcatcctggtggacgacgcgatcatcgtggtggagaacgtcgagcggctgatggccgaggaaggcctgtcgccgcacgacgccacggtcaaggcgatgcgccagatcagcggggccatcgtcggcatcaccgtagtgctggtctcggtgttcgtgccgatggcgttcttcagcggcgcggtgggcaacatctaccgccagttcgcggtgaccctggcggtctccatcggcttctcggcgttcctcgcgctgtcgctgaccccggccctgtgcgccaccctgctgcgcccgatcgacgccgaccaccacgagaagcgcggcttcttcggctggttcaaccgcgccttcctgcgcctgaccggacgctaccgcaacgcggtggccggcatcctcgcccggccgatccgctggatgctggtctacaccctggtcatcggcgtggtcgccctgctcttcgtgcgcctgccgcaggcgttcctgccggaagaggaccagggcgacttcatgatcatggtgatgcagcccgaaggcacgccgatggcggagaccatggccaacgtcggcgacgtcgagcgctacctggcggagcacgaaccggtggcctacgcctatgcggtcggcggcttcagcctgtacggcgacggcaccagctcggcgatgatcttcgccaccctgaaggactggtcggaacgccgggaggccagccagcacgtcggcgccatcgtcgagcgcatcaaccagcgcttcgccggcctgcccaaccgtacggtgtatgcgatgaactcgccgccgctgccggacctgggttccaccagcggcttcgacttccgcctgcaggaccgtggcggggttggctacgaggccctggtcaaggcccgcgaccagttgctggcgcgcgccgccgaggacccgcgcctggccaacgtgatgttcgccggccagggcgaggcgccgcagatccgcctggacatcgaccggcgcaaggcggagacccttggcgtgagcatggacgagatcaacaccaccctggcggtgatgttcggctcggactacatcggcgacttcatgcacggcagccaggtgcgcaaggtggtggtccaggccgacggcgccaagcgcctgggcatcgacgacatcggccggcttcacgtgcgcaacgagcagggcgagatggtgccgctggcgacgttcgccaaggccgcctggaccctcggcccgccgcaactgacccgctacaacggctatccctcgttcaacctcgagggccaggccgcgccgggctacagcagcggcgaagccatgcaggcgatggagcaattgatgcagggactgcccgagggcatcgcccacgagtggtccggccagtccttcgaagaacgcctgtccggcgcccaggcgccggcgctgttcgccctctcggtgttgatcgtgttcctcgccctggccgccctctacgaaagctggtcgatcccgctggcggtgatcctggtggtgccgctgggcgtactcggcgcactgctcggggtgagcctgcgcggtctgcccaacgacatctacttcaaggtcggcctgatcaccatcatcggcctctcggcgaagaacgccatcctcatcatcgaggtggccaaggaccattaccaggaaggcatgagcctgctgcaggcgaccctggaggccgcgcgcctgcgcctgcgaccgatcgtcatgacctcgctggcgttcggtttcggcgtggtcccgctggcgctctccagcggcgccggttccggcgcccaggtcgccatcggcaccggggtgctcggcgggatcgtcaccgccacggtactcgcggtgttcctggtaccgctgttcttcctggtggtcgggcgcctgttccggttgcgcaaggcgccgcgcaccggcaactcgccccagatccccacggagcaagcctga

SEQ ID NO: 18 - DNA, 27165 bp, Pseudomonas aeruginosa
  misc_feature 1..27165: note = Description of sequence: polynucleotide, name: MRSN2101 R1 pyocin
  source 1..27165: mol_type = genomic DNA
tgaagcaccggaacccggccctggcgccgggttttttattgcccgggaaaacgttcgtgcggacgtgcccggcgctgcctgccaaggagggacccatgggcaacgaaccgcagacactgacggaaatgccgctctgggtactgatcctgctcgccgcgctgggcggcgtcagcggcgagatgtggcgtgccgacaaggccggtctcggcggctgggccttgttgcggcgcctggcgctgcgctccggcgcgtcgattgtctgcggcgtggcggtgatgctgctggcattggcttgcggcgccgcgctgctgttcgccgcggcgctgggcagcctgaccgccgcggccggcgcggagatcgcagtcggtctctacgaacgctgggccgcccggcgcctgggggtttgcgagctgcccgaggaacagtcgggcgaccgtggcccgcattgaaactgatcgaaggagtcaaccatgcctgaacaggctgtcacgctcgaggctctgtacgcggccatcgagcaggtactgcgtgagcgtctgccggaggcgcagttgatcggtttctggccaggcgtgccggaaaataccccggcggtttccctggaaatagccgagctgctgcccgagcgcgatcccggtaccggcgagagcgccctgctgtgccgcctgcaggcgcggataatggtgccgcttggtggcgatcgccaggcggtatccattgcttgcggaatcgttcggacattgcgcgagcagacctggaacctgtctctggaaccggcgcgcttcgtacgctcggccgtcgacggcagtcgcgaggagctgaagagcctgcgtgtctggctggtcgagtggacgcagtcgctgcgtctcggagacccggagtgggcctgggaggaccagccgccgggcagcctgatgctgggcttcgacccgcagaccggccccggccatgagccggactactttgctccggaggcgttggcatgagctatgtcagtgcggagcatgaccgcatgctcgccgcgatgatcctgccctgcgtggtggtcgccgtggacctggcagcggcgcgggtacgggtgcgctccggcgactggaccagcggctggctgcgctggcactccctggcagccggcaaggttcgccactggcgtgcgccgagcataggcgaacagggggtactgctcagtccgtcgggcggagtgtcaatgggtacctttattcccggtctgtacggcgatgcgggcacggcgccggacaacagcgccagcagtgagacctggcgtttcgacgacggcgcctcgttgagttacgactgggctgcgcatcgctaccgcgtcgagctgcccagcggcaccgtggaagtgagggtcggcgccagcgaggtgcgggtcagcgacggggcggtcagtctcaaggcgccgaagatcagcctggaaggaccggtggagatcgccgggacactgacggtcagcggagacatcctcggcggcggctcgatcatcgacaccgccggcaacagcaaccaccacacccattgaacatacgacggggctgccgaagggcggtccgtcgaacattcaaccggcccgcgcgagcgggccttttcgtttgcggagttcgccatgggcaacactcacagccattcggagccaggcgcggcctgtctctgccggagcgggagggcagggcgatgatcgggatggatcgccgtagcgggctacccctgtccggcctggctcatcttaaacagtccgtcgaggacatcctgaccaccccgttgggcagcaggcgcatgcgccccgagtacggcagcaagctgcggcggatggtcgacatgccggtgagcgaaggctggaaaagcgccgtgcaggccgaggtagcccgttccctggggcgctgggaaccgcgcatcagattgtctgccgtgcgagtcgtcgcggtcgtcgatggccgcgtggatctgctcctgagcggcgtgttcgagggcgagaacatcaatatggaggtctcggcgtgatcatcgatctttcccagttgccggagccagaggttatcgaaaacctcgatttcgagacgatttaccaagagctgttgggcgacttccgcgaagccatggctggcgaatggacagcggaggtggagtccgatccggttctcaagcttctgcaactggcggcctatcgagaactgctgctgcgggcgcggatcaacgatgcggcgcgggcggtgatgctggcatacgccagcggtgccgatctcgaccagatcggtgccggcttcaatgtgcagcgtttgctgatcaggcccgctcagcccgaggcggtaccgccggtggaggcgcaatacgagagcgacaagtcgctgcgcaatcgcatccagctcgcgttcgagcagctatccgtcgcaggaccgcggaacgcctatatagcccatgcgctgggcgcggatggaagggtggcggatgcctctgcgaccagtccggcgccctgcgaagtgctgatcagcgtgctcggggtggaaggcaacgggcaggcaccggaagcggtgttgcaggcagtgcgcctggcgctgaacgcggaggacgtgcgtcctgtcgcggatcgggtaacggtgcgctcggcaggcatcgttccctatcaggtcaaggcgcagctctacctgtttcccggtcccgaggccgaactgatccgtgccgccgccgaggcttcgctgcgcgactacatttccgcccagcgccgcctgggccgcgacatccggcgttcggccctgttcgccaccctgcatgtcgaaggcgtgcagcgcgtcgaactgcaggagcctgcggccgacgtggtcctggatgaaacccaggcggcctattgcacggggtacgcgatcaccttgggaggcgtcgatgagtagccgcctgctgccgccaaacaggagttctctggaacgctctctgggtgatgtattgcctgccgaactgccggtgccgcttcgtgagcttcacgatccggcacgctgtgaggcggccttgttgccctacctggcctggacgcgctcggtggaccgctgggacccggactggagcgacgaggccaagcgcaatgcggtagcgacgtccttcgtcctgcaccagcgcaaaggcacgctgaccgcgttgcgccaagtggtcgagccgatcggtgcgctgagcgaggtcaccgaatggtggcagcgaagcccgaccggcgtgccggggaccttcgagatcaccgtggacgtcagcgaccgtggcatcgacgaaggcaccgtactggagctggagcgcttgctcgatgacgtccgcccggtgagcagacacctgacccggctggacctgcgcattaccccggtaatccggtcccgtcacggactggccgtgaccgacggcgacaccctggaaatcttcccttggaaacagtgacatgacgaccaatactccgaaatacggtggcctgctcaccgacataggtgccgctgcgctggctgcggccagtgcagcaggcaagaaatggcagccgactcatatgctgatcggcgatgccggcggtgcgccgggcgacacgccggatccattgccttctgcggcgcagaagagcctgatcaaccaacgccatcgggctcagctgaatcggctgttcgtttccgacaagaacgccaataccttggttgccgaggtggtgctgccagttgaggtaggtggcttctggatccgcgagatcggcctgcaggatgccgacggcaagttcgtcgcggtatccaactgcccgcccagctacaaggctgcaatggaaagtggcagtgcgcggacccagaccattcgggtgaacatcgcgctctccggcctggagaatgtccagctgctgatcgacaacggcatcatctacgccactcaggactgggtgaaggaaaaggtcgctgccgatttcaagggccgcaagattctggctggcaatggcttggtcggtgggggcgatctttctgccgaccgcagcattggtctggcgccttccggcgtgacggcgggcagctatcgttcggtcacggtgaacgccaacggggtggtcacccagggcagcaatccgaccaccctggccggctatgcgatcggagatgcctataccaaggccgataccgatggaaaactggcgcagaaagcgaacaaggccaccaccctggccggctatggcattaccgatgcgctgcgagtcgatggcaacgccgtgtcatccagcaggctggccgcaccgcgtagcctggcagccagtggcgatgcctcctggtcggtgaccttcgacggcagtgccaatgtttctgcgccgctgagtctttccgctaccggtgtggcggcgggcagctatccgaaggtgaccgtggatacgaagggaagggtgactgctggaatggcgctggcggcgacggacattcccgggctggatgcttcgaagttggtcagcggggtgctggccgagcagcgtttgccggtattcgcgcgcgggttggctactgctgtctcgaacagtagcgatccgaacaccgcgaccgtgccgttgatgctgaccaatcatgcgaacggacctgttgccggacgatacttctacatccagtcgatgttctatccggatcagaacggcaatgcttcgcagattgcaacgagctacaacgctacatccgagatgtatgtacgggtgtcctacgcggccaaccctagcatccgggagtggttgccctggcagcgttgcgacattggaggctccttcacgaagacgactgacggatccattggaaatggcgtcaatataaacagcttcgtcaattccggatggtggttgcaatcgacatcggaatgggcggcgggtggagctaactatcccgtggggctggccggtttgctgattgtctaccgcgcacatgcagaccatatctatcagacctacgtaacactcaacggaagcacatattcgcgctgctgctatgcgggctcttggcgtccgtggcggcagaactgggacgatggaaacttcgatccggccagctacctgccaaaggcgggatttacctgggcggctttgccgggtaagccggcaactttcccgccctcagggcataaccacgataccagccagatcacctccggcatcttgcctctcgctcgtggtggccttggcgccaatacagcggccggagcacgcaacaacattggtgccggagtgccggccacggcgagccgggcgctcaatggttggtggaaggacaacgataccggtctgatcgtccagtggatgcaggtgaacgtaggagatcatcccggtggaataatcgatcgtaccttgacgttcccgatcgcgttccctggcgcctgtctgcatgtcgtaccgactgtcaaggaggtggggcgaccagcgacgtccgcgtcgaccgttacggtcgccgatgtcagcgtcagcaacacgggatgtgtgatcgtttcctccgagtactacggactggctcaaaactatggcatcagagtgatggccatcggctattgaggtgaaaatgatttttttccatgccgccacgggcggcttttattcgaaggaaattcatgggtcccgcatgcctctggaggatgaaatgcatcctctcgaagacgccgagtaccaggctcttcttcgtgcgcagagcgaagggaaacgaattgtcacggaccacaccggtcgccctatctgcgtcgatccaccggctccggccaaggacattctggtccaacgggaacgcatctggcgcgaccggcagttacagctcaccgacgggcctctcgctcggcatcgtgacgagcaggacctgggaaaaactacgactctgagccaggagcagcttcgtgagctaactctctatcgcgccgttcttcgcgactggcctattgccgcggagtttcccgacctgaacgcaaggcccgagccgcctgcctggctccaatcgctcatcaccccctgaaccccgccttgtgcggggtttttcattaatggagatctacctatgagtttcttccacggcgttacggtaaccaacgtcgatatcggtgcgcgcaccatcgcgctgccggccagttccgtcatcggcctctgcgatgtgttcacgccgggggcgcaggcaagcgccaagcccaacgtcccggtgctgctcaccagcaagaaggacgccgccgcggcgttcggcatcggctcgtcgatctacctggcctgcgaggccatctataaccgtgcccaggccgtgatcgtggcggtgggcgtggaggccgcggagactcccgaggcccaggccagcgccgtcataggtggtgtcagtgccgccggcgagcgcaccgggctgcaggctttgctagacggcaagagccgcttcaatgcccagccacgtctactagttgcgccgggtcattcggcccagcaagcggtggccaccgccatggacgggctggccgagaaactgcgggccatcgccattctcgatggtcccaatagcaccgacgaggcggccgtcgcctacgccaagaacttcggcagcaagcgcctgttcatggtcgacccgggcgttcaggtttgggacagcgccaccaatgccgcgcgcaacgccccggcttcggcctacgccgccggcctgttcgcctggaccgacgccgagtacggcttctggtcctcgccgtcgaacaaggagatcaagggcgtcaccggcaccagccgtccggtggagttcctcgacggcgatgagacctgtcgcgccaacctgctcaacaacgccaatatcgcgacgatcattcgcgacgatggctatcgcctgtggggcaaccgcaccttgtccagcgacagcaagtgggctttcgtcacccgtgttcggaccatggacctggtaatggatgcgatcctcgccgggcacaagtgggcggtggaccgcggtatcaccaagacctacgtgaaggatgtcaccgagggcctgcgcgccttcatgcgcgatctgaagaaccagggagcggtgatcaacttcgaggtctatgccgacccggacctgaacagcgccagccagctggcccagggcaaggtgtactggaacatccgcttcaccgatgtgccgcctgccgagaaccccaatttccgtgtcgaggtgaccgatcagtggctcaccgaagttctggatgtcgcctaaggagcgcccgtgatgattccgcaaaccctgaccaataccaatctgttcatcgacggcgtgagcttcgccggtgacgtgccatccctgaccctgcccaagctggctgtgaagaccgagcaataccgtgccggcggcatggatgcgccggtatccatcgacatgggcctggaggcgatggaagccaagttctccaccaacggtgcccgccgagaagcgctgaatttcttcggcctggccgaccagagcgctttcaatggcgtgttccgtggctccttcaagggccagaagggcgccagcgtgccagtggtggctaccctgcgcggcctgctcaaggaggtcgacccgggcgactggaaagccggcgagaaagccgagttcaagtacgccgttgcggtcagctactacaagctggaagtcgatggccgcgaggtctacgagatcgatccggtcaacggtgtccgcgcgatcaacggtgtcgaccagttggccggcatgcgcaacgacctcggcctgtaagaggagctccggacatgacccaagagaatcgactgccgggctggctgacgctggatgccgacgccgccctcgttcgtctctcgcgtccggcacagtgcaacggggtcagcgtcgacacgctgaccctgcgtgcacccaccgtgcgtgatatccgcctggccggcaaggtggccggcgacgatgccgaggagcgcgaactgcaactgttcgcctcgctggcgcaggtcagccgccaggacctggaggggctgaagctgagcgactaccagcggctgcaaggcgcctactttcgcctggtgcaagacgacacggacgacaccttcgcgtatgcgtcaactggcgaggcgcctggccattgagctgggcttccaggccagcgaactggagcgcatgaccctgggcgacctgctctggtggctcgccgagggcgaggagtgagcgacggcgccgtccgccaggcgagcggcgcccgcgctacttcagtaggtccgcggaggcggacatcgataacagacgagacgaaccgtcatgagtaaagacatggacctggtggtctccatcggcggcatcgccgacccctccttgggcaaggcattcgagaccgtcaaggcacggctcgacagtctccaggaacgttcccgccaggcttccagcctgagggatgtgctgggagacgccatacgcctggagcgagagctcgccgatatgcgcaaggtcggggaccgcggcgttgctgagcatgcccggcagcttggcgaacgccaggagcaactgaagcgactcggcatcgaggccagggccgcgggcgatgcctacgctcgactgggcgagatgcagcgtggcctggatatgcaggtccgcggcctgcaacggctggagcaggccagccaggcaatgccattggctagcgcattttccggactggtcgtggaagccagcaagacggctgccggttatcaagcgcggttgcgcgacctggcgatccgcaacggcctggacgtcggccgggagccagccttggcatccctgatccaggacagcgccaaccagagcggcctgggacgcacggcgacgctggacatgctggagcacttgaacgccaccggcatggggttcgccgccgcgcaaatgaatctgggactggcgggccgcttcggctttggccaagggattgcttcagccgaggttgcggggctggttcgagcgttgcaactggcccagggttcggactcgccagagcaattgtccgcctccctcgaccgtctggtcgtcctgggtaaaggcagagtcggcagtgaggccctggcgcgtcgcctgcctgccttgttgtcagcgctgggcaatgcgggagaggccacggccggcgatgtcggtgcactgggtgccttgctggagattcaggcaaagaacaccacgccagacaaagcggacgtgcggatgaaggcctggctggagttcgtcggcagcggcagcttgaaacgcgcttatggccaggactacgaccgggacctggaagcgctgcgcaaggacggagcgagtctgctggaggccaacctggagctggctgcgcgctatcgggataagggcggcaagctcagcgccggcgtggcgtcgccggcgcttgaagcctatcgagcgtcgcgcggcgagttccagggcttgctcgaatcccagcagtcttccgtaggctcttcggagcgcgatgcgcagcgtcgcaaggggatgtcccaggagctgtggaaggcttccagcgacagttgggagagggcgcagaccgctttgggcagcgccctgaatccatatctggacaacctggccaaggggagcgcggtactcggcgagtcgactgcggagctgctcgaagcctatccgcggacgacggccggtcttaccgccgccgcaggtgcggtgttatccgggtatctcgcctacaagggagggcgcggcgctatcgacgtgctgcgtggcggtcggctcggtcggcgagggaccgctgccgtcggcgacctgatcgaacggggtgcaggccgggtatcgggtggtagcgaaatacagcgcgtgttcgttaccaactggccggtgccgggaggcgactctacgctggagtccgcgaggaggccagcacaacgaaagcgcggtcaaacaccacgcaggaagagaggcaagggcggtgggctgaaggcccgctcccttccatcccttggcttctcggctggaggaggcttgggggcgatggcaggaaagctgccgcgcctgtcgcgtttgcccatacgcaacgcgccgctgcaagtggcctcgtcgttgatcgatgttgctgaggtctactccagcgacctgtcggagagcgaaaagaccgttgcctatggcgaggcaggtggctccctggctggttccctggctggcgccgctctgggagcgagcatcggctcggtggtgccggtggtcggtacgctgatcggtggattggttggcggcgctatcggcgcctggggcggtagcgaactgggggggcgcctggggcgcagtctggctggcgatccgccggcggcctcggacaacaagccggcggtggccgtaccacaggccggacccgtcgcggctgcgcccaactggaccttcgcgccgcagatcaacctgacggtgcaaggcaacgtgcacgagccgcagcgcctggccgacgagttgctgccctacctgcaacgcatgcttgtcgacttcgccgacgagcggcagcggcgcagcctctacgacccggcgatggtttaaggagtccccatggcatatctggaacaattgcaggccggcctgaggtacctgggtcgcgccggcgagtccggacgcaagagtctggacaaggtggtcgctccggtgaacggcgcgatcagcgagatccgcggcgcagccgcggagctggagaacctgcccggcgtatcgccggaaatggctgcccggctgcagcgtgccatgcgcggcatcggccaggcgcagggcaaggtgaaccgcgtggtctccacctatgaccgggcgagccgggcgttgcttggtatcgacgaacgcctggatgcgctgaaggtgcaggtgaacagtgccgcgcaggcggtcggcaaggtcgctggcgacatcagtccgacgctggcgggggtgctgccgtcgtggctgctggcaccctcggcgacgcccccgagcgaggccgcagcgcccttgccgcacctgctggtactgcagccgctgaccgccaatgcccaaccgttctacttcaacctgaatactgccgccttcgacgccctgcagcgcaacagtgcctacaactggagcgggcaggtgcgcctgggtcggcggccggcgctgcagagcgtcggcatgggcgaggagagcatcctgctcaagggtgcggtattcccgctgcgtcgacaggtaggtaaccaggaaaaggtcgtcggtctggagcagctcgaagcgctgcgccggttggcggagcggcgtgagccgctgatcctgagcagcggctacggcgaggtgcagatgggcctctggtgtctggtgcggatcagcgagaaccagagcgccctactgggcaacggcgctccccgcaaacaaaccttcgacctggagttcaagcgctatggcgacgacctgccgaaccgctgacggcgagatgctggacagcctctgctaccacgtctatggccatctgttgggctgcgtcgaggcgaccctcgacgccaatcccgggctggccgatgagcagcagccattccgcgccggcttgctgatcagtttccctgacatgccggtggtcaatgtcgaacaggtgcgcctgtgggattgatcgaccgctcacccgcaaccccgccttggcggggtttttcttttctggagaaaccaggtgcaaccgagtttccgtatcgttgccgacggcaccgacgtcacccagcggctgaatgaccgcctgctcaagctgaccctgctggacaagccgggcatggagtccgacagcctgaccttgaggatcgacgatcgcgatggacaggtggccttacccaggcgcggtgcggtgctggaggttcatctcggctatgccggcgagccactgatgcgcatgggacgctttaccgtggacaccttgcagtgggctggtccgccggactgcctgaccgtcactgccaaggccggcgacatgcgcggcagtggcaagacgatacgcagcggaggttgggagggcactaccctggctcaggtctgccgcgatgttggcgcacgcaacggctggcgcgtggagtgtccgttgcaggtggcgatcgcccgggtcgaccaggtcaacgagtccgactaccacttcgtcacccgtctggcgcgccaatacgactgcaccgccaagctggccgagggcatgctcatggtgctgccgcgacagagcgggcagagcgccacggggcgtcggatcgaacctttggtgttgggacgtgctgacgtcggcagcttcgacgttacctacgacgaccgcagcctgatgagaacggtgaagacccgctaccaattgcccggcagcggcgaggtcaagagcgtcgagctgaagaacccgaaggcaccggctacggctacgggcgagcatgtcgaccggcatctctatgccagccgtggagaggccgagcaggcggcgaaggctcgcctggcgagcttcagccgctccagtgccagtgtgcgcctggagctgccagggcgtggcgacctgttcgccgagcgcagcctgctgctccaaggcttcaaggcgggaatcgacggcgagttcctgatcgactcggtggagcacacctacagctccagcggatggaccactgtcgtgcaatgcaacggcggccgaggcggcaaggggtgacgcctgaaacccaccggagtgcaggaggatcgatgaaactgaccgagcagcaattgctgcgcatttttcccaacgcccgcctcgtcgcgggcgttttcgttgcggcgttgcaacgggccatggacgagagggagatcgacacaccggcgcggcgtaccgcgtttctcgcccaggtcggccacgaaagcagccagttgacccggctggtggagaacctcaattacagcgcccaaggcttggcggcgacctggccgggtcgctatctcggccccgacgggcagcccaacgcgttggccttgcggctggcgcgcaatccgcaggcgattgccgacaacacctacgccacgcgcaacggcaatggcgacgaagcgtccggcgatggctggcgcttccgtgggcgtggcttgctacagatcaccgggcgtgccaactaccggttggtcggcgaggccctcggcgagccgctggaagccgagccctggcgcctggagcagcccgtgcgggcggcccgcagcgccgcctggtggtgggccggtcacgggctcaacgagctggccgaccgcggcgagttcgctgccatcacccgccgcatcaacggcggcctgaatggccaggcggagcgcctggcgttgtggcagcgggccagggcggtgctgtcatgagccggcttgctctgctcctgccggccgtgttgctggtcttgctggccggcgccttgctcggcggcggcctggttgcccgccattatcgtccgcaactggaggaggccctgggccaactcactgccagccgcgtcgccagcggccagctcgaggctttgctcgatgagcagcaacgagcgctggcggcggtgcgggcgagcgccgagaggcgcgcgaaggatgccgagcaggcactcggcgaggccagggcgcaagccgcggagcagtatgccgcggccgtgcgtctgctccaggaacccgactttggcacggactgccaggcggcaggtgcggcgatcgaccgggagctgggactatgactcgtctcctgctggggctttgtctgcttttcgcgggctgcgcagcctcaccgacgacacctcgcccagtgcgcgtcgaggttcccctggcagtgccctgccgtgtacctgacgtgcgcccgccgagctgggccggcgccacgctgaaggccggcgattcgctgcaggccaaggttcgcgcattgctcgccgagcgccgccagcggcagggctacgaactcgaattgcaggcggcattgcgtgcctgccgctgagacactgggcgcatatggacgtcgatggacgtgtgtccggcgcatattgccgtgtattgattccgcagcgttgtcgagccgggaatcggtcggtacaacgtagtcatgcttgtacaggtgtgtccccccagggatgtcacctgcaacctcagagcccggccagtgtgccgggctttttcgtttgcatccgacaacggctcgggacgtggaggctcctcgccgaccgcgtaccgcgccacggctgaccgtcctggaggcggggccagacgacccgcctggtgcgggtcttttcatgtgcataaaggagagttttccatgtccatcctgactcaaggtacccagatctacgccctggttccgccggtatccggtaccggtgccgctaccgtcctggagatcgaaggcgtgacttcgttcaacccgggcggcaatccggccgaccagatcgaagacccgtgcctgagcgacacttcgcgcaagtacaagaaaggcctgcgcactcctggccaggcgaccctcggcatcaacgccgatccacgcctggccagtcacgttcggttgttccagctgtccgagaaagatggcgagacgagcgtcaagtgggccatcggctggtccgacggaatcgacgtaaagccgaccgtcagcaccgaaggcgacgatttcgtgctgccgccggcgcgcacctggttcaccttcgaaggctacgtcagcgacttccccttcgatttcgccagcaacacgctggtcgctacccaggccaccatccagcgttccggcgccggcaagtggacgccgaagtcggcttgaggagcgcgtagatgaatctcaacgaacttcgtgcggcgggcggtttcatcgagtcggcgctggtgcgcaaggagattacctggacccgcgttcccgccggcaggaagaaggcggtcagcgacaccttccaggtgttcgtccgacgcaacagctttggcgcggtggagcgcctgttctccgccgaaggcgaccagcagagccgcaacgcgcgctacctcgccgaatgcattcgcctgggcgagacgggagaggaaagcctgacctacgaacaggcctacgacctcgacccggcgctgggcttcctgctgctgcaggcggtcggcgaggtcaaccgggtcgaggacgcggaaaaaaactgacccccgccgacgaggtttggcacgaactggtgctgaacggcgtcggcgggtcgaccattgccgaggccaaggagcggctcagctacgccgagtaccgcgcctgggtcgcctatctgaacaagcgcggctcgctccatccgggacaccggctggagcttgcgctggctcggatcgctgcgttgctcgggcatgcgctgggagcggacgccgatcccgacgcgttccgtccgcatatggctctccagcccctttcgctgcatcaggcgatggatcaatgggcatgacaggccaccccgctgcggcggggtctttttctggaagacatgaatcatggccacgaataccgacggcagcctgacgctcgacctggtccttcgcagcgaagggtacagggccgggatggacaaggttggcaggatcaacgatcagaaaatgcgtgccatggaggcgcgcgcggaaaaggctggcaaggctatcggcaaaagcctggacagttcggcactgattgccagcagcgtgctggaccaggcgctggacatgctgggcaggaccagtcgccaggcgggtcaggccaagaagcctgtgcagagcgcccaggacaaggtactggccgagtggaagacccggcagaaggagctgggcgaagcctggaagagctatcgcgaaccactccaggatctgtccaagctcaacgaagcactactgaagaactcttccgacaagctcgacaaggcgctgctcaatctcagcgagaccggcaagctgtcgcttgccaacgtgggcaaggccgcctacgccgatgccgcgcgcctcgcctcgcggcagatgacgctgatgctgctggacgggctgtttggctgggtcgccagcgtcggtaccgagaagcccaaggtcgacgacaagacgggcaagggacaggcgaaggccggcgacgacgagaaggaacagccgtcgctccagtcgcaggtcttcaagcagtggctgttgcagatgaacagtgtctggggcgcctaccgcgcgccgctgcaggatatctccgggatgaccgacgagctgttcaggaatgcgtcggagaagctcgagaagtcgctgttcaatttcgccactaccggaaagctgtccttgagcaacttcgccaagacggtgatcgacgatgtcgcccggatcgccgcgcggcagctttcaatgctcgccctggacggattgttcggctggatgaatggcaaggccggcatcaccgaggcgcaactggccagccagaagccctatacctcgctactggaaaaggcccgcgcagctgcgggacaagcggcagcgggcgctcccgcggcccagggtgccgcgccaatgccggcagcggcgatggatgtcggcgcgatggtggccactgcttccgggcagaccggggacggttccaaggtatcggctggaggggcttcggcgagcgctggcaagccggtgggcagttgggtcgaacagatggacgcctcctgggcgagcttgcgcgaccaggcgcaggacgtctcgggaatgatggacatgctgtttaccaacgccttcaccaatatggagaacgccctgttcacctttgccaccacgggcaagctgtcgttcaaggatttcgccgactcggtgatccaggatatggcgcggatcgccgcgcggcaggcgacgctgcagatcatcggcggcatcgtcggtgcggtcagcgggttcttcggtagcggcgcaacggcgggctcgcggatttccgactacaccggctcggacatggccaattgggtcagcaagcaacgcgccggaggcatgcctgggttcgccaggggcggtgctttcaacgatggcatccagagcgcgccggcgctgttcagcatggccggcggtcgtccggcgctgatcggcgagcgtgggccggaagccatcatgccgctgagtcgcggttccgatggcgtgctcggcgtgcgcgcgctcggcggcggcgagggtggcaacgtcttcaatttctccaccagcgtcagcctgggcggcggccgcgagggcgcggcgacggccagcggcgacgacggtacgggacagcagctggcgggaatgatcaacgatgccgcgcgcaacgtggtggcgcaggagctgcgccccggcggcctggtatggaggatggtgaatggctgatctggaacgctttacctgggacatctcgatcgattccgccggccaggcgaaccaactggtgcgccaggtgcagtacggcggcggctacagccaggcgctcggcgacgggctgaacaacctcagcgagacctggcaggtttcgcgtaccggcgatctcgcgctgatcggcccgatccgcgatttcctcaagcgccacggcggctaccgctcgttcctctggaccttgcccactggcgaaccggtacgggtgcgcgcccagggctggcaattgcgaccgcgcggcaacggcgtgttcaccctgaacaccaccttccagcaagtcttcaatccgtgaggtaagcatgaccatcacagccgatgaccaggccctcgagcctggggcgctggtgcgcctgttcgacctggattgcaccgggttcggcggcgagatgctgcgcttccacggccacctgcagcaggggccgatccactggcagggcaacgtctaccaagcttggccgctggaggcgcgcggcttcgagcagcgcggcgacggccgggccagttcgccgacccttagcgtgggcaacattgacggcagcatcagcgcgctctgcctgttcttcgatggcctggtaggcgcgcgcctgaccgtgcgcgagacctatgcgcactacctggatgcggccaacttcgccgaaggcaacccgcaggccgacccctcccaggagcgcctgaacatctggttcctcgagcagaagaccgccgagaacagcgtccaggtgacctgggagctgtccgctccgccggacttccagggccagcagatcccggcgcgccagatcacctcgctgtgccactggtgcatcaccaacgagtaccgcgggcaggactgcaactacaccggcacggcgatgttcgatgccgacggcaatccggtggacgatccggcgctggaccgctgcggcggccgggtcagcgattgcaagctgcgcttcggcgcggacaacccgctgtcccacggcggcttcgccggcgccggcctggtcaggatgtgagcatggaactgagccgcagcctgcagcgggccatcgccgcacacgccgcccgcgagcatccgcgcgaatgctgcgggctgatcgttcgcggtgtgcgtcaacgccgctacgtggcctgtcgcaacgcagccggatcgcccagcgagcacttcgtgatcgatcaccaggactggtgcgctgccgaggaccagggcgaggtcctggccatcgtccacagccacccggacgttccggccacgccgagcatggccgatcgggtcagttgcgaactgcatggtctgccctgggtgatcctgtcctggcctgaaggcgatgtcgcgcatctagcgccggagggctatcgggcgccgctgctcggccgcgagttcgcccacggcgtgctcgactgctggagcctctgtcgcgactggtaccgccgcgaggcaggtttggagcttccggactatccgcgccgcgacggttggtgggaaaccggcgagagcctctacgagcagcactatgcggcggccggattccggccggtgccgctggccggaatccgccgcggcgacatgctggtgatgcaggtcgggagggcgctgcaccctaaccacgcgggcatctacctgggcaatgactggcgtctggacagcgagccggtccaggcgctcggcggcgacggaccgttcctgctgcaccacctgtacggacggctgtcgacccgcgacgtgttcggcggaccctggatcgaacgcacgcgcctggtcttgcggcacacgcagatgccgcagtgaacgacatattcaagcgagccgtcggaatcggctcttcacgagaggaacaggtccatgagtgacaccctgagtcagggcctcaccaccatccgtctatacggggttctgggcaagcgcttcggccgcatgcacggccggttgttggaaagcggcacggtacgcgaggcgatgagtgccctgaagcacaccatggagggattcgagacgttcatgcgcgaggcggagtcgaaagggctgaccttcgccgtgttccgcgggcgtaccaacctgtccggcgagcaactggacatgcgcggacgcgaggatatccgcatcgtgccgttggtgatcgggagcaagcaggcggggcttttccagacgattctgggggctgccttgattgtcgtgggtggattcacgacgttcttctccggtggaaccagttcgttcctagtaaccgttggagtcagcatgttggcgggcggcgtcatgcagatgctcagcccccaacccaagggcctgaagggccgagaggcccccgagaacgcccccagctatgccttcggcggcccggtcaacaccatcgcccagggccatccggtcggcgtgctctatggcaagcgccgcatcggcggcgcggtgatcagcgccggcatctatgccgaggaccggctgtagccggcaacgccgtaacaggcccgccatgcgcgggcgtttttttgcctgaaggaacgtcatgaacaagaccatcacgggccacaagggtggcagcaagaagccgcgccagccggtggagatgccggactcggtgcgctcgatcgcgcgggcgaagattctcctggcactgggcgaaggcgagttcgacggtggcgtcgacggccgttcgatctacctggacgatacgccgctgctggcggcggacggctcggtgaacttccccggagtgacctgggagttccgtccgggctcggtggaccaggaacacattgccggtgtgcccgccgtggaaaacgaactggcggtcggcgtcgaactcaagagtgacgcgccctgggtccgcgcggtgaacaacacccagctctcggcggtgcgcctgcgcctgtcctggccggccatccagcgccagcaggaaaacggtgacgtggtcggctaccgcatcgactacgcgatcgacatcgccgtcgacggcggtgcctggcaggaagcgctgaaggcttcgctggacgacaagtccaccagccgctacgagcgctcccaccgtgtcgacctgccggaggcgcggagcggctggcaggtgcgcgtgcgccgcctgacgccgaaccagaacaacaaccgcatcgccgacaccatgcgggtcgaggcgatcaccgaggtgatcgacgccaagctgcgctacccgaacaccgcgctgctgttcgtcgagttcgatgccagccagttccagagcattccgcagatatcggtggaagcgcgcggccggcgggtgcgggtgccgagcaactacgatccgcagacccgtagctacagcggcacctgggacggctcgttcaagtcggcctggaccagcaacccggcctggcactggtacgacatcgtgttgcacaagcgcttcggcctcggtcggcggatcgacgcgagcatggtcgacaagtggtcgctgtaccgcatcgcccagtactgcgaccagtcggtgcccgacggcaagggcggccaggagccgcgcttcagctgcaacctgtacctgcagagtcgcgccgaagcctggaccgtgctgcgcgacctggcagcgatcttccgcggcatgtcctactggtccggcgcggaaatggtggcggtatccgacatgccggaggacgaggcctacaccttctcgccgtcgaacaccgtgcgtggcgacgacggcagccacttcaactacagcagcagccgccagcgcgatcgccacaccctggccctggtcaactacgacaatccgggcaacggttaccagagccaaccggtagcggtgaacaatgaccgcgcgcagcgccgctacggcatcagccagttggagatcaccgcgatcggctgcacctccgagggcgaggcgcagcgtcgtggccagtgggcgctgctgaccgaggagctggagcaggacgcggtgaccttccgcaccggcatggatggccgtgggctggcgccggggaagatcatcgccgtagccgacccggtcaagtccggcaagcagatcggcggacgcctgagcgcggtggatggccgcgcgctgaccctcgaccgcgacgtcgaggcccgacccggcgatcgcctgctggtcaacctgccgaacggcaaggccgaggcgcgcagcgtccagtcggtggtaggccgcgtgctgagcgtgaccgccgcctattcggagacgcctcggccccaggggcagtgggcgctgcagagcaacagcctgaccacccagcgcttccgcatcatgagcatcacccggccggaggacaatcttttcgagatcaccgcgctgcaacacaacgcgagcaagttcgacgccatcgacaacggtgcgcgcatcgagctgccgccggtcaccagcattccgccgggcgtgcaggcgccgccgcagaacgtgcggatcaaggctttcaccaaggtcgaccaggggttggcggtgaccagcctgtcggcctcctgggatgccgcgccgaacgcggtggcctacgaggccgaatggcgcaaggactcgggcaactgggtgcgggtgccgcgaacctcggcgctcggtttcgacgtgccgggcatctatgccggtcgctatctggtgcgggtacgcgccttgaacgtgatggaggtcggttcggtctacgccagcagtgtggagaccgctcttgagggcaagaccacgccgccgccggcgctggcctacctgcgctgcgtggccggcccctggcgcatcggcctggagtgggggttcccgaccagcggcgcagcggacaccgcctacaccgagatccagcagtccgccacgcccggcggcagcgaggagaccgcacgggcgctgggcctgttcgcctacccaggcaatacccacctggtatcgccgataccggccggcgaacggctggcgttccgcggtcgcttgatcgaccgtagcggcaacgtcggcgcctggtcgaactgggtcaccggcaccagctccagcgacgccagcgaatacaaccagttgatcacccaggagtacgtcgagtcggcgctgggccagcagttcttctccgatatcgaacggatgcaggtggatatcgggggcttgcagaagcaggtcggcgacctcgccgacgttctgctgtacgacccggccaaggtctacgcgaagaacgacatggtgcgacaggggcagcggttgtaccaggcactgaaggctgtgccggcgaagacggcgccgccgaacgcggcctactggtccgatatcggccagtcgctggaaaccgccaacgggctggcgcagcaggtggcgagccataccgctgaaatcagcgaactcgacggcagccttaccgcccaggcatcgcgccttggcgtactgcaggcggcgacccgtgacgacgcggatgacggcaatggcgccatggccgatgccctgcgcggctggaagaccgttgcccgggcagcccaggaggaaaccgtacgggccaccgaaaacgaggcccaggccactcgcacgacgctactggaggcgcgcaccgccgatgccgaagggcgaatcgccacggtggaacgggtcgcgaccagcgatcgccaggccaccgcgcaacgtctggaccagctctcggcctcgatcggtggcaccgccgccagcctacagagcgaacagaccgcccgcgctaacgccgacagcgccctcgcacagcggatcgacaccgtgcaggcgcgtaccgacaccaacagcgcggcgatccagaccacctcccaggcggtcacctcgctggatggcaacgtcaaggcgatgtacagcgtgaagctccaggcgcatgccaacggacaaaagtatgcggcggggtggcagctggggttcgacagtgggaccagtgtgtcaaccatggcgtttcaggcggatcggtttatctggttcgacagttcgagtggggcaacggtagcgccggtttcaatagttaacgggcagatgtttatcaagagcgccctgattcaggacggagcgatagataacgcgaaaattggaaatgtcattcagtcaaatgcaatatcaaataatggtatgccgatatggaggctggataaatcaggtacttatacagtgcgagacagtgcaggccaagtcagagttgagatggggcttctacagtcatgagtatctatgggttacgtatctatcgagagaatggggatgtcgctgtcgatataactgacagggcgttgcgggttgtatattccaggcgggtcgatgcagtttcgaaaggagaagctgccacacccggctttggcccgaacaatgcttcagtctatgtgattggtgatcagtacaataagcgtccggtctgggcccgaatgggtgatggcgttgttcaatggggctacgaggattggtggccctcactatttcacacttctggaaccttatatgtggtggccaaggtatgagtgagtatggggtcttggtaaggaatggcctggggcagactgtaatagatggagagtttcataatctttctcttctgttggaaagaaatattgaagtcgagacaagtcagtggttctcactggattttccatatgcggttacctcagctgcacctcctgttctagcagttcaagcttggaagaataaatttctttacttcgactcagtacagtatcgaggcgggccaggaaactggacaggcgccagtctgagtttttcggggtatggagcacatacatccggctccgtaagagttcgagtctatgcctatgcattgcctttgctgagaggatatgggcttcgtgttcgaaactcggcgggaagtgtcgttttcgattctctaagacttcccttggtttttagtgcggaactgggcggggcgccagaagattgggttagggtatctggggaaccgattataggagctggccgcatagacatatatcgcccagcgacatgggggcagcctgccgatagctacattgcagtaggcatggcgcttgacgtggaatttggcagagtggcggtatctggtggcacggccaacgcaattatttatatacgctggggtttcactgaggagggagtgccaaggcttatgcagcatgcatataccgggcttccaagttcttatcctggaattccagctgctgtttattatgctatgccttcaattccaataataaaggcttgagttgatttttgggagataatgctatggcttggcattcgaaaggttcggtttccgtcacgctgaattccgaagcggtgttaggaagcgctactgattttatcgccaatgtgcgaaccggcgatgcgttccgtggccctgacggccggtggtacgaaatcaccaatgtgaccagtgcaacggttatttctatcaagcccaattatcagggcgcgaccgctagcggccaagtttatgcggttgtcccagtacatggttattcaaaaaacttggcagaccaattccgcgatatcaacaaccagtggggagccaccctggcagggatcaaaccttgggcggtgtcctcgacgggccagcaggcgcaggccgacatgggaatctcggctgttggtcgggccctgaacaatgcctcgacgccggccaacgccttgagttacctggggggcgtcgcgcccaatcagatgggctgggctggcaacgcgatgaatacggcggacctcgatttgctgaccgtctcggggctatacgctcatggcacggcggtgccttcgccggtgaacaatgcccagggctatgtcttgcatatgcagcacggcaacccggacttcgccgtccagcagtggtaccagttgaattccgccactggccagtacatgcggatcaaggccgggggtaactggtcgcgctgggtgttgcagtacagccagttcaaccttgtcggactggcgagcttcgatgcatcgaacaatccatccggcgccatcatccagcgcggcgggactgtcggtttcaacgagtatgtacgctacgccgacggaacccagatctgctggggcaacaccactaccaatgtgggagccaccatggcctaccagccggctggtacgttgtcgttctatatcactccggttgcttattcgtgggggttcccggtctcgttctcccggcctccgtctgtcatggtcaacccgatgagagcagcaggaaacaacgcttcccgtccctggggttcgacgatgtcggtcaccgaaacgctgttttcctggtacggctacgacaccgccagcgtggccagcgggatggccgccagctatgtcgccatggggaggtggaattgatgaagctgttgttgaaacctttgttgcagcagggcatcacgcctgacagggaacgtctcagcgatgtccaggtgcgagggtcggtgtttatcctggatggcgtcgaatacgatttcggaagaatgcagcctggcgggtacctgcctccggaggcataccatggtacgccattcattgatattcgcttcgtggacggcgaccgcctgtacctgcactacatccaccaggtgacctctgaagtgatgatgcagtttcccagggaggtcgagtcaattccggtcgaccaggatggaaaagtcgaggtagcgttcagctatgaacatccaatgggataagtacgtcagccctgcaaaggccgcggcagatgtgcgcgatcaggccttggccagtgctcaggccaagcgccttctggcctatcgggaagaaagcgatccgctcaagaccgaagccgagttcgatgcgatcaaggccggcgtcgagccggactatggcgcctggatcgccaaggtggaagagatcaagtcacgatacccgatgcctgaatagccgaactccaggctcgcctgggcgtcagccctgtggttccgaactaaccctatcaagcccgccgactgcgggtttttcattatctggagaacgtcatgccttggtattccacaggcacggtttccgtcgtcctcaattcggacacggtgaccggcagcggcaccgccttcagcgccaatgcgcgcgccggcgatgccttcagagggccggatggccgttggtacgagatcggcaacgtcaccagcgccaccgtgttgaccatcaagcccgcatatcaaggagctaccgccaacgggcaggcatattcgatcaccccggtgcaggggtactcgaagacactggcggatcagttccgtgacctcagcaatcaatggggttccctcctggcggcagtgaagccttgggcgatagcgtctaccggttcgcaggcgcaggctgacatggggatcaccgaagttggccgtgctatcaatggagcctctaccgtaggcaatgcattggggtttttgggaggtgtttccaagacccaagcccccatggccctggatatggacacggtgaacgaaagcggatggttctcgataacccccaatacttacaacgtgcctctcggaaacaacaatatcagcggtgtgaacggccatgtcgctctgtcgatggtattcgacgccagtacccgctatcagctgttcttcgtgagaaataccaacctcccagaggtttggtacaggagctgtaccaatggaacctggaaggaatgggtcaggttttatacgacagacaatatcgtggggaccgtaacgaggcgacttgttacgggtaagcccaccggggctgtgatggagagcgggacaacttccaacggctggtacgttcgctttgcggatggtacccagatggcagcggcaagatccgaaccgggcctttcgtttggagctaacgtgatacagctaccagctgccttcgttacgggcttcaataccggggtgacctgcaattggattccctccagcggatggcctgccactgcagggcagggggttcgtggagcctatctcaacggtagcagctctgtttctttcgcgacggcgcaggcactgggggctaacgacaccattacggtgatggctgtggggaggtggtactga

SEQ ID NO: 19 - DNA, 2106 bp, Pseudomonas aeruginosa
  misc_feature 1..2106: note = Description of sequence: polynucleotide, name: MRSN2101 R1 pyocin tail fiber
  source 1..2106: mol_type = genomic DNA
atgacgaccaatactccgaaatacggtggcctgctcaccgacataggtgccgctgcgctggctgcggccagtgcagcaggcaagaaatggcagccgactcatatgctgatcggcgatgccggcggtgcgccgggcgacacgccggatccattgccttctgcggcgcagaagagcctgatcaaccaacgccatcgggctcagctgaatcggctgttcgtttccgacaagaacgccaataccttggttgccgaggtggtgctgccagttgaggtaggtggcttctggatccgcgagatcggcctgcaggatgccgacggcaagttcgtcgcggtatccaactgcccgcccagctacaaggctgcaatggaaagtggcagtgcgcggacccagaccattcgggtgaacatcgcgctctccggcctggagaatgtccagctgctgatcgacaacggcatcatctacgccactcaggactgggtgaaggaaaaggtcgctgccgatttcaagggccgcaagattctggctggcaatggcttggtcggtgggggcgatctttctgccgaccgcagcattggtctggcgccttccggcgtgacggcgggcagctatcgttcggtcacggtgaacgccaacggggtggtcacccagggcagcaatccgaccaccctggccggctatgcgatcggagatgcctataccaaggccgataccgatggaaaactggcgcagaaagcgaacaaggccaccaccctggccggctatggcattaccgatgcgctgcgagtcgatggcaacgccgtgtcatccagcaggctggccgcaccgcgtagcctggcagccagtggcgatgcctcctggtcggtgaccttcgacggcagtgccaatgtttctgcgccgctgagtctttccgctaccggtgtggcggcgggcagctatccgaaggtgaccgtggatacgaagggaagggtgactgctggaatggcgctggcggcgacggacattcccgggctggatgcttcgaagttggtcagcggggtgctggccgagcagcgtttgccggtattcgcgcgcgggttggctactgctgtctcgaacagtagcgatccgaacaccgcgaccgtgccgttgatgctgaccaatcatgcgaacggacctgttgccggacgatacttctacatccagtcgatgttctatccggatcagaacggcaatgcttcgcagattgcaacgagctacaacgctacatccgagatgtatgtacgggtgtcctacgcggccaaccctagcatccgggagtggttgccctggcagcgttgcgacattggaggctccttcacgaagacgactgacggatccattggaaatggcgtcaatataaacagcttcgtcaattccggatggtggttgcaatcgacatcggaatgggcggcgggtggagctaactatcccgtggggctggccggtttgctgattgtctaccgcgcacatgcagaccatatctatcagacctacgtaacactcaacggaagcacatattcgcgctgctgctatgcgggctcttggcgtccgtggcggcagaactgggacgatggaaacttcgatccggccagctacctgccaaaggcgggatttacctgggcggctttgccgggtaagccggcaactttcccgccctcagggcataaccacgataccagccagatcacctccggcatcttgcctctcgctcgtggtggccttggcgccaatacagcggccggagcacgcaacaacattggtgccggagtgccggccacggcgagccgggcgctcaatggttggtggaaggacaacgataccggtctgatcgtccagtggatgcaggtgaacgtaggagatcatcccggtggaataatcgatcgtaccttgacgttcccgatcgcgttccctggcgcctgtctgcatgtcgtaccgactgtcaaggaggtggggcgaccagcgacgtccgcgtcgaccgttacggtcgccgatgtcagcgtcagcaacacgggatgtgtgatcgtttcctccgagtactacggactggctcaaaactatggcatcagagtgatggccatcggctattga

SEQ ID NO: 20 - DNA, 14756 bp, Pseudomonas aeruginosa
  misc_feature 1..14756: note = Description of sequence: polynucleotide, name: MRSN317 R2 pyocin
  source 1..14756: mol_type = genomic DNA
caccccttgccgcctcggccgccgttgcattgcacgacagtggtccatccgctggagctgtaggtgtgctccaccgagtcgatcaggaactcgccgtcgattcccgccttgaagccttggagcagcaggctgcgctcggcgaacaggtcaccacgccccggcaactccaggcgcacactggcactggagcggctgaagctcgccaggcgagccttcgccgcctgctcggcctctccacggctggcatagagatgccggtcgacatgctcgcccgtagccgtagccggtgccttcgggttcttcagctcgacgctcttgacctcgccgctgccgggcaattggtagcgggtcttcaccgttctcatcaggctgcggtcgtcgtaggtaacgtcgaagctgccgacgtcagcacgtcccaacaccaaaggttcgatccgacgccccgtggcgctctgcccgctctgtcgcggcagcaccatgagcatgccctcggccagcttggcggtgcagtcgtattggcgcgccagacgggtgacgaagtggtagtcggactcattgacctggtcgacccgggcgatcgccacctgcaacggacactccacgcgccagccgttgcgtgcgccaacatcgcggcagacctgagccagggtagtgccctcccaacctccgctgcgtatcgtcttgccactgccgcgcatgtcgccggccttggcagtgacggtcaggcagtccggcggaccagcccactgcaaggtgtccacggtaaagcgtcccatgcgcatcagtggctcgccggcatagccgagatgaacctccagcaccgcaccgcgcctgggtaaggccacctgtccatcgcgatcgtcgatcctcaaggtcaggctgtcggactccatgcccggcttgtccagcagggtcagcttgagcaggcggtcattcagccgctgggtgacgtcggtgccgtcggcaacgatacggaaactcggttgcacctggtttctccagaaaagaaaaaccccgccaaggcggggttgcgggtgagcggtcgatcaatcccacaggcgcacctgttcaacattgaccaccggcatgtcagggaaactgatcagcaagccggcgcggaatggctgctgctcatcggccagcccgggattggcgtcgagggtcgcctcgacgcagcccaacagatggccatagacgtggtagcagaggctgtccagcatgtcgccgtcagcggttcggcaggtcgtcgccatagcgcttgaactccaggtcgaaggtttgtttgcggggagcgccgttgcccagcagggcgctctggttctcgctgatccgcaccagacaccagaggcccatctgcacctcgccgtagccgctgctcaggatcagcggctcacgccgctccgccaaccggcgcagcgcttcgagctgctccagaccgacgaccttttcctggttgcctacctgtcgacgcagcgggaataccgcccccttgagcaggatgctctcctcgcccatgccgacgctctgcagcgccggccgccggcccaggcgcacctgcccgctccagttgtaggcactgttgcgctgcagggcgtcgaaggcggcagtattcaggttgaagtagaacggttgggcattggcggtcagcggctgcagtaccagcaggtgcggcaagggcgctgcggcctcgctcgggggcgtcgccgagggtgccagcagccacgacggcagcacccccgccagcgtcggactgatgtcgccagcgaccttgccgaccgcctgcgcggcactgttcacctgcaccttcagcgcatccaggcgttcgtcgataccaagcaacgcccggctcgcccggtcataggtggagaccacgcggttcaccttgccctgcgcctggccgatgccgcgcatggcacgctgcagccgggcagccatttccggcgatacgccgggcaggttctccagctccgcggctgcgccgcggatctcgctgatcgcaccgttcaccggagcgaccaccttgtccagactcttgcgtccggactcgccggcgcgacccaggtacctcaggccggcctgcaattgttccagatatgccatggggactccttagaccatcgccgggtcgtagaggctgcgccgctgccgctcgtcggcgaagtcgacaagcatgcgttgcaggtagggcagcaactcgtcggccaggcgctgcggctcgtgtacgttgccttgcaccgtcaggttgatctgcggcgcgaaggtccagttgggcgcagccgcgacgggctcggcctgtggtgcgggcaccgccggcttgttgtccgaggccgccggcggatcgccagccagactgcgtcccagacgcccccccagttcgctaccgccccaggcgccgatagcgccgccaaccaatccaccgatcagcgtaccgaccaccggcaccaccgagccgatgctcgctcccagagcggcgccagccagggaaccagccagggagccaccggcctcgccataggcgatggtcttttcgctctccgacaggtcgctggagtaaacctcagcaacatcgatcaacgacgaggccacttgcagcggcgcgttgcgtatgggcaaacgcgacaggcgcggcagctttcctgccatcgcccccaagcctcctccagccgagaagccaagggatggaagggagcgggccttcagcccaccgcccttgcctctcttcctgcgtggtgtttgaccgcgctttcgttgtgccggcctcctcgcggactccagcgtagagtcgcttcccggcaccggccagttggtaacgaacacgcgctgtatttcgctaccacccgatacccggcctgcgccctgttcgatcggatcgccgacggcagcgctccctcgccgaccgagccgaccgccacgcaacacgtcgatagcgccgcgccctcccttgtaggcgagatacccggataacaccgcacctgcggcggcggtaagaccggccgtcgtccgcggataggcttcgagcagttccgcagtcgactcgccgagtaccgcgctccccttggccaggttgtccagatatggattcagggcgctgcccaaagcggtctgcgccctctcccaactgtcgctggaagccttccacagctcctgggacatccccttgcgacgccgcgcatcgcgctccgaagagcctacggaagactgctgggattcgagcaagccctggaactcgccgcgcgacgctcgataggcttcaagcgccggcgacgccgcaccggcgctgagcttgccgcccttatcccgatagcgcgcagccagctccaggttggcctccagcagactcgctccgtccttgcgcagcgcttccaggtcccggtcgtagtcctggccataagcgcgtttcaagctgccgctgctgacgaactctagccaggccttcatccgcacgtccgctttgtctggcgtggtgttctttgcctgaatctccagcaaggcccccagtgcaccgacatcgccggccgtggcctctcccgcattgcccagcgctgacaacaaggcaggcaggcgacgcgccagggcctcactgccgactctgcctttacccaggacgaccaggcggtcgagggaggcggacaattgctctggcgagtccgaaccctgggccagttgcaacgctcgaaccagccccgcagcctcggctgaagcaatcccttggccaaagccgaagcggcccgccagtcccagattcatttgcgcggcggcgaaccccatgccggtggcgttcaagtgctccagcatgtccagcgtcgccgtgcgtcccaggccgctctggctggcactgtcctggatcagggatgccaaggctggctcccggccgacgtccaggccgttgcggatcgccaggtcgcgcaaccgcgcttgataaccggcagccgtcttgctggcttccacgaccagtccggaaaatgcgctagccaatggcattgcctggctggcctgctccagccgttgcaggccgcggacctgcatatccaggccacgctgcatctcgcccagtcgagcgtaggcatcacccgcagccctggcctcgatgccgagtcgcttcagttgctcctggcgttcgccaagctgccgggcatgctcagcaacgccgcggtccccgagcttgcgcatatcggcgagttctcgctccaggcgtatggcgtctcccagcacatcccttaggctggacgcctggcgggaacgttcctggagactgtcgagccgtgccttgacggtctcgaatgccttgcccaaggaggggtcggcgatgccgccgatggagaccaccaggtccatgtctttactcatgacggttcgtctcgtctgttatcgatgtccgcctccgcggacctactgaagtagcgcgggcgccgctcgcctggcggacggcgccgtcgctcactcctcgccctcggcgagccaccagagcaggtcgcccagggtcatgcgctccagttcgctggcctggaagcccagctcaatggccaggcgcctcgccagttgacgcatacgcgaaggtgtcgtccgtgtcgtcttgcaccaggcgaaagtaggcgccttgcagccgctggtagtcgctcagcttcagcccctccaggtcctggcggctgacctgcgccagcgaggcgaacagttgcagttcgcgctcctcggcatcgtcgccggccaccttgccggccaggcggatatcacgcacggtgggtgcacgcagggtcagcgtgtcgacgctgaccccgttgcactgtgccggacgcgagagacgaacgagggcggcgtcggcatccagcgtcagccagcccggcagtcgattctcttgggtcatgtccggagctcctcttacaggccgaggtcgttgcgcatgccggccaactggtcgacaccgttgatcgcgcggacaccgttgaccggatcgatctcgtagacctcgcggccatcgacttccagcttgtagtagctgaccgcaacggcgtacttgaactcggctttctcgccggccttccagtcgcccgggtcgacctccttgagcaggccgcgcagggtagccaccactggcacgctggcgcccttctggcccttgaaggagccgcggaacacgccattgaaagcgctctggtcggccaggccgaagaaattcagtgcttctcggcgggcaccgttggtggagaacttggcctccatcgcctccaggcccatgtcgatggataccggcgcatccatgccgccggcacggtattgctcggtcttcacagccagcttgggcagggtcagggatggcacgtcaccggcgaagctcacgccgtcgatgaacaggttggtattggtcagggtttgcggaatcatcacgggcgctccttaggcgacatccagaacttcggtgagccactgatcggtcacctcgacacggaaattggggttctcggcaggcggcacatcggtgaagcggatgttccagtacaccttgccctgggccagctggctggcgctgttcaggtccgggtcggcatagacctcgaagttgatcaccgcgccctggttcttcagatcgcgcatgaaggcgcgcaggccctcggtgacatccttcacgtaggtcttggtgatgccgcggtccaccgcccacttgtgcccggcgaggatcgcatccatcaccaggtccatggtccgaacgcgggtgacgaaagcccacttgctgtcgctggacaaggtgcggttgccccacaggcgatagccatcgtcgcgaatgatcgtggcgatattggcgttgttgagcaggttggcgcgacaggtctcatcgccgtcgaggaactccaccggacggctggtgccggtgatgcccttgatctccttgttcgacggcgaggaccagaagccgtactcggcgtcggtccaggcgaacaggccggcggcgtaggccgaagccggggccttgcgtgcggcattggtggcgctgtcccaaacctgcacgcccgggtcgaccatgaacaggcgcttgctgccgaagttcttggcgtaggcgacggccgcctcgtcggtgctattgggaccatcgagaatggcgatggcccgcagtttctcggccagcccgtccatggcggtggccaccgcttgctgggccgaatgacccggcgcaactagtagacgtggctgggcattgaagcggctcttgccgtctagcagagcctgaagtccggtgcgctcgccggcggcgctgacaccacctatgacggcgctggcctgggcctcgggagtctccgcggcttccacgcccaccgccacgatcacggcctgggcacggttatagatggcctcgcaggccaggtagatagacgagccgatgccgaacgccgcggcggcgtccttcttgctggtgagcagcaccggaacgttgggcttggcgcttgcctgcgcccccggcgtgaacacatcgcagaggccgatgacggagctggccggcagcgcgatggtgcgcgcaccgatatcgacgttggttaccgtaacgccgtggaagaaactcataggtagatctccattaatgaaaaaccccgcacgaggcggggttcagggggtgacataacgtctgagccaatctggtacggacggacgtccgccactgtcgggaaataaaggctcttccggccaatcccgtaagctggctctgtacgacatgagttcatggtattggacagggagtaaggtcgtttcctttccggtttccaattgatctcgatggcgaacaactaggccgtcggtcgccaacagctgactatcccgccatctccgctcatgggcgctacgttgctgacgggaaagcggagggcgctcgatcgctactgggttccccttcacgtcggaactgattgctcggcctgccgcctgttcgttcatcagttgttcatgtagctctgcgctgacccactcgccatcgactggccagcagcctgcgtattcataaacctctcgcaaggaggccggatagaatgccacctggcttggagagaaataatattcgcccttcatcgtccaaatgcctcccatgaaagaaccgcctgagcaaagtagccattgcgaatcaccgcggtagtggtagtcgctccacggaaacctgtactggcatccgttcccggatggaaagcactcgtctgattcgcatatccgccgaggcatagcgtagggaaaggaatcgggaacgtaatcgaagcatcggcatcggcggggcaagtgacctgcccccattgcctaatgaggccagtgtcattgtcccgccaccatccgctcgccccaagggaagcagtcgcaggaacccctgctccgatagtgctacgtgctcccgctgccgtattcgaaccgacgccgccacgtgccagtgggagaatgcccgaagtaagctgtccgacgtcatggttatgtgcggatggtggaaaagtggcaggttttcccggtaacgcattccaatagaaccccgacttcaacagatagtcactggggttgaagtctccgccatgccacatgcgacgccagggaaaccaggtatttgaatgccggcaccggaaatagaaactctcaccatcgtaggcttgatacgtctgatagatgaaattgctactcgcggcgtacacatgaagcaggccggcccgaactatagggtagttggctccactggcagcttgggcagtaaaactctggctccaccaccctgaggtcaccatcgaatccaggttgacgcctccaggcagttcaccatcggcctctttggtgaaggaacctccaatgtcgcagcgctgccagggcaaccactcccggatgctagggttggccgcgtaggacacccgtacatacatctcggatgtagcgttgtagctcgttgcaatctgcgaagcattgccgttctgatccggatagaatatcgactggatgtagaagtatcgtccggcaacaggtccgttcgcatgattggtcagcatcaacggcacggtcgcggtgttcggatcgctactgttcgagacagcagtagccaacccgcgcgcgaataccggcaaacgctgctcggccagcaccccgctgaccagcttcgacgcatccagcccgggaatgtccgtcgccgccagcgccattccagcagtcacccttcccttcgtatccacggtcaccttcggatagctgcccgccgccacaccggtagcggaaagactcagcggcgcagaaacattggcactgccgtcgaaggtcaccgaccaggaggcatcgccactggctgccaggctgcgcggtgcggccagcctgctggatgacacggcgttgccatcgactcgcagcgcatcggtgatgccatagccggccagggtggtggccttgttcgctttctgcgccagttttccgtcggtatcggccttggtataggcatctccgatcgcatagccggccagggtggtcggattgctgccctgggtgaccaccccgttggcgttcaccgtgaccgaacgatagctgcccgccgtcacgccggaaggcgccaggccaatgctgcggtcggcagaaagatcgcccccaccgagcaggccattgccagccaggatcttgcggcccttgaaatcggcagcgaccttttccttcacccagtcctgagtggcgtagatgatgccgttgtcgatcagcagctggacattctccaggccggagagcgcgatgttcacccgaatggtctgggtccgcgcactgccactttccatcgcagccttgtagctgggcgggcagttggataccgcgacgaacttgccgtcggcatcctgcaggccgatctcgcggatccagaagccacctacctcaactggcagcaccacctcggcaaccaaggtattggcgttcttgtcggaaacgaacagccgattcagctgggcccgatggcgttggttgatcaggctcttctgcgccgccgaaggcaatggatccggcgtgtcgcccggcgcaccgccagcatcgccgatcagcatatgagtcggctgccatttcttgcctgctgcgctggccgcagccagcgcagcggcacctatgtcggtgagcaggccaccgtatttcggagtattggtcgtcatgtcactgtttccaggggaagatttccagggtgtcgccgtcggtcacggccagtccgtgacgggaccggattaccggggtgatgcgcaggtccaggcgggtcaggtgtcggctcaccgggcggacgtcatcgagcaagcgctccagctccagtacggtgccttcgtcgatgccacggtcgctgacgtccacggtgatctcgaaggtccccggcacgccggtcgggcttcgctgccaccattcggtgacctcgctcagcgcaccgatcggctcgaccacttggcgcaacgcggtcagcgtgcctttgcgctggtgcaggacgaaggacgtcgctaccgcattgcgcttggcctcgtcgctccagtccgggtcccagcggtccaccgagcgcgtccaggccaggtagggcaacaaggccgcctcacagcgtgccggatcgtgaagctcacgaagcggcaccggcagttcggcaggcaatacatcacccagagagcgttccagagaactcctgtttggcggcagcagtcggctactcatcgacgcctcccaaggtgatcgcgtaccccgtgcaataggccgcctgggtttcatccaggaccacgtcggccgcaggctcctgcagttcgacgcgctgcacgccttcgacatgcagggtggcgaacagggccgaacgccggatgtcgcggcccaggcggcgctgggcggaaatgtagtcgcgcagcgaagcctcggcggcggcacggatcagctcggcctcgggaccgggaaacaggtagagctgcgccttgacctgatagggaacgatgcctgccgagcgcaccgttacccgatccgcgacaggacgcacgtcctccgcgttcagcgccaggcgcactgcctgcaacaccgcttccggtgcctgcccgttgccttccaccccgagcacgctgatcagcacttcgcagggcgccggactggtcgcagaggcatccgccacccttccatccgcgcccagcgcatgggctatataggcgttccgcggtcctgcgacggatagctgctcgaacgcgagctggatgcgattgcgcagcgacttgtcgctctcgtattgcgcctccaccggcggtaccgcctcgggctgagcgggcctgatcagcaaacgctgcacattgaagccggcaccgatctggtcgagatcggcaccgctggcgtatgccagcatcaccgcccgcgccgcatcgttgatccgcgcccgcagcagcagttctcgataggccgccagttgcagaagcttgagaaccggatcggactccacctccgctgtccattcgccagccatggcttcgcggaagtcgcccaacagctcttggtaaatcgtctcgaaatcgaggttttcgataacctctggctccggcaactgggaaagatcgatgatcacgccgagacctccatattgatgttctcgccctcgaacacgccgctcaggagcagatccacgcggccatcgacgaccgcgacgactcgcacggcagacaatgcgatgcgcggttcccagcgccccagggaacgggctacctcggcctgcacggcgcttttccagccttcgctcaccggcatgtcgaccatccgccgcagcttgctgccgtactcggggcgcatgcgcctgctgcccaacggggtggtcaggatgtcctcgacggactgtttaagatgagccaggccggacaggggcagcccgctacggcgatccatcccgatcatcgccccgtcctcccgctccggcagagacagaccgcgcctggctccgaatggctgtgagtgttgcccatggcgaactccgcaaacgaaaaggcccgctcgcgcgggccggttgaatgttcgacggaccgcccttcggcagccccgtcgtatgttcaatgggtgtggtggttgctgttgccggcggtgtcgatgatcgagccgccgccgaggatgtctccgctgaccgtcagtgtcccggcgatctccaccggtccttccaggctgatcttcggcgccttgagactgaccgccccgtcgctgacccgcacctcgctggcgccgaccctcacttccacggtgccgctgggcagctcgacgcggtagcgatgcgcagcccagtcgtaactcaacgaggcgccgtcgtcgaaacgccaggtctcactgctggcgctgttgtccggcgccgtgcccgcatcgccgtacaaaccgggaataaaggtacccattgacactccgcccgacggactgagcagtaccccctgttcgcctatgctcggcgcacgccagtggcgaaccttgccggctgccagggagtgccagcgcagccagccgctggtccagtcgccggagcgcacccgtacccgcgccgctgccaggtccacggcgaccaccacgcagggcaggatcatcgcggcgagcatgcggtcatgctccgcactgacatagctcatgccaacgcctccggagcgaagtagtccggctcatagccggggccggtctgcgggtcgaagcccagcatcaggctgcccggcggctggtcctcccaggcccactccgggtctccgagacgcagcgactgcgtccactcgaccagccagacacgcaggctcttcagctcctcgcgactgccgtcgacggccgagcgtacgaagcgcgccggttccagagacaggttccaggtctgctcgcgcaatgtccgaacgattccgcaagcaatggataccgcctggcgatcggcaccaggcggcaccattatccgcgcctgcaggcggcacagcagggcactctcgccggtaccgggatcgcgctcgggcagtagctcggctatttccagggaaaccgccggggtattttccggcacgcctggccagaagccgatcaactgcgcctccggcagacgctcacgcagtacctgctcgatggccgcgtacagagcctcgagcgtaacagcctgttcaggcatggttgactccttcgatcagtttcaatgcgggccacggtcgcccgactgttcctcgggcagctcgcaaacccccaggcgccgggcggcccagcgttcgtagagaccgacggcgatctccgcgccggccgcggcggtcagactgcccagcgccgcggcgaacagcagcgcggcaccgcaagccaacgccagcagcatcaccgccacgccgcagacgatcgacgcgccggagcgcagcgccaggcgccgcaacaaggcccagccgccgagaccggccttgtcggcacgccacatctcgccgctgacgccgcccagcgcggcgagcaggatcagtacccagagcggcatttccgtcagtgtctgcggttcgttgcccatgggtccctccttggcaggcagcgccgggcacgtccgcacgaacgttttcccgggcaataaaaaacccggcgccagggccgggttccggtgcttcacgagtgcctccctggggacgcacctttacaagaatgactactttttaccgcccgattcccagcccggcaatgccttctcgacaatccactgcaataggagggagacgcaccacgatatgagggtgatataccgccttgggaaccttcgccaggcatccgggcaagtgcgtacccacttccgatatggaaaagcccggcatcgactgacgaaaaggacggctccaccagcccgaacccgcctctaccgtgcagggtccgctgctgtgcatgggattcgccgcctccgtgggcgacaggcataaaaaaaaccggcccgaaggccggttggaaaattccaggggggaccgggatccggtccaggaactcaggccgcccgtcgcagcaggccgtcctggataccctggtgcgccacatgcaggcgcagatagaaggtgttgcggctgcaaccgcagtgacggtacttctgctcggggaagctgtcgcgattgagatagtgctccaccaccacggtcttcagttcctcgggtaggcgatttactaggcgatcgacctccgccacacggtcgaggatcacccggctgccacggtggccgcggaccacctcgcccttgctggcgatcagcatggcgatcaggttgccgccggcgtagccgcctccgttgctgcctggcgcgtgcatttcctcggcccagagcttgagcatttcatcgatggctttgatcacggtcgtgtctcctttcactgcccgcgagggctcggcatgaggtcggttcaacggcgctcgtggcggtcctggcagtcgatgcagcgactgcaacccggtgccgcccggcggcgcgcctggggaatgggctcgccgcagtcctcgcagtcttccgcggactcgcggatggccagcgccgggcgcgccgccaggaggccgtcgaggcgagccaggaccagttcgttggcgtgatcggcaaggtcagccatggaaagcctccctggcgtgcccgacgcccgacggccgaatggatggaacatgacagatgcgcggcgccggttcggcgacctgcgttcctctgcgcggaaatggcgagtgcgtatctcgcgacatggcaatgactccctgtacgacgccgccgtacctgaagcaggtcggcgacagaatgaatgagcgggacggagcgtctgcatggacggcgctccggccctccaggtgccggagcgctttcccgtggccccggcacccaagcagtcggcacctgcatcgtcggtcaccgacaaccgggtgcgtccctgcacccatgtgaaatctctgaaaggcgggtgggaagttgtggaactcacccccttccgctttctgacggaagaaaaattacaaccaaagctattaatcgtcaacacctaggctctttacagagaatccatcggtctgtagattgccgaacatggacaagagcacccagatcccgcccgacagcttcgccgctcgcctcaagcaggccatggcgatgcgcaacctgaagcaggaaaccctcgccgaagcggcaggggtttcgcagaacaccattcacaagctgacctcgggcaaggcccagagcacccgcaagctgatcgagatcgcggcggccctgggcgtctcgccggtctggctgcagaccggcgaaggcgctccagccgcgcgcagtgccgtgtccgtggccgatggcagcccattggtgctggaaccgctgcatccgtgggacagcgacacaccgctggacgaagacgaagtggaactgccgctgtacaaggaagtggagatgtccgccggcgccggacgcactgcggtacgcgagatagaggggcgcaagctgcgtttttcctatgccacgctgcgagcctcgggcgtcgatccgtcggcggcgatctgcgcccaactcaccggcaacagcatggaaccgctgatcatggatggctccaccatcggcgtggacaccgccaccacccatatcaccgatggcgagatctacgccctcgaacatgacggcatgctgcgggtgaagttcgtctatcgcctgcccggcggcggcattcggctgcgcagcttcaaccgcgaggaatacccggacgaggagtactcgccggaggacatgcgcagccgccagatcagcatgatcggctgggtcttctggtggtccaccgtacgccaccggcgcgccccgtccctggtgcggtga

SEQ ID NO: 21 - DNA, 2076 bp, Pseudomonas aeruginosa
  misc_feature 1..2076: note = Description of sequence: polynucleotide, name: MRSN317 R2 pyocin tail fiber
  source 1..2076: mol_type = genomic DNA
atgacgaccaatactccgaaatacggtggcctgctcaccgacataggtgccgctgcgctggctgcggccagcgcagcaggcaagaaatggcagccgactcatatgctgatcggcgatgctggcggtgcgccgggcgacacgccggatccattgccttcggcggcgcagaagagcctgatcaaccaacgccatcgggcccagctgaatcggctgttcgtttccgacaagaacgccaataccttggttgccgaggtggtgctgccagttgaggtaggtggcttctggatccgcgagatcggcctgcaggatgccgacggcaagttcgtcgcggtatccaactgcccgcccagctacaaggctgcgatggaaagtggcagtgcgcggacccagaccattcgggtgaacatcgcgctctccggcctggagaatgtccagctgctgatcgacaacggcatcatctacgccactcaggactgggtgaaggaaaaggtcgctgccgatttcaagggccgcaagatcctggctggcaatggcctgctcggtgggggcgatctttctgccgaccgcagcattggcctggcgccttccggcgtgacggcgggcagctatcgttcggtcacggtgaacgccaacggggtggtcacccagggcagcaatccgaccaccctggccggctatgcgatcggagatgcctataccaaggccgataccgacggaaaactggcgcagaaagcgaacaaggccaccaccctggccggctatggcatcaccgatgcgctgcgagtcgatggcaacgccgtgtcatccagcaggctggccgcaccgcgcagcctggcagccagtggcgatgcctcctggtcggtgaccttcgacggcagtgccaatgtttctgcgccgctgagtctttccgctaccggtgtggcggcgggcagctatccgaaggtgaccgtggatacgaagggaagggtgactgctggaatggcgctggcggcgacggacattcccgggctggatgcgtcgaagctggtcagcggggtgctggccgagcagcgtttgccggtattcgcgcgcgggttggctactgctgtctcgaacagtagcgatccgaacaccgcgaccgtgccgttgatgctgaccaatcatgcgaacggacctgttgccggacgatacttctacatccagtcgatattctatccggatcagaacggcaatgcttcgcagattgcaacgagctacaacgctacatccgagatgtatgtacgggtgtcctacgcggccaaccctagcatccgggagtggttgccctggcagcgctgcgacattggaggttccttcaccaaagaggccgatggtgaactgcctggaggcgtcaacctggattcgatggtgacctcagggtggtggagccagagttttactgcccaagctgccagtggagccaactaccctatagttcgggccggcctgcttcatgtgtacgccgcgagtagcaatttcatctatcagacgtatcaagcctacgatggtgagagtttctatttccggtgccggcattcaaatacctggtttccctggcgtcgcatgtggcatggcggagacttcaaccccagtgactatctgttgaagtcggggttctattggaatgcgttaccgggaaaacctgccacttttccaccatccgcacataaccatgacgtcggacagcttacttcgggcattctcccactggcacgtggcggcgtcggttcgaatacggcagcgggagcacgtagcactatcggagcaggggttcctgcgactgcttcccttggggcgagcggatggtggcgggacaatgacactggcctcattaggcaatgggggcaggtcacttgccccgccgatgccgatgcttcgattacgttcccgattcctttccctacgctatgcctcggcggatatgcgaatcagacgagtgctttccatccgggaacggatgccagtacaggtttccgtggagcgactaccactaccgcggtgattcgcaatggctactttgctcaggcggttctttcatgggaggcatttggacgatga

SEQ ID NO: 22 - DNA, 2109 bp, Pseudomonas aeruginosa
  misc_feature 1..2109: note = Description of sequence: polynucleotide, name: TuD199 R5 pyocin tail fiber
  source 1..2109: mol_type = genomic DNA
atgacgaccaatactccgaaatacggtggcctgctcaccgacataggtgccgctgcgctggctgcggccagcgcagcaggcaagaaatggcagccgactcatatgctgatcggcgatgctggcggcgcaccgggtgctactccggatccgatacccgcagccacgcagacgaaactgatcaaccagcgctaccgggcgcagctcaatcgcttgttcgtgtcggacaagaacatcaatactctggttgccgaggtggtgctaccagttgaggtaggtggcttctggatccgcgagatcggcctgcaggatgccgacggcaagttcgtcgcggtatccaactgcccgcccagctacaaggcagcaatggaaagtggcagtgcgcggacccagaccattcgggtgaacatcgcgctctccggcctggagaatgtccagttgctgatcgacaacggcatcatctacgccactcaggactgggtgaaggaaaaggtcgctgccgatttcaagggccgcaagatcctggctggcaatggcctggtcggtgggggcgatctttctgccgaccgcagcattggcctggcgccttccggcgtgacggcgggcagctatcgctcggtcacggtgaacgccaacggggtggtcacccagggcagcaatccgagcaccctggccggctatgcgatcggagatgcctataccaaggccgataccgacggaaaactggcgcagaaagcgaacaaggccaccaccctggccggctatggcatcaccgatgcgctgcgagtcgatggcaacgccgtgtcatccagcaggctggccgcaccgcgcagcctggcagccagtggcgatgcctcctggtcggtgaccttcgacggcagtgccaatgtttctgcgccgctgagtctttccgctaccggtgtggcagcgggcagctatccgaaggtgaccgtggatacgaagggaagggtgactgctggaatggcgctggcggcgacggacattcccgggctggatgcttcgaagttggtcagcggggtgctggccgagcagcgtttgccggtattcgcgcgcgggttggctactgctgtctcgaccactagcgatccgaacaccgcgaccgtgccgttgatgctgaccaatcatgcgaacggacctgttgccggacggtacttctacatccagtcgatgttctatccggatcagaacggcaatgcttcgcagattgcaacgagctacaacgctacatccgagatgtatgtacgggtgtcctacgcggccaaccctagcgcccgggactggctgccctggaagcgctgcgacataggtggttcgttcagcaaggaggcggacggggccttgggcggtgcggtcaatctcaactcgctcatcacgtcgggatggtggtaccagacggccaatgcacaggccgaaagtggggcgaactacccggtcccccgggccggcttgcttcaagtgcataatgcaggcaccaatttcatctaccagacataccaggtttatgacggtgaagggttctattttcgttgccgctacaccaacacctggtatccatggcggcgtgtatggcatggagcggacttcaatccgaacgactatctgctgaaaagtggctttacgtgggccgccctgccaggaaagcccgcgacctttccgcctactggccacaaccacgacgccgcccagatcacgtcgggcatcctgcctctggcgcgcggcggtcttggttcgaatacggcagcgggggcgcgcaacaatatcggggctggggttcccgcgacggcgaaccgatcgctgaatggctggtggaaggataacgatacaggactcatcgtgcagtggatgacagtgagtgtcggcgatcatccgggtggaatcgtcaaccgctcgctgacctttccgatcgcgtttcccactacctgcctgcacgtggtgccgagcgtcaaggaactggggcggccggcgacgtcggcatcgaccgtcaccctcgcagatgtcagcgtcagcaccacggggtgtgtgattgtcgctaccgagtatcacggtgcggtccagaactatgcgatcaggcttgtggccattggctgttga

SEQ ID NO: 23 - protein, 701 aa, Pseudomonas aeruginosa
  REGION 1..701: note = Description of sequence: peptide, name: MRSN2101 R1 pyocin tail fiber
MTTNTPKYGGLLTDIGAAALAAASAAGKKWQPTHMLIGDAGGAPGDTPDPLPSAAQKSLINQRHRAQLNRLFVSDKNANTLVAEVVLPVEVGGFWIREIGLQDADGKFVAVSNCPPSYKAAMESGSARTQTIRVNIALSGLENVQLLIDNGIIYATQDWVKEKVAADFKGRKILAGNGLVGGGDLSADRSIGLAPSGVTAGSYRSVTVNANGVVTQGSNPTTLAGYAIGDAYTKADTDGKLAQKANKATTLAGYGITDALRVDGNAVSSSRLAAPRSLAASGDASWSVTFDGSANVSAPLSLSATGVAAGSYPKVTVDTKGRVTAGMALAATDIPGLDASKLVSGVLAEQRLPVFARGLATAVSNSSDPNTATVPLMLTNHANGPVAGRYFYIQSMFYPDQNGNASQIATSYNATSEMYVRVSYAANPSIREWLPWQRCDIGGSFTKTTDGSIGNGVNINSFVNSGWWLQSTSEWAAGGANYPVGLAGLLIVYRAHADHIYQTYVTLNGSTYSRCCYAGSWRPWRQNWDDGNFDPASYLPKAGFTWAALPGKPATFPPSGHNHDTSQITSGILPLARGGLGANTAAGARNNIGAGVPATASRALNGWWKDNDTGLIVQWMQVNVGDHPGGIIDRTLTFPIAFPGACLHVVPTVKEVGRPATSASTVTVADVSVSNTGCVIVSSEYYGLAQNYGIRVMAIGY

SEQ ID NO: 24 - protein, 691 aa, Pseudomonas aeruginosa
  REGION 1..691: note = Description of sequence: peptide, name: MRSN317 R2 pyocin tail fiber
MTTNTPKYGGLLTDIGAAALAAASAAGKKWQPTHMLIGDAGGAPGDTPDPLPSAAQKSLINQRHRAQLNRLFVSDKNANTLVAEVVLPVEVGGFWIREIGLQDADGKFVAVSNCPPSYKAAMESGSARTQTIRVNIALSGLENVQLLIDNGIIYATQDWVKEKVAADFKGRKILAGNGLLGGGDLSADRSIGLAPSGVTAGSYRSVTVNANGVVTQGSNPTTLAGYAIGDAYTKADTDGKLAQKANKATTLAGYGITDALRVDGNAVSSSRLAAPRSLAASGDASWSVTFDGSANVSAPLSLSATGVAAGSYPKVTVDTKGRVTAGMALAATDIPGLDASKLVSGVLAEQRLPVFARGLATAVSNSSDPNTATVPLMLTNHANGPVAGRYFYIQSIFYPDQNGNASQIATSYNATSEMYVRVSYAANPSIREWLPWQRCDIGGSFTKEADGELPGGVNLDSMVTSGWWSQSFTAQAASGANYPIVRAGLLHVYAASSNFIYQTYQAYDGESFYFRCRHSNTWFPWRRMWHGGDFNPSDYLLKSGFYWNALPGKPATFPPSAHNHDVGQLTSGILPLARGGVGSNTAAGARSTIGAGVPATASLGASGWWRDNDTGLIRQWGQVTCPADADASITFPIPFPTLCLGGYANQTSAFHPGTDASTGFRGATTTTAVIRNGYFAQAVLSWEAFGR

SEQ ID NO: 25 - protein, 702 aa, Pseudomonas aeruginosa; identical to SEQ ID NO: 13
  REGION 1..702: note = Description of sequence: peptide, name: TuD199 R5 pyocin tail fiber

(19) United States
(12) Patent Application Publication
(10) Pub. No.: US 2026/0191928 A1
(43) Pub. Date: Jul. 9, 2026

(54) R-PYOCIN COMPOSITIONS AND METHODS OF USE THEREOF

(71) Applicant: Georgia Tech Research Corporation, Atlanta, GA (US)

(72) Inventors: Stephen Paul Diggle, Atlanta, GA (US); Isaac Michael Estrada, Atlanta, GA (US)

(21) Appl. No.: 19/441,036

(22) Filed: Jan. 6, 2026

Related U.S. Application Data
(60) Provisional application No. 63/742,212, filed on Jan. 6, 2025.

(51) Int. Cl. A61K 38/16; A61K 31/496; A61K 31/7036; A61P 31/04; C12Q 1/18

(52) U.S. Cl. CPC A61K 38/164; A61K 31/496; A61K 31/7036; A61P 31/04; C12Q 1/18

(57) ABSTRACT

Disclosed herein are therapeutic compositions comprising an R-pyocin and methods of use thereof in treating multidrug-resistance bacterial infection, in re-sensitizing a multidrug-resistant bacteria to an antibiotic, and in identifying R-pyocins with antimicrobial action against high-risk strains.

### CROSS-REFERENCE TO RELATED APPLICATIONS

This application claims priority to, and the benefit of, U.S. Provisional Patent Application No. 63/742,212, filed Jan. 6, 2025, which is incorporated by reference herein in its entirety.

### STATEMENT OF GOVERNMENT SUPPORT

This invention was made with government support under AI184449, and AI153116 awarded by the National Institutes of Health. The government has certain rights in the invention.

### SEQUENCE LISTING

A Sequence Listing conforming to the rules of WIPO Standard ST.26 is herby incorporated by reference. Said Sequence Listing has been filed as an electronic document via Patent Center encoded as XML in UTF-8 text. The electronic document, created on Jan. 5, 2026, is entitled “10034-411US1_ST26.xml”, and is 162,342 bytes in size.

### BACKGROUND

Pseudomonas aeruginosa (Pa) is a gram-negative, facultative anaerobic bacterium that is known to cause a wide range of infections in humans. Data from the National Healthcare Safety Network (NHSN) reveals that Pa is responsible for 10.3% of catheter-associated urinary tract infections, 4% of central line-associated bloodstream infections, 16.5% of ventilator-associated pneumonia, and 5.7% of surgical site infections. In total, Pa contributes to 7.3% of these difficult-to—treat infections and 10% of hospital-acquired infections worldwide. Furthermore, Pa is the primary bacterial pathogen that colonizes and persistently infects the lungs of individuals with cystic fibrosis (CF). A significant challenge in treating Pa infections is their ability to form multicellular biofilms, which are responsible for over 80% of chronic infections and are extremely difficult to treat. Chronic wounds, in particular, represent a significant burden on healthcare systems, with an estimated 6.5 million Americans receiving treatment annually at a cost of around $50 billion per year. As rates of obesity and Type 2 diabetes continue to rise, the prevalence of chronic wounds and associated healthcare costs are expected to increase further.

In 2021, it was estimated that 4.7 million deaths were associated with bacterial antimicrobial resistance (AMR), including 1.14 million deaths directly attributable to AMR. The same report predicts that this will increase to 8.22 million associated and 1.91 million direct deaths by 2050. AMR presents a significant challenge in treating Pa infections, as the bacterium becomes resistant to most antibiotic classes. The prevalence of multidrug-resistant (MDR) Pa has been rising over the past decade, causing concern due to limited treatment options and increased morbidity and mortality associated with these strains. In 2017, the World Health Organization (WHO) placed carbapenem-resistant Pa strains on the high-priority list for new antibiotic research and development. Pa has been linked to outbreaks containing the Verona integron-encoded metallo-beta-lactamase (blaVIM) gene, which is concerning because carbapenem antibiotics are often a primary treatment for hospital-acquired Pa infections. MDR, extensively drug-resistant (XDR) and pan-drug resistant (PDR) strains are increasing and associated with higher mortality rates. Certain Pa strain-types (ST) are considered ‘high-risk’ due to their widespread dissemination and resistance to treatment. The top 10 most widespread high-risk Pa strain-types worldwide are currently ST235, ST111, ST233, ST244, ST357, ST308, ST175, ST277, ST654, and ST298. ST235, a globally widespread strain of Pa, is known to carry numerous beta-lactamase isolates and horizontally-acquired antibiotic resistance.

Due to the lack of new antibiotics in development, what is urgently needed are novel antimicrobials against gram-negative organisms, particularly Pseudomonas aeruginosa; otherwise, there is a risk of returning to the pre-antibiotic era. This need is at least partially fulfilled by the present application.

### SUMMARY

Disclosed herein are therapeutic compositions and kits comprising an R-pyocin and the methods of use thereof in treating multidrug-resistance bacterial infection and in re-sensitizing a multidrug-resistant bacteria to an antibiotic.

Accordingly, in one aspect, disclosed herein is a therapeutic composition comprising an R-pyocin and a pharmaceutically acceptable carrier. In some embodiments, the R-pyocin is isolated from an engineered strain of Pseudomonas aeruginosa. In some embodiments, the pharmaceutically acceptable carrier comprises an oil-in-water emulsion, a nano-emulsion, a nanoparticle, an excipient, a diluent, a salt, a buffer, a stabilizer, a lipid, or combinations thereof. In some embodiments, the engineered strain of Pseudomonas aeruginosa is selected from PAK, PAO1, Pa-Tud-199, PA-14, PA7, UCBPP-PA14 or RP73. In some embodiments, the R-pyocin comprises SEQ ID NO: 1, SEQ ID NO: 4, SEQ ID NO: 7, SEQ ID NO: 9, SEQ ID NO:11, SEQ ID NO: 18, SEQ ID NO: 20, or a sequence at least 70% identical to SEQ ID NO: 1, SEQ ID NO: 4, SEQ ID NO: 7, SEQ ID NO: 9, SEQ ID NO:11, SEQ ID NO: 18, or SEQ ID NO: 20.

In one aspect, disclosed herein is a method of treating a multidrug-resistant bacterial infection in a subject, comprising administering to the subject the therapeutic composition comprising an R-pyocin and a pharmaceutically acceptable carrier. In some embodiments, the R-pyocin is isolated from an engineered strain of Pseudomonas aeruginosa. In some embodiments, the R-pyocin is isolated from a sample obtained from the subject. In some embodiments, the sample is selected from the group consisting of blood, CSF, serum, tissue, wound exudate, abscess exudate, burn exudate, sputum, eye sample, and ear sample.

In some embodiments, the multidrug-resistant bacterial infection is caused by Pseudomonas aeruginosa. In some embodiments, the method further comprises administering an antibiotic. In some embodiments the antibiotic is an aminoglycoside or fluoroquinolone. In a further embodiment, the aminoglycoside is amikacin, gentamicin, and/or tobramycin. In some embodiments, the pharmaceutically acceptable carrier comprises an oil-in-water emulsion, a nano-emulsion, a nanoparticle, an excipient, a diluent, a salt, a buffer, a stabilizer, a lipid, or combinations thereof. In some embodiments, the engineered strain of Pseudomonas aeruginosa is selected from PAK, PAO1, Pa-Tud-199, PA-14, PA7, UCBPP-PA14 or RP73. In some embodiments, the R-pyocin comprises SEQ ID NO: 1, SEQ ID NO: 4, SEQ ID NO: 7, SEQ ID NO: 9, SEQ ID NO:11, SEQ ID NO: 18, SEQ ID NO: 20, or a sequence at least 70% identical to SEQ ID NO: 1, SEQ ID NO: 4, SEQ ID NO: 7, SEQ ID NO: 9, SEQ ID NO:11, SEQ ID NO: 18, or SEQ ID NO: 20.

In some embodiments, the method increases the antibiotic sensitivity of the bacteria in a treated subject compared to an untreated control. In some embodiments, the method decreases biofilm formation, by the bacteria in a treated subject compared to an untreated control. In some embodiments, the method decreases the virulence of the bacteria in a treated subject compared to an untreated control. In some embodiments, increased antibiotic sensitivity of the bacteria treated with the R-pyocin is indicated by the presence of pyomelanin pigment.

In a further aspect, disclosed herein is a method of re-sensitizing a multidrug-resistant bacteria to an antibiotic, comprising, isolating an R-pyocin from an engineered strain of Pseudomonas aeruginosa, contacting the multidrug-resistant bacteria with the R-pyocin, contacting the multidrug-resistant bacteria with an antibiotic, and measuring colony forming unit (CFU) ability of the multidrug-resistant bacteria, wherein a decrease in CFU ability of the multidrug-resistant bacteria denotes increased antibiotic re-sensitization. In some embodiments, the antibiotic is an aminoglycoside or a fluoroquinolone. In some embodiments, the multidrug-resistant bacteria is Pseudomonas aeruginosa. In a further embodiment, the aminoglycoside is amikacin, gentamicin, and/or tobramycin. In some embodiments, the engineered strain of Pseudomonas aeruginosa is selected from PAK, PAO1, Pa-Tud-199, PA-14, PA7, UCBPP-PA14 or RP73. In some embodiments, the R-pyocin comprises SEQ ID NO: 1, SEQ ID NO: 4, SEQ ID NO: 7, SEQ ID NO: 9, SEQ ID NO:11, SEQ ID NO: 18, SEQ ID NO: 20 or a sequence at least 70% identical to SEQ ID NO: 1, SEQ ID NO: 4, SEQ ID NO: 7, SEQ ID NO: 9, SEQ ID NO:11, SEQ ID NO: 18, or SEQ ID NO: 20. In some embodiments, the multidrug-resistant bacteria comprise a deletion comprising SEQ ID NO: 14, SEQ ID NO: 15, SEQ ID NO: 16, SEQ ID NO: 17, or a sequence at least 70% identical to SEQ ID NO: 14, SEQ ID NO: 15, SEQ ID NO: 16, or SEQ ID NO: 17. In some embodiments, the method decreases biofilm formation, by the bacteria in a treated subject compared to an untreated control.

In another aspect, disclosed herein is a method of identifying R-pyocins with antimicrobial properties against high-risk bacterial strains, the method comprising a) collecting bacterial samples; b) sequencing said samples and predicting the presence of R-pyocins; c) extracting and purifying R-pyocins; and d) testing purified R-pyocins' antimicrobial use against high-risk bacterial strains. In some embodiments the bacterial sample comprises Pseudomonas aeruginosa. In some embodiments, the high-risk bacterial strain comprises Pseudomonas aeruginosa.

In another aspect, disclosed herein is a kit for testing pyocin mediated collateral susceptibility of an infection-causing bacteria, wherein the kit comprises an R-pyocin isolated from an engineered strain of Pseudomonas aeruginosa. In a further embodiment, the kit comprises a sample collection tool, infection-causing bacteria isolation media, infection-causing bacteria growth media. In some embodiments, the R-pyocin comprises SEQ ID NO: 1, SEQ ID NO: 4, SEQ ID NO: 7, SEQ ID NO: 9, SEQ ID NO:11, SEQ ID NO: 18, SEQ ID NO: 20, or a sequence at least 70% identical to SEQ ID NO: 1, SEQ ID NO: 4, SEQ ID NO: 7, SEQ ID NO: 9, SEQ ID NO:11, SEQ ID NO: 18, or SEQ ID NO: 20.

### BRIEF DESCRIPTION OF DRAWINGS

FIGS. 1A-1B depict R-pyocins. FIG. 1A shows R-type specificity is determined by the sequence on the foot of the tail fiber, which can recognize glycosylated structures on the outer membrane (such as the LPS). FIG. 1B shows TEM of a Pa cell decorated with R-pyocins.

FIGS. 2A-2B depict anti-biofilm efficacy of R-pyocins. FIG. 2 shows a 15 h biofilm of A018 that was treated with R-pyocins extracted from A026 (FIG. 2A) and A026ΔR (R-pyocin mutant) (FIG. 2B). A significant portion of the biomass was killed (red cells) after 2 h and full-depth lethal effects on the biomass was achieved after 4 h. This effect was absent in the control experiment, using extractions from A026ΔR.

FIG. 3 depicts R-pyocin susceptibility of 100 high-risk Pa strains. 96 were susceptible to at least one R-pyocin type produced by laboratory strains: PAK (R1, SEQ ID NO: 1), PAO1 (R2, SEQ ID NO: 4), TuD-199 (R5, SEQ ID NO: 22), and clinical MRSN strains 2102 (R1, SEQ ID NO: 18), 317 (R2, SEQ ID NO: 20), 1344 (R5, SEQ ID NO: 11).

FIGS. 4A-4C depict disk diffusion tests. FIG. 4A shows Kirby-Bauer Disk diffusion test of MRSN 6220, a PDR strain showing only limited susceptibility to Colistin. FIG. 4B shows Kirby-Bauer Disk diffusion test of a brown mutant of MRSN 6220, (6220-2A2), which shows increased sensitivity to aminoglycoside antibiotics Amikacin, Gentamicin, and Tobramycin. FIG. 4C shows a comparison of the zones of inhibition of MRSN 6220 and 6220-2A2.

FIGS. 5A-5D depict that genomic diversity as measured by core genome SNPs varies greatly from one population to another. Populations are presented in order of decreasing genomic diversity: Patient 1 (FIG. 5A), Patient 2 (FIG. 5B), Patient 3 (FIG. 5C), and Patient 4 (FIG. 5D). Each matrix represents the pairwise comparison of SNPs across all 75 isolates within a population against each other, and each population is composed of a single strain type. Isolates with one DNA mismatch repair mutation are highlighted in yellow on phylogenies. Isolates with two DNA repair mutations are highlighted in red.

FIG. 6 depicts preliminary assessment of cytotoxic effects of R-pyocins on immune cell lines. FIG. 6 shows that purified R-pyocins from control strains and clinical strains of Pa were tested against Human Lung Epithelial cells (A549) at 50 μg/ml and 100 μg/ml. No significant cytotoxic effects were observed using any of the purified R-pyocins when compared to the negative control (NC), 1X PBS, pH 7.4. Cell death was observed using 1% Triton-X-100 as a positive control (PC).

FIGS. 7A-7D depict in vitro and in vivo models of wound infections. FIG. 7A shows that the wound microcosm model incorporates a chopped-meat-based media (Bolton's broth) and formulated to best represent the conditions of human wounds and containing physiological concentrations of blood components. FIG. 7B shows that upon coagulation, bacterial communities exhibit spatial biogeography within the fibrin matrix, similar to that seen in chronic wound infections. FIG. 7C shows that the area of a murine surgical excision wound can be measured using a SilhouetteStar laser scanning wound imaging, 3D measurement and documentation system (ARANZ Medical). FIG. 7D shows a murine surgical excision wound after 4 days of infection.

### DETAILED DESCRIPTION

The following description of the disclosure is provided as an enabling teaching of the disclosure in its best, currently known aspects. Many modifications and other aspects disclosed herein will come to mind to one skilled in the art to which the disclosed compositions and methods pertain, benefiting from the teachings presented in the descriptions herein and the associated drawings. Therefore, it is understood that the disclosures are not limited to the specific aspects disclosed and that modifications and other aspects are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As apparent to those of skill in the art upon reading this disclosure, each of the individual aspects described and illustrated herein has discrete components and features that may be readily separated from or combined with the features of any of the other several aspects without departing from the scope or spirit of the present disclosure.

Any recited method can be carried out in the order of events recited or any other order that is logically possible. Unless otherwise expressly stated, it is in no way intended that any method or aspect set forth herein be construed as requiring that its steps be performed in a specific order. Accordingly, where a method claim does not explicitly state in the claims or descriptions that the steps are to be limited to a particular order, it is in no way intended that an order be inferred in any respect. This holds for any possible non-express basis for interpretation, including logic concerning arrangement of steps or operational flow, meaning derived from grammatical organization or punctuation, or the number or type of aspects described in the specification.

All publications mentioned herein are incorporated by reference to disclose and describe the methods or materials in connection with which the publications are cited. The publications discussed herein are provided solely for their disclosure before the filing date of the present application. Further, the dates of publication provided herein can be different from the actual publication dates, which can require independent confirmation.

It is also to be understood that the terminology herein describes particular aspects only and is not intended to be limiting. Unless defined otherwise, all technical and scientific terms herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Before describing the various aspects of the present disclosure, the following definitions are provided and should be used unless otherwise indicated. Additional terms may be defined elsewhere in the present disclosure.

As used herein, “comprising” is interpreted as specifying the presence of the stated features, integers, steps, or components but does not preclude the presence or addition of one or more features, integers, steps, components, or groups thereof. Moreover, each of the terms “by,” “comprising,” “comprises,” “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, “consisting essentially of” is intended to include examples encompassed by the term “consisting of.”

As used in the specification and the appended claims, the singular forms “a,” “an,” and “the” include plural referents unless the context dictates otherwise.

Ratios, concentrations, amounts, and other numerical data can be expressed herein in a range format. Further, the endpoints of each of the ranges are significant both in relation to the other endpoint and independently of the other endpoint. There are many values disclosed herein, and each value is also disclosed as “about” that particular value in addition to the value itself. For example, if the value “10” is disclosed, then “about 10” is also disclosed. Ranges can be expressed herein as from “about” one particular value and to “about” another particular value. Similarly, when values are expressed as approximations, using the antecedent “about,” the particular value forms a further aspect. For example, if the value “about 10” is disclosed, then “10” is also disclosed.

When a range is expressed, a further aspect includes from the one particular value and to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x,’ ‘about y,’ and ‘about z’ as well as the ranges of ‘less than x,‘ ’less than y.‘ and’less than z.’ Likewise, the phrase ‘about x, y, z, or greater’ should be interpreted to include the specific ranges of ‘about x,’ ‘about y,’ and ‘about z’ as well as the ranges of ‘greater than x,’ greater than y,‘ and’greater than z.’ In addition, the phrase “about ‘x’ to ‘y’,” where ‘x’ and ‘y’ are numerical values, includes “about ‘x’ to about ‘y’.”

Such a range format is used for convenience and brevity and thus, should be interpreted flexibly to include not only the numerical values explicitly recited as the limits of the range but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, amounts, sizes, formulations, parameters, and other quantities and characteristics are not and need not be exact but may be approximate, larger or smaller, as desired, reflecting tolerances, conversion factors, rounding, measurement error, and the like, and other factors known to those of skill in the art such that equivalent results or effects are obtained. In some circumstances, the value that provides equivalent results or effects cannot be reasonably determined. In such cases, as used herein, “about” and “at or about” mean the nominal value indicated ±10% variation unless otherwise indicated or inferred. In general, an amount, size, formulation, parameter, or other quantity or characteristic is “about,” “approximate,” or “at or about,” whether or not expressly stated to be such. Where “about,” “approximate,” or “at or about” is used before a quantitative value, the parameter also includes the specific quantitative value itself unless expressly stated otherwise.

Compounds are described using standard nomenclature. Unless defined otherwise, all technical and scientific terms used herein have the same meaning as is commonly understood by one of skill in the art to which this disclosure belongs.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers, such as Sigma-Aldrich (formally MilliporeSigma, Burlington, MA) or Thermo Fisher Scientific Inc. (Waltham, MA), or are prepared by methods known to those skilled in the art following procedures set forth in references such as Fieser and Fieser's Reagents for Organic Synthesis (John Wiley and Sons, 2007); Organic Reactions (John Wiley and Sons, 2004); March's Advanced Organic Chemistry, (John Wiley and Sons, 8^{th} Edition); and Larock's Comprehensive Organic Transformations (John Wiley and Sons, 3^{rd} edition, 2017).

A number of aspects of the disclosure have been described. Nevertheless, it will be understood that various modifications may be made without departing from the spirit and scope of the disclosure. Accordingly, other aspects are within the scope of the following claims.

By way of non-limiting illustration, examples of certain aspects of the present disclosure are given below.

The following examples are set forth below to illustrate the compounds, compositions, articles, devices, and methods claimed herein, along with associated methods and results according to the disclosed subject matter. These examples are not intended to be inclusive of all aspects of the subject matter disclosed herein, but rather to illustrate representative methods and results. These examples are not intended to exclude equivalents and variations of the present disclosure, which are apparent to one skilled in the art.

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition, or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, or stabilize a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

A “control” is an alternative subject or sample used in an experiment for comparison purposes. A control can be “positive” or “negative.”

As used herein, “enhance”, “enhanced”, “enhancement”, “enhancing”, and any grammatical variations thereof as used herein, refers to an act of intensifying, increasing, or further improving the quality, value, or extent of a biological function, composition, compound, cell, or tissue.

As used herein, “diagnose”, “diagnosed”, “diagnosing”, and any grammatical variations thereof as used herein, refers to the act of process of identifying the nature of an illness, disease, disorder, or condition in a subject by examination or monitoring of symptoms.

A “protein”, “polypeptide”, or “peptide” each refer to a polymer of amino acids and does not imply a specific length of a polymer of amino acids. Thus, for example, the terms peptide, oligopeptide, protein, antibody, and enzyme are included within the definition of polypeptide. This term also includes polypeptides with post-expression modification, such as glycosylation (e.g., the addition of a saccharide), acetylation, phosphorylation, and the like.

Reference also is made herein to peptides, polypeptides, proteins, and compositions comprising peptides, polypeptides, and proteins. As used herein, a polypeptide and/or protein is defined as a polymer of amino acids, typically of length≥100 amino acids (Garrett & Grisham, Biochemistry, 2nd edition, 1999, Brooks/Cole, 110). A peptide is defined as a short polymer of amino acids, of a length typically of 20 or less amino acids, and more typically of a length of 12 or less amino acids (Garrett & Grisham, Biochemistry, 2nd edition, 1999, Brooks/Cole, 110).

The peptides, polypeptides, and proteins disclosed herein may be modified to include non-amino acid moieties. Modifications may include but are not limited to carboxylation (e.g., N-terminal carboxylation via addition of a di-carboxylic acid having 4-7 straight-chain or branched carbon atoms, such as glutaric acid, succinic acid, adipic acid, and 4,4-dimethylglutaric acid), amidation (e.g., C-terminal amidation via addition of an amide or substituted amide such as alkylamide or dialkylamide), PEGylation (e.g., N-terminal or C-terminal PEGylation via additional of polyethylene glycol), acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine, or histidine).

This specification discusses various nucleic acid sequences. It is understood that the proteins and protein sequences encoded by these nucleic acids are also disclosed.

The phrases “percent identity” and “% identity,” as applied to polypeptide sequences or nucleic acid sequences, refers to the percentage of residue matches between at least two polypeptide sequences or at least two nucleic acid sequences aligned using a standardized algorithm. In some embodiments disclosed herein is a sequence at least 70%, 75%, 80%, 85%, 90%, 91%, 92%, 93%, 94%, 95%, 96%, 97%, 98%, 99%, or 100% identical SEQ ID NO: 1. In some embodiments disclosed herein is a sequence at least 70%, 75%, 80%, 85%, 90%, 91%, 92%, 93%, 94%, 95%, 96%, 97%, 98%, 99%, or 100% identical SEQ ID NO: 4. In some embodiments disclosed herein is a sequence at least 70%, 75%, 80%, 85%, 90%, 91%, 92%, 93%, 94%, 95%, 96%, 97%, 98%, 99%, or 100% identical SEQ ID NO: 7. In some embodiments disclosed herein is a sequence at least 70%, 75%, 80%, 85%, 90%, 91%, 92%, 93%, 94%, 95%, 96%, 97%, 98%, 99%, or 100% identical SEQ ID NO: 9. In some embodiments disclosed herein is a sequence at least 70%, 75%, 80%, 85%, 90%, 91%, 92%, 93%, 94%, 95%, 96%, 97%, 98%, 99%, or 100% identical SEQ ID NO: 11. In some embodiments disclosed herein is a sequence at least 70%, 75%, 80%, 85%, 90%, 91%, 92%, 93%, 94%, 95%, 96%, 97%, 98%, 99%, or 100% identical SEQ ID NO: 14. In some embodiments disclosed herein is a sequence at least 70%, 75%, 80%, 85%, 90%, 91%, 92%, 93%, 94%, 95%, 96%, 97%, 98%, 99%, or 100% identical SEQ ID NO: 15. In some embodiments disclosed herein is a sequence at least 70%, 75%, 80%, 85%, 90%, 91%, 92%, 93%, 94%, 95%, 96%, 97%, 98%, 99%, or 100% identical SEQ ID NO: 16. In some embodiments disclosed herein is a sequence at least 70%, 75%, 80%, 85%, 90%, 91%, 92%, 93%, 94%, 95%, 96%, 97%, 98%, 99%, or 100% identical SEQ ID NO: 17. In some embodiments disclosed herein is a sequence at least 70%, 75%, 80%, 85%, 90%, 91%, 92%, 93%, 94%, 95%, 96%, 97%, 98%, 99%, or 100% identical SEQ ID NO: 18. In some embodiments disclosed herein is a sequence at least 70%, 75%, 80%, 85%, 90%, 91%, 92%, 93%, 94%, 95%, 96%, 97%, 98%, 99%, or 100% identical SEQ ID NO: 20. Methods of polypeptide and nucleic acid sequence alignment are well-known. Some alignment methods consider conservative amino acid substitutions. Such conservative substitutions, explained in more detail above, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastp,” that is used to align a known amino acid sequence with other amino acids sequences from a variety of databases.

Percent identity may be measured over the length of an entire defined polypeptide sequence or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length may be used to describe a length over which percentage identity may be measured.

The term “variant” means a polypeptide derived from a parent polypeptide by one or more (several) alteration(s), i.e., a substitution, insertion, and/or deletion, at one or more (several) positions. A substitution means a replacement of an amino acid occupying a position with a different amino acid; a deletion means removal of an amino acid occupying a position; and an insertion means adding 1 or more, such as 1, 2, 3, 4, 5, 6, 7, 8, 9 or 10, preferably 1-3 amino acids immediately adjacent an amino acid occupying a position. In relation to substitutions, ‘immediately adjacent’ may be to the N-side (‘upstream’) or C-side (‘downstream’) of the amino acid occupying a position (‘the named amino acid’). Therefore, for an amino acid named/numbered ‘X,’ the insertion may be at position ‘X+1’ (‘downstream’) or at position ‘X-1′ (‘upstream’).

A “variant” of a particular polypeptide sequence may be defined as a polypeptide sequence having at least 50% sequence identity to the particular polypeptide sequence over a certain length of one of the polypeptide sequences using blastp with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). In some embodiments a variant polypeptide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polypeptide.

A variant polypeptide may have substantially the same functional activity as a reference polypeptide. For example, a variant polypeptide may exhibit one or more biological activities associated with binding a ligand and/or binding DNA at a specific binding site.

As used herein, the term, “deletion,” also called gene deletion, deficiency, or deletion mutation, refers to part of a chromosome or a sequence of DNA being left out during DNA replication. Deletion, or gene deletions can cause any number of nucleotides to be deleted from a single base to an entire piece of chromosome.

Variants comprising deletions relative to a reference amino acid sequence or nucleotide sequence are contemplated herein. A “deletion” refers to a change in the amino acid or nucleotide sequence that results in the absence of one or more amino acid residues or nucleotides relative to a reference sequence. A deletion removes at least 1, 2, 3, 4, 5, 10, 20, 50, 100, or 200 amino acids residues or nucleotides. A deletion may include an internal deletion or a terminal deletion (e.g., an N-terminal truncation or a C-terminal truncation or both of a reference polypeptide or a 5′-terminal or 3′-terminal truncation or both of a reference polynucleotide).

“Culture” or “cell culture” is the process by which cells are grown under controlled conditions, generally outside their natural environment. After the cells of interest have been isolated from living tissue, they can subsequently be maintained under carefully controlled conditions. These conditions vary for each cell type but generally consist of a suitable vessel with a substrate or medium that supplies the essential nutrients (amino acids, carbohydrates, vitamins, minerals), growth factors, hormones, and gases (CO₂, 02), and regulates the physio-chemical environment (pH buffer, osmotic pressure, temperature). Most cells require a surface or an artificial substrate to form an adherent culture as a monolayer (one single-cell thick), whereas others can be grown free floating in a medium as a suspension culture. “Cell culture” also refers to the culturing of cells derived from multicellular eukaryotes, especially animal cells, in contrast with other types of culture that also grow cells, such as plant tissue culture, fungal culture, and microbiological culture (of microbes).

The term “administer,” “administering”, or derivatives thereof refer to delivering a composition, substance, inhibitor, or medication to a subject or object by one or more the following routes: oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation or via an implanted reservoir. The term “parenteral” includes subcutaneous, intravenous, intramuscular, intra-articular, intra-synovial, intrasternal, intrathecal, intrahepatic, intralesional, and intracranial injections or infusion techniques.

The term “detect” or “detecting” refers to an output signal released for the purpose of sensing of physical phenomenon. An event or change in environment is sensed and signal output released in the form of light.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., multidrug-resistant bacterial infection) The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc. In some aspects, the composition disclosed herein comprises R-pyocin.

The term “kit” describes a wide variety of bags, containers, carrying cases, and other portable enclosures which may be used to carry and store solid substances, liquid substances, and other accessories necessary to test R-pyocin susceptibility of an infection-causing bacteria. Such kits and their contents along with any applicable procedures may be used to provide access to testing R-pyocin susceptibility of an infection-causing bacteria in accordance with the teachings of the present disclosure.

A “gene” refers to a polynucleotide containing at least one open reading frame that is capable of encoding a particular polypeptide or protein after being transcribed and translated. Any of the polynucleotides sequences described herein may be used to identify larger fragments or full-length coding sequences of the gene with which they are associated. Methods of isolating larger fragment sequences are known to those of skill in the art, some of which are described herein.

A “chromosome” refers to a long DNA molecule comprising part or all of the genetic material of an organism. Most chromosomes comprise very long thin DNA strands coated with packaging proteins, including but not limited to histone proteins and other chaperone proteins, critical for binding and condensing the DNA strands into the tightly compacted chromosome structures. Such chromosomes are formed to maintain and preserve genetic stability and integrity.

A “pharmaceutically effective amount” of a drug necessary to achieve a therapeutic effect may vary according to factors such as the age, sex, and weight of the subject. Dosage regimens can be adjusted to provide the optimum therapeutic response. For example, several divided doses may be administered daily, or the dose may be proportionally reduced as indicated by the exigencies of the therapeutic situation.

A “therapeutic composition” refers to at least one substance, molecule, or compound suitable for administering to a subject, wherein the composition further includes a pharmaceutical carrier.

The term “antimicrobial” refers to an agent that kills microorganisms or stops their growth.

The term “antibacterial” refers to an agent that is proven to kill bacteria or stops bacterial growth.

The term “antibiotic” refers to a type of antimicrobial substance active against bacteria.

These are the most important type of antimicrobial agent for fighting bacterial infections, and antibiotics medications are widely used in the treatment and prevention of such infections. They may either kill or inhibit the growth of bacteria.

“Antibiotic resistance” as used herein refers to when microbes evolve mechanisms that protect them from the effects of antimicrobials. This specifically refers to bacteria that become resistant to antibiotics.

The term “biofilm” refers to any syntrophic microorganisms in which cells stick to each other and often also to a surface. The adherent cells become embedded within a slimy extracellular matrix that is composed of extracellular polymeric substances. The cells within the biofilm produce the extracellular polymeric substances components, which are typically a polymeric combination of polysaccharides, proteins, lipids, and DNA. Biofilms may form on living or non-living surfaces and can be prevalent in natural, industrial, and hospital settings.

As used herein, the term infection refers to the invasion of tissues by pathogens, their multiplication, and reaction of host tissues to the infectious agent and any toxins they release.

Infections can be caused by a wide range of pathogen, most common are bacteria and viruses.

As used herein, “monitoring” refers to the actions of observing and checking the progress or quality of a treatment or procedure over a period of time. “Monitoring” also refers to observing the course of a disease or condition, such as a cancer, over a period of time.

As used herein, a “therapeutic regimen” refers to a structured treatment plan or strategy designed to improve and maintain health. Generally, a therapeutic regimen will be designed, prescribed, and/or administered by a licensed medical practitioner. The therapeutic regimen generally specifies the treatment dosage, the treatment scheduling, and the duration of the treatment. In some embodiments, the therapeutic regimen comprises one or more therapeutic compositions. In some embodiments, the therapeutic regimen comprises one or more therapeutic agents. In some embodiments, the therapeutic regimen comprises any combination of therapeutic compositions and therapeutic agents, such as for example the combination of an inhibitor and an antibody. In some embodiments, a therapeutic regimen comprises modifying, continuing, and/or initiating at least one therapeutic agent and/or therapeutic composition. In some embodiments, a therapeutic regimen comprises treating and/or preventing a disease, disorder, and/or condition.

As used herein, the term “expand”, “expanding”, and any grammatical variations thereof as used herein, refers to the cellular processes of cell growth, proliferation, and/or differentiation, wherein the processes are allowed to occur naturally or are accelerated for the purpose of increasing cell numbers, cell size, and/or cell maturity.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

### Pseudomonas Aeruginosa

Pseudomonas aeruginosa (Pa) is an opportunistic gram-negative bacteria that poses a significant threat to immunocompromised patients. Pseudomonas aeruginosa is known for its ability to form robust biofilms and thrive in harsh conditions and is a major cause of hospital-acquired infections. Multidrug-resistant Pa poses a critical clinical challenge and severely limits treatment options, highlighting the urgent need for new antimicrobial strategies.

Pyocins are bacteriocins, protein-based antimicrobial molecules, produced by Pa to compete against closely related bacterial strains. Structurally and functionally, many pyocins resemble bacteriophages and are classified into R-type, F-type, and S-type pyocins. R- and F-type pyocins act like contractile or flexible tail-like structures that disrupt the membrane of susceptible bacteria, while S-type pyocins are soluble proteins that can contain enzymatic domains capable of degrading nucleic acids. Pyocins are of interest as antimicrobial agents because of their specificity and reduced off-target effects compared with broad-spectrum antibiotics.

The present application discloses bacteria that are susceptible to R-pyocin mediated death as well as bacteria that are resistant to R-pyocins. These R-pyocin resistant bacteria have gene deletions of hmgA (SEQ ID NO: 15), galU (SEQ ID NO: 14), and/or MexXY (SEQ ID NO:16, SEQ ID NO: 17). While deletion of these genes confers resistance to death by R-pyocins, bacteria with these deletions are more susceptible to antibiotics as these genes play a role in mediating antibiotic resistance. For example, the MexXY efflux pump is known to remove antibiotics such as aminoglycosides.

### Methods of Use

Disclosed herein is a method of treating a multidrug-resistant bacterial infection in a subject, comprising administering to the subject the therapeutic composition comprising an R-pyocin and a pharmaceutically acceptable carrier. In some embodiments, the R-pyocin is isolated from an engineered strain of Pseudomonas aeruginosa. In some embodiments, the R-pyocin is isolated from a sample obtained from the subject. In some embodiments, the sample is selected from the group consisting of blood, CSF, serum, tissue, wound exudate, abscess exudate, burn exudate, sputum, eye sample, and ear sample.

In some embodiments, the multidrug-resistant bacterial infection is caused by Pseudomonas aeruginosa. In some embodiments, the method further comprises administering an antibiotic, such as an aminoglycoside (amikacin, gentamicin, and/or tobramycin) or fluoroquinolone. In some embodiments, the pharmaceutically acceptable carrier comprises an oil-in-water emulsion, a nano-emulsion, a nanoparticle, an excipient, a diluent, a salt, a buffer, a stabilizer, a lipid, or combinations thereof. In some embodiments, the engineered strain of Pseudomonas aeruginosa is selected from PAK, PAO1, Pa-Tud-199, PA-14, PA7, UCBPP-PA14 or RP73. In some embodiments, the R-pyocin comprises SEQ ID NO: 1, SEQ ID NO: 4, SEQ ID NO: 7, SEQ ID NO: 9, SEQ ID NO:11, SEQ ID NO: 18, SEQ ID NO: 20, or a sequence at least 70% identical to SEQ ID NO: 1, SEQ ID NO: 4, SEQ ID NO: 7, SEQ ID NO: 9, SEQ ID NO:11, SEQ ID NO: 18, or SEQ ID NO: 20.

In some embodiments, the method increases the antibiotic sensitivity of the bacteria in a treated subject compared to an untreated control. In some embodiments, the method decreases biofilm formation, by the bacteria in a treated subject compared to an untreated control. In some embodiments, the method decreases the virulence of the bacteria in a treated subject compared to an untreated control. In some embodiments, the multidrug-resistant bacteria comprise a deletion comprising SEQ ID NO: 14, SEQ ID NO: 15, SEQ ID NO: 16, SEQ ID NO: 17, or a sequence at least 70% identical to SEQ ID NO: 14, SEQ ID NO: 15, SEQ ID NO: 16, or SEQ ID NO: 17.

In a further aspect, disclosed herein is a method of re-sensitizing a multidrug-resistant bacteria to an antibiotic, comprising, a) isolating an R-pyocin from an engineered strain of Pseudomonas aeruginosa, b) contacting the multidrug-resistant bacteria with the R-pyocin, c) contacting the multidrug-resistant bacteria with an antibiotic, and d) measuring colony forming unit (CFU) ability of the multidrug-resistant bacteria, wherein a decrease in CFU ability of the multidrug-resistant bacteria denotes increased antibiotic re-sensitization. In some embodiments, the antibiotic is an aminoglycoside or a fluoroquinolone. In some embodiments, the multidrug-resistant bacteria is Pseudomonas aeruginosa. In a further embodiment, the aminoglycoside is amikacin, gentamicin, and/or tobramycin. In some embodiments, the engineered strain of Pseudomonas aeruginosa is selected from PAK, PAO1, Pa-Tud-199, PA-14, PA7, UCBPP-PA14 or RP73. In some embodiments, the R-pyocin comprises SEQ ID NO: 1, SEQ ID NO: 4, SEQ ID NO: 7, SEQ ID NO: 9, SEQ ID NO:11, SEQ ID NO: 18, SEQ ID NO: 20, or a sequence at least 70% identical to SEQ ID NO: 1, SEQ ID NO: 4, SEQ ID NO: 7, SEQ ID NO: 9, SEQ ID NO:11, SEQ ID NO: 18, or SEQ ID NO: 20. In some embodiments, the multidrug-resistant bacteria comprise a deletion comprising SEQ ID NO: 14, SEQ ID NO: 15, SEQ ID NO: 16, SEQ ID NO: 17, or a sequence at least 70% identical to SEQ ID NO: 14, SEQ ID NO: 15, SEQ ID NO: 16, or SEQ ID NO: 17. In some embodiments, the method decreases biofilm formation, by the bacteria in a treated subject compared to an untreated control.

Further disclosed herein is a method of treating a bacterial infection in a subject, comprising administering to a subject a therapeutic composition comprising an R-pyocin and a pharmaceutically acceptable carrier.

Disclosed herein is a method of identifying R-pyocins with antimicrobial properties against high-risk bacterial strains, the method comprising a) collecting a bacterial sample; b) sequencing said sample and determining the presence of an R-pyocin; c) extracting and purifying the R-pyocin; and d) testing the purified R-pyocin's antimicrobial effect against a high-risk bacterial strain. In some embodiments the bacterial sample comprises Pseudomonas aeruginosa. In some embodiments, the high-risk bacterial strain comprises Pseudomonas aeruginosa.

Also, disclosed herein is a kit that is drawn to reagents that can be used in practicing the methods disclosed herein. The kits can include any reagent or combination of reagents discussed herein or that would be understood to be required or beneficial in the practice of the disclosed methods. For example, the kit could include media for growing bacteria. Disclosed herein is a kit for testing pyocin mediated collateral susceptibility of an infection-causing bacteria, comprising an R-pyocin comprising SEQ ID NO: 1, SEQ ID NO: 4, SEQ ID NO: 7, SEQ ID NO: 9, SEQ ID NO:11, SEQ ID NO: 18, SEQ ID NO: 20, or a sequence at least 70% identical to SEQ ID NO: 1, SEQ ID NO: 4, SEQ ID NO: 7, SEQ ID NO: 9, SEQ ID NO:11, SEQ ID NO: 18, or SEQ ID NO: 20 isolated from an engineered strain of Pseudomonas aeruginosa. In some embodiments the kit further comprises a sample collection tool, infection-causing bacteria isolation media, and infection-causing bacteria growth media.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g. amounts, temperature, etc.), but some errors and deviations should be accounted for. Unless indicated otherwise, parts are parts by weight, temperature is in ° C. or is at ambient temperature, and pressure is at or near atmospheric. Only reasonable and routine experimentation will be required to optimize such process conditions.

### Example 1

R-Pyocins: antimicrobial bacteriocins of Pseudomonas aeruginosa (Pa).

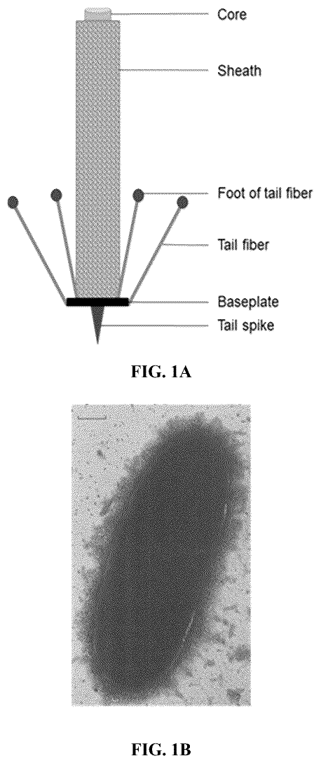

Bacteriocins are narrow-spectrum, protein-based antimicrobial substances generated by one strain of bacteria to kill other strains of the same species. Pyocins are antimicrobial bacteriocins produced by Pa specifically to target and kill other strains of Pa. Pa produces three main types of pyocin: S-pyocins, F-pyocins, and R-pyocins, each with distinct structures and killing mechanisms. R-pyocins are narrow-spectrum, contractile tailocins (protein complexes resembling phage tails) with subtypes R1-R5, differentiated by their killing spectrum. It was previously shown that the main killing activity of Pa clinical strains is through R-pyocins. Each Pa strain likely only produces one R-pyocin type, indicating strain specificity. Commonly only R1, R2 and R5 strains are identified in strains, as R3 and R4 pyocins are highly similar to R2-pyocins. R-pyocins share structural and functional similarities with the Myoviridae phage family's phage tails. The proposed killing mechanism involves the tail fiber's foot binding to a lipopolysaccharide (LPS) receptor specific to each R-subtype. The sheath then contracts, forcing the tail spike and core to puncture the outer membrane, leading to membrane depolarization and ultimately cell death (FIGS. 1A-1B).

For decades it has been assumed that a strain is resistant to its own R-pyocin subtype, although it has been shown that this is not always the case. Recent research has shed light on R-pyocin contraction after binding, however, only the receptor for the R3-type pyocin has been clearly identified. It remains unclear whether the remaining subtypes' receptor residues are located on the common antigen (A-band), 0-specific antigen (B-band), or the uncapped core of the LPS. It is also important to note that most mechanism studies have used lab strains like PAO1, and it is unknown whether the same receptor mechanisms exist in high-risk strains.

R-Pyocins and their use as therapeutic agents.

R-pyocins are structurally similar to bacteriophages although they lack capsid containing nucleic acids. For decades, phages have been explored as alternative therapies against bacteria in infections with notorious difficult to eradicate biofilms. More recently, phages have been successfully used in combination with antibiotics to treat Pa patients as a “last-ditch effort” and there is increasing interest in their use for treatment as an alternative therapy to accompany antibiotics. As narrow-spectrum, phage-like particles, R-pyocins also have specificity and the capability to target specific bacterial species in infection. While they can be effectively used to kill target species without modification, they have also been successfully engineered to broaden their spectrum of killing activity against other Pa strains and even other pathogenic bacterial species. Given the similarities to phage and the promise of phage therapy, these phage tail-like bacteriocins have been generally overlooked as therapeutic agents for use against Pa in human infections and yet, R-pyocins have a number of benefits as a therapeutic that are distinct from phage.

R-pyocins tend to be highly specific to a particular species of bacteria. Therefore, in a clinical setting there is a low risk of undesirable, non-specific killing of other species.

They do not replicate like phage. Hence, they may be used in doses, allowing for more precise and controlled treatment regimens than with self-replicating phage. They are also not impacted by CRISPR-Cas immunity mechanisms, which is a significant issue for phage resistance.

For many years, R-pyocin typing has been used as an epidemiological method of strain-typing Pa in human infections. Rapid diagnostics of strain-type using R-pyocins, thus quickly determining which R-pyocin type (or combination of types) can be used for treatment, can be less time consuming, more efficient and specific than determining which phage to use.

High-risk strains of Pa present a hugely significant healthcare burden that is increasing worldwide each year. Despite this, little progress has been made in successfully treating high-risk strains and new insights into treating them are hampered by the propensity of Pa researchers to work primarily on the commonly studied, and lab-adapted, PAO1 and PA14 strains.

The present application discloses the use of R-pyocins as a therapeutic for treating Pa infections caused by high-risk, antibiotic-resistant strains. The present application discloses (i) characterizing the diversity and bactericidal activity of R-pyocins sourced from different Pa strains against a panel of Pa high-risk strains; (ii) investigating mechanisms of R-pyocin resistance and evolutionary trade-offs with antibiotic resistance, and (iii) evaluating the therapeutic efficacy of R-pyocins in a murine model of chronic wound infection. The present application provides insights into the use of R-pyocins as a new class of antimicrobial agent for treating Pa infections. R-pyocins are narrow-spectrum bacteriocins with high specificity to Pa and represent a therapeutic alternative for treating high-risk strains of Pa which are challenging to manage with conventional antibiotics.

Current development of novel therapies against Pa usually involves studies on the two most commonly used laboratory strains PAO1 and PA14, both of which were originally isolated from infection sites, but which have also now become adapted to laboratory environments around the world. This is problematic for two main reasons: (i) laboratory strains often initially exhibit intrinsic sensitivity to antibiotics and new therapeutics, unlike many high-risk clinical strains. This can create a false sense of efficacy for new treatments; (ii) therapies developed using laboratory strains may not always translate well to clinical settings due to the differences in genetic makeup, virulence, and antibiotic resistance between laboratory and clinical strains. This may result in decreased effectiveness of novel therapies in real-world clinical settings. Here it can be determined whether R-pyocins can be developed as a therapy against high-risk strains. Little is currently known about whether these strains produce, can be killed by or resist R-pyocins. Thus, there is a critical knowledge gap in the understanding of how R-pyocins can be useful as a therapeutic measure against high-risk strains during human infection.

The present application investigates the diversity of R-pyocins produced by different Pa strains and their bactericidal activity against high-risk Pa strains. 100 high-risk strains from a Walter Reed Pa isolate panel have been sourced and a 300 Pa strain biobank has been sourced from CF patients.

The present application can develop a bioinformatic pipeline for the rapid molecular R-pyocin typing of Pa strain genomes, enabling efficient tail sequence alignment with the R-pyocin types R1 (SEQ ID NO: 2), R2 (SEQ ID NO: 5) and R5 (SEQ ID NO: 12). This approach, which is called “PyocinTyper,” can facilitate the assessment of binding affinity for potency and the optimization of pyocin tail sequences to enhance killing efficacy.

The present application can contribute a greater understanding of how some strains develop resistance to R-pyocins which is critical for optimizing their use as therapeutic agents and to develop strategies to enhance R-pyocin efficacy and circumvent resistance. Further, evolutionary trade-offs between R-pyocin resistance and antibiotic resistance and the use of R-pyocins to reverse AMR in high-risk strains can be investigated.

The present application can translate the in vitro activity of R-pyocins to in vivo therapeutic efficacy. The use of R-pyocins as a treatment option in a murine model of chronic wound infection can be evaluated, providing crucial information about their therapeutic use for clinical applications against high-risk strains.

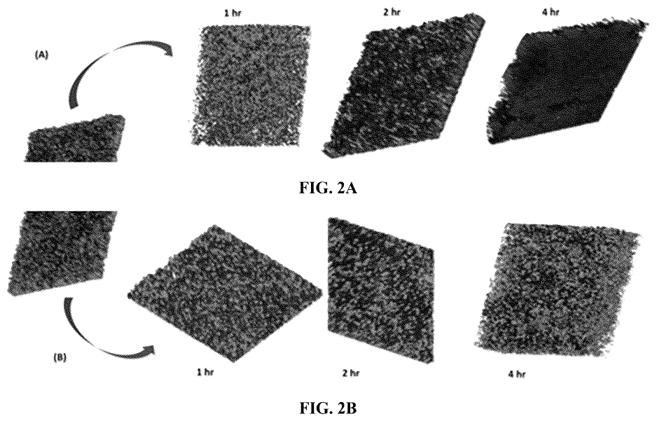

Highly effective R-pyocins against high-risk Pa strains grown in in vitro planktonic cultures and biofilms and in vivo murine chronic wounds can be identified. While most prior research has concentrated on utilizing R-pyocins to kill lab strains in planktonic cultures, the treatment of clinical isolated grown as biofilms or in infection has largely been overlooked. The use of R-pyocins produced by a set of cystic fibrosis (CF) Pa strains to combat established biofilms of other CF strains was demonstrated. It was shown that applying R-pyocins from A026 (R1-producer) to mature A018 (R2—producer) 15 h flow cell biofilms, led to a significant decrease in live A018 cell populations over time, accompanied by an increase in dead cells. This process resulted in a continuous decline in viable cells, ultimately achieving full-thickness biomass eradication 4 h post-treatment when utilizing purified R-pyocins from A026, but not with extractions from an A026 R-pyocin mutant (A026ΔR) (FIGS. 2A-2B). These findings strongly support the premise that R-pyocins produced by clinical strains possess the capacity to eliminate other clinical strains in mature biofilms.

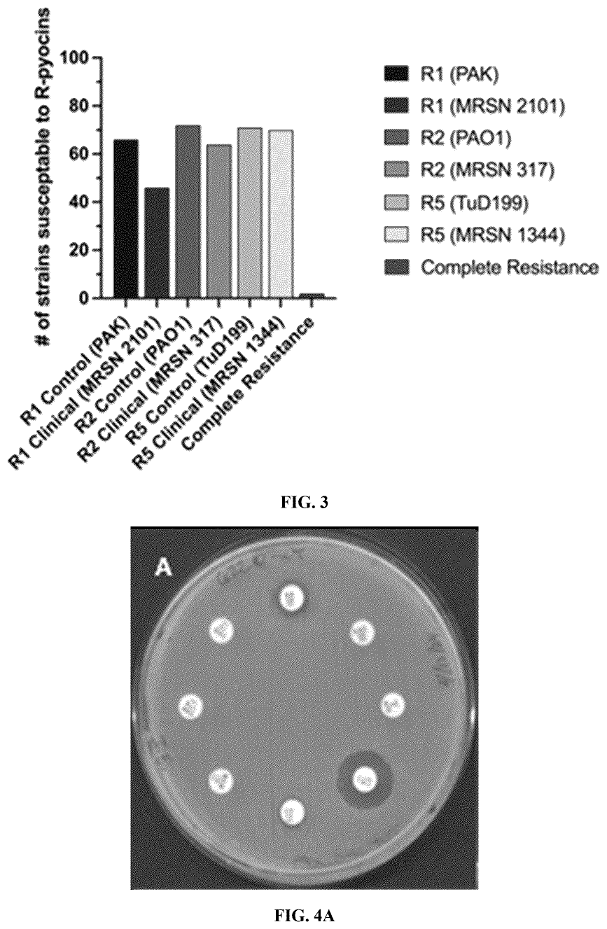

Disclosed herein are methods to examine the diversity and effectiveness of R-pyocins produced by different clinical Pa strains. The potency of R-pyocins produced by clinical isolates when targeting 100 high-risk strains sourced from the Multidrug-Resistant Organism Repository and Surveillance Network (MRSN) can be assessed. Using a standard spot test assay, findings revealed that 90 out of 100 high-risk strains were highly susceptible to at least one R-pyocin from the PAK (R1, SEQ ID NO: 1), PAO1 (R2, SEQ ID NO: 4), or TuD-199 (R5, SEQ ID NO: 22) strains. Further purification using ammonium sulphate precipitation on 6 R-pyocins (including 3 clinical high-risk strains), revealed that 96/100 of the high-risk strains were killed by at least 1 R-pyocin (FIG. 3). This indicates that these strains exhibit varying sensitivity to R-pyocins from other clinical isolates, and that clinical isolates produce novel R-pyocin ‘sub-types’ with differing potencies. This stands in stark contrast to six phages used in lab capable of killing PAO1. Notably, many high-risk strains displayed inherent and complete resistance to 6 Pa phages tested. To discover R-pyocins with diverse potencies, the panel of 100 high-risk strains and a CF-clinical biobank of 300 strains can be used. Since traits like antibiotic resistance and phage sensitivity differ significantly among these isolates, it is thought that R-pyocins can be produced at different levels and demonstrate varying effectiveness against high-risk strains. The well-organized, characterized and sequenced Pa biobanks offer a unique opportunity to investigate this in detail. A quick and efficient diagnostic pipeline called “PyocinTyper” has been developed to identify the R-pyocin types in sequenced Pa isolates. The pipeline utilizes the prophage identification program, PhiSpy (v2.3), to pinpoint R-pyocin regions. This is based on the strong genomic similarity between prophage and R-pyocin genes, as well as the presence of adjacent direct terminal repeat sequences. To confirm the identified regions as R-pyocins, a BLAST search using the known R-pyocin sequence from the PAO1 strain is performed. Once R-pyocin regions have been positively matched, their subtype specificity (R1, R2, or R5) can be determined by conducting another BLAST search on the identified regions. This search utilizes unique 800 bp tail fiber sequences specific to each R-pyocin subtype. After classifying the R-pyocin regions from each strain, they can be analyzed to identify SNPs and INDELs within sequences of the same subtype. The PyocinTyper tool was used to analyze the R-pyocin types present in the biobank of 100 high-risk strains. The results identified 6 strains with R1-type pyocins, 14 strains with R2-type pyocins, 29 strains with R5-type pyocins, and 11 strains that contain R-pyocin subtypes that were not typeable. 40 strains did not have R-pyocin genes present. These findings are consistent with previous research, which demonstrated that a significant number of clinical and environmental Pa strains do not actually possess R-pyocin genes. Nevertheless, these strains often remain highly susceptible to R-pyocins produced by other strains. Interestingly, R5 producers were previously thought to be rare, but these findings show that they are the most common type in high-risk strains. PyocinTyper can accurately and rapidly identify and characterize the R-pyocin types in 300 fully sequenced Pa isolates from the CF biobank, revealing genetic variations within sequences of the same subtype and contributing to a better understanding of R-pyocin diversity. This can firstly allow for the determination of which CF isolates are likely to be making R-pyocins and secondly, the method also can be used to quickly predict the susceptibility of high-risk strains to specific R-pyocins by analyzing their genomic sequences. As a result, the PyocinTyper diagnostic pipeline can function as a rapid diagnostic tool in identifying targeted treatment options for antimicrobial-resistant Pa strains.

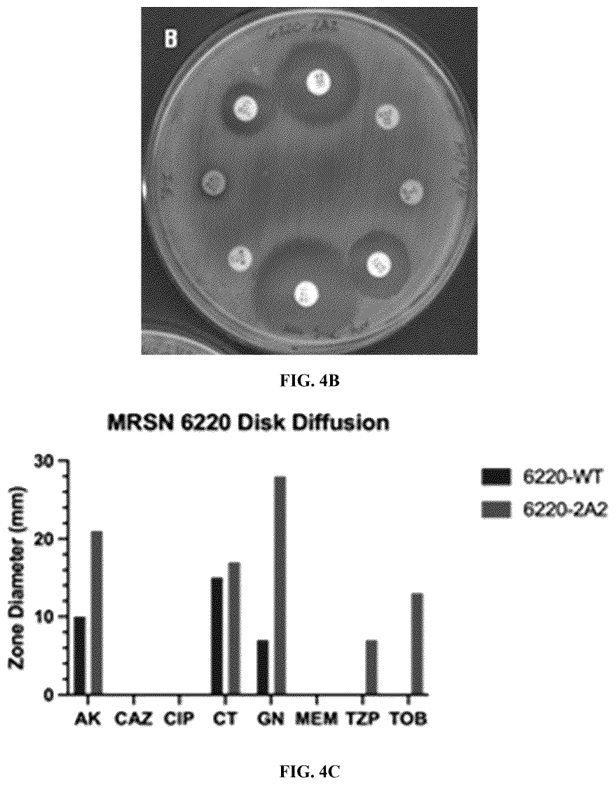

In studies investigating resistance mechanisms to R-pyocins in PAO1 and a PDR strain (MRSN 6220), it was observed that after 3 days of exposure to R-pyocins, the culture wells often became hyper-pigmented for both strains. Isolating bacteria from these wells revealed that they consistently produced a brown pigment, which was hypothesized to be pyomelanin (FIG. 4B). This brown coloration has been previously noted in some strains isolated from human infections. Additionally, other research has shown that bacteriophage predation on PAO1 also leads to this brown phenotype. Mutants with the R-pyocin-induced brown phenotype exhibited cross-resistance to all of the purified R-pyocin subtypes. Previous studies on phage interactions with PAO1 indicate that the observed phenotypic changes are likely due to a large (~200 kb) chromosomal deletion that includes the hmgA (SEQ ID NO: 15) and galU (SEQ ID NO: 14) genes, as well as the important mexXYZ multi-drug efflux pump. The hmgA (SEQ ID NO: 15) gene encodes homogentisate-1,2-dioxygenase. A deletion in this gene results in increased production of pyomelanin, which causes the brown coloration. The galU gene encodes UDP-glucose pyrophosphorylase, an enzyme essential for synthesizing polysaccharides by converting UTP and glucose-1-phosphate into UDP-glucose. UDP-glucose is crucial for building polysaccharides in the bacterial cell wall and extracellular matrix. Disruption of galU impairs the bacterium's ability to form a robust biofilm, reducing its virulence and persistence in infections. The brown mutants of PAO1 and 6220 were complemented with hmgA (SEQ ID NO: 15) and restored normal coloration, and with galU, which fully restored R-pyocin sensitivity. Antibiotic susceptibility tests on the brown mutant of 6220 revealed that this highly antibiotic-resistant strain became re-sensitized to several antibiotics, likely due to the deletion of the mexXYZ genes (FIG. 4C). Brown pigmented strains have been generated in 5 other clinical isolates which show the same antibiotic resistance-reversal phenotype. While cross-resistance to R-pyocins was not the goal, the reversal of antibiotic resistance in a highly-resistant clinical strain with R-pyocins is exciting and novel. This is termed ‘pyocin-mediated collateral susceptibility (PMCS)’, which is where exposure to an R-pyocin can lead to increased susceptibility of bacteria to antibiotics. This suggests a two-pronged strategy: cells can be killed by R-pyocins, and if resistance arises, an antibiotic treatment strategy becomes available. Further, brown strains are likely to be poorer biofilm performers and less virulent (though galU mutation), both of which are clinically beneficial to patients during infection.

R-Pyocin Diversity and their Bactericidal Use Against Antimicrobial-Resistant High-Risk Pa Strains

Existing treatments often fail against these high-risk strain infections, and the problem is exacerbated by the formation of bacterial biofilms, leading to prolonged hospitalizations and increased resistance to antibiotic therapies. Given this situation, it's imperative to investigate the variety of R-pyocins and their ability to eradicate high-risk Pa strains, as they can offer alternative or supplementary solutions to the antibiotic resistance problem. R-pyocins, derived from Pa CF and high-risk clinical isolates can demonstrate a wide host range and varying levels of efficacy. This can be tested by: (i) examining R-pyocin diversity within 300-isolate CF and 100 high-risk strain biobanks by evaluating R-pyocin activity from each isolate in vitro, and determining their host range and efficacy against high-risk Pa strains in planktonic cultures and biofilms; (ii) utilizing a bioinformatics pipeline for molecular typing of Pa strains to help decipher the genomic diversity of R-pyocin sequences in CF isolates and predict high-risk strain susceptibility to specific R-pyocins; (iii) comparing the antimicrobial efficacy of R-pyocins to several key antibiotics routinely used to treat Pa based on the Clinical and Laboratory Standards Institute (CLSI) guidelines. By implementing these steps, a comprehensive understanding of the range of R-pyocin diversity, their impact on high-risk Pa strains, and the use of R-pyocins as a therapeutic can be attained.

R-Pyocin Types in Pa Isolates from Cystic Fibrosis and High-Risk Biobanks

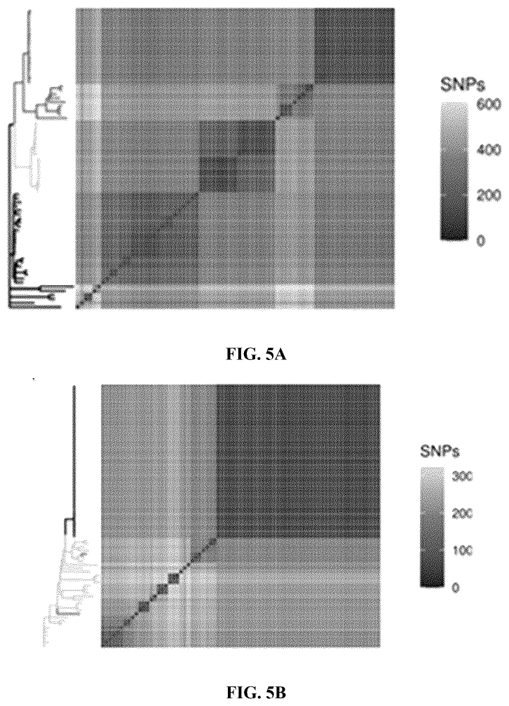

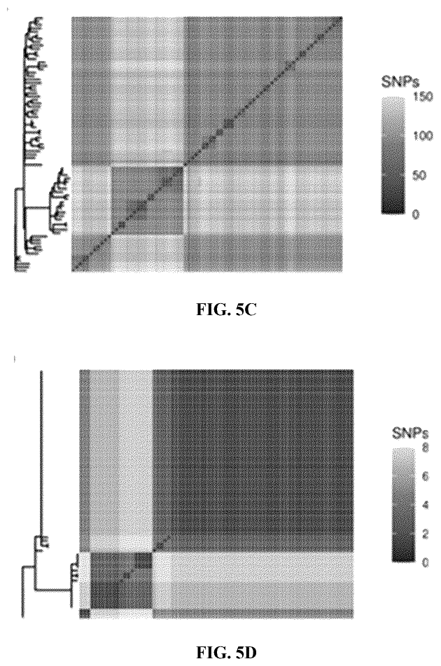

The diversity of R-pyocins and their efficacy in eliminating high-risk strains can be discerned. To do this, the biobank of 300 fully sequenced Pa isolates from four patients (75 per patient), can be employed. The primary focus of these isolates was on AMR, and they have not yet been analyzed or typed for R-pyocins. Interestingly, a recent large-scale genomic analysis conducted on these 300 isolates revealed considerable genetic diversity between Pa isolates in some patients, despite them being infected by a single Pa ‘strain’ (FIGS. 5A-5D). This observation suggests the presence of unique R-pyocin ‘sub-types’ within a single patient that may exhibit differential killing activities against high-risk strains.

A biobank of 100 high-risk strains from the Walter Reed Army Institute of Research can also be leveraged. This biobank includes various strain types, featuring six of the top ten most widespread high-risk strain types: ST235, ST111, ST244, ST357, ST175, and ST654. These strains were isolated from diverse human infection sites, with 26 strains specifically isolated from human tissue and wounds. PyocinTyper can be used to identify the R-pyocin-type produced by each of the 300 CF and 100 high-risk strains. Strains from both the CF and high-risk Pa biobanks that produce identifiable R-pyocins can be selected for R-pyocin extraction and purification. Purified R-pyocins can then be used to characterize their antimicrobial use against the 26 high-risk strains isolated from human wound infections.

Extraction and Purification of R-Pyocins from Typed Pa Strains

To evaluate the antimicrobial efficacy against high-risk wound strains, R-pyocins from laboratory and clinical Pa strains that were identified as R-pyocin producers using PyocinTyper can be extracted and purified. For purification, a previously published method can be used. Briefly, R-pyocin expression can be induced by adding mitomycin C to log-phase cultures (100 ml) at an OD600 of 0.25. These cultures can be incubated in LB broth medium at 37° C. and 200 rpm for 2.5 h to allow for lysis and R-pyocin release. At this point, 3 μl of DNase I treatment can be introduced to the culture and incubated for 30 mins. Following this, the culture can be centrifuged at 22,000 g at 4° C. for 1 h to separate cellular debris and the crude R-pyocin lysate in the supernatant. The crude lysate can be collected and purified using ammonium sulphate precipitation. To achieve this, 65 ml of saturated ammonium sulphate can slowly be added to the crude R-pyocin lysate while stirring on ice, then stored overnight at 4° C. The precipitate can then be sedimented by centrifugation at 35,500 g at 4° C. for 1 h, followed by resuspension in 16 ml of TN50 buffer. These resuspended R-pyocins can undergo a final sedimentation step at 50,000 g at 4° C. for 1 h, before being resuspended in 6 ml of TN50 buffer and stored at 4° C. The concentration (mg/L of total protein) of the purified R-pyocins can be determined using a Bradford protein assay (Bio-Rad Laboratories, Inc.) and stored at 4° C. in TN50 buffer for further antimicrobial efficacy testing. Six different subtypes of R-pyocins have been successfully purified (FIG. 3) (SEQ ID NO: 1, SEQ ID NO: 4, SEQ ID NO: 11, SEQ ID NO: 18, SEQ ID NO: 20, SEQ ID NO: 22).

### Evaluate the In Vitro Antimicrobial Activity of R-Pyocins Against Wound Infection Strains

To assess the antimicrobial use of purified R-pyocins sourced against high-risk wound strains, both qualitative and quantitative assays can be conducted. For qualitative evaluation, spot assays using 26 high-risk strains isolated from human wound infections, along with three indicator strains with known R-pyocin susceptibility, can be conducted. The procedure involves growing overnight cultures and diluting them to an optical density (OD600 nm) of 0.5. Five microliters of each culture can be mixed with 5 ml of top agar (0.4% LB agar) and spread onto fresh LB medium plates. R-pyocins and their serial dilutions can be applied to the agar plates and incubated overnight at 37° C. Clear halos indicating bacterial lysis can be considered a positive result, while lack thereof can indicate resistance to R-pyocin. Negative controls can include TN50 buffer-only conditions and lysates from R-pyocin deletion strains PAK-ΔR1, PAO1-ΔR2, and Tud-199-ΔR5. Spot assays using purified R-pyocins from PAK, PAO1, TuD-199 and 3 clinical strains, have been performed, yielding promising killing results against 100 high-risk strains (FIG. 3). R-pyocins from high-risk and CF strains can continue to be purified and quantitative evaluation of the antimicrobial activity of the 10 most potent R-pyocins against the 26 wound strains can be performed. Specifically, time-kill assays and minimum inhibitory concentration (MIC) assays can be performed. Time-kill curves can be plotted using previously described methodology, with cultures grown at 37° C. in LB broth. Each culture can be inoculated into fresh LB containing varying concentrations of R-pyocins, then incubated under specified conditions. Negative controls can include buffer-only and R-pyocin deletion strain extracts. The bacterial counts can be determined to calculate the time-kill curve. The MICs of each R-pyocin can be assessed using the Clinical and Laboratory Standards Institute (CLSI) broth microdilution method. Each high-risk Pa isolate can be diluted in cation-adjusted Mueller Hinton media. R-pyocin concentrations can be measured, diluted, and applied to a 96-well tissue culture plate. After incubation, OD600 measurements can be taken to define the MIC, MIC50, and MIC90. MIC assays can be conducted to determine the minimum bactericidal concentration (MBC).

To examine R-pyocins' effectiveness against biofilms produced by the 26 wound strains, Crystal Violet Biofilm assays with the 10 most effective R-pyocins alone or in combination as cocktails can be performed. The strains can be cultured under specific conditions to allow biofilm formation. The antimicrobial treatments can be applied, and the biofilms can be stained with crystal violet. After incubation and rinsing, crystal violet can be solubilized and its absorbance measured.

The percent inhibition of biofilm formation for each treatment, as well as the MIC and minimum biofilm eradication concentration (MBEC) of R-pyocins, can be determined. Lastly, the antimicrobial efficacy of R-pyocins can be compared to several key antibiotics routinely used to treat Pa (meropenem, piperacillin-tazobactam, ceftazidime, cefepime, gentamicin, tobramycin, ciprofloxacin and colistin) using standard disk diffusion and MIC measurements based on CLSI guidelines.

Several measures can be implemented to maintain scientific rigor. For in silico analysis, PyocinTyper can be used, which utilizes PhiSpy (v2.3) and BLAST, bioinformatics packages specifically designed for data analysis. A threshold of 70% sequence homology to the PAO1 R-pyocin for sequence comparisons can be set. The categorization of R-pyocin type identity can require 95% sequence homology to the unique reference tail fiber sequences of each R-pyocin subtype: R1 (PAK, SEQ ID NO: 1), R2 (PAO1, SEQ ID NO:4), and R5 (Pa Tud-199, SEQ ID NO: 11). R-pyocin extraction and purification can be conducted in triplicate, following established methods. This process can include control R-pyocin deletion strains and buffer controls. All R-pyocin conditions tested in the spot assays, MIC assays, and crystal violet biofilm assays can also be carried out in triplicate. Uniform conditions can be maintained across all experiments, including growth media, temperature, and incubation time, to minimize biases or inconsistencies in the results. Both positive and negative controls can be included in the MIC assessments to account for background growth and sterility, ensuring the reliability of the MIC data. Finally, statistical analyses can be performed on the MIC data and the crystal violet biofilm assay data to ensure the statistical significance of the results.

These results can provide an in-depth understanding of the antimicrobial activity of R-pyocins against high-risk strains in both planktonic and biofilm states. A systematic procedure for molecular typing of clinical strains can be created, which can produce R-pyocins with varied potency against high-risk strains. The antimicrobial activity and killing efficiency of R-pyocins against high-risk Pa strains can also be determined. For drug-resistant strains, R-pyocins can show significantly higher efficiency of killing than routinely used antibiotics. These outcomes can lead to the development of innovative antimicrobial treatments that address the challenges posed by antibiotic-resistant high-risk Pa strains.

### Characterizing R-Pyocin Resistance in High-Risk Pa Strains and the Impact on Antibiotic Susceptibility

Pa can evolve in response to R-pyocins. Understanding of resistance mechanisms to these bacteriocins is limited, particularly beyond the PAO1 strain. Data suggests that the galU gene (SEQ ID NO: 14) plays a crucial role in R-pyocin resistance in both lab and clinical strains (FIG. 4A). The galU (SEQ ID NO: 14) gene encodes a UDP-glucose pyrophosphorylase essential for synthesizing a complete LPS core and O antigen. Mutations or deletions in galU (SEQ ID NO: 14) result in a truncated LPS core, causing a rough LPS phenotype. These alterations, often caused by double-strand breaks from the non-canonical nicking function of MutL, can lead to the loss of neighboring genes like hmgA (SEQ ID NO: 15), in which mutations produce hyper-pigmented mutants through increased pyomelanin production. The altered LPS structure also likely impacts susceptibility to antibiotics. Notably, the mexXY (SEQ ID NO:16, SEQ ID NO: 17) efflux pump genes, located near galU (SEQ ID NO: 14), significantly contribute to aminoglycoside resistance. It is proposed that using R-pyocins as a selective pressure can promote chromosomal deletions which include the hmgA (SEQ ID NO: 15), galU (SEQ ID NO: 14), and mexXY(SEQ ID NO:16, SEQ ID NO: 17) genes. High-risk strains of Pa can develop resistance to R-pyocins, characterized by specific genetic and phenotypic changes, which can in turn enhance their susceptibility to antibiotics (pyocin-mediated collateral sensitivity; PMCS). To test this, (i) 26 high-risk wound strains from the Walter Reed biobank which are sensitive to R-pyocins can be selected, using R-pyocins with different target specificities to create varied selective pressures; (ii) parallel evolution experiments for each strain and R-pyocin type can be established; (iii) hyper-pigmented mutants can be isolated and the resistance levels of each can be assessed by measuring the minimum inhibitory concentration (MIC) of R-pyocins; (iv) whole-genome sequencing can be performed to identify common and specific mutations related to hyper-pigmentation and R-pyocin resistance and (v) the antibiotic susceptibility profiles of these mutants compared to their ancestral wild-types can be tested.

### Isolating Hyper-Pigmented Mutants Through R-Pyocin Exposure Using Experimental Evolution

To generate hyper-pigmented mutants, an experimental evolution study can be conducted. 26 drug-resistant wound strains can be evolved in Lysogeny broth (LB) using 96-well plates. This approach can help determine if the evolutionary processes related to R-pyocin resistance are consistent across different strains. Findings indicate that these strains are highly susceptible to R1-pyocins from PAK, R2-pyocins from PAO1, and R5-pyocins from Pa-Tud-199, as well as R-pyocins from other clinical strains (FIG. 3). To initiate the experimental evolution, the strains can be grown on LB agar plates. From each plate, a single colony can be inoculated into 5 ml of fresh LB and incubated for up to 6 h to reach the mid-log phase. These mid-log-phase cells can serve as ancestral strains for comparison with the evolved populations regarding phenotypic and genomic properties. The mid-log-phase cells can then be inoculated to an optical density (OD600) of approximately 0.05 in 200 μl of LB across three wells per strain. Different types of R-pyocins (R1s, R2s and R5s) can be added to the wells at 100 μg/ml. While the R-pyocins kill the majority of the bacterial cells, past experience using this concentration, suggests that a single dose does not eliminate all cells, allowing for the emergence of resistance. The cultures can be incubated for 18 h at 37° C. with shaking at 200 rpm. After this incubation, 10 μl from each well can be transferred to fresh wells containing 200 μl of LB, along with an additional dose of R-pyocin. This incubation process can be repeated for another 18 h. Hyper-pigmented mutants, which data suggests can confer cross-resistance to R-pyocins and reverse antibiotic resistance can be identified. To monitor this, samples can be plated after each passage onto LB agar to screen for brown colonies. Once brown mutants for a strain are identified, the evolution experiments can be discontinued. To assess the R-pyocin resistance levels of hyper-pigmented mutants, a series of twofold R-pyocin dilutions in LB broth using a 96-well microtiter plate can be prepared. This dilution range can cover the minimum inhibitory concentration (MIC) values. Each well can be inoculated with a standardized bacterial suspension (adjusted to OD600 ~0.05) from the respective strains, ensuring a consistent bacterial inoculum across all wells for accurate comparison. The controls can be included in the microtiter plate: a positive control (bacteria without R-pyocins) to monitor background growth, and a negative control (R-pyocins without bacteria) to ensure sterility. After incubating the plate at 37° C. for 18 h, the MIC can be determined for each sample as the lowest concentration of R-pyocin that inhibits bacterial growth, measured by OD600 readings in an automated plate reader. Hyper-pigmented mutants can exhibit cross-resistance to R-pyocins, likely due to a chromosomal deletion affecting the galU (SEQ ID NO: 14) gene. To confirm this, the mutants can be complemented with an intact plasmid-borne copy of galU (SEQ ID NO: 14) and it can be assessed whether R-pyocin sensitivity is restored.

### Genomic Sequencing of Hyper-Pigmented Strains

To sequence identified brown mutants, they can first be cultivated overnight in 15 ml conical tubes with LB at 37° C. and shaking at 200 rpm. DNA can be extracted using the Promega Wizard Genomic DNA Purification Kit, following the manufacturer's guidelines. For sequencing libraries, the Nextera XT DNA Library Preparation Kit can be used and the Illumina Novaseq platform can be employed to obtain 250 bp paired-end reads with a mean coverage of 70×. Reference genomes for each high-risk strain can be generated by acquiring Oxford Nanopore long-read sequences from SeqCoast Genomics (GridION flowcells, chemistry type R9.4.1, Guppy high accuracy base calling version 4.2.2) at 35× coverage. Unicycler can be used to produce high-quality long-read assemblies for each reference isolate. Then one round of long-read polishing can be performed on these assemblies with Medaka to generate preliminary consensus sequences. Quality control on all Illumina reads can be conducted using the Bactopia pipeline. Two additional short-read assembly polishing steps can be performed on the long-read assemblies by aligning the quality-adjusted short reads of each reference isolate to its respective consensus sequence using Polypolish and Pilon. The final consensus sequences can be validated by mapping the Illumina reads of each reference to its respective assembly using Snippy and ensuring that 0 isolates are called. To annotate the reference strains, Prokka can be used, by employing a custom Pa pan-genome database. In order to perform variant calling, Snippy can be used to identify variants (mutations and microindels) in relation to their respective reference strains. By combining Snippy-core and PhyML, a core SNP alignment and a maximum likelihood phylogeny can be generated. Then, VCFtools and Disty can be used to create a pairwise SNP matrix for each isolate. Next, SnpEff and SnpSift can be used to categorize the variants based on their predicted effects. It is predicted that hyper-pigmented mutants contain a chromosomal deletion that includes the hmgA (SEQ ID NO: 15), galU (SEQ ID NO: 14), and mexXY genes (SEQ ID NO:16, SEQ ID NO: 17). However, the size of these deletions may vary depending on the type of R-pyocin used to induce them.

### Antibiotic Resistance Reversal in R-Pyocin Resistant Hyper-Pigmented Mutants

To investigate antibiotic resistance reversal in hyper-pigmented mutants (FIG. 4C), the minimum inhibitory concentrations (MICs) of several clinically used antibiotics (tobramycin, amikacin, ciprofloxacin, ceftazidime, meropenem, piperacillin-tazobactam, gentamicin, colistin, and ceftolozane-tazobactam) can be determined. Both the mutants and their parental strains can be tested using disk diffusion plates and the broth microdilution method, following CLSI guidelines. All selected antibiotics are commonly used in clinical settings to treat Pa infections. Additionally, time-kill assays can be conducted using these antibiotics at concentrations of 1×, 2×, and 4× MIC, measuring colony-forming units per milliliter (CFU/mL) at intervals of 0, 1, 2, 4, 6, and 24 h. To assess efflux activity, an ethidium bromide accumulation assay, can be conducted, where bacterial cells are incubated with ethidium bromide (a substrate for the MexXY efflux pump) and fluorescence is measured over time using a plate reader. Each mutant can be complemented back with a copy of the mexXY (SEQ ID NO:16, SEQ ID NO: 17) genes either through a single copy on the chromosome or on a plasmid.

A comprehensive and systematic approach to investigate how Pa evolves in response to R-pyocins has been developed. The rationale emphasizes the critical role of the galU (SEQ ID NO: 14) gene in resistance mechanisms. 26 high-risk wound strains from the Walter Reed biobank can be selected, which can introduce a diverse genetic background and enhance the external validity of the findings. By conducting parallel evolution experiments across various strains and R-pyocin types, confounding variables can be minimized and reproducibility improved. Using specific concentrations of R-pyocins can allow for the control of the emergence of resistance and gain insights into adaptive responses. The minimum inhibitory concentrations (MICs) of R-pyocins can be measured using standardized techniques, ensuring data accuracy with appropriate controls. For genomic analysis, established techniques can be utilized, including Illumina and Oxford Nanopore sequencing, to obtain comprehensive and high-resolution genetic data. The characterization of antibiotic resistance can involve multiple antibiotics and methodologies, including disk diffusion and time-kill assays, in accordance with CLSI guidelines. Additionally, ethidium bromide accumulation assays can be performed to assess efflux activity, further deepening the understanding of resistance mechanisms. Overall, this approach provides valuable insights into the evolutionary dynamics of R-pyocin resistance in clinically relevant strains of Pa.

The results of the present application can provide a thorough understanding of how high-risk strains of Pa develop resistance to R-pyocins, particularly focusing on the role of the galU (SEQ ID NO: 14) gene. The genetic mechanisms underlying R-pyocin resistance can be clarified, emphasizing how mutations in galU (SEQ ID NO: 14) and nearby genes affect resistance. By isolating hyper-pigmented mutants through experimental evolution, specific phenotypic changes associated with R-pyocin resistance can be uncovered. This can allow for the exploration of cross-resistance to both R-pyocins and other treatments. Whole-genome sequencing of these hyper-pigmented strains can yield detailed genetic data, highlighting common and unique mutations linked to R-pyocin resistance. Identifying significant chromosomal deletions containing key genes such as hmgA (SEQ ID NO: 15), galU (SEQ ID NO: 14), and mexXY (SEQ ID NO:16, SEQ ID NO: 17), can deepen the understanding of how resistance evolves. Additionally, the antibiotic susceptibility of the hyper-pigmented mutants can be characterized compared to their ancestral strains. This involves determining the minimum inhibitory concentrations (MICs) for various clinically relevant antibiotics, revealing effects related to pyocin-mediated collateral sensitivity (PMCS). Therefore, the present application can show that the addition of R-pyocins can reverse antibiotic-resistance in multi-drug-resistant clinical isolates. High-quality annotated reference genomes can be generated for each high-risk strain, enabling accurate and comprehensive genomic comparisons between ancestral and evolved mutants. Generation of core SNP alignments and maximum likelihood phylogenies can contribute to a better understanding of the relationships among the different high-risk strains and their R-pyocin resistant isolates. The results of antibiotic susceptibility testing can be analyzed in conjunction with genomic data, enabling the correlation of specific genetic changes with shifts in resistance profiles, thus providing a more complete understanding of resistance mechanisms.

### Assessing the Preclinical Efficacy of R-Pyocins to Treat Pa Wound Infections

Safety assessments of R-pyocins against human lung epithelial, murine macrophage and murine fibroblast cells have been performed and no cytotoxic effects were observed (FIG. 6). In vitro and in vivo models have been developed for testing the pre-clinical efficacy of antimicrobials against biofilm-associated wound infections and has performed numerous efficacy studies of antimicrobial and anti-biofilm compounds for academic collaborators and private companies, the data for which have been used to justify Phase II clinical trials. An in vitro wound microcosm model, as well as a chronic mouse wound model can be used to help determine the optimal dose and frequency of administration. How hyper-pigmented mutants induced by R-pyocins colonize chronic wounds and how effectively they can be treated with conventional antibiotics can be assessed. For example, strains containing galU (SEQ ID NO: 14) and mexXY (SEQ ID NO:16, SEQ ID NO: 17) mutations can demonstrate reduced biofilm formation and increased antibiotic sensitivity in vivo.

### Screening R-Pyocins for Efficacy in an In Vitro Wound Environment.

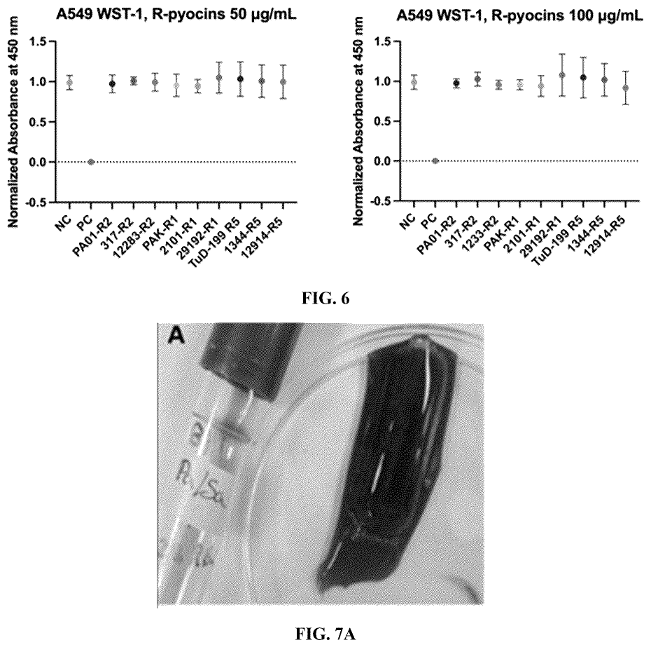

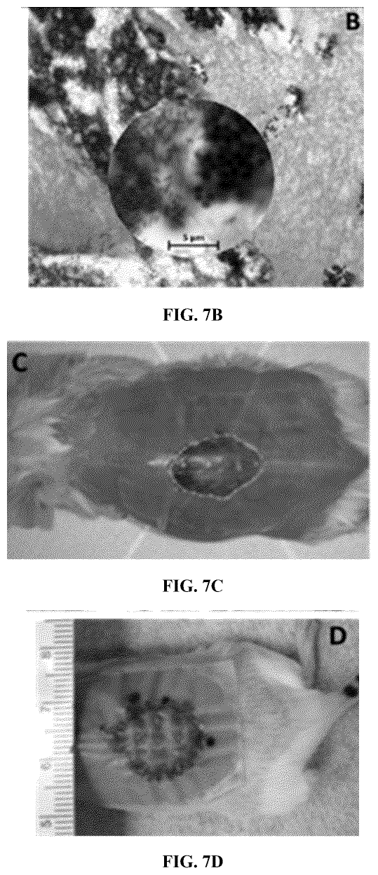

The efficacy of R-pyocins to kill Pa in an in vitro wound microcosm model can be tested. This model has been used extensively to establish stable polymicrobial communities that exhibit the spatial distribution, biofilm formation, and increased antibiotic tolerance seen in wound infections. This simple and effective method to grow biofilms in vitro was formulated to best represent the conditions of human wounds and contains physiological concentrations of blood components (FIGS. 7A-7D) and can be used in a semi-high throughput manner to screen isolates and R-pyocins at different doses and in different formulations. Using this model, the ability of R-pyocins to access and kill Pa in biofilm aggregates and withstand the proteolytic and other inhibitory effects that serum proteins often display on therapeutics can be assessed. PA01 and the pan drug-resistant 6220 strain (and their corresponding hyper-pigmented mutants) can be grown in wound-like media as described previously (FIGS. 7A-7D). After 24, 48 and 120 h the media containing the bacterial aggregates can be treated with the six most effective R-pyocins against these strains or a vehicle control for 2, 4 or 8 h. These time points are based on previous studies using this model for antimicrobial efficacy assessment and can determine whether R-pyocin efficacy is influenced by the age of the biofilms and how long treatments must be applied. The wound microcosms can then be thoroughly washed to remove treatments and the number of surviving bacteria can be assessed by determination of colony forming units (CFU). Dosing experiments can also be performed to determine the effective concentrations of R-pyocins required to decrease the bacterial load at least 3 logs, which is the rate of reduction suggested by the FDA for topical wound treatments.

Determining the Efficacy of R-Pyocins, with and without Antibiotics, to Treat Pa Mouse Wound Infections.

For in vivo studies, a murine surgical excision model to simulate human chronic wounds can be utilized (FIGS. 7A-7D). This established model has been widely used to study chronic wound infections, showing biofilm formation, increased antimicrobial tolerance, delayed healing, and ease of monitoring. All experiments can be conducted under IACUC protocol #07044. Mice can undergo surgery under anesthesia to create a full-thickness, dorsal, 1.0×1.0 cm excisional wound down to the panniculus muscle. Each wound can be infected with approximately 105 CFU of Pa. PA01 and the pan-drug resistant 6220 strain can be used along with the six most effective R-pyocins (based on MIC data). Post-infection, wounds can be covered with a semipermeable polyurethane dressing to protect against contamination and simulate a chronic infection environment. To assess whether R-pyocins can eliminate established Pa infections, wounds can be irrigated after four days with R-pyocin solutions at optimal doses, or with vehicle controls. Mice can be euthanized 24 h post-treatment, and wounds can be excised for bacterial load assessment via CFU determination. Achieving the efficacy seen in vitro may require higher concentrations or repeated dosing in vivo. Thus, additional tests involving daily treatments and varying R-pyocin concentrations may be conducted based on initial results. The effectiveness of R-pyocin and antibiotic treatments against wild-type PAO1 and pan-drug resistant clinical strain MRSN 6220, along with their brown hyper-pigmented mutants can be compared (FIG. 4C). Gentamicin is the primary focus because it is a standard antibiotic used in wound infection studies. The strain 6220 is completely resistant to gentamicin, while its corresponding hyper-pigmented mutant is highly sensitive to this antibiotic. This difference enables the investigation of how resistance to R-pyocins may lead to the emergence of strains that can be more effectively treated with antibiotics in vivo. Treatments with R-pyocins (100 μg/wound), antibiotics (gentamicin, 40 mg/kg), or combinations can commence after 24 h and continue daily for seven days. Bacterial loads can be monitored daily by swabbing wounds and culturing on selective media, while systemic spread can be assessed through blood and organ homogenate cultures on day 7. Wound healing can be evaluated by measuring the wound area daily and through histological analysis on day 7. On days 1, 3, 5, and 7 post-treatment, bacteria can be isolated from wound swabs to identify any in vivo generated brown phenotype isolates (from wild-type initiated infections) and their antibiotic susceptibility profiles can be determined. Any such isolates can undergo whole-genome sequencing, focusing on the galU (SEQ ID NO: 14) and mexXY (SEQ ID NO:16, SEQ ID NO: 17) regions.

### Determining the Safety of R-Pyocins to Treat Pa Mouse Wound Infections.

Since experimental drug-toxicity more typically arises from systemic exposure, the topical application of R-pyocins poses a minimal risk. However, toxicity studies are performed in order to determine the safety of the treatments. The six R-pyocins can be administered topically to mice that have been administered surgical excision wounds but are not infected. The lowest dose given can be the highest therapeutic dose that has been determined experimentally, and the highest dose can be the maximum feasible dose. R-pyocins can be applied daily for 14 days, and mice can be observed. All mortalities, clinical signs of toxicity and clinical signs of dermal sensitivity (erythema, edema, pruritus or ichthyosis), including time of onset, and duration can be recorded. Gross necropsies can be performed on all animals, including those euthanized as moribund or terminated at 14 days. Hematology, clinical chemistry, gross and microscopic pathology can also be evaluated. These data can help identify doses causing no adverse effects and doses causing major (life-threatening) toxicity. Wound closure can also be assessed every 2 days in mice that are administered daily R-pyocins or vehicle controls, using a SilhouetteStar laser scanning wound imaging, 3D measurement and documentation system. A significant delay in wound closure in R-pyocin-treated mice can indicate an adverse effect, likely inflammatory. If wound closure delays are observed, the inflammatory response elicited by R-pyocin treatment can be assessed by measuring the levels of pro-inflammatory mediators in the tissue at different time points post-treatment.

Replicates can be performed except for assessing Pa numbers in the murine chronic wound model in which a minimum of 8 animals (2 biological replicates of 4 animals) of each sex can be used to provide appropriate statistical power with sex included as a biological variable (95% power to detect differences among groups at P<0.05). Mice can be randomly assigned into experimental groups upon arrival, and treatments and assessments can be blinded. To assess statistical significance, student's t-test for pair-wise comparisons of groups, and multi-isolate analyses using ANOVA followed by a posteriori test using Sigma Stat software can be used. Data, including bacterial load and cytokine levels can be analyzed with ANOVA and Tukey's multiple comparison test (if normally distributed) or by the Kruskal-Wallis test with Dunn's multiple comparison test (if not normally distributed).

SEQUENCES
PAKAF_R1_PYOCIN
SEQ ID NO: 1
TGATCAAAGCCATCGATGAAATGCTCAAGCTCTGGGCCGAGGAAATGCACGCGCCAG
GCAGCAACGGAGGCGGCTACGCCGGCGGCAACCTGATCGCCATGCTGATCGCCAGCA
AGGGCGAGGTGGTCCGCGGCCACCGTGGCAGCCGGGTGATCCTCGACCGTGTGGCG
GAGGTCGATCGCCTGGTAAATCGCCTACCCGAGGAACTGAAGAACGTGGTGGTGGA
GCACTATCTCAATCGCGACAGCTTCCCCGAGCAGAAGTACCGCCACTGCGGTTGCAG
CCGCAACACCTTCTATCTGCGCCTGCATGTGGCGCACCAGGGTATCCAGGACGGCCT
GCTGCGGCGGGTGGCCTGAGTTCCTGGACCGGATACCGTGTCCCCCCTGGAATTTTCC
AACCGGCCTTCGAGCCGGTTTTTTTATGCCTGTCGCCCATGGAGGCGGTGAGTCCCAT
GCACAGCAGCGGACCCTGCACGGTAGAGGCGGATTCGGGCTGGCGTAGCCATCCTTT
TCGTCAGTCGATGCCGGGCTTTCCACATCGGAAGTGGGTATGCGCTTGCCCGGATGCC
TGGCGAAGGTTCCCAGGGCGGTATATCACCCTCATATCGTGGTGCGTCTCCCTCCTATT
GCAGTGGATTGTCGAGAAGGCATTGCCGGGCTGGGAATCGGGCGGTAAAAAGTAGTC
ATTCTTGTAAAGGTGCGTCCCCAGGGAGGCACTCGTGAAGCACCGGAACCCGGCCCT
GGCGCCGGGTTTTTTATTGCCCGGGAAAACGTTCGTGCGGACGTGCCCGGCGCTGCC
TGCCAAGGAGGGACCCATGGGCAACGAACCGCAGACACTGACGGAAATGCCGCTCT
GGGTACTGATCCTGCTCGCCGCGCTGGGCGGCGTCAGCGGCGAGATGTGGCGTGCCG
ACAAGGCCGGTCTCGGCGGCTGGGCCTTGTTGCGGCGCCTGGCGCTGCGCTCCGGCG
CGTCGATTGTCTGCGGCGTGGCGGTGATGCTGCTGGCGTTGGCTTGCGGCGCCGCGC
TGCTGTTCGCCGCGGCGCTGGGCAGCCTGACCGCCGCGGCCGGCGCGGAGATCGCA
GTCGGTCTCTACGAACGCTGGGCCGCCCGGCGCCTGGGGGTTTGCGAGCTGCCCGAG
GAACAGTCGGACGACCGTGGCCCGCATTGAAACTGATCGAAGGAGTCAACCATGCCT
GAACAGGCTGTTACGCTCGAGGCTCTGTACGCGGCCATCGAGCAGGTACTGCGTGAG
CGTCTGCCGGAGGCGCAGTTGATCGGCTTCTGGCCAGGCGTGCCGGAAAATACCCCG
GCGGTTTCCCTGGAGATAGCCGAGCTACTGCCCGAGCGCGATCCCGGTACCGGCGAG
AGTGCCCTGCTGTGCCGCCTGCAGGCGCGGATAATGGTGCCACCTGGTGCCGATCGC
CAGGCGGTATCCATTGCTTGCGGAATCGTTCGGACATTGCGCGAGCAGACCTGGAAC
CTGTCTCTGGAACCGGCGCGCTTCGTACGCTCGGCCGTCGACGGCAGTCGCGAGGAG
CTGAAGAGCCTGCGTGTCTGGCTGGTCGAGTGGACGCAGTCGCTGCGTCTCGGAGAT
CCGGAGTGGGCCTGGGAGGACCAGCCGCCGGGCAGCCTGATGCTGGGCTTCGACCC
GCAGACCGGCCCCGGCCATGAGCCGGACTACTTCGCTCCGGAGGCGTTGGCATGAGC
TATGTCAGTGCGGAGCATGACCGCATGCTCGCCGCGATGATCCTGCCCTGCGTGGTGG
TCGCCGTGGACCTGGCAGCGGCGCGGGTACGGGTGCGCTCCGGCGACTGGACCAGC
GGCTGGCTGCGCTGGCACTCCCTGGCAGCCGGCAAGGTTCGCCACTGGCGTGCGCC
GAGCATAGGCGAACAGGGGATACTGCTCAGCCCGTCGGGCGGAGTGTCAATGGGCA
CCTTTATTCCCGGTCTGTACGGCGATGCGGGCACGGCGCCGGACAACAGCGCCAGCA
GTGAGACCTGGCGTTTCGACGACGGCGCCTCGTTGAGTTACGACTGGGCTGCGCATC
GCTACCGCGTCGAGCTGCCCAGCGGCACCGTGGAAGTGAGGGTCGGCGCCAGCGAG
GTGCGGGTCAGCGACGGGGCGGTCAGTCTCAAGGCGCCGAAGATCAGCCTGGAAGG
ACCGGTGGAGATCGCCGGGACACTGACGGTCAGCGGAGACATCCTCGGCGGCGGCT
CGATCATCGACACCGCCGGCAACAGCAACCACCACACCCATTGAACATACGACGGGG
CTGCCGAAGGGCGGTCCGTCGAACATTCAACCGGCCCGCGCGAGCGGGCCTTTTCGT
TTGCGGAGTTCGCCATGGGCAACACTCACAGCCATTCGGAGCCAGGCGCGGCCTGTC
TCTGCCGGAGCGGGAGGGCAGGGCGATGATCGGGATGGATCGCCGTAGCGGGCTGCC
CCTGCCCGGCCTGGCTCATCTTAAACAGTCCGTTGAGGACATCCTGACCACCCCGTTG
GGCAGCAGGCGCATGCGCCCCGAGTACGGCAGCAAGCTGCGGCGGATGGTCGACAT
GCCGGTGAGCGAAGGCTGGAAAAGCGCCGTGCAGGCCGAGGTAGCCCGTTCCCTGG
GGCGCTGGGAACCGCGCATCGCATTGTCTGCCGTGCGAGTCGTCGCGGTCGTCGATG
GCCGCGTGGATCTGCTCCTGAGCGGCGTGTTCGAGGGCGAGAACATCAATATGGAGG
TCTCGGCGTGATCATCGATCTTTCCCAGTTGCCGGAGCCAGAGGTTATCGAAAACCTC
GATTTCGAGACGATTTACCAAGAGCTGTTGGGCGACTTCCGCGAAGCCATGGCTGGC
GAATGGACAGCGGAGGTGGAGTCCGATCCGGTTCTCAAACTTCTGCAACTGGCGGCC
TATCGAGAACTGCTGCTGCGGGCGCGGATCAACGATGCGGCGCGGGCGGTGATGCTG
GCATATGCCAGCGGTGCCGATCTCGACCAGATCGGTGCCGGCTTCAATGTGCAGCGTT
TGCTGATCAGGCCCGCACAGCCCGAGGCGGTACCGCCGGTGGAGGCGCAGTACGAG
AGCGACAAGTCGCTGCGCAATCGCATCCAGCTCGCGTTCGAGCAGCTATCCGTCGCA
GGACCGCGGAACGCCTATATAGCCCATGCACTGGGCGCGGATGGAAGGGTGGCGGAT
GCCTCTGCGACCAGTCCGGCGCCCTGCGAAGTGCTGATCAGCGTGCTCGGGGTGGAA
GGCAACGGGCAGGCACCGGAAGCGGTGTTGCAGGCAGTGCGCCTGGCGCTGAACGC
GGAGGACGTGCGTCCTGTCGCGGATCGGGTAACGGTGCGCTCGGCAGGAATCGTTCC
CTATCAGGTCAAGGCGCAGCTCTACCTGTTTCCCGGCCCCGAGGCCGAGCTGATCCG
TGCCGCCGCCGAGGCTTCGCTGCGCGACTACATTTCCGCCCAGCGCCGCCTGGGCCG
CGACATCCGGCGTTCGGCCCTGTTCGCCACCCTGCATGTCGAAGGCGTGCAGCGCGT
CGAACTGCAGGAGCCTGCGGCCGACGTGGTCCTGGATGAAACCCAGGCGGCCTATTG
CACGGGGTACGCGATCACCTTGGGAGGCGTCGATGAGTAGCCGCCTGCTGCCGCCAA
ACAGGAGTTCTCTGGAACGCTCTCTGGGTGATGTATTGCCTGCCGAGCTGCCGGTGC
CGCTTCGTGAGCTTAACGATCCGGCACGCTGTGAGGCGGCCTTGCTGCCCTACCTGG
CCTGGACGCGCTCGGTGGACCGCTGGGACCCGGACTGGAGCGACGAGGCCAAGCGC
AATGCGGTAGCGACGTCCTTCGTCCTGCACCAGCGCAAAGGCACGCTGACCGCGTTG
CGCCAAGTGGTCGAGCCGATCGGTGCGCTGAGCGAGGTCACCGAATGGTGGCAGCG
AAGCCCGCTTGGCGTGCCGGGGACCTTCGAGATCACCGTGGACGTCAGCGACCGTG
GCATCGACGAAGGCACCGTACTGGAGCTGGAGCGCTTGCTCGATGACGTCCGCCCGG
TGAGCAGACACCTGACCCGGCTGGACCTGCGCATTACCCCGGTAATCCGGTCCCGTC
ACGGACTGGCCGTGACCGACGGCGACACCCTGGAAATCTTCCCCTGGAAACAGTGA
CATGACGACCAATACTCCGAAATACGGTGGCCTGCTCACCGACATAGGTGCCGCTGC
GCTGGCTGCGGCCAGTGCAGCAGGCAAGAAATGGCAGCCGACTCATATGCTGATCGG
CGATGCCGGCGGTGCGCCGGGCGACACGCCGGATCCATTGCCTTCTGCGGCGCAGAA
GAGCCTGATCAACCAACGCCATCGGGCTCAGCTGAATCGGCTGTTCGTTTCCGACAA
GAACGCCAATACCTTGGTTGCCGAGGTGGTGCTGCCAGTTGAGGTAGGTGGCTTCTG
GATCCGCGAGATCGGCCTGCAGGATGCCGACGGCAAGTTCGTCGCGGTATCCAACTG
CCCGCCCAGCTACAAGGCTGCAATGGAAAGTGGCAGTGCGCGGACCCAGACCATTC
GGGTGAACATCGCGCTCTCCGGCCTGGAGAATGTCCAGCTGCTGATCGACAACGGCA
TCATCTACGCCACTCAGGACTGGGTGAAGGAAAAGGTCGCTGCCGATTTCAAGGGCC
GCAAGATTCTGGCTGGCAATGGCTTGGTCGGTGGGGGCGATCTTTCTGCCGACCGCA
GCATTGGTCTGGCGCCTTCCGGCGTGACGGCGGGCAGCTATCGTTCGGTCACGGTGA
ACGCCAACGGGGTGGTCACCCAGGGCAGCAATCCGACCACCCTGGCCGGCTATGCG
ATCGGAGATGCCTATACCAAGGCCGATACCGATGGAAAACTGGCGCAGAAAGCGAAC
AAGGCCACCACCCTGGCCGGCTATGGCATTACCGATGCGCTGCGAGTCGATGGCAAC
GCCGTGTCATCCAGCAGGCTGGCCGCACCGCGTAGCCTGGCAGCCAGTGGCGATGCC
TCCTGGTCGGTGACCTTCGACGGCAGTGCCAATGTTTCTGCGCCGCTGAGTCTTTCCG
CTACCGGTGTGGCGGCGGGCAGCTATCCGAAGGTGACCGTGGATACGAAGGGAAGG
GTGACTGCTGGAATGGCGCTGGCGGCGACGGACATTCCCGGGCTGGATGCTTCGAAG
TTGGTCAGCGGGGTGCTGGCCGAGCAGCGTTTGCCGGTATTCGCGCGCGGGTTGGCT
ACTGCTGTCTCGAACAGTAGCGATCCGAACACCGCGACCGTGCCGTTGATGCTGACC
AATCATGCGAACGGACCTGTTGCCGGACGATACTTCTACATCCAGTCGATGTTCTATCC
GGATCAGAACGGCAATGCTTCGCAGATTGCAACGAGCTACAACGCTACATCCGAGAT
GTATGTACGGGTGTCCTACGCGGCCAACCCTAGCATCCGGGAGTGGTTGCCCTGGCA
GCGTTGCGACATTGGAGGTTCCTTCACGAAGACGACTGACGGATCCATTGGAAATGG
CGTCAATATAAACAGCTTCGTCAATTCCGGATGGTGGTTGCAATCGACATCGGAATGG
GCGGCGGGTGGAGCTAACTATCCCGTGGGGCTGGCCGGTTTGCTGATTGTCTACCGC
GCACATGCAGACCATATCTATCAGACCTACGTAACACTCAACGGAAGCACATATTCGC
GCTGCTGCTATGCGGGCTCTTGGCGTCCGTGGCGGCAGAACTGGGACGATGGAAACT
TCGATCCGGCCAGCTACCTGCCAAAGGCGGGATTTACCTGGGCGGCTTTGCCGGGTA
AGCCGGCAACTTTCCCGCCCTCAGGGCATAACCACGATACCAGCCAGATCACCTCCG
GCATCTTGCCTCTCGCTCGTGGTGGCCTTGGCGCCAATACAGCGGCCGGAGCACGCA
ACAACATTGGTGCCGGAGTGCCGGCCACGGCGAGCCGGGCGCTCAATGGTTGGTGG
AAGGACAACGATACCGGTCTGATCGTCCAGTGGATGCAGGTGAACGTAGGAGATCAT
CCCGGTGGAATAATCGATCGTACCTTGACGTTCCCGATCGCGTTCCCTAGCGCCTGTC
TGCATGTCGTACCGACTGTCAAGGAGGTGGGGCGACCAGCGACGTCCGCGTCGACC
GTTACGGTCGCCGATGTCAGCGTCAGCAACACGGGATGTGTGATCGTTTCCTCCGAGT
ACTACGGACTGGCTCAAAACTATGGCATCAGAGTGATGGCCATCGGCTATTGAGGTGA
AAATGATTTTTTTCCATGCCGCCACGGGCGGCTTTTATTCGAAGGAAATTCATGGGTC
CCGCATGCCTCTGGAGGATGAAATGCATCCTCTCGAAGACGCCGAGTACCAGGCTCT
TCTTCGTGCGCAGAGCGAAGGGAAACGAATTGTCACGGACCACACCGGTCGCCCTAT
CTGCGTCGATCCACCGGCTCCGGCCAAGGACATTCTGGTCCAACGGGAACGCATCTG
GCGCGACCGGCAGTTACAGCTCACCGACGGGCCTCTCGCTCGGCATCGTGACGAGCA
GGACCTGGGAAAAACTACGACTCTGAGCCAAGAGCAGCTTCGTGAGCTAACTCTCTA
TCGCGCCGTTCTTCGCGACTGGCCTATTGCCGCGGAGTTTCCCGACCTGAACGCAAG
GCCCGAGCCGCCTGCCTGGCTCCAATCGCTCATCACCCCCTGAACCCCGCCTTGTGC
GGGGTTTTTCATTAATGGAGATCTACCTATGAGTTTCTTCCACGGCGTTACGGTAACCA
ACGTCGATATCGGTGCGCGCACCATCGCGCTGCCGGCCAGTTCCGTCATCGGCCTCTG
CGATGTGTTCACGCCGGGGGCGCAGGCAAGCGCCAAGCCCAACGTCCCGGTGCTGC
TCACCAGCAAGAAGGACGCCGCCGCGGCGTTCGGCATCGGCTCGTCTATCTACCTGG
CCTGCGAGGCCATCTATAACCGTGCCCAGGCCGTGATCGTGGCGGTGGGCGTGGAAG
CCGCGGAGACTCCCGAGGCCCAGGCCAGCGCCGTCATAGGTGGTATCAGTGCCGCCG
GCGAGCGCACCGGGCTGCAGGCTTTGCTCGACGGCAAGAGCCGCTTCAATGCCCAG
CCACGTCTACTAGTTGCGCCGGGTCATTCGGCCCAGCAAGCGGTGGCCACCGCCATG
GACGGGCTGGCCGAGAAACTGCGGGCCATCGCCATTCTCGATGGTCCCAATAGCACC
GACGAGGCGGCCGTCGCCTACGCCAAGAACTTCGGCAGCAAGCGCCTTTTCATGGTC
GACCCGGGCGTTCAGGTTTGGGACAGCGCCACCAATGCCGCGCGCAACGCCCCGGC
TTCGGCCTACGCCGCCGGCCTGTTCGCCTGGACCGACGCCGAGTACGGCTTCTGGTC
CTCGCCGTCGAACAAGGAGATCAAGGGCGTCACCGGCACCAGCCGTCCGGTGGAGT
TCCTCGACGGCGATGAGACCTGTCGCGCCAACCTGCTCAACAACGCCAATATCGCGA
CGATCATTCGCGACGATGGCTATCGCCTGTGGGGCAACCGCACCTTGTCCAGCGACA
GCAAGTGGGCTTTCGTCACCCGTGTTCGGACCATGGACCTGGTAATGGATGCGATCCT
CGCCGGGCACAAGTGGGCGGTGGACCGCGGTATCACCAAGACCTACGTGAAGGATG
TCACCGAGGGCCTGCGCGCCTTCATGCGCGATCTGAAGAACCAGGGAGCGGTGATCA
ACTTCGAGGTCTATGCCGACCCGGACCTGAACAGCGCCAGCCAGCTGGCCCAGGGC
AAGGTGTACTGGAACATCCGCTTCACCGATGTGCCGCCTGCCGAGAACCCCAATTTC
CGTGTCGAGGTGACCGATCAGTGGCTCACCGAAGTTCTGGATGTCGCCTAAGGAGCG
CCCGTGATGATTCCGCAAACCCTGACCAATACCAATCTGTTCATCGACGGCGTGAGCT
TCGCCGGTGACGTGCCATCCCTGACCCTGCCCAAGCTGGCTGTGAAGACCGAGCAAT
ACCGTGCCGGCGGCATGGATGCGCCGGTATCCATCGACATGGGCCTGGAGGCGATGG
AAGCCAAGTTCTCCACCAACGGTGCCCGCCGAGAAGCGCTGAATTTCTTCGGCCTGG
CCGACCAGAGCGCTTTCAATGGCGTGTTCCGTGGCTCCTTCAAGGGCCAGAAGGGCG
CCAGCGTGCCAGTGGTGGCTACCCTGCGCGGCCTGCTCAAGGAGGTCGACCCGGGC
GACTGGAAAGCCGGCGAGAAAGCCGAGTTCAAGTACGCCGTTGCGGTCAGCTACTA
CAAGCTGGAAGTCGATGGCCGCGAGGTCTACGAGATCGATCCGGTCAACGGTGTCCG
CGCGATCAACGGTGTCGACCAGTTGGCCGGCATGCGCAACGACCTCGGCCTGTAAGA
GGAGCTCCGGACATGACCCAAGAGAATCGACTGCCGGGCTGGCTGACGCTGGATGCT
GACGCCGCCCTCGTTCGTCTCTCGCGTCCGGCACAGTGCAACGGGGTCAGCGTCGAC
ACGCTGACCCTGCGTGCACCCACCGTGCGTGATATCCGCCTGGCCGGCAAGGTGGCC
GGCGACGATGCCGAGGAGCGCGAACTGCAACTGTTCGCCTCGCTGGCGCAGGTCAG
CCGCCAGGACCTGGAGGGGCTGAAGCTGAGCGACTACCAGCGGCTGCAAGGAGCCT
ACTTTCGCCTGGTGCAAGACGACACGGACGACACCTTCGCGTATGCGTCAACTGGCG
AGGCGCCTGGCCATTGAGCTGGGCTTCCAGGCCAGCGAACTGGAGCGCATGACCCTG
GGCGACCTGCTCTGGTGGCTCGCCGAGGGCGAGGAGTGAGCGACGGCGCCGTCCGC
CAGGCGAGCGGCGCCCGCGCTACTTCAGTAGGTCCGCGGAGGCGGACATCGATAACA
GACGAGACGAACCGTCATGAGTAAAGACATGGACCTGGTGGTCTCCATCGGCGGCAT
CGCCGACCCCTCCTTGGGCAAGGCATTCGAGACCGTCAAGGCACGGCTCGACAGTCT
CCAGGAACGTTCCCGCCAGGCGTCCAGCCTGAGAGATGTGCTGGGAGACGCCATACG
CCTGGAGCGAGAACTCGCCGATATGCGCAAGGTCGGGGACCGCGGCGTTGCTGAGC
ATGCCCGGCAGCTTGGCGAACGCCAGGAGCAACTGAAGCGACTCGGCATCGAGGCC
AGGGTCGCGGGCGATGCCTACGCTCGACTGGGCGAGATGCAGCGTGGCCTGGATATG
CAGGTCCGCGGCCTGCAACGGCTGGAGCAGGCCAGCCAGGCAATGCCATTGGCTAG
CGCATTTTCCGGACTGGTCGTGGAGGCCAGCAAGACGGCTGCCGGTTATCAAGCGCG
GTTGCGCGACCTGGCGATCCGCAACGGCCTGGACGTCGGCCGGGAGCCAGCCTTGG
CATCCCTGATCCAGGACAGTGCCAGCCAGAGCGGCCTGGGACGCACGGCGACGCTG
GACATGCTGGAGCACTTGAACGCCACCGGCATGGGGTTCGCCGCCGCGCAAATGAAT
CTGGGACTGGCGGGCCGCTTCGGCTTTGGCCAAGGGATTGCTTCAGCCGAGGTTGCG
GGGCTGGTTCGAGCGTTGCAACTGGCCCAGGGTTCGGACTCGCCAGAGCAATTGTCC
GCCTCCCTCGACCGCCTGGTCGTCCTGGGTAAAGGCAGAGTCGGCAGTGAGGCCCTG
GCGCGTCGCCTGCCTGCCTTGTTGTCAGCGCTGGGCAATGCGGGAGAGGCCACGGCC
GGCGATGTCGGTGCACTGGGGGCCTTGCTGGAGATTCAGGCAAAGAACACCACGCC
AGACAAAGCGGACGTGCGGATGAAGGCCTGGCTGGAGTTCGTCGGCAGCGGCAGCT
TGAAACGCGCTTATGGCCAGGACTACGACCGGGACCTGGAAGCGCTGCGCAAGGAC
GGAGCGAGTCTGCTGGAGGCCAACCTGGAGCTGGCTGCGCGCTATCGGGATAAGGG
CAGCAAGCTCAGCGCCGGCGTGGCGTCGCCGGCGCTTGAAGCCTATCGAGCGTCGCG
CGGCGAGTTCCAGGGCTTGCTCGAATCCCAGCAGTCTTCCGTAGGCTCTTCGGAGCG
CGATGCGCAGCGTCGCAAGGGGATGTCCCAGGAGCTGTGGAAGGCTTCCAGCGACA
GTTGGGAGAGGGCGCAGACCGCTTTGGGCAGCGCCCTGAATCCATATCTGGACAACC
TGGCCAAGGGGAGCGCGGTACTCGGCGAGTCGACTGCGGAACTGCTCGAAGCCTAT
CCGCGGACGACGGCCGGTCTTACCGCCGCCGCAGGTGCGGTGTTATCCGGGTATCTC
GCCTACAAGGGAGGGCGCGGCGCTATCGACGTGTTGCGTGGCGGTCGGCTCGGTCGG
CGAGGGACCGCTGCCGTCGGCGACCTGATCGAACGGGGTGCAGGCCGGGTATCGGG
TGGTAGCGAAATACAGCGCGTGTTCGTTACCAACTGGCCGGTGCCGGGAGGCGACTC
TACGCTGGAGTCCGCGAGGAGGCCAGCACAACGAAAGCGCGGTCAAACACCACGCA
GGAAGAGAGGCAAGGGCGGTGGGCTGAAGGCCCGCTCCCTTCCATCCCTTGGCTTCT
CGGCTGGAGGAGGCTTGGGGGCGATGGCAGGAAAGCTGCCGCGCCTGTCGCGTTTG
CCCATACGCAACGCGCCGCTGCAAGTGGCCTCGTCGTTGATCGATGTTGCTGAGGTCT
ACTCCAGCGACCTGTCGGAGAGCGAAAAGACCGTTGCCTATGGCGAGGCAGGTGGC
TCCCTGGCTGGTTCCCTGGCTGGCGCCGCTCTGGGAGCGAGCATCGGCTCGGTGGTG
CCGGTGGTCGGTACGCTGATCGGTGGATTGGTTGGCGGCGCTATCGGCGCCTGGGGC
GGTAGCGAACTGGGGGGGCGCCTGGGGCGCAGTCTGGCTGGCGATCCGCCGGCGGC
CTCGGACAACAAGCCGGCGGTGGCCGTACCACAGGCCGGACCCGTCGCGGCTGCGC
CCAACTGGACCTTCGCGCCGCAGATCAACCTGACGGTGCAAGGCAACGTGCACGAG
CCGCAGCGCCTGGCCGACGAGTTGCTGCCCTACCTGCAACGCATGCTTGTCGACTTC
GCCGACGAGCGGCAGCGGCGCAGCCTCTACGACCCGGCGATGGTTTAAGGAGTCCC
CATGGCATATCTGGAACAATTGCAGGCCGGCCTGAGGTACCTGGGTCGCGCCGGCGA
GTCCGGACGCAAGAGTCTGGACAAGGTGGTCGCTCCGGTGAACGGCGCGATCAGCG
AGATCCGCGGCGCAGCCGCGGAGCTGGAGAACCTGCCCGGCGTATCGCCGGAAATG
GCTGCCCGGCTGCAGCGTGCCATGCGCGGCATCGGCCAGGCGCAGGGCAAGGTGAA
CCGCGTGGTCTCCACCTATGACCGGGCGAGCCGGGCGTTGCTTGGTATCGACGAACG
CCTGGATGCGCTGAAGGTGCAGGTGAACAGTGCCGCGCAGGCGGTCGGCAAGGTCG
CTGGCGACATCAGTCCGACGCTGGCGGGGGTGCTGCCGTCGTGGCTGCTGGCACCCT
CGGCGACGCCCCCGAGCGAGGCCGCAGCGCCCTTGCCGCACCTGCTGGTACTGCAG
CCGCTGACCGCCAATGCCCAACCGTTCTACTTCAACCTGAATACTGCCGCCTTCGACG
CCCTGCAGCGCAACAGTGCCTACAACTGGAGCGGGCAGGTGCGCCTGGGTCGGCGG
CCGGCGCTGCAGAGCGTCGGCATGGGCGAGGAGAGCATCCTGCTCAAGGGTGCGGT
ATTCCCGCTGCGTCGACAGGTAGGTAACCAGGAAAAGGTCGTCGGTCTGGAGCAGCT
CGAAGCGCTGCGCCGGTTGGCGGAGCGGCGTGAGCCGCTGATCCTGAGCAGCGGCT
ACGGCGAGGTGCAGATGGGCCTCTGGTGTCTGGTGCGGATCAGCGAGAACCAGAGC
GCCCTGCTGGGCAACGGCGCTCCCCGCAAACAAACCTTCGACCTGGAGTTCAAGCG
CTATGGCGACGACCTGCCGAACCGCTGACGGCGACATGCTGGACAGCCTCTGCTACC
ACGTCTATGGCCATCTGTTGGGCTGCGTTGAGGCGACCCTCGACGCCAATCCCGGGCT
GGCCGATGAGCAGCAGCCATTCCGCGCCGGCTTGCTGATCAGTTTCCCTGACATGCC
GGTGGTCAATGTCGAACAGGTGCGCCTGTGGGATTGATCGACCGCTCACCCGCAACC
CCGCCTTGGCGGGGTTTTTCTTTTCTGGAGAGATCAGGTGCAACCGAGTTTCCGTATC
GTTGCCGACGGCACCGACGTCACCCAGCGGCTGAATGACCGCCTGCTCAAGCTGACC
CTGCTGGACAAGCCGGGCATGGAGTCCGACAGCCTGACCTTGAGGCTCGACGATCGC
GATGGACAGGTGGCCTTACCCAGGCGCGGTGCGGTGCTGGAGGTTCATCTCGGCTAT
GCCGGCGAGCCACTGATGCGCATGGGACGCTTTACCGTGGACACCTTGCAGTGGGCT
GGTCCGCCGGACCGCCTGACCGTTACTGCCAAGGCCGGCGACATGCGCGGCAGTGG
CAAGACGATACGCAGCGGCGGTTGGGAGGGCACTACCCTGGCTCAGGTCTGCCGCG
ATGTTGGCGCACGCAACGGCTGGCGCGTGGAGTGTCCGTTGCAGGTGGCGATCGCCC
GGGTCGACCAGGTCAACGAGTCCGACTACCACTTCGTCACCCGTCTGGCGCGCCAGT
ACGACTGCACCGCCAAGCTGGCCGAGGGCATGCTCATGGTGCTGCCGCGACAGAGC
GGGCAGAGCGCCACGGGGCGTCGGATCGAACCTTTGGTGTTGGGACGTGCTGATGTC
GGCAGCTTCGACGTTACCTTCGACGACCGCAGCCTGATGAGAACGGTGAAGACCCGC
TACCAATTGCCCGGCAGCGGCGAGGTCAAGAGCGTCGAGTTGAAGAACCCGAAGGC
ACCGGCTACGGCTACGGGCGAGCATGTCGACCGGCATCTCTATGCCAGCCGTGGAGA
GGCCGAGCAGGCGGCGAAGGCTCGCCTGGCGAGCTTCAGCCGCTCCAGTGCCAGTG
TGCGCCTGGAGCTGCCAGGGCGTGGCGACCTGTTCGCCGAGCGCAGCCTGCTGCTCC
AAGGCTTCAAGGCGGGAATCGACGGCGAGTTCCTGATCGACTCGGTGGAGCACACC
TACAGCTCCAGCGGATGGACCACTGTCGTGCAATGCAACGGCGGCCGAGGCGGCAA
GGGGTGACGCCTGAGACCCATCGGAGTGCAGGAGGATCGATGAAACTGACCGAGCA
GCAATTGCTGCGCATTTTTCCCAACGCCCGCCTCGTCGCGGGCGTTTTCGTTGCGGCG
TTGCAACGGGCCATGGACGAGAGGGAGATCGACACACCGGCGCGGCGTGCCGCGTT
TCTCGCCCAGGTCGGCCACGAAAGCAGCCAGTTGACCCGGCTGGTGGAGAACCTCA
ACTACAGTGCCCAAGGCTTGGCGGCGACCTGGCCGAGCCGCTATCTCGGCCCCGACG
GGCAGCCCAACGCGTTGGCCTTGCGGCTGGCGCGCAATCCGCAGGCGATTGCCGACA
ACACCTACGCCACGCGCAACGGCAATGGCGACGAAGCGTCCGGCGATGGCTGGCGC
TTCCGTGGGCGTGGCTTGCTACAGATCACCGGGCGTGCCAACTACCGGTTGGTCGGC
GAGGCCCTCGGCGAGCCGCTGGAAGCCGAGCCCTGGCGCCTGGAGCAACCCGTGCC
GGCGGCCCGCAGCGCCGCCTGGTGGTGGGCCGGTCACGGGCTCAACGAGCTGGCCG
ACCGCGGCGAGTTCGCTGCCATCACCCGCCGCATCAACGGCGGCCTGAATGGCCAGG
CGGAGCGCCTGGCGTTGTGGCAGCGGGCCAGGGCGGTGCTGTCATGAGCCGGCTCG
CTCTGCTTCTGCTGGCCGTGTTGCTGGTCCTGCTGGCCGGCGCCTTGCTCGGCGGCG
GCCTGGTTGCCCGCCACTATCGTCCGCAACTGGAGGAGGCCCTGGGCCAACTCACTG
CCAGCCGCGTCGCCAGCGGCCAGCTCGAGGCTTTGCTCGATGAGCAGCAGCGAGCG
CTGGCGGCGGTGCGGGCGAGCGCCGAGAGGCGCGCGAAGGACGCCGAGCAGGCAC
TCGGCGAGGCCAGGGCGCAGGCCGCGGAGCAGTATGCCGCGGCCGTGCGTCTGCTC
CAGGAACCCGACATTGGTGTGGACTGCCAGGCGGCAGGTGCGGCGATCGACCGGGA
GCTGGGACTATGACTCGTCTCCTGCTGGGGCTTTGTCTGCTTTTCGCGGGCTGCGCAG
CCTCGCCGACGACACCTCGCCCCGTGCGCGTCGAGGTTCCCCTCGCAGTGCCCTGCC
GTGTACCTGACGTGCGCCCGCCGAGCTGGGCCGGCGCCACGCTGAAGGCCGGCGATT
CGCTGCAGGCCAAGGTTCGCGCATTGCTCGCCGAGCGCCGCCAGCGGCAGGGCTAC
GAACTCGAATTGCAGGCGGCGTTGCGTGCCTGCCGCTGAGACACTGGGCGCATATGG
ACGTCGATGGACGTGTGTCCGGCGCATATTGCCGTGTATTGATTCCGCAGCGTTGTCG
AGCCGGGAATCGGCCGGTACAACGTAGTCATGCTTGTACAGGTGTGTCCCCCCAGGG
ATGTCACCTGCAACCTCAGAGCCCGGCACGTGTGCCGGGCTTTTTCGTTTGCATCCGA
CAACGGCTCGGGACGTGGAGGCTCCTCGCCGACCGCGTACCGCGCCACGGCTGACC
GTCCTGGGGACGGGGCCAGACGACCCGCCTGGTGCGGGTCTTTTCATGTGCATAAAG
GAGAGTTTTCCATGTCCATCCTGACTCAAGGTACCCAGATCTACGCCCTGGTTCCGCC
GGTATCCGGTACCGGTGCCGCTACCGTCCTGGAGATCGAAGGCGTGACCTCGTTCAA
CCCGGGCGGCAATCCGGCCGACCAGATCGAAGACCCGTGCCTGAGCGACACTTCGC
GCAAGTACAAGAAAGGCCTGCGCACCCCTGGCCAGGCGACCCTCGGCATCAACGCC
GATCCACGCCTGGCCAGTCACGTTCGGTTGTTCCAGCTGTCCGAGAAAGATGGCGAG
ACGAGCGTCAAGTGGGCCATCGGCTGGTCCGACGGAATCGACGTAAAGCCGACCGT
CAGCACCGAAGGCGACGATTTCGTGTTGCCGCCGGCGCGCACCTGGTTCACCTTCGA
AGGCTACGTCAGCGACTTCCCCTTCGATTTCGCCAGCAACACGCTGGTCGCTACCCA
GGCCACCATCCAGCGTTCCGGCGCCGGCAAGTGGACGCCGAAGTCGGCTTGAGGAG
CGCGTAGATGAATCTCAACGAACTTCGTGCGGCGGGCGGTTTCATCGAGTCGGCGCT
GGTGCGCAAGGAGATTAGCTGGACCCGCGTTCCCGCCGGCAGGAAGAAGGCGGTCA
GCGACACCTTCCAGGTGTTCGTCCGACGCAACAGTTTTGGCGCGGTGGAGCGCCTGT
TCTCCGCCGAAGGCGACCAGCAGAGCCGCAACGCGCGCTACCTCGCCGAATGCATTC
GCCTGGGCGAGACGGGAGAGGAAAGCCTGACCTACGAACAGGCCTACGACCTCGAC
CCGGCGCTGGGCTTCCTGCTGCTGCAGGCGGTCGGTGAGGTCAACCGGGTCGAGGA
CGCGGAAAAAAACTGACCCCCGCCGACGAGGTTTGGCACGAGCTGGTGCTGAACGG
CGTCGGCGGGTCGACCATTGCCGAGGCCAAGGAGCGGCTCAGCTACGCCGAGTACC
GCGCCTGGGTCGCCTACCTGAACAAGCGCGGCTCGCTCCATCCGGGACACCGGCTGG
AGCTTGCGCTGGCTCGGATCGCTGCGTTGCTCGGGCATGCGCTGGGAGCGGACGCCG
ATCCCGACGCGTTCCGTCCGCATATGGCTCTCCAGCCCCTTTCGCTGCACCAGGCGAT
GGATCAATGGGCATGACAGGCCACCCCGCTGCGGCGGGGTCTTTTTCTGGAAGACAT
GAATCATGGCCACGAATACCGACGGCAGCCTGACGCTCGACCTGGTCCTTCGCAGCG
AAGGGTACAGGGCCGGGATGGACAAGGTTGGCAGGATCAACGATCAGAAAATGCAC
GCCATGGAGGCGCGCGCGGAAAAGGCTGGCAAGGTTATCGGCAAAAGCCTGGACAG
TTCGGCGCTGATTGCCAGCAGCGTGCTGGACCAGGCGCTGGACATGCTGGGCAGGAC
CAGTCGCCAGGCGGGTCAGGCCAAGAAGCCTGTGCAGAGCGCCCAGGACAAGGTAC
TGGCCGAGTGGAAGACCCGGCAGAAGGAACTGGGCGAAGCCTGGAAGAGCTATCGC
GAACCACTCCAGGATCTGTCCAAGCTCAACGAAGCACTACTGAAGAACTCTTCCGAC
AAGCTCGACAAGGCGCTGCTCAATCTCAGCGAGACCGGCAAGCTGTCGCTTGCCAA
CGTGGGCAAGGCCGCCTACGCCGATGCCGCGCGCCTCGCCTCGCGGCAGATGACGCT
GATGCTGCTGGACGGGCTGTTTGGCTGGGTCGCCAGCGTCGGTACCGAGAAGCCCAA
GGTCGACGACAAGGCGGGCAAGGGACAGGCGAAGGCCGGCGACGACGAGAAGGAA
CAGCCGTCGCTCCAGTCGCAGGTCTTCAAGCAGTGGCTGTTGCAGATGAACAGTGTC
TGGGGCGCCTACCGCGCGCCGCTGCAGGATATCTCCGGGATGACCGACGAGCTGTTC
AGGAATGCGTCGGAGAAGCTCGAGAAGTCGCTGTTCAATTTCGCCACTACCGGAAAG
CTGTCCTTGAGCAACTTCGCCAAGACGGTGATCGACGATGTCGCCCGGATCGCCGCG
CGGCAGCTTTCAATGCTCGCCCTGGACGGATTGTTCGGCTGGATGAATGGCAAGGCC
GGCATCACCGAGGCGCAACTGGCCAGCCAGAAGCCCTATACCTCGCTACTGGAAAAG
GCCCGAGCAGATGCGGGACAAGCGGCTGCGGGCGCTCCCGCGGCCCAGGGTGCCGC
GCCAATGCCGGCAGCGGCGATGGATGTCGGCGCGATGGTGGCCACTGCTTCCGGGCA
GACCGGGGACGGTTCCAAGGTATCGGCTGGAGGGGCTTCGGCGAGCGCTGGCAAGC
CGGTGGGCAGTTGGGTCGAACAGATGGACGCCTCCTGGGCGAGCCTGCGCGACCAG
GCGCAGGACGTCTCGGGAATGATGGACATGCTGTTTACCAACGCCTTCACCAATATGG
AGAACGCCCTGTTCACCTTTGCCACCACGGGCAAGCTGTCGTTCAAGGATTTCGCCG
ACTCGGTGATCCAGGATATGGCGCGGATCGCCGCGCGGCAGGCGACGCTGCAGATCA
TCGGCGGCATCGTCGGTGCGGTCAGCGGGTTCTTCGGTAGCGGCGCAACGGCGGGCT
CGCGGATTTCCGACTATACCGGCTCGGACATGGCCAATTGGGTCAGCAAGCAACGCG
CCGGAGGCATGCCTGGGTTCGCCAGGGGCGGTGCCTTCAACGATGGCATCCAGAGCG
CGCCGGCGCTGTTCAGCATGGCCGGCGGTCGTCCGGCGCTGATCGGCGAGCGTGGGC
CGGAAGCCATCATGCCGCTGAGTCGCGGTTCCGATGGCGTGCTCGGCGTGCGCGCGC
TCGGCGGCGGCGAGGGGGGCAACGTCTTCAATTTCTCCACCAGCGTCAGCCTGGGCG
GCGGCCGCGAGGGCGCGGCGACGGCCAGCGGCGACGACGGTACGGGACAGCAGCT
GGCGGGAATGATCAACGATGCCGCGCGCAACGTGGTGGCGCAGGAGCTGCGCCCCG
GCGGCCTGGTATGGAGGATGGTGAATGGCTGATCTGGAACGCTTTACCTGGGACATCT
CGATCGATTCCGCCGGCCAGGCGAACCAACTGGTACGCCAGGTGCAGTACGGCGGCG
GCTACAGCCAGGCGCTCGGCGACGGGCTGAACAACCTCAGCGAGACCTGGCAGGTT
TCGCGTACCGGCGATCTCGCGCTGATCGGCCCGATCCGCGATTTCCTCAAGCGCCACG
GCGGCTACCGCTCGTTCCTCTGGACCTTGCCCACTGGCGAACCGGTACGGGTGCGCG
CCCAGGGCTGGCAATTGCGGCCGCGCGGCAACGGCGTGTTCACCCTGAACACCACCT
TCCAGCAAGTCTTCAATCCGTGAGGTAAACATGACCATCACAGCCGATGACCAGGCC
CTCGAGCCTGGGGCGCTGGTGCGCCTGTTCGACCTGGATTGCACCGGGTTCGGCGGC
GAGATGCTGCGCTTCCACGGCCACCTGCAGCAGGGGCCGATCCACTGGCAGGGCAA
CGCCTACCAAGCTTGGCCGCTGGAGGCGCGCGGCTTCGAGCAGCGCGGCGACGGCC
GGGCCAGTTCGCCGACCCTTAGCGTGGGCAACATCGACGGCAGCATCAGCGCGCTCT
GCCTGTTCTTCGATGGCCTGGTAGGCGCGCGCCTGACCGTGCGCGAGACCTATGCGC
ACTACCTGGATGCGGCCAACTTCGCCGAAGGCAACCCGCAGGCCGACCCCTCCCAGG
AGCGCCTGAACATCTGGTTCCTCGAGCAGAAGACCGCCGAGAACAGCGTCCAGGTG
ACCTGGGAGCTGTCCGCTCCGCCGGATTTCCAGGGCCAGCAGATCCCGGCGCGCCAG
ATCACCTCGCTGTGCCACTGGTGCATCACCAACGAGTACCGCGGGCAGGACTGCAAC
TACACCGGCACGGCGATGTTCGATGCCGACGGCAATCCGGTGGACGATCCGGCGCTG
GACCGCTGCGGCGGCCGGGTCAGCGATTGCAAGCTGCGCTTCGGCGCGGACAACCC
GCTGTCCCACGGCGGCTTCGCCGGCGCCGGCCTGGTCAGGATGTGAGCATGGAACTG
AGCCGCAGCCTGCAGCGGGCCATCGCCGCACACGCCGCCCGCGAGCATCCGCGCGA
ATGCTGCGGGCTGATCGTTCGCGGTGCGCGTCAACGCCGCTACGTGGCCTGTCGCAA
CGCAGCCGGATCGCCCAGCGAGCACTTCGTGATCGATCACCAGGACTGGTGCGCCGC
CGAGGACCAGGGCGAGGTCCTGGCCATCGTCCACAGCCACCCGGACGTTCCGGCCA
CGCCGAGCATGGCCGATCGGGTCAGTTGCGAACTGCATGGTCTGCCCTGGGTGATCC
TGTCCTGGCCGGAAGGCGATGTCGCGCATCTAGCGCCGGAGGGCTATCGGGCGCCGC
TGCTCGGCCGCGAGTTCGCCCACGGCGTGCTCGACTGCTGGAGCCTCTGTCGCGACT
GGTACCGCCGCGAGGCAGGTTTGGAGCTTCCGGACTATCCGCGCCGCGACGGTTGGT
GGGAAACCGGCGAGAGCCTCTACGAGCAGCACTATGCGGCGGCCGGATTCCGGCCG
GTGCCGCTGGCCGGAATCCGCCGCGGCGACATGCTGGTGATGCAGGTCGGGAGGGC
GCTGCACCCGAACCACGCGGGCATCTACCTGGGCAATGACTGGCGTCTGGACAGCGA
GCCGGTCCAGGCGCTTGGCGGCGACGGACCGTTCCTGCTGCACCACCTGTACGGACG
GCTGTCGACCCGCGACGTGTTCGGCGGACCCTGGATCGAACGCACGCGCCTGGTCTT
GCGGCACACGCGGATGCCGCAGTGAACGACATATTCAAGCGAGCCGTCGGAATCGGC
TCTTCACGAGAGGAACAGGTCCATGAGTGACACCCTGAGTCAGGGCCTCACCACCAT
CCGTCTGTACGGGGTTCTGGGCAAGCGCTTCGGCCGCATGCACGGCCGGTTGTTGGA
AAGCGGCACGGTACGCGAGGCGATGAGTGCCCTGAAGCACACCATGGAGGGATTCG
AGACGTTCATGCGCGAGGCGGAATCGAAAGGGCTGACCTTCGCCGTGTTCCGTGGGC
GTACCAACCTGTCCGGCGAGCAACTGGACATGCGCGGACGCGAGGATATCCGCATCG
TGCCGTTGGTGATCGGAAGCAAGCAGTCCGGATTGTTTCAGACGGTATTAGGAGCTG
CACTCATTGCCGTAGGTGTCTTCGCAACGAGTCTTACGCTCGGGACAAGCACGTTTCT
GATCTCTGCCGGCGCCAGCATGATGCTCGGCGGCGTCATGCAGATGCTCAGCCCCCA
ACCCAAGGGCCTGAAGGGCCGAGAGGCCCCCGAGAACGCCCCCAGCTATGCCTTCG
GCGGCCCGGTCAACACCATCGCCCAGGGCCATCCGGTCGGCGTGCTCTACGGCAAGC
GCCGCATCGGCGGCGCGGTGATCAGCGCCGGCATCTATGCCGAGGACCGGCTGTAGC
CGGCAACGCCGTAACAGGCCCGCCATGCGCGGGCGTTTTTTTGCCTGAAGGAACGTC
ATGAACAAGACCATCACGGGCCACAAGGGTGGCAGCAAGAAGCCGCGCCAGCCGGT
GGAGATGCCGGACTCGGTGCGCTCGATCGCGCGGGCGAAGATTCTCCTGGCACTGGG
CGAAGGCGAGTTCGACGGTGGCGTCGACGGCCGTTCGATCTACCTGGACGATACGCC
GCTGCTGGCGGCGGACGGCTCGGTGAACTTCCCTGGAGTGACCTGGGAGTTCCGTCC
GGGCTCGGTGGACCAGGAACACATTGCCGGTGTGCCCGCCGTGGAAAACGAACTGG
CGGTCGGCGTCGAGCTCAAGAGTGACGCGCCCTGGGTCCGCGCGGTGAGCAACACC
CAGCTCTCGGCGGTGCGCCTGCGCCTGTCCTGGCCGGCCATCCAGCGCCAGCAGGAA
AACGGTGACGTGGTCGGCTACCACATCGACTACGCGATCGACATCGCCGTCGACGGC
GGTGCCTGGCAGGAAGCGCTGAAGGCTTCGCTGGACGACAAGTCCACCAGCCGCTA
CGAGCGCTCCCACCGTGTCGACCTGCCGGAGGCGCGGAGCGGCTGGCAGGTGCGCG
TGCGCCGCCTGACGCCGAACCAGAACAACAACCGCATCGCCGACACCATGCGGGTC
GAGGCGATCACCGAGGTGATCGACGCCAAGCTGCGCTACCCGAACACCGCGCTGCTG
TTCGTCGAGTTCGATGCCAGCCAGTTCCAGAGCATTCCGCAGATATCGGTGGAAGCG
CGCGGCCGGCGGGTGCGGGTGCCGAGCAACTACGATCCGCAGACCCGTAGCTACAG
CGGCACCTGGGACGGCTCGTTCAAGTCGGCCTGGACCAGCAACCCGGCCTGGCACT
GGTACGACATCGTGTTGCACAAGCGCTTCGGCCTCGGTCGGCGGATCGACGCGAGCA
TGGTCGACAAGTGGTCGCTGTACCGCATCGCCCAGTACTGCGACCAGTCGGTGCCCG
ACGGCAAGGGCGGCCAGGAGCCGCGCTTCAGCTGCAACCTGTACCTGCAGAGTCGC
GCCGAAGCCTGGACCGTGCTGCGCGACCTGGCAGCGATCTTCCGCGGCATGTCCTAC
TGGTCCGGCGCGGAAATGGTGGCGGTATCCGACATGCCGGAGGACGAGGCCTACACC
TTCTCACCGTCGAACACCGTGCGTGGCGACGACGGCAGCCACTTCAACTACAGCAGC
AGCCGCCAGCGCGATCGCCACACCCTGGCCCTGGTCAACTACGACAATCCGGGCAAC
GGTTACCAGAGCCAACCGGTAGCGGTGAACAATGACCGCGCGCAGCGCCGCTACGG
CATCAGCCAGTTGGAGATCACCGCGATCGGTTGCACCTCCGAGGGCGAGGCGCAGCG
GCGTGGCCAGTGGGCGCTGCTGACCGAGGAGCTGGAGCAGGACGCGGTGACCTTCC
GCACCGGCATGGATGGCCGTGGGCTGGCGCCGGGGAAGATCATCGCCGTAGCCGACC
CGGTCAAGTCCGGCAAGCAGATCGGCGGACGCCTGAGCGCGGTGGATGGCCGCGCG
CTGACCCTCGACCGCGACGTCGAGGCCCGACCCGGCGATCGCCTGCTGGTCAACCTG
CCGAACGGCAAGGCCGAGGCGCGCAGCGTCCAGTCGGTGGTAGGCCGCGTGCTGAG
CGTGACCGCCGCCTATTCGGAGACGCCTCGGCCCCAGGGGCAGTGGGCGCTGCAGA
GCAACAGCCTGACCACCCAGCGCTTCCGCATCATGAGCATCACCCGGCCGGAGGACA
ATCTTTTCGAGATCACCGCGCTGCAACACAACGCGAGCAAGTTCGACGCCATCGACA
ACGGTGCGCGCATCGAGCTGCCGCCGGTCACCAGCATTCCGCCGGGCGTGCAGGCGC
CGCCGCAGAACGTGCGGATCAAGGCTTTCACCAAGGTCGACCAGGGGTTGGCGGTG
ACCAGCCTGTCGGCCTCCTGGGATGCCGCGCCGAACGCGGTGGCCTACGAGGCCGA
ATGGCGCAAGGACTCGGGCAACTGGGTGCGGGTGCCGCGAACCTCGGCGCTCGGTT
TCGACGTGCCGGGCATCTATGCCGGTCGCTACCTGGTGCGGGTACGCGCCTTGAACGT
GATGGAGGTCGGTTCGGTCTACGCCAGCAGTGTGGAAACCGCTCTCGAGGGCAAGA
CCACACCGCCGCCGGCGCTGGCCTACCTGCGCTGCGTGGCCGGCCCCTGGCGCATCG
GCCTGGAGTGGGGGTTTCCGGCCAGCGGCGCGGCGGACACCGCCTACACCGAGATC
CAGCAGTCCGCCACGCCCGGCGGCAGCGAGGAGACCGCACGGGCGCTGGGCCTGTT
CGCCTACCCAGGCAATACCCACCTGGTATCGCCGATACCGGCCGGCGAACGGCTGGC
GTTCCGCGGTCGCTTGATCGACCGTAGCGGCAACGTCGGCGCCTGGTCGAACTGGGT
CACCGGCACCAGCTCCAGCGACGCCAGCGAATACAACCAGTTGATCACCCAGGAGTA
CGTCGAGTCGGCGCTGGGCCAGCAATTTTTCTCCGATATCGAGCGGATGCAGGTGGAT
ATCGGGGGCTTGCAGAAGCAGGTCGGCGACCTCGCCGACATTCTGCTGTACGACCCG
GCCAAGATCTACGCGAAGAACGACATGGTGCGACAGGGGCCGCGGTTGTACCAGGC
ACTGAAGGCTGTGCCGGCGAAGACGGCGCCGCCGAACGCGGCCTACTGGTCCGATAT
CGGCCAGTCGCTGGAGACCGCCAACGGGCTGGCGCAGCAGGTGGCGAGCCATACCG
CTGAAATCAGCGAACTCGACGGCAGGATCGAAGCAGCGGTATCGAATCTGGATGTGC
TGCAAGCTGCCGCCCGCGGGGAGCCGGCGACCGGAGAGAAGGCGGATGCGCTGAA
GGGCTGGGACACCATTGCTCGAGCCGCCACCGAAGTCACCGTGCGGGCGAACGAGG
ACGAAGCGCAGGCGAAGCGGGCGAGCTTGCTTGAAGCGCGGACCGGGACCGCGGA
GGGCAGGATCGCCACCGTCGAGTCGGTCGTTGCGTCGAACAATGCCGTAACCGTCCA
GCGATTGGATCAGCTCACCGGCCAGGTTGCGAGCAACGCCTCGGCCATCAGCACCGA
ACAGACCGTCCGCGCCAACGCGGACAGCGCACTGGGGCAGCGGGTGGATACCGTCA
GCGCGCGCACCGATACCAACGAGGCGAACATCCAGACCACATCTCAAGCGGTTACCT
CGCTGGATGGCAACGTCAAGGCGCTCTACAGCGTGAAGCTCCAGGCGCATGCCAACG
GGCAGAAGTACGCCGCTGGCTGGCAACTGGGCTTCGACAGCGGTACGAGCGTGACG
ACCATGGCGTTCCAGGCTGATCGGTTCCTCTGGTTCAACAGTTCCAGCGGGCAGACC
GTGGCGCCGGTCTCGATCGTCGGCGGCCAGATGTTCATCAACAACGCGATGATTCAG
GATGGGTCAATCACCAATGCGAAGATCGGCAACGTGATCCAGTCGACCGCCCTCGGT
GCCAACGGCGAGCCGCTGTGGAAGTTGGATAAGGGCGGCGCGTTCACAATGAACAG
CGCCACGTCTGGCGGCTTCATGAGGCAGACAGCGGAGGCCACCAAGGTCTACGACG
CGAATCTTGTGCTGCGGGTACAGATCGGGAATCTAGACGTATGAGCTACGGAATCCGC
CTGAGAAATGCGGCCGGCTCCATCCTGATGGAGCTCACCGGCCAATCGGCGCGCACG
GTCTACCGGCAGTCGCTCGGCGCCATCACCAACGGGATGACGGTGACGGTGCCGGGT
TTTGATCCTGCGCGCGGTGTTGTGTTCATCATCGCGAGCGGAAACGAATTCGGTGAAG
TGCCCCGATACACAATTTCCGGAAGCGTGGTGACGTTCCACTGGAACGGTTCATCCG
GAACAACTTATGTACTGCATGCGGTGATGTTCTCATGAGCTACGGAGTATTAATTCGCG
GGGATGCTGGGCAAACAATAATCGACGATAGTAATCCGTGCATTCATTTCGCTGCGTC
GGGAACTTATGGACATACGACCGGCAGAGAAACTGTTATTCAATATGCCTTTCCAATA
CAGTCCCCGTATGAGCCGTATGTCTTCGTGCGCCCAAATGGTCCGCATCAAATCTATTT
GTTCAGGCATATCGGCGCCCCGGGGAACTGGACTGGATTTGCATTCTGGCAGACGATC
TATCGGGACGTGGACCCTCCAATCTACGGCGGAAAGTGGAAAGCTGGCGCGGTCATG
TTGCCGAAAACCGGTGGGTGGGGAATGCAGGTTTTCGACTCCCAGTCGCGTGTGATG
TTCGACAGTAACCGGGACATCGTTCGCTATGTCGGTGGCGCACAGGTTTGGAATAAGT
ATTCGTACAACCCGAGCTGGCCAGGCGGGATGGCACTACAAACGTGGTATCTGCCGT
TCACATATGGAGTTGAGGCCTACTTCCAAGTCAGCCATTTCAATGTCAAAGCATTCAT
AACGTTAGAAGCGCCGCGCATAGGTTTTCTTGAGAACTCAATGAGCTTGATATTTGTT
TCATCAGTTGTAGAGTTTGAAACTAACCATCAGTTCAATTGGCCGCTTATTGTAGTGG
CGTAAATATATCTGGAGGACTATATGGCTTGGTATTCCACAGGCACGGTTGCTGTCACG
CTGAATTCGCCGACAGTCACCGGCACTGGGACCGCATTTTCCGCCAACGCCCGGGTC
GGCGATGCATTTCGCGGACCCGATGGGCGTTGGTACGAGGTCACAAACGTCGCCAGT
TCGACGGTGATCTCGATCAAACCCAACTACCAGGGCAGCACGGCCAGCGGCCAGCCC
TATGCAGTGGCGCCGATCCTGGGCTACGACAAGGACCTATCAGATCGTTTCAACCAGA
TCGCGATGGACTGGGGGGCGACCCTTGCGGGCATAAAGCCGTGGGCCCTGTCCAATA
CCGGCACGCAAGCGCAGGCGGACATGGGAATGACGGCGGTGGGGCGGGGACTCAAC
GCCGCAGCGACTGCTGAGAATGCCCTGAGCTTTATTGGTGGCATGCCGAAGTCGATG
TCCAATCTGCGGGCTGTCAGCGACGCCAATAATGTCCCGAACGAATGTGGGTTCTACG
GTATCGGAGCGTCGCCTTGGGCGAACTTGCCGCCGGGGGTCGACGGTATCAACCCTA
TCGGATCCATGCTCTACCACCATCCGTACGATGTGAGCACCGCCGTGCAGATGCTCAT
TCCGCGAACCTCTGACCTCATGTACTTCCGCCGGAAGCTCTCCGGCAACTGGAGCGC
ATGGGTGCGGCTACTCTCGGATAAGCAGCTTGTAGGTACGGTATCTGTTGACGGGTCG
AATGTTCCGAACGGTGCGGTCATGCAGCAGAACGGGACCACGGCTATCAACGTGGGC
ACCAGTCTGCGTTTCGCCGACGGCACTCAGATTATCTACGCGAAGCTCCGTCTGGAAT
TCAGCGCGGTAGACATCTTGACCCGCCAGTACACGTTCCCCATGAGCTTTTTCGAACC
CCCGAATGTCACCGCTACTTTAATTCAAGGTCAGCAGTCGGATATCAATCCATTGCAG
TTCCAGCAACTCGGTCCGGTATTGGTCGCTGCTACTACTGTTAGCGCGTGCAACGTAC
GAGTCATGCGCCCTACTTACGTATCGAGTGGCTGGGCTTCTGGGAACTTCATCGACTG
TTCTGTCAACGCGGTAGGGAGATGGCGCTAATGAAGTTCTTGCTAAAACCCGACCTG
CAAGTCGGATTGCCTGGCCAAGAACGGGTAAGCTCGGTTTCTGTAAACGGATTGCGG
TTGACTATCGACGGCGTAGAGTTCGATTTTTCTCCACTTGCAGTGGGTGGGTACTTGC
CTCCGGAGGCATACATCAACATAACCCCCCTGCAAGAGGTGGAGGTTCGAAGCGACT
TTCTTCTCGTACGCTACATCCACCAAGTGACAGCAGACATTCTGACTGCTTATCGCGC
CGAGATTGAACCAATTTTGATGGAAGTTGACGGACCTGTGGAGCTACCGAAATGAAC
ATTGACTGGACCCAACTGAGAACCCCCGAGCAGCAGGCCGCCGAACGCTTGCAGGC
TGAGTACGATGCCGCAGCCGCGGCGCGGGCAAATGCCTACCGCCTGGAGAGTGACCC
GCTCAAGACCGAGGCTGAATTCGATGCGATCAAGGCCGGCACCGAGCCGGACTACTC
TGACTGGATCGCCAAGGTAGAAGAGATCAAGGGGCGATATCCACTTCCTTGA
PAKAF_R1_pyocin_tail
SEQ ID NO: 2
ATGACGACCAATACTCCGAAATACGGTGGCCTGCTCACCGACATAGGTGCCGCTGCG
CTGGCTGCGGCCAGTGCAGCAGGCAAGAAATGGCAGCCGACTCATATGCTGATCGGC
GATGCCGGCGGTGCGCCGGGCGACACGCCGGATCCATTGCCTTCTGCGGCGCAGAAG
AGCCTGATCAACCAACGCCATCGGGCTCAGCTGAATCGGCTGTTCGTTTCCGACAAG
AACGCCAATACCTTGGTTGCCGAGGTGGTGCTGCCAGTTGAGGTAGGTGGCTTCTGG
ATCCGCGAGATCGGCCTGCAGGATGCCGACGGCAAGTTCGTCGCGGTATCCAACTGC
CCGCCCAGCTACAAGGCTGCAATGGAAAGTGGCAGTGCGCGGACCCAGACCATTCG
GGTGAACATCGCGCTCTCCGGCCTGGAGAATGTCCAGCTGCTGATCGACAACGGCAT
CATCTACGCCACTCAGGACTGGGTGAAGGAAAAGGTCGCTGCCGATTTCAAGGGCCG
CAAGATTCTGGCTGGCAATGGCTTGGTCGGTGGGGGCGATCTTTCTGCCGACCGCAG
CATTGGTCTGGCGCCTTCCGGCGTGACGGCGGGCAGCTATCGTTCGGTCACGGTGAA
CGCCAACGGGGTGGTCACCCAGGGCAGCAATCCGACCACCCTGGCCGGCTATGCGAT
CGGAGATGCCTATACCAAGGCCGATACCGATGGAAAACTGGCGCAGAAAGCGAACA
AGGCCACCACCCTGGCCGGCTATGGCATTACCGATGCGCTGCGAGTCGATGGCAACG
CCGTGTCATCCAGCAGGCTGGCCGCACCGCGTAGCCTGGCAGCCAGTGGCGATGCCT
CCTGGTCGGTGACCTTCGACGGCAGTGCCAATGTTTCTGCGCCGCTGAGTCTTTCCGC
TACCGGTGTGGCGGCGGGCAGCTATCCGAAGGTGACCGTGGATACGAAGGGAAGGG
TGACTGCTGGAATGGCGCTGGCGGCGACGGACATTCCCGGGCTGGATGCTTCGAAGT
TGGTCAGCGGGGTGCTGGCCGAGCAGCGTTTGCCGGTATTCGCGCGCGGGTTGGCTA
CTGCTGTCTCGAACAGTAGCGATCCGAACACCGCGACCGTGCCGTTGATGCTGACCA
ATCATGCGAACGGACCTGTTGCCGGACGATACTTCTACATCCAGTCGATGTTCTATCCG
GATCAGAACGGCAATGCTTCGCAGATTGCAACGAGCTACAACGCTACATCCGAGATG
TATGTACGGGTGTCCTACGCGGCCAACCCTAGCATCCGGGAGTGGTTGCCCTGGCAG
CGTTGCGACATTGGAGGTTCCTTCACGAAGACGACTGACGGATCCATTGGAAATGGC
GTCAATATAAACAGCTTCGTCAATTCCGGATGGTGGTTGCAATCGACATCGGAATGGG
CGGCGGGTGGAGCTAACTATCCCGTGGGGCTGGCCGGTTTGCTGATTGTCTACCGCGC
ACATGCAGACCATATCTATCAGACCTACGTAACACTCAACGGAAGCACATATTCGCGC
TGCTGCTATGCGGGCTCTTGGCGTCCGTGGCGGCAGAACTGGGACGATGGAAACTTC
GATCCGGCCAGCTACCTGCCAAAGGCGGGATTTACCTGGGCGGCTTTGCCGGGTAAG
CCGGCAACTTTCCCGCCCTCAGGGCATAACCACGATACCAGCCAGATCACCTCCGGC
ATCTTGCCTCTCGCTCGTGGTGGCCTTGGCGCCAATACAGCGGCCGGAGCACGCAAC
AACATTGGTGCCGGAGTGCCGGCCACGGCGAGCCGGGCGCTCAATGGTTGGTGGAA
GGACAACGATACCGGTCTGATCGTCCAGTGGATGCAGGTGAACGTAGGAGATCATCC
CGGTGGAATAATCGATCGTACCTTGACGTTCCCGATCGCGTTCCCTAGCGCCTGTCTG
CATGTCGTACCGACTGTCAAGGAGGTGGGGCGACCAGCGACGTCCGCGTCGACCGTT
ACGGTCGCCGATGTCAGCGTCAGCAACACGGGATGTGTGATCGTTTCCTCCGAGTAC
TACGGACTGGCTCAAAACTATGGCATCAGAGTGATGGCCATCGGCTATTGA
PAKAF_R1_pyocin_tail_fiber
SEQ ID NO: 3
MTTNTPKYGGLLTDIGAAALAAASAAGKKWQPTHMLIGDAGGAPGDTPDPLPSAAQKS
LINQRHRAQLNRLFVSDKNANTLVAEVVLPVEVGGFWIREIGLQDADGKFVAVSNCPPS
YKAAMESGSARTQTIRVNIALSGLENVQLLIDNGIIYATQDWVKEKVAADFKGRKILAGN
GLVGGGDLSADRSIGLAPSGVTAGSYRSVTVNANGVVTQGSNPTTLAGYAIGDAYTKAD
TDGKLAQKANKATTLAGYGITDALRVDGNAVSSSRLAAPRSLAASGDASWSVTFDGSA
NVSAPLSLSATGVAAGSYPKVTVDTKGRVTAGMALAATDIPGLDASKLVSGVLAEQRLP
VFARGLATAVSNSSDPNTATVPLMLTNHANGPVAGRYFYIQSMFYPDQNGNASQIATSYN
ATSEMYVRVSYAANPSIREWLPWQRCDIGGSFTKTTDGSIGNGVNINSFVNSGWWLQST
SEWAAGGANYPVGLAGLLIVYRAHADHIYQTYVTLNGSTYSRCCYAGSWRPWRQNWD
DGNFDPASYLPKAGFTWAALPGKPATFPPSGHNHDTSQITSGILPLARGGLGANTAAGAR
NNIGAGVPATASRALNGWWKDNDTGLIVQWMQVNVGDHPGGIIDRTLTFPIAFPSACLH
VVPTVKEVGRPATSASTVTVADVSVSNTGCVIVSSEYYGLAQNYGIRVMAIGY
PAO1_R2_Pyocin
SEQ ID NO: 4
TCAGGATGCGATGCTGTCAGCGCCCGGCTGTGCTCGACGGGCGCTTCCGGCTTGGTT
CGCGCGCTGGTCGACGTAGGCGGCGAGGTGATCGAGGGCCACGAACTGCATGGCCTT
GTGACTATCGTCGAGGGTTGTCACCGGCAGGGCGATCCGCCCGCATGCCAGGGCCTT
GCTGAAGTTTTCCTTGTTCAGGTTGCGAAACCAGCGTTCGCGAACCTGCTCCAGCGG
CACCAGGACGTCGCCGAAGATGCGGTAGACCAATTCCACGGTTTCCTGGCGTGGAAT
CGGAGTGCTAGGGGCGATGGATGGCTGCATGGTATTCCCTCCTGCGGCTACACGTCGT
TGAGGGAAATATAGCTCAGGTTGTTTTCTTGTTCAATAGCTGAAGTTGTAGAGCGGGC
GAGCGCCAGGCGCTCACCGCACCAGGGACGGGCCGCGCCGGTGGCGTACGGTGGAC
CACCAGAAGACCCAACCGATCATGCTGATCTGGCGGCTGCGCATGTCCTCCGGCGAG
TACTCCTCGTCCGGATATTCCTCGCGGTTGAAGCTGCGCAGGCGAATGCCGCCGCCGG
GCAGGCGATAGACGAACTTCACCCGCAGCATGCCGTCATGTTCGAGGGCGTAGATCT
CGCCATCGGTGATATGGGTGGTGGCGGTGTCCACGCCGATGGTGGAGCCATCCATGAT
CAGCGGTTCCATGCTGTTGCCGGTGAGTTGGGCGCAGATCGCCGCCGACGGATCGAC
GCCCGAGGCACGCAGCGTGGCGTAGGAAAAACGCAGCTTGCGCCCCTCTATCTCGCG
CACCGCAGTGCGTCCGGCGCCGGCGGACATCTCCACTTCCTTGTACAGCGGCAGTTC
CACTTCGTCTTCGTCCAGCGGTGTGTCGCTGTCCCACGGATGCAGCGGTTCCAGCAC
CAATGGGCTGCCATCGGCCACGGACACGGCACTGCGCGCGGCTGGAGCGCCTTCGCC
GGTCTGCAGCCAGACCGGCGAGACGCCCAGGGCCGCCGCGATCTCGATCAGCTTGC
GGGTGCTCTGGGCCTTGCCCGAGGTCAGCTTGTGAATGGTGTTCTGCGAAACCCCTG
CCGCTTCGGCGAGGGTTTCCTGCTTCAGGTTGCGCATCGCCATGGCCTGCTTGAGGC
GAGCGGCGAAGCTGTCGGGCGGGATCTGGGTGCTCTTGTCCATGCTCGGCAATCTAC
AGACCGATGGATTTTCTGTAAAGAGCCTAGGTGTTGACGATAAATAGCTTTGGTTGTA
ATTTCTCTTCCGTCAGAAAGCGGAAGGGGTGAGTTCCACAACTTCCCACCCGCCTTT
CAGAGATTTCACATGGGTGCAGGGACGCACCCGGTTGTCGGCGACCGACGATGCAG
GTGCCGACTGCTTGGGTGCCGGGGCCATGGGAAAGCGCTCCGGCACCTGGAGGGCC
GGAGCGCCGTCCATGCAGACGCTCCGTCCCGCTCATTCATTCTGTCGCCGACCTGCTT
CAGGTACGGCGGCGTCGTACAGGGAGTCATTGCCATGTCGCGAGATACGCACTCGCC
ATTTCCGCGCAGAGGAACGCAGGTCGCCGAACCGGCGCCGCGCATCTGTCATGTTCC
ATCCATTCGGCCGTCGGGCGTCGGGCACGCCAGGGAGGCTTTCCATGGCTGACCTTG
CCGATCACGCCAACGAACTGGTCCTGGCTCGCCTCGACGGCCTCCTGGCGGCGCGCC
CGGCGCTGGCCATCCGCGAGTCCGCGGAAGACTGCGAGGACTGCGGCGAGCCCATT
CCCCAGGCGCGCCGCCGGGCGGCACCGGGCTGCAGTCGCTGCATCGACTGCCAGGA
CCGCCACGAGCGCCGTTGAACCGACCTCATGCCGAGCCCTCGCGGGCAGTGAAAGG
AGACACGACCGTGATCAAAGCCATCGATGAAATGCTCAAGCTCTGGGCCGAGGAAAT
GCACGCGCCAGGCAGCAACGGAGGCGGCTACGCCGGCGGCAACCTGATCGCCATGC
TGATCGCCAGCAAGGGCGCGGTGGTCCGCGGCCACCGTGGCAGCCGGGTGATCCTC
GACCGTGTGGCGGAGGTCGATCGCCTGGTAAATCGCCTACCCGAGGAACTGAAGAAC
GTGGTGGTGGAGCACTATCTCAATCGCGACAGCTTCCCCGAGCAGAAGTACCGCCAC
TGCGGTTGCAGCCGCAACACCTTTTATCTGCGCCTGCATGTGGCGCACCAGGGTATCC
AGGACGGCCTGCTGCGGCGGGTGGCCTGAGTTCCTGGACCGGATACCGTGTCCCCCC
TGGAATTTTCCAACCGGCCTTCGAGCCGGTTTTTTTATGCCTGTCGCCCATGGAGGCG
GTGAGTCCCATGCACAGCAGCGGACCCTGCACGCTAGAGGCGGGTTCGGGCTGGCG
GAGCCGTCCTTTTCGTCAGTCGATGCCGGGCTTTTCCATATCGGAAGTGGGTACGCGC
TTGCCCGGATGCCTGGCGAAGGTTCCCAGGGCGGTATATCACCCTCATATCGTGGTGC
GTCTCCCTCCTATTGCAGTGGATTGTCGAGAAGGCATTGCCGGGCTGGGAATCGGGC
GGTAAAAAGTAGTCATTCTTGTAAAGGTGCGTCCCCAGGGAGGCACTCGTGAAGCAC
CGGAACCCGGCCCTGGCGCCGGGTTTTTTATTGCCCGGGAAAACGTTCGTGCGGACG
TGCCCGGCGCTGCCTGCCAAGGAGGGACCCATGGGCAACGAACCGCAGACACTGAC
GGAAATGCCGCTCTGGGTACTGATCCTGCTCGCCGCGCTGGGCGGCGTCAGCGGCGA
GATGTGGCGTGCCGACAAGGCCGGTCTCGGCGGCTGGGCCTTGTTGCGGCGCCTGGC
GCTGCGCTCCGGCGCGTCGATTGTCTGCGGCGTGGCGGTGATGCTGCTGGCATTGGC
TTGCGGCGCCGCGCTGCTGTTCGCCGCGGCGCTGGGCAGCCTGACCGCCGCGGCCG
GCGCGGAGATCGCAGTCGGTCTCTACGAACGCTGGGCCGCCCGGCGCCTGGGGGTTT
GCGAGCTGCCCGAGGAACAGTCGGACGACCGTGGCCCGCATTGAAACTGATCGAAG
GAGTCAACCATGCCTGAACAGGCTGTTACGCTCGAGGCTCTGTACGCGGCCATCGAG
CAGGTACTGCGTGAGCGTCTGCCGGAGGCGCAGTTGATCGGCTTCTGGCCAGGCGTG
CCGGAAAATACCCCGGCGGTTTCCCTGGAAATAGCCGAGCTACTGCCCGAGCGCGAT
CCCGGTACCGGCGAGAGTGCCCTGCTGTGCCGCCTGCAGGCGCGGATAATGGTGCCG
CCTGGTGCCGATCGCCAGGCGGTATCCATTGCTTGCGGAATCGTTCGGACATTGCGCG
AGCAGACCTGGAACCTGTCTCTGCAGCCGGCGCGCTTCGTACGCTCGGCCGTCGACG
GCAGTCGCGAGGAGCTGAAGAGCCTGCGTGTCTGGCTGGTCGAGTGGACGCAGTCG
CTGCGTCTCGGAGACCCGGAGTGGGCCTGGGAGGACCAGCCGCCCGGCAGCCTGAT
GCTGGGCTTCGACCCGCAGACCGGCCCCGGCCATGAGCCGGACTACTTCGCTCCGGA
GGCGTTGGCATGAGCTATGTCAGTGCGGAGCATGACCGCATGCTCGCCGCGATGATCC
TGCCCTGCGTGGTGGTCGCCGTGGACCTGGCAGCGGCGCGGGTACGGGTGCGCTCCG
GCGACTGGACCAGCGGCTGGCTGCGCTGGCACTCCCTGGCAGCCGGCAAGGTTCGC
CACTGGCGTGCGCCGAGCATAGGCGAACAGGGGGTACTGCTCAGCCCGTCGGGCGG
AGTGTCAATGGGCACCTTTATTCCCGGTCTGTACGGCGATGCGGGCACGGCGCCGGA
CAACAGCGCCAGCAGTGAGACCTGGCGTTTCGACGACGGCGCCTCGTTGAGTTACG
ACTGGGCTGCGCATCGCTACCGCGTCGAGCTGCCCAGCGGCACCGTGGAAGTGAGG
GTCGGCGCCAGCGAGGTGCGGGTCAGCGACGGGGCGGTCAGTCTCAAGGCGCCGAA
GATCAGCCTGGAAGGACCGGTGGAGATCGCCGGGACACTGACGGTCAGCGGAGACA
TCCTCGGCGGCGGCTCGATCATCGACACCGCCGGCAACAGCAACCACCACACCCATT
GAACATACGACGGGGCTGCCGAAGGGCGGTCCGTCGAACATTCAACCGGCCCGCGC
GAGCGGGCCTTTTCGTTTGCGGAGTTCGCCATGGGCAACACTCACAGCCATTCGGAG
CCAGGCGCGGCCTGTCTCTGCCGGAGCGGGAGGGCAGGGCGATGATCGGGATGGAT
CGCCGTAGCGGGCTACCCCTGTCCGGCCTGGCTCATCTTAAACAGTCCGTCGAGGAC
ATCCTGACCACCCCGTTGGGCAGCAGGCGCATGCGCCCCGAGTACGGCAGCAAGCTG
CGGCGGATGGTCGACATGCCGGTGAGCGAAGGCTGGAAAAGCGCCGTGCAGGCCGA
GGTAGCCCGTTCCCTGGGGCGCTGGGAACCGCGCATCGGATTGTCTGCCGTGCGAGT
CGTCGCGGTCGTCGATGGCCGCGTGGATCTGCTCCTGAGCGGCGTGTTCGAGGGCGA
GAACATCAATATGGAGGTCTCGGCGTGATCATCGATCTTTCCCAGTTGCCGGAGCCAG
AGGTTATCGAAAACCTCGATTTCGAGACGATTTACCAAGAGCTGTTGGGCGACTTCC
GCGAAGCCATGGCTGGCGAATGGACAGCGGAGGTGGAGTCCGATCCGGTTCTCAAG
CTTCTGCAACTGGCGGCCTATCGAGAACTGCTGCTGCGGGCGCGGATCAACGATGCG
GCGCGGGCGGTGATGCTGGCATACGCCAGCGGTGCCGATCTCGACCAGATCGGTGCC
GGCTTCAATGTGCAGCGTTTGCTGATCAGGCCCGCTCAGCCCGAGGCGGTACCGCCG
GTGGAGGCGCAATACGAGAGCGACAAGTCGCTGCGCAATCGCATCCAGCTCGCGTTC
GAGCAGCTGTCCGTCGCAGGACCGCGGAACGCCTATATAGCCCATGCGCTGGGCGCG
GATGGAAGGGTGGCGGATGCCTCTGCGACCAGTCCGGCGCCCTGCGAAGTGCTGATC
AGCGTGCTCGGGGTGGAAGGCAACGGGCAGGCACCGGAAGCGGTGTTGCAGGCAGT
GCGCCTGGCGCTGAACGCGGAGGACGTGCGTCCTGTCGCGGATCGGGTAACGGTGC
GCTCGGCAGGAATCGTTCCCTATCAGGTCAAGGCGCAGCTCTACCTGTTTCCCGGTCC
CGAGGCCGAGCTGATCCGTGCCGCCGCCGAGGCTTCGCTGCGCGACTACATTTCCGC
CCAGCGCCGCCTGGGCCGCGACATCCGGCGTTCGGCCCTGTTCGCCACCCTGCATGT
CGAAGGCGTGCAACGCGTCGAACTGCAGGAGCCTGCGGCCGACGTGGTCCTGGATG
AAACCCAGGCGGCCTATTGCACGGGGTACGCGATCACCTTGGGAGGCGTCGATGAGT
AGCCGACTGCTGCCGCCAAACAGGAGTTCTCTGGAACGCTCTCTGGGTGATGTATTG
CCTGCCGAACTGCCGGTGCCGCTTCGTGAGCTTCACGATCCGGCACGCTGTGAGGCG
GCCTTGTTGCCCTACCTGGCCTGGACGCGCTCGGTGGACCGCTGGGACCCGGACTGG
AGCGACGAGGCCAAGCGCAATGCGGTAGCGACGTCCTTCGTCCTGCACCAGCGCAA
AGGCACGCTGACTGCGTTGCGCCAAGTGGTCGAGCCGATCGGTGCGCTGAGCGAGG
TCACCGAATGGTGGCAGCGAAGCCCGACCGGCGTGCCGGGGACCTTCGAGATCACC
GTGGACGTCAGCGACCGTGGCATCGACGAAGGCACCGTACTGGAGCTGGAGCGCTT
GCTCGATGACGTCCGCCCGGTGAGCAGACACCTGACCCGGCTGGACCTGCGCATTAC
CCCGGTAATCCGGTCCCGTCACGGACTAGCCGTGACCGACGGCGACACCCTGGAAAT
CTTCCCCTGGAAACAGTGACATGACGACCAATACTCCGAAATACGGTGGCCTGCTCA
CCGACATAGGTGCCGCTGCGCTGGCTACGGCCAGCGCAGCAGGCAAGAAATGGCAG
CCGACTCATATGCTGATCGGCGATGCCGGCGGTGCGCCGGGCGACACGCCGGATCCA
TTGCCTTCTGCGGCGCAGAAGAGCCTGATCAACCAACGCCATCGGGCCCAGCTGAAT
CGGCTGTTCGTTTCCGACAAGAACGCCAATACCTTGGTTGCCGAGGTGGTGCTGCCA
GTTGAGGTAGGTGGCTTCTGGATCCGCGAGATCGGCCTGCAGGATGCCGACGGCAAG
TTCGTCGCGGTATCCAACTGCCCGCCCAGCTACAAGGCTGCAATGGAAAGTGGCAGT
GCGCGGACCCAGACCATTCGGGTGAACATCGCGCTCTCCGGCCTGGAGAATGTCCAG
CTGCTGATCGACAACGGCATCATCTACGCCACTCAGGACTGGGTGAAGGAAAAGGTC
GCTGCCGATTTCAAGGGCCGCAAGATCCTGGCTGGCAATGGCCTGCTCGGTGGGGGC
GATCTTTCTGCCGACCGCAGCATTGGCCTGGCGCCTTCCGGCGTGACGGCGGGCAGC
TATCGTTCGGTCACGGTGAACGCCAACGGGGTGGTCACCCAGGGCAGCAATCCGACC
ACCCTGGCCGGCTATGCGATCGGAGATGCCTATACCAAGGCCGATACCGACGGAAAA
CTGGCGCAGAAAGCGAACAAGGCCACCACCCTGGCCGGCTATGGCATCACCGATGCG
CTGCGAGTCGATGGCAACGCCGTGTCATCCAGCAGGCTGGCCGCACCGCGCAGCCTG
GCAGCCAGTGGCGATGCCTCCTGGTCGGTGACCTTCGACGGCAGTGCCAATGTTTCT
GCGCCGCTGAGTCTTTCCGCTACCGGTGTGGCGGCGGGCAGCTATCCGAAGGTGACC
GTGGATACGAAGGGAAGGGTGACTGCTGGAATGGCGCTGGCGGCGACGGACATTCC
CGGGCTGGATGCGTCGAAGCTGGTCAGCGGGGTGCTGGCCGAGCAGCGTTTGCCGG
TATTCGCGCGCGGGTTGGCTACTGCTGTCTCGAACAGTAGCGATCCGAACACCGCGA
CCGTGCCGTTGATGCTGACCAATCATGCGAACGGACCTGTTGCCGGACGATACTTCTA
CATCCAGTCGATGTTCTATCCGGATCAGAACGGCAATGCTTCGCAGATTGCAACGAGC
TACAACGCTACATCCGAGATGTATGTACGGGTGTCCTACGCGGCCAACCCTAGCATCC
GGGAGTGGTTGCCCTGGCAGCGCTGCGACATTGGAGGTTCCTTCACCAAAGAGGCC
GATGGTGAACTGCCTGGAGGCGTCAACCTGGATTCGATGGTGACCTCAGGGTGGTGG
AGCCAGAGTTTTACTGCCCAAGCTGCCAGTGGAGCCAACTACCCTATAGTTCGGGCC
GGCCTGCTTCATGTGTACGCCGCGAGTAGCAATTTCATCTATCAGACGTATCAAGCCTA
CGATGGTGAGAGTTTCTATTTCCGGTGCCGGCATTCAAATACCTGGTTTCCCTGGCGT
CGCATGTGGCATGGCGGAGACTTCAACCCCAGTGACTATCTGTTGAAGTCGGGGTTC
TATTGGAATGCGTTACCGGGAAAACCTGCCACTTTTCCACCATCCGCACATAACCATG
ACGTCGGACAGCTTACTTCGGGCATTCTCCCCCTGGCACGTGGCGGCGTCGGTTCGA
ATACGGCAGCGGGAGCACGTAGCACTATCGGAGCAGGGGTTCCTGCGACTGCTTCCC
TTGGGGCGAGCGGATGGTGGCGGGACAATGACACTGGCCTCATTAGGCAATGGGGGC
AGGTCACTTGCCCCGCCGATGCCGATGCTTCGATTACGTTCCCGATTCCTTTCCCTACG
CTATGCCTCGGCGGATATGCGAATCAGACGAGTGCTTTCCATCCGGGAACGGATGCCA
GTACAGGTTTCCGTGGAGCGACTACCACTACCGCGGTGATTCGCAATGGCTACTTTGC
TCAGGCGGTTCTTTCATGGGAGGCATTTGGACGATGAAGGGCGAATATTATTTCTCTC
CAAGCCAGGTGGCATTCTATCCGGCCTCCTTGCGAGAGGTTTATGAATACGCAGGCTG
CTGGCCAGTCGATGGCGAGTGGGTCAGCGCAGAGCTACATGAACAACTGATGAACG
AACAGGCGGCAGGCCGAGCAATCAGTTCCGACGTGAATGGGAACCCAGTAGCGATC
GAGCGCCCTCCGCTTTCCCGTCAGCAACGTAGCACCCATGAGCGGAGATGGCGGGAT
AGTCAGCTGTTGGCGACCGACGGCCTAGTTGTTCGCCATCGAGATCAATTGGAAACC
GGAAAGGAAACGACCTTACTCCCTGTCCAATACCATGAACTCATGTCGTACAGAGCC
AGCTTACGGGATTGGCCGGAAGAGCCTTTATTTCCCGACAGTGGCGGACGCCCGTCC
GTACCAGATTGGCTCAGACGTTATGTCACCCCCTGAACCCCGCCTCGTGCGGGGTTTT
TCATTAATGGAGATCTACCTATGAGTTTCTTCCACGGCGTTACGGTAACCAACGTCGAT
ATCGGTGCGCGCACCATCGCGCTGCCGGCCAGCTCCGTCATCGGCCTCTGCGATGTGT
TCACGCCGGGGGCGCAGGCAAGCGCCAAGCCCAACGTTCCGGTGCTGCTCACCAGC
AAGAAGGACGCCGCCGCGGCGTTCGGCATCGGCTCGTCTATCTACCTGGCCTGCGAG
GCCATCTATAACCGTGCCCAGGCCGTGATCGTGGCGGTGGGCGTGGAAACCGCGGAG
ACTCCCGAGGCCCAGGCCAGCGCCGTCATAGGTGGTATCAGTGCCGCCGGCGAGCGC
ACCGGGCTGCAGGCTTTGCTCGACGGCAAGAGTCGCTTCAATGCCCAGCCGCGTCTA
CTAGTTGCGCCGGGTCATTCGGCCCAGCAAGCGGTGGCCACCGCCATGGACGGGCTG
GCCGAGAAACTGCGGGCCATCGCCATTCTCGATGGTCCCAATAGCACCGACGAGGCG
GCCGTCGCCTACGCCAAGAACTTCGGCAGCAAGCGCCTGTTCATGGTCGACCCGGGC
GTGCAGGTTTGGGACAGCGCCACCAATGCCGCGCGCAACGCCCCGGCTTCGGCCTAC
GCCGCCGGCCTGTTCGCCTGGACCGACGCCGAGTACGGCTTCTGGTCCTCGCCGTCG
AACAAGGAGATCAAGGGCGTCACCGGCACCAGCCGTCCGGTGGAGTTCCTCGACGG
CGATGAGACCTGTCGCGCCAACCTGCTCAACAACGCCAATATCGCGACGATCATTCGC
GACGATGGCTATCGCCTGTGGGGCAACCGCACCTTGTCCAGCGACAGCAAGTGGGCT
TTCGTCACCCGTGTTCGGACCATGGACCTGGTAATGGATGCGATCCTCGCCGGGCACA
AGTGGGCGGTGGACCGCGGCATCACCAAGACCTACGTGAAGGATGTCACCGAGGGC
CTGCGCGCCTTCATGCGCGATCTGAAGAACCAGGGAGCGGTGATCAACTTCGAGGTC
TATGCCGACCCGGACCTGAACAGCGCCAGCCAGCTGGCCCAGGGCAAGGTGTACTG
GAACATCCGCTTCACCGATGTGCCGCCTGCCGAGAACCCCAATTTCCGTGTCGAGGT
GACCGATCAGTGGCTCACCGAAGTTCTGGATGTCGCCTAAGGAGCGCCCGTGATGAT
TCCGCAAACCCTGACCAATACCAACCTGTTCATCGACGGCGTGAGCTTCGCCGGTGA
CGTGCCATCCCTGACCCTGCCCAAGCTGGCTGTGAAGACCGAGCAATACCGTGCCGG
CGGCATGGATGCGCCGGTATCCATCGACATGGGCCTGGAGGCGATGGAGGCCAAGTT
CTCCACCAACGGTGCCCGCCGAGAAGCGCTGAATTTCTTCGGCCTGGCCGACCAGAG
CGCTTTCAATGGCGTGTTCCGTGGCTCCTTCAAGGGCCAGAAGGGCGCCAGCGTGCC
AGTGGTGGCTACCCTGCGCGGCCTGCTCAAGGAGGTCGACCCGGGCGACTGGAAAG
CCGGCGAGAAAGCCGAGTTCAAGTACGCCGTTGCGGTCAGCTACTACAAGCTGGAA
GTCGATGGCCGCGAGGTCTACGAGATCGATCCGGTCAACGGTGTCCGCGCGATCAAC
GGTGTCGACCAGTTGGCCGGCATGCGCAACGACCTCGGCCTGTAAGAGGAGCTCCG
GACATGACCCAAGAGAATCGACTGCCGGGCTGGCTGACGCTGGATGCCGACGCCGC
CCTCGTTCGTCTCTCGCGTCCGGCACAGTGCAACGGGGTCAGCGTCGACACGCTGAC
CCTGCGTGCACCCACCGTGCGTGATATCCGCCTGGCCGGCAAGGTGGCCGGCGACGA
TGCCGAGGAGCGCGAACTGCAACTGTTCGCCTCGCTGGCGCAGGTCAGCCGCCAGG
ACCTGGAGGGGCTGAAGCTGAGCGACTACCAGCGGCTGCAAGGCGCCTACTTTCGC
CTGGTGCAAGACGACACGGACGACACCTTCGCGTATGCGTCAACTGGCGAGGCGCCT
GGCCATTGAGCTGGGCTTCCAGGCCTGCGAACTGGAGCGCATGACCCTGGGCGACCT
GCTCTGGTGGCTCGCCGAGGGCGAGGAGTGAGCGACGGCGCCGTCCGCCAGGCGAG
CGGCGCCCGCGCTACTTCAGTAGGTCCGCGGAGGCGGACATCGATAACAGACGAGAC
GAACCGTCATGAGTAAAGACATGGACCTGGTGGTCTCCATCGGCGGCATCGCCGACC
CCTCCTTGGGCAAGGCATTCGAGACCGTCAAGGCACGGCTCGACAGTCTCCAGGAA
CGTGCCCGCCAGGCTTCCAGCCTGAGGGATGTGCTGGGAGACGCCATACGCCTGGAG
CGAGAGCTCGCCGATATGCGCAAGGTCGGGGATCGCGGCGTTGCTGAGCATGCCCGG
CAGCTTGGCGAACGCCAGGAGCAACTGAAGCGACTCGGCATCGAGGCCAGGGCCGC
GGGCGATGCCTACGCTCGACTGGGCGAGATGCAGCGTGGCCTGGATATGCAGGTCCG
CGGCCTGCAACGGCTGGAGCAGGCCAGCCAGGCAATGCCATTGGCTAGCGCATTTTC
CGGACTGGTCGTGGAAGCCAGCAAGACGGCTGCCGGTTATCAAGCGCGGTTGCGCG
ACCTGTCGATCCGCAACGGCCTGGACGTCGGCCGGGAGCCAGCCTTGGCATCCCTGA
TCCAGGACAGCGCCAACCAGAGCGGCCTGGGACGCACGGTGACGCTGGACATGCTG
GAGCACTTGAACGCCACCGGCATGGGGTTCGCCGCCGCGCAAATGAATCTGGGACTG
GCGGGCCGCTTCGGCTTTGGCCAAGGGATTGCTTCAGCCGAGGTTGCGGGGCTGGTT
CGAGCGTTGCAACTGGCCCAGGGTTCGGACTCGCCAGAGCAATTGTCCGCCACCCTC
GACCGCCTGGTCGTCCTGGGTAAAGGCAGAGTCGGCAGTGAGGCCCTGGCGCGTCG
CCTGCCCGCCTTGTTGTCAGCGCTGGGCAATGCGGGAGAGGCCACGGCCGGCGATGT
CGGTGCATTGGGTGCCTTGCTGGAGATTCAGGCAAAGAACACCACGCCAGACAAAG
CGGACGTGCGGATGAAGGCCTGGCTGGAGTTCGTCGGCAGCGGCAGCTTGAAACGC
GCTTATGGCCAGGACTACGACCGGGACCTGGAAGCGCTGCGCAAGGACGGAGCGAG
TCTGCTGGAGGCCAACCTGGAGCTGGCTGCGCGCTATCGGGATAAGGGCGGCAAGCT
CAGCGCCGGCGTGGCGTCGCCGGCGCTTGAAGCCTATCGAGCGTCGCGCGGCGAGTT
CCAGGGCTTGCTCGAATCCCAGCAGTCTTCCGTAGGCTCTTCGGAGCGCGATGCGCA
GCGTCGCAAGGGGATGTCCCAGGAGCTGTGGAAGGCTTCCAGCGACAGTTGGGAGA
GGGCGCAGACCGCTTTGGGCAGCGCCCTGAATCCATATCTGGACAACCTGGCCAAGG
GGAGCGCGGTACTCGGCGAGTCGACTGCGGAGCTGCTCGAAGCCTATCCGCGGACG
ACGGCCGGTCTTACCGCCGCCGCAGGTGCGGTGTTATCCGGGTATCTCGCCTACAAGG
GAGGGCGCGGCGCTATCGACGTGCTGCGTGGCGGTCGGCTCGGTCGGCGAGGGACC
GCTGCCGTCGGCGACCTGATCGAACGGGGTGCAGGCCGGGTATCGGGTGGTAGCGA
AATACAGCGCGTGTTCGTTACCAACTGGCCGGTGCCGGGAGGCGACTCTACGCTGGA
GTCCGCGAGGAGGCCAGCACAACGAAAGCGCGGTCAAACACCACGCAGGAAGAGA
GGCAAGGGCGGTGGGCTGAAGGCCCGCTCCCTTCCATCCCTTGGCTTCTCGGCTGGA
GGAGGCTTGGGGGCGATGGCAGGAAAGCTGCCGCGCCTGTCGCGTTTGCCCATACGC
AACGCGCCGCTGCAAGTGGCCTCGTCGTTGATCGATGTTGCTGAGGTCTACTCCAGC
GACCTGTCGGAGAGCGAAAAGACCGTTGCCTATGGCGAGGCAGGTGGCTCCCTGGC
TGGTTCCCTGGCTGGCGCCGCTCTGGGAGCGAGCATCGGCTCGGTGGTGCCGGTGGT
CGGTACGCTGATCGGTGGATTGGTTGGCGGCGCTATCGGCGCCTGGGGCGGTAGCGA
ACTGGGGGGGGCCTGGGGCGCAGTCTGGCTGGCGATCCGCCGGCGGCCTCGGACA
ACAAGCCGGCGGTGGCCGTACCACAGGCCGGACCCGTCGCGGCTGCGCCCAACTGG
ACCTTCGCGCCGCAGATCAACCTGACGGTGCAAGGCAACGTGCACGAGCCGCAGCG
CCTGGCCGACGAGTTGCTGCCCTACCTGCAACGCATGCTTGTCGACTTCGCCGACGA
GCGGCAGCGGCGCAGCCTCTACGACCCGGCGATGGTTTAAGGAGTCCCCATGGCATA
TCTGGAACAATTGCAGGCCGGCCTGAGGTACCTGGGTCGCGCCGGCGAGTCCGGAC
GCAAGAGTCTGGACAAGGTGGTCGCTCCGGTGAACGGCGCGATCAGCGAGATCCGC
GGCGCAGCCGCGGAGCTGGAGAACCTGCCCGGCGTATCGCCGGAAATGGCTGCCCG
GCTGCAGCGTGCCATGCGCGGCATCGGCCAGGCGCAGGGCAAGGTGAACCGCGTGG
TCTCCACCTATGACCGGGCGAGCCGGGCGTTGCTTGGTATCGACGAACGCCTGGATG
CGCTGAAGGTGCAGGTGAACAGTGCCGCGCAGGCGGTCGGCAAGGTCGCTGGCGAC
ATCAGTCCGACGCTGGCGGGGGTGCTGCCGTCGTGGCTGCTGGCACCCTCGGCGACG
CCCCCGAGCGAGGCCGCAGCGTCCTTGCCGCACCTGCTGGTACTGCAGCCGCTGACC
GCCAATGCCCAACCGTTCTACTTCAACCTGAATACTGCCGCCTTCGACGCCCTGCAGC
GCAACAGTGCCTACAACTGGAGCGGGCAGGTGCGCCTGGGTCGGCGGCCGGCGCTG
CAGAGCGTCGGCATGGGCGAGGAGAGCATCCTGCTCAAGGGTGCGGTATTCCCGCTG
CGTCGACAGGTAGGTAACCAGGAAAAGGTCGTCGGTCTGGAGCAGCTCGAAGCGCT
GCGCCGGTTGGCGGAGCGGCGTGAGCCGCTGATCCTGAGCAGCGGCTACGGCGAGG
TGCAGATGGGCCTCTGGTGTCTGGTGCGGATCAGCGAGAACCAGAGCGCCCTACTGG
GCAACGGCGCTCCCCGCAAACAAACCTTCGACCTGGAGTTCAAGCGCTATGGCGACG
ACCTGCCGAACCGCTGACGGCGACATGCTGGACAGCCTCTGCTACCACGTCTATGGC
CATCTGTTGGGCTGCGTCGAGGCGACCCTCGACGCCAATCCCGGGCTGGCCGATGAG
CAGCAGCCATTCCGCGCCGGCTTGCTGATCAGTTTCCCTGACATGCCGGTGGTCAATG
TCGAACAGGTGCGCCTGTGGGATTGATCGACCGCTCACCCGCAACCCCGCCTTGGCG
GGGTTTTTCTTTTCTGGAGAAACCAGGTGCAACCGAGTTTCCGTATCGTTGCCGACGG
CACCGACGTCACCCAGCGGCTGAATGACCGCCTGCTCAAGCTGACCCTGCTGGACAA
GCCGGGCATGGAGTCCGACAGCCTGACCTTGAGGATCGACGATCGCGATGGACAGGT
GGCCTTACCCAGGCGCGGTGCGGTGCTGGAGGTTCATCTCGGCTATGCCGGCGAGCC
ACTGATGCGCATGGGACGCTTTACCGTGGACACCTTGCAGTGGGCTGGTCCGCCGGA
CTGCCTGACCGTCACTGCCAAGGCCGGCGACATGCGCGGCAGTGGCAAGACGATAC
GCAGCGGAGGTTGGGAGGGCACTACCCTGGCTCAGGTCTGCCGCGATGTTGGCGCAC
GCAACGGCTGGCGCGTGGAGTGTCCGTTGCAGGTGGCGATCGCCCGGGTCGACCAG
GTCAATGAGTCCGACTACCACTTCGTCACCCGTCTGGCGCGCCAGTACGACTGCACC
GCCAAGCTGGCCGAGGGCATGCTCATGGTGCTGCCGCGACAGAGCGGGCAGAGCGC
CACGGGGCGTCGGATCGAACCTTTGGTGTTGGGACGTGCTGACGTCGGTAGCTTCGA
CGTTACCTTCGACGACCGCAGCCTGATGAGAACGGTGAAGACCCGCTACCAATTGCC
CGGCAGCGGCGAGGTCAAGAGCGTCGAGTTGAAGAACCCGAAGGCACCGGCTACGG
CTATGGGCGAGCATGTCGACCGGCACCTCTATACCAGCCGTGGAGAGGCCGAGCAGG
CGGCGAAGGCTCGCCTGGCGAGCTTCAGCCGCTCCAGCGCCAGTGTGCGCCTGGAA
CTGCCAGGGCGTGGCGACCTGTTCGCCGAGCGCAGCCTGCTGCTCCAAGGCTTCAAG
GCGGGAATCGACGGCGAGTTCCTGATCGACTCGGTGGAGCACACCTACAGCTCCAGC
GGATGGACCACTGTCGTGCAATGCAACGGCGGCCGAGGCGGCAAGGGGTGACGCCT
GAAACCCATCGGAGTGCAGGAGGATCGATGAAACTGACCGAGCAGCAATTGCTGCG
CATTTTTCCCAACGCCCGCCTCGTCGCGGGCGTTTTCGTTGTGGCGTTGCAACGGGCC
ATGGACGAGAGGGAGATCGACACACCGGCGCGGCGTGCCGCGTTTCTCGCCCAGGT
CGGCCACGAAAGCAGCCAGTTGACCCGGCTGGTGGAGAACCTCAATTACAGCGCCC
AAGGCTTGGCGGCGACCTGGCCGGGTCGCTATCTCGGCCCCGACGGGCAGCCCAAC
GCGTTGGCCTTGCGGCTGGCGCGCAATCCGCAGGCGATTGCCGACAACACCTACGCC
ACGCGCAACGGCAATGGCGACGAAGCGTCCGGCGATGGCTGGCGCTTCCGCGGGCG
TGGCTTGCTACAAATCACCGGGCGTGCCAACTACCGGTTGGTCGGCGAGGCCCTCGG
CGAGCCGCTGGAAGCCGAGCCCTGGCGCCTGGAGCAGCCCGTGCCGGCGGCCCGCA
GCGCCGCCTGGTGGTGGGCCGGTCACGGGCTCAACGAGCTGGCCGACCGCGGCGAG
TTCGCTGCCATCACCCGCCGCATTAACGGCGGCCTGAATGGCCAGGCGGAGCGCCTG
GCGTTGTGGCAGCGGGCCAGGGCGGTGCTGTCATGAGCCGGCTCGCTCTGCTCCTGC
CGGCCGTGTTGTTGGTCCTGCTGGCCGGCGCCTTGCTCGGCGGCGGCCTGGTTGCCC
GCCATTATCGTCCGCAACTGGAGGAGGCCCTGGGACAACTCACTGCCAGCCGCGTCG
CCAGCGGCCAGCTCGAGGCTTTGCTCGATGAGCAGCAGCGCGCGCTGGCGGCGGTG
CGGGCGAGCGCCGAGAGGCGCGCGAAGGACGTCGAGCAGGCACTCGGCGAGGCCA
GGGCGCAGGCCGCGGAGCAGTATGCCGCGGCCGTGCGTCTGCTCCAAGAACCCGAC
TTTGGCACGGACTGCCAGGCGGCAGGTGCGGCGATCGACCGGGAGCTGGGACTATG
ACTCGTCTCCTGCTGGGGCTTTGTCTGCTTTTCGCGGGCTGCGCAGCCTCACCGACG
ACACCTCGCCCAGTGCGCGTCGAGGTTCCCCTGGCAGTGCCCTGCCGTGTACCTGAC
GTGCGCCCGCCGAGCTGGGCCGGCGCCACGCTGAAGGCCGGCGATTCGCTGCAGGC
CAAGGTTCGCGCATTGCTCGCCGAGCGCCGCCAGCGGCAGGGCTACGAACTCGAATT
GCAGGCGGCATTGCGTGCCTGCCGCTGAGACACTGGGCGCATATGGACGTCGATGGA
CGTGTGTCCGGCGCATATTGCCGTGTATTGATTCCGCAGCGTTGTCGAGCCGGGAATC
GGTCGGTACAACGTAGTCATGCTTGTACAGGTGTGTCCCCCCAGGGATGTCACCTGCA
ACCTCAGAGCCCGGCCAGTGTGCCGGGCTTTTTCGTTTGCATCCGACAACGGCTCGG
GACGTGGAGGCTCCTCGCCGACCGCGTACCGCGCCACGGCTGACCGTCCTGGAGGC
GGGGCCAGACGACCCGCCTGGTGCGGGTCTTTTCATGTGCATAAAGGAGAGTTTTCC
A
PAO1_R2_Pyocin_tail
SEQ ID NO: 5
ATGACGACCAATACTCCGAAATACGGTGGCCTGCTCACCGACATAGGTGCCGCTGCG
CTGGCTACGGCCAGCGCAGCAGGCAAGAAATGGCAGCCGACTCATATGCTGATCGGC
GATGCCGGCGGTGCGCCGGGCGACACGCCGGATCCATTGCCTTCTGCGGCGCAGAAG
AGCCTGATCAACCAACGCCATCGGGCCCAGCTGAATCGGCTGTTCGTTTCCGACAAG
AACGCCAATACCTTGGTTGCCGAGGTGGTGCTGCCAGTTGAGGTAGGTGGCTTCTGG
ATCCGCGAGATCGGCCTGCAGGATGCCGACGGCAAGTTCGTCGCGGTATCCAACTGC
CCGCCCAGCTACAAGGCTGCAATGGAAAGTGGCAGTGCGCGGACCCAGACCATTCG
GGTGAACATCGCGCTCTCCGGCCTGGAGAATGTCCAGCTGCTGATCGACAACGGCAT
CATCTACGCCACTCAGGACTGGGTGAAGGAAAAGGTCGCTGCCGATTTCAAGGGCCG
CAAGATCCTGGCTGGCAATGGCCTGCTCGGTGGGGGCGATCTTTCTGCCGACCGCAG
CATTGGCCTGGCGCCTTCCGGCGTGACGGCGGGCAGCTATCGTTCGGTCACGGTGAA
CGCCAACGGGGTGGTCACCCAGGGCAGCAATCCGACCACCCTGGCCGGCTATGCGAT
CGGAGATGCCTATACCAAGGCCGATACCGACGGAAAACTGGCGCAGAAAGCGAACA
AGGCCACCACCCTGGCCGGCTATGGCATCACCGATGCGCTGCGAGTCGATGGCAACG
CCGTGTCATCCAGCAGGCTGGCCGCACCGCGCAGCCTGGCAGCCAGTGGCGATGCCT
CCTGGTCGGTGACCTTCGACGGCAGTGCCAATGTTTCTGCGCCGCTGAGTCTTTCCGC
TACCGGTGTGGCGGCGGGCAGCTATCCGAAGGTGACCGTGGATACGAAGGGAAGGG
TGACTGCTGGAATGGCGCTGGCGGCGACGGACATTCCCGGGCTGGATGCGTCGAAGC
TGGTCAGCGGGGTGCTGGCCGAGCAGCGTTTGCCGGTATTCGCGCGCGGGTTGGCTA
CTGCTGTCTCGAACAGTAGCGATCCGAACACCGCGACCGTGCCGTTGATGCTGACCA
ATCATGCGAACGGACCTGTTGCCGGACGATACTTCTACATCCAGTCGATGTTCTATCCG
GATCAGAACGGCAATGCTTCGCAGATTGCAACGAGCTACAACGCTACATCCGAGATG
TATGTACGGGTGTCCTACGCGGCCAACCCTAGCATCCGGGAGTGGTTGCCCTGGCAG
CGCTGCGACATTGGAGGTTCCTTCACCAAAGAGGCCGATGGTGAACTGCCTGGAGGC
GTCAACCTGGATTCGATGGTGACCTCAGGGTGGTGGAGCCAGAGTTTTACTGCCCAA
GCTGCCAGTGGAGCCAACTACCCTATAGTTCGGGCCGGCCTGCTTCATGTGTACGCCG
CGAGTAGCAATTTCATCTATCAGACGTATCAAGCCTACGATGGTGAGAGTTTCTATTTC
CGGTGCCGGCATTCAAATACCTGGTTTCCCTGGCGTCGCATGTGGCATGGCGGAGACT
TCAACCCCAGTGACTATCTGTTGAAGTCGGGGTTCTATTGGAATGCGTTACCGGGAAA
ACCTGCCACTTTTCCACCATCCGCACATAACCATGACGTCGGACAGCTTACTTCGGGC
ATTCTCCCCCTGGCACGTGGCGGCGTCGGTTCGAATACGGCAGCGGGAGCACGTAGC
ACTATCGGAGCAGGGGTTCCTGCGACTGCTTCCCTTGGGGCGAGCGGATGGTGGCGG
GACAATGACACTGGCCTCATTAGGCAATGGGGGCAGGTCACTTGCCCCGCCGATGCC
GATGCTTCGATTACGTTCCCGATTCCTTTCCCTACGCTATGCCTCGGCGGATATGCGAA
TCAGACGAGTGCTTTCCATCCGGGAACGGATGCCAGTACAGGTTTCCGTGGAGCGAC
TACCACTACCGCGGTGATTCGCAATGGCTACTTTGCTCAGGCGGTTCTTTCATGGGAG
GCATTTGGACGATGA
PAO1_R2_pyocin_tail_fiber
SEQ ID NO: 6
MTTNTPKYGGLLTDIGAAALATASAAGKKWQPTHMLIGDAGGAPGDTPDPLPSAAQKS
LINQRHRAQLNRLFVSDKNANTLVAEVVLPVEVGGFWIREIGLQDADGKFVAVSNCPPS
YKAAMESGSARTQTIRVNIALSGLENVQLLIDNGIIYATQDWVKEKVAADFKGRKILAGN
GLLGGGDLSADRSIGLAPSGVTAGSYRSVTVNANGVVTQGSNPTTLAGYAIGDAYTKAD
TDGKLAQKANKATTLAGYGITDALRVDGNAVSSSRLAAPRSLAASGDASWSVTFDGSA
NVSAPLSLSATGVAAGSYPKVTVDTKGRVTAGMALAATDIPGLDASKLVSGVLAEQRLP
VFARGLATAVSNSSDPNTATVPLMLTNHANGPVAGRYFYIQSMFYPDQNGNASQIATSYN
ATSEMYVRVSYAANPSIREWLPWQRCDIGGSFTKEADGELPGGVNLDSMVTSGWWSQS
FTAQAASGANYPIVRAGLLHVYAASSNFIYQTYQAYDGESFYFRCRHSNTWFPWRRMW
HGGDFNPSDYLLKSGFYWNALPGKPATFPPSAHNHDVGQLTSGILPLARGGVGSNTAAG
ARSTIGAGVPATASLGASGWWRDNDTGLIRQWGQVTCPADADASITFPIPFPTLCLGGYA
NQTSAFHPGTDASTGFRGATTTTAVIRNGYFAQAVLSWEAFGR
R3_tail
SEQ ID NO: 7
ATGACGACTAATACTCCGAAATACGGTGGCCTGCTCACCGACATAGGTGCCGCTGCGC
TGGCTGCGGCCAGCGCAGCAGGCAAGAAATGGCAGCCGACTCATATGCTGATCGGCG
ATGCCGGCGGTGCGCCGGGCGACACGCTGGATCCATTGCCTTCTGCGGCGCAGAAGA
GCCTGATCAACCAACGCCATCGGGCCCAGCTGAATCGGCTGTTCGTTTCCGACAAGA
ACGCCAATACCTTGGTTGCCGAGGTGGTGCTGCCAGTTGAGGTAGGTGGCTTCTGGA
TCCGCGAGATCGGCCTGCAGGATGCCGACGGCAAGTTCGTCGCGGTATCCAACTGCC
CGCCCAGCTACAAGGCTGCGATGGAAAGTGGCAGTGCGCGGACCCAGACCATTCGG
GTGAACATCGCGCTCTCCGGCCTGGAGAATGTCCAGCTGCTGATCGACAACGGCATC
ATTTACGCCACTCAGGACTGGGTGAAGGAAAAGGTCGCTGCCGATTTCAAGGGCCGC
AAGATCCTGGCTGGCAATGGCCTGCTCGGTGGGGGGGATCTTTCTGCCGACCGCAGC
ATTGGCCTGGCGCCTTCCGGCGTGACGGCGGGCAGCTATCGTTCGGTCACGGTGAAC
GCCAACGGGGTGGTCACCCAGGGCAGCAATCCGACCACCCTGGCCGGCTATGCGATC
GGAGATGCCTATACCAAGGCCGATACCGACGGAAAACTGGCGCAGAAAGCGAACAA
GGCCACCACCCTGGCCGGCTATGGCATCACCGATGCGCTGCGAGTCGATGGCAACGC
CGTGTCATCCAGCAGGCTGGCCGCACCGCGCAGCCTGGCAGCCAGTGGCGATGCCTC
CTGGTCGGTGACCTTCGACGGCAGTGCCAATGTTTCTGCGCCGCTGAGTCTTTCCGCT
ACCGGTGTGGCGGCGGGCAGCTATCCGAAGGTGACCGTGGATACGAAGGGAAGGGT
GRCTGCTGGAATGGCGCTGGCGGCGACGGACATTCCCGGGCTGGATGCGTCGAAGCT
GGTCAGCGGGGTGCTGGCCGAGCAGCGTTTGCCGGTATTCGCGCGCGGGTTGGCTAC
TGCTGTCTCGAACAGTAGCGATCCGAACACCGCGACCGTGCCGTTGATGCTGACCAA
TCATGCGAACGGACCTGTTGCCGGACGATACTTCTACATCCAGTCGATGTTCTATCCG
GATCAGAACGGCAATGCTTCGCAGATTGCAACGAGCTACAACGCTACATCCGAGATG
TATGTACGGGTGTCCTACGCTGCCAACCCTAGCATCCGGGAGTGGTTGCCCTGGCAGC
GCTGCGACATTGGCGGTTCCTTCACCAAAGAGGCCGATGGTGAACTGCCTGGAGGCG
TCAACCTGGATTCGATGGTGACCTCGGGGTGGTGGAGCCAAAGTTTTACTGCCCAAG
CTGCCAGTGGAGCCAACTACCCTATAGCTCGGGCCGGCTTGCTTCATGTGTACGCCGC
GAGTAGCAATTTCATCTATCAGACGTATCAAGCCTACGACGGTGAGAGTTTCTATTTCC
GGTGCCGGTATTCAAATACCTGGCTTCCCTGGCGTCGTATGTGGCATGGCGGAGACTT
CAACCCCAGTGACTATCTGTTGAAGTCGGGGTTCTATTGGAATGCGCTACCGGGAAA
ACCTGCCACTTTTCCACCATCCGCACATAACCATGACGTCGGACAGCTTACTTCGGGC
ATCCTCCCCCTGGCACGTGGCGGCGTCGGTTCGAATACGGCAGCGGGAGCACGTAGC
ACTATCGGAGCAGGGGTTCCTGCGACTGCTTCCCTTGGGGCGAGCGGATGGTGGCGG
GACAATGACACTGGCCTCATTAGGCAATGGGGACAGGTCACTTGCCCCGCCGATGCC
GATGCTTCGATTACGTTCCCGATTCCTTTCCCTACGCTATGCCTCGGCGGATATGCGAA
TCAGACGAGTGCTTTCCAGCCGGGAACGGATGCCAGTACAGGTTTCCGTGGAGCGAC
TACCACTACCGCGGTGATTCGCAATGGCTACTTTGCTCAGGCGGTTCTCTCATGGGAG
GCATTTGGACGATGAAGGGTGAATATTATTTCTCGCCAAGCCAGGTGGCATTCTATCC
GGCCTCCTTGCGAGAGGTTTATGAACACGCAGGCTGCTGGCCAGTCGATGGGGAGTG
GGTCAGCGCAGAGCTACATGAACAACTGATGAACGAACAGGCGGCAGGCCGAGCAA
TCAGTTCCGACGTGAATGGGAACCCAGTAGCGATCGAGCGCCCTCCGCTTTCCCGTC
AGCAACGTAGCACCCATGAGCGGAGATGGCGGGATAGTCAGCTGTTGGCGACCGAC
GGCCTAGTTGTTCGCCATCGAGATCAATTGGAAACCGGAAAGGAAACGACCTTACTC
CCTGTCCAATACCATGAACTCATGTCGTACAGAGCCAGCTTACGGGATTGGCCGGAAG
AGCCTTTATTTCCCGACAGTGGCGGACGTCCGTCCGTACCAGATTGGCTCAGACGTTA
TGTCACCCCCTGA
R3_pyocin_tail_fiber
SEQ ID NO: 8
MTTNTPKYGGLLTDIGAAALAAASAAGKKWQPTHMLIGDAGGAPGDTLDPLPSAAQKS
LINQRHRAQLNRLFVSDKNANTLVAEVVLPVEVGGFWIREIGLQDADGKFVAVSNCPPS
YKAAMESGSARTQTIRVNIALSGLENVQLLIDNGIIYATQDWVKEKVAADFKGRKILAGN
GLLGGGDLSADRSIGLAPSGVTAGSYRSVTVNANGVVTQGSNPTTLAGYAIGDAYTKAD
TDGKLAQKANKATTLAGYGITDALRVDGNAVSSSRLAAPRSLAASGDASWSVTFDGSA
NVSAPLSLSATGVAAGSYPKVTVDTKGRVXAGMALAATDIPGLDASKLVSGVLAEQRLP
VFARGLATAVSNSSDPNTATVPLMLTNHANGPVAGRYFYIQSMFYPDQNGNASQIATSYN
ATSEMYVRVSYAANPSIREWLPWQRCDIGGSFTKEADGELPGGVNLDSMVTSGWWSQS
FTAQAASGANYPIARAGLLHVYAASSNFIYQTYQAYDGESFYFRCRYSNTWLPWRRMW
HGGDFNPSDYLLKSGFYWNALPGKPATFPPSAHNHDVGQLTSGILPLARGGVGSNTAAG
ARSTIGAGVPATASLGASGWWRDNDTGLIRQWGQVTCPADADASITFPIPFPTLCLGGYA
NQTSAFQPGTDASTGFRGATTTTAVIRNGYFAQAVLSWEAFGR
R4_tail
SEQ ID NO: 9
ATGACGACCAATACTCCGAAATACGGTGGCCTGCTCACCGACATAGGTGCCGCTGCG
CTGGCTGCGGCCAGTGCAGCAGGCAAGAAATGGCAGCCGACTCATATGCTGATCGGC
GATGCCGGCGGTGCGCCGGGCGACACGCCGGATCCATTGCCTTCTGCGGCGCAGAAG
AGCCTGATCAACCAACGCCATCGGGCTCAGCTGAATCGGCTGTTCGTTTCCGACAAG
AACGCCAATACCTTGGTTGCCGAGGTGGTGCTGCCAGTTGAGGTAGGTGGCTTCTGG
ATCCGCGAGATCGGCCTGCAGGATGCCGACGGCAAGTTCGTCGCGGTATCCAACTGC
CCGCCCAGCTACAAGGCTGCAATGGAAAGTGGCAGTGCGCGGACCCAGACCATTCG
GGTGAACATCGCACTCTCCGGGCTGGAGAATGTCCAGCTGCTGATCGACAACGGCAT
TATCTACGCCACTCAGGACTGGGTGAAGGAAAAGGTCGCTGCCGATTTCAAGGGCCG
CAAGATCCTGGCTGGCAATGGCCTGGTCGGTGGGGGCGATCTTTCTGCCGACCGCAG
CATTGGCCTGGCGCCTTCCGGCGTGACGGCGGGCAGCTATCGCTCGGTCACGGTGAA
CGCCAACGGGGTGGTCACCCAGGGCAGCAATCCGACCACCCTGGCCGGCTATGCGAT
CGGAGATGCCTATACCAAGGCCGATACCGACGGAAAACTGGCGCAGAAAGCGAACA
AGGCCACCACCCTGGCCGGCTATGGCATCACCGATGCGCTGCGAGTCGATGGCAACG
CCGTGTCATCCAGCAGGCTGGCCGCACCGCGCAGCCTGGCAGCCAGTGGCGATGCCT
CCTGGTCGGTGACCTTCGACGGCAGTGCCAATGTTTCTGCGCCGCTGAGTCTTTCCGC
TACCGGTGTGGCGGCGGGCAGCTATCCGAAGGTGACCGTGGATACGAAGGGAAGGG
TGACTGCTGGAATGGCGCTGGCGGCGACGGACATTCCCGGGCTGGATGCGTCGAAGC
TGGTCAGCGGGGTGCTGGCCGAGCAGCGTTTGCCGGTATTCGCGCGCGGGTTGGCTA
CTGCTGTCTCGAACAGTAGCGATCCGAACACCGCGACCGTGCCGTTGATGCTGACCA
ATCATGCGAACGGACCTGTTGCCGGGCGATACTTCTACATCCAGTCGATGTTCTATCCG
GATCAGAACGGCAATGCTTCGCAGATTGCAACGAGCTACAACGCTACATCCGAGATG
TATGTACGGGTGTCCTATGCGGCCAACCCTAGCATCCGGGAGTGGTTGCCCTGGCAGC
GCTGCGACATTGGAGGTTCCTTCACCAAAGAGGCCGATGGTGAACTGCCTGGAGGCG
TCAACCTGGATTCGATGGTGACCTCAGGGTGGTGGAGCCAAAGTTTTACTGCCCAAG
CTGCCACTGGAGCCAACTACCCTATAGTTCGGGCCGGCCTGCTTCATGTGTACGCCGC
GAGTAGCAATTTCATCTATCAGACGTATCAAGCCTACGATGGTGAGAGTTTCTATTTCC
GGTGCCGGCATTCAAATACCTGGTTTCCCTGGCGTCGCATGTGGCATGGCGGAGACTT
CAACCCCAGTGACTATCTGTTGAAGTCGGGGTTCTATTGGAATGCGTTACCGGGAAAA
CCTGCCACTTTTCCACCATCCGCACATAACCATGACGTCGGACAGCTTACTTCGGGCA
TTCTCCCCCTGGCACGTGGCGGCGTCGGTTCGAATACGGCAGCGGGAGCACGTAGCA
CTATCGGAGCAGGGGTTCCTGCGACTGCTTCCCTTGGGGCGAGCGGATGGTGGCGGG
ACAATGACACTGGCCTCATTAGGCAATGGGGGCAGGTCACTTGCCCCGCCGATGCCG
ATGCTTCGATTACGTTCCCGATTCCTTTCCCTACGCTATGCCTCGGCGGATATGCGAAT
CAGACGAGTGCTTTCCATCCGGGAACGGATGCCAGTACAGGTTTCCGTGGAGCGACT
ACCACTACCGCGGTGATTCGCAATGGCTACTTTGCTCAGGCGGTTCTTTCATGGGAGG
CATTTGGACGATGAAGGGCGAATATTATTTCTCTCCAAGCCAGGTGGCATTCTATCCGR
CCTCCTTGCGAGAGGTTTATGAATACGCAGGCTGCTGGCCAGTCGATGGCGAGTGGG
TCAGCGCAGAGCTACATGAACAACTGATGAACGAACAGGCGGCAGGCCGAGCAATC
AGTTCCGACGTGAATGGGAACCCAGTAGCGATCGAGCGCCCTCCGCTTTCCCGTCAG
CAACGTAGCGCCCATGAGCGGAGATGGCGGGATAGTCAGCTGTTGGCGACCGACGGC
CTAGTTGTTCGCCATCGAGATCAATTGGAAACCGGAAAGGAAACGACCTTACTCCCT
GTCCAATACCATGAACTCATGTCGTACAGAGCCAGCTTACGGGATTGGCCGGAAGAG
CCTTTATTTCCCGACAGTGGCGGACGTCCGTCCGTACCAGATTGGCTCAGACGTTATG
TCACCCCCTGA
R4_pyocin_tail_fiber
SEQ ID NO: 10
MTTNTPKYGGLLTDIGAAALAAASAAGKKWQPTHMLIGDAGGAPGDTPDPLPSAAQKS
LINQRHRAQLNRLFVSDKNANTLVAEVVLPVEVGGFWIREIGLQDADGKFVAVSNCPPS
YKAAMESGSARTQTIRVNIALSGLENVQLLIDNGIIYATQDWVKEKVAADFKGRKILAGN
GLVGGGDLSADRSIGLAPSGVTAGSYRSVTVNANGVVTQGSNPTTLAGYAIGDAYTKAD
TDGKLAQKANKATTLAGYGITDALRVDGNAVSSSRLAAPRSLAASGDASWSVTFDGSA
NVSAPLSLSATGVAAGSYPKVTVDTKGRVTAGMALAATDIPGLDASKLVSGVLAEQRLP
VFARGLATAVSNSSDPNTATVPLMLTNHANGPVAGRYFYIQSMFYPDQNGNASQIATSYN
ATSEMYVRVSYAANPSIREWLPWQRCDIGGSFTKEADGELPGGVNLDSMVTSGWWSQS
FTAQAATGANYPIVRAGLLHVYAASSNFIYQTYQAYDGESFYFRCRHSNTWFPWRRMW
HGGDFNPSDYLLKSGFYWNALPGKPATFPPSAHNHDVGQLTSGILPLARGGVGSNTAAG
ARSTIGAGVPATASLGASGWWRDNDTGLIRQWGQVTCPADADASITFPIPFPTLCLGGYA
NQTSAFHPGTDASTGFRGATTTTAVIRNGYFAQAVLSWEAFGR
R5_pyocin
SEQ ID NO: 11
CACCGCACCAGGGACGGGGCGCGCCGGTGGCGTACGGTGGACCACCAGAAGACCCA
GCCGATCATGCTGATCTGGCGGCTGCGCATGTCCTCCGGCGAGTACTCCTCGTCCGGG
TATTCCTCGCGGTTGAAGCTGCGCAGCCGAATGCCGCCGCCGGGCAGGCGATAGACG
AACTTCACCCGCAGCATGCCGTCATGTTCGAGGGCGTAGATCTCGCCATCGGTGATAT
GGGTGGTGGCGGTGTCCACGCCGATGGTGGAGCCATCCATGATCAGCGGTTCCATGC
TGTTGCCGGTGAGTTGGGCGCAGATCGCCGCCGACGGATCGACGCCCGAGGCACGC
AGCGTGGCGTAGGAAAAACGCAGCTTGCGCCCCTCTATCTCGCGCACCGCAGTGCGT
CCGGCGCCGGCGGACATCTCCACTTCCTTGTACAGCGGCAGTTCCACTTCGTCTTCGT
CCAGCGGTGTGTCGCTGTCCCACGGATGCAGCGGTTCCAGCACCAATGGGCTGCCAT
CGGCCACGGACACGGCACTGCGCGCGGCTGGAGTGCCTTCGCCGGTCTGCAGCCAG
ACCGGCGAAACGCCCAGGGCCGCCGCGATCTCGATCAGCTTGCGGGTGCTCTGGGCC
TTGCCCGAGGTCAGCTTGTGAATGGTGTTCTGCGAAACCCCTGCCGCTTCGGCGAGG
GTTTCCTGCTTCAGGTTGCGCATCGCCATGGCCTGCTTGAGGCGAGCGGCGAAGCTG
TCGGGCGGGATCTGGGTGCTCTTGTCCATGCTCGACAATCTACAGACCGATGGATTTT
CTGTAAAGAGCCTAGGCGTTGACGATAAATAGCTTTGGTTGTAATTTCTCTTCCGTCA
GAAAGCGGAAGGGGTGAGTTCCTCAACTTCCCACCCGCCTTTCAGAGATTTCACATG
GGTGCAGGGACGCACCCGGTTGTCGGCGACCGACGATGCAGGTGCCGACTGCTTGG
GTGCCGGGGCCATGGGAAAGCGCTCCGGCACCTGGAGGGCCGGAGCGCCGTCCATG
CAGACGCTCCGTCCCGCTCATTCATTCTGTCGCCGACCTGCTTCAGGTACGGCGGCGT
CGTACAGGGAGTCATTGCCATGTCGCGAGATACGCACTCGCCATTTCCGCGCAGAGG
AACGCAGGTCGCCGAACCGGCGCCGCGCATCTGTCATGTTCCATCCATTCGGCCGTC
GGGCGTCGGGCACGCCAGGGAGGCTTTCCATGGCTGACCTTGCCGATCACGCCAACG
AACTGGTCCTGGCTCGCCTCGACGGCCTCCTGGCGGCGCGCCCGGCGCTGGCCATCC
GCGAGTCCGCGGAAGACTGCGAGGACTGCGGCGAGCCCATTCCCCAGGCGCGCCGC
CGGGCGGCACCGGGCTGCAGTCGCTGCATCGACTGCCAGGACCGCCACGAGCGCCG
TTGAACCGACCTCATGCCGAGCCCTCGCGGGCAGTGAAAGGAGACACGACCGTGAT
CAAAGCCATCGATGAAATGCTCAAGCTCTGGGCCGAGGAAATGCACGCGCCAGGCA
GCAACGGAGGCGGCTACGCCGGCGGCAACCTGATCGCCATGCTGATCGCCAGCAAG
GGCGAGGTGGTCCGCGGCCACCGTGGCAGCCGGGTGATCCTCGACCGTGTGGCGGA
GGTCGATCGCCTGGTAAATCGCCTACCCGAGGAACTGAAGAACGTGGTGGTGGAGCA
CTATCTCAATCGCGACAGCTTCCCCGAGCAGAAGTACCGCCACTGCGGTTGCAGCCG
CAACACCTTCTATCTGCGCCTGCATGTGGCGCACCAGGGTATCCAGGACGGCCTGCTG
CGGCGGGTGGCCTGAGTTCCGGGACCGGATACCGTGTCCCCCCTGGAATTTTCCAAC
CGGCCTTCGAGCCGGTTTTTTTATGCCTGTCGCCCATGGAGGCGGCGAGCCCCATGCA
CAGCAGCGGACCCTGCACGCTAGAGGCGGGTTCGGGCTGGCGGAGCCGTCCTTTTCG
TCAGTCGATGCCGGGCTTTTCCATATCGGAAGTGGGTACGCGCTTGCCCGGATGCCTG
GCGAAGGTTCCCAGGGCGGTATATCACCCTCATATCGTGGTGCGTCTCCCTCCTATTGC
AGTGGATTGTCGAGAAGGCATTGCCGGGCTGGGAATCGGGCGGTAAAAAGTAGTCAT
TCTTGTAAAGGTGCGTCCCCAGGGAGGCACTCGTGAAGCACCGGAACCCGGCCCTG
GCGCCGGGTTTTTTATTGCCCGGGAAAACGTTCGTGCGGACGTGCCCGGCGCTGCCT
GCCAAGGAGGGACCCATGGGCAACGAACCGCAGACACTGACGGAAATGCCGCTCTG
GGTACTGATCCTGCTCGCCGCGCTGGGCGGCGTCAGCGGCGAGATGTGGCGTGCCGA
CAAGGCCGGTCTCGGCGGCTGGGCCTTGTTGCGGCGCCTGGCGCTGCGCTCCGGCGC
GTCGATTGTCTGCGGCGTGGCGGTGATGCTGCTGGCATTGGCTTGCGGCGCCGCGCT
GCTGTTCGCCGCGGCGCTGGGCAGCCTGACCGCCGCGGCCGGCGCGGAGATCGCAG
TCGGTCTCTACGAACGCTGGGCCGCCCGGCGCCTGGGGGTTTGCGAGCTGCCCGAGG
AACAGTCGGACGACCGTGGCCCGCATTGAAACTGATCGAAGGAGTCAACCATGCCTG
AACAGGCTGTTACGCTCGAGGCTCTGTACGCGGCCATCGAGCAGGTACTGCGTGAGC
GTCTGCCGGAGGCGCAGTTGATCGGCTTCTGGCCAGGCGTGCCGGAAAATACCCCGG
CGGTTTCCCTGGAAATGGCCGAGCTACTGCCCGAGCGCGATCCCGGTACCGGCGAGA
GTGCCCTGCTGTGCCGCCTGCAGGCGCGGATAATGGTGCCGCCTGGTGCCGATCGCC
AGGCGGTATCCATTGCTTGCGGAATCGTTCGGACATTGCGCGAGCAGACCTGGAACC
TGTCTCTGCAGCCGGCGCGCTTCGTACGCTCGGCCGTCGACGGCAGTCGCGAGGAGC
TGAAGAGCCTGCGTGTCTGGCTGGTCGAGTGGACGCAGTCGCTGCGTCTCGGAGACC
CGGAGTGGGCCTGGGAGGACCAGCCGCCCGGCAGCCTGATGCTGGGCTTCGACCCG
CAGACCGGCCCCGGCCATGAGCCGGACTACTTCGCTCCGGAGGCGTTGGCATGAGCT
ATGTCAGTGCGGAGCATGACCGCATGCTCGCCGCGATGATCCTGCCCTGCGTGGTGGT
CGCCGTGGACCTGGCAGCGGCGCGGGTACGGGTGCGCTCCGGCGACTGGACCAGCG
GCTGGCTGCGCTGGCACTCCCTGGCAGCCGGCAAGGTTCGCCACTGGCGTGCGCCG
AGCATAGGCGAACAGGGGGTACTGCTCAGTCCGTCGGGCGGAGTGTCAATGGGTACC
TTTATTCCCGGTCTGTACGGCGATGCGGGCACGGCGCCGGACAACAGCGCCAGCAGT
GAGACCTGGCGTTTCGACGACGGCGCCTCGTTGAGTTACGACTGGGCTGCGCATCGC
TACCGCGTCGAGCTGCCCAGCGGCACCGTGGAAGTGAGGGTCGGCGCCAGCGAGGT
GCGGGTCAGCGACGGGGCGGTCAGTCTCAAGGCGCCGAAGATCAGCCTGGAAGGAC
CGGTGGAGATCGCCGGGACACTGACGGTCAGCGGAGACATCCTCGGCGGCGGCTCG
ATCATCGACACCGCCGGCAACAGCAACCACCACACCCATTGAACATACGACGGGGCT
GCCGAAGGGCGGTCCGTCGAACATTCAACCGGCCCGCGCGAGCGGGCCTTTTCGTTT
GCGGAGTTCGCCATGGGCAACACTCACAGCCATTCGGAGCCAGGCGCGGCCTGTCTC
TGCCGGAGCGGGAGGGCAGGGCGATGATCGGGATGGATCGCCGTAGCGGGCTGCCC
CTGTCCGGCCTGGCTCATCTTAAACAGTCCGTCGAGGACATCCTGACCACCCCGTTGG
GCAGCAGGCGCATGCGCCCCGAGTACGGCAGCAAGCTGCGGCGGATGGTCGACATG
CCGGTGAGCGAAGGCTGGAAAAGCGCCGTGCAGGCCGAGGTAGCCCGTTCCCTGGG
GCGCTGGGAACCGCGCATCGCATTGTCTGCCGTGCGAGTCGTCGCAGTCGTCGATGG
CCGCGTGGATCTGCTCCTGAGCGGCGTGTTCGAGGGCGAGAACATCAATATGGAGGT
CTCGGCGTGATCATCGATCTTTCCCAGTTGCCGGAGCCAGAGGTTATCGAAAACCTCG
ATTTCGAGACGATTTACCAGGAGCTGTTGGGTGACTTCCGCGAAGCCATGGCTGGCG
AATGGACAGCGGAGGTGGAGTCCGATCCGGTTCTCAAGCTTCTGCAACTGGCGGCCT
ATCGAGAACTGCTGCTGCGGGCGCGGATCAACGATGCGGCGCGGGCGGTGATGCTGG
CATACGCCAGCGGTGCCGATCTCGACCAGATCGGTGCCGGCTTCAATGTGCAGCGTTT
GCTGATCAGGCCCGCTCAGCCCGAGGCGGTACCGCCGGTGGAGGCGCAATACGAGA
GCGACAAGTCGCTGCGCAATCGCATCCAGCTCGCGTTCGAGCAGCTATCCGTCGCAG
GACCGCGGAACGCCTATATAGCCCATGCGCTGGGCGCGGATGGAAGGGTGGCGGATG
CCTCTGCGACCAGTCCGGCGCCCTGCGAAGTGCTGATCAGCGTGCTCGGGGTGGAAG
GCAACGGGCAGGCACCGGAAGCGGTGTTGCAGGCAGTGCGCCTGGCGCTGAACGCG
GAGGACGTGCGTCCTGTCGCGGATCGGGTAACGGTGCGCTCGGCAGGAATCGTTCCC
TATCAGGTCAAGGCGCAGCTCTACCTGTTTCCCGGTCCCGAGGCGGAACTGATCCGT
GCCGCCGCCGAGGCTTCGCTACGCGACTACATTTCCGCCCAGCGCCGCCTGGGCCGC
GACATCCGGCGTTCGGCCCTGTTCGCCACCCTGCATGTCGAAGGCGTGCAGCGCGTC
GAACTGCAGGAGCCTGCGGCCGACGTGGTCCTGGATGAAACCCAGGCGGCCTATTGC
ACGGGGTACGCGATCACCTTGGGAGGCGTCGATGAGTAGCCGACTGCTGCCGCCAAA
CAGGAGTTCACTGGAACGCTCTCTGGGTGATGTATTGCCTGCCGAACTGCCGGTGCC
GCTTCGTGAGCTTAACGATCCGGCACGCTGTGAGGCGGCCTTGTTGCCCTACCTGGC
CTGGACGCGCTCGGTGGACCGCTGGGACCCGGACTGGAGCGACGAGGCCAAGCGCA
ATGCGGTAGCGACGTCCTTCGTCCTGCACCAGCGCAAAGGCACGCTGACCGCGTTGC
GCCAAGTGGTCGAGCCGATCGGTGCGCTGAGCGAGGTCACCGAATGGTGGCAGCGA
AGCCCGACCGGCGTGCCGGGGACCTTCGAGATCACCGTGGACGTCAGCGACCGTGG
CATCGACGAAGGCACCGTACTGGAGCTGGAGCGCTTGCTCGATGACGTCCGCCCGGT
GAGCCGACACCTGACCCGCCTGGACCTGCGCATCACCCCGGTAATCCGGTCCCGTCA
CGGACTGGCCGTGACCGACGGCGACACCCTGGAAATCTTCCCCTGGAAACAGTGAC
ATGACGACCAATACTCCGAAATACGGTGGCCTGCTCACCGACATAGGTGCCGCTGCG
CTGGCTGCGGCCAGCGCAGCAGGCAAGAAATGGCAGCCGACTCATATGCTGATCGGC
GATGCTGGCGGCGCACCGGGTGCTACTCCGGATCCGATACCCGCAGCCACGCAGACG
AAACTGATCAACCAGCGCTACCGGGCGCAGCTCAATCGCTTGTTCGTGTCGGACAAG
AACATCAATACTCTGGTTGCCGAGGTGGTGCTACCAGTTGAGGTAGGTGGCTTCTGG
ATCCGCGAGATCGGCCTGCAGGATGCCGACGGCAAGTTCGTCGCGGTATCCAACTGC
CCGCCCAGCTACAAGGCAGCAATGGAAAGTGGCAGTGCGCGGACCCAGACCATTCG
GGTGAACATCGCGCTCTCCGGCCTGGAGAATGTCCAGTTGCTGATCGACAACGGCAT
CATCTACGCCACTCAGGACTGGGTGAAGGAAAAGGTCGCTGCCGATTTCAAGGGCCG
CAAGATCCTGGCTGGCAATGGCCTGGTCGGTGGGGGCGATCTTTCTGCCGACCGCAG
CATTGGCCTGGCGCCTTCCGGCGTGACGGCGGGCAGCTATCGCTCGGTCACGGTGAA
CGCCAACGGGGTGGTCACCCAGGGCAGCAATCCGAGCACCCTGGCCGGCTATGCGAT
CGGAGATGCCTATACCAAGGCCGATACCGACGGAAAACTGGCGCAGAAAGCGAACA
AGGCCACCACCCTGGCCGGCTATGGCATCACCGATGCGCTGCGAGTCGATGGCAACG
CCGTGTCATCCAGCAGGCTGGCCGCACCGCGTAGCCTGGCAGCCAGTGGCGATGCCT
CCTGGTCGGTGACCTTCGACGGCAGTGCCAATGTTTCTGCGCCGCTGAGTCTTTCCGC
TACCGGTGTGGCAGCGGGCAGCTATCCGAAGGTGACCGTGGATACGAAGGGAAGGG
TGACTGCTGGAATGGCGCTGGCGGCGACGGACATTCCCGGGCTGGATGCTTCGAAGT
TGGTCAGCGGGGTGCTGGCCGAGCAGCGTTTGCCGGTATTCGCGCGCGGGTTGGCTA
CTGCTGTCTCGACCACTAGCGATCCGAACACCGCGACCGTGCCGTTGATGCTGACCA
ATCATGCGAACGGACCTGTTGCCGGACGGTACTTCTACATCCAGTCGATGTTCTATCC
GGATCAGAACGGCAATGCTTCGCAGATTGCAACGAGCTACAACGCTACATCCGAGAT
GTATGTACGGGTGTCCTACGCGGCCAACCCTAGCGCCCGGGACTGGCTGCCCTGGAA
GCGCTGCGACATAGGTGGTTCGTTCAGCAAGGAGGCGGACGGGGCCTTGGGCGGTG
CGGTCAATCTCAACTCGCTGATCACGTCGGGATGGTGGTACCAGACGGCCAATGCAC
AGGCCGAAAGTGGGGCGAACTACCCGGTCCCCCGGGCCGGCTTGCTTCAAGTGCATA
ATGCAGGCACCAATTTCATCTACCAGACATACCAGGTTTATGACGGTGAAGGGTTCTA
TTTTCGTTGCCGCTACACCAACACCTGGTATCCATGGCGGCGTGTATGGCATGGAGCG
GACTTCAATCCGAACGACTATCTGCTGAAAAGTGGCTTTACGTGGGCCGCCCTGCCA
GGAAAGCCAGCGACCTTTCCGCCTACTGGCCACAACCACGACGCCGCCCAGATCACG
TCGGGCATCCTGCCTCTGGCGCGCGGCGGTCTTGGTTCGAATACGGCAGCGGGGGCG
CGCAACAATATCGGGGCTGGGGTTCCCGCGACGGCGAACCGATCGCTGAATGGCTGG
TGGAAGGATAACGATACAGGACTCATCGTGCAGTGGATGACAGTGAGTGTCGGCGAT
CATCCGGGTGGAATCGTCAACCGCTCGCTGACCTTTCCGATCGCGTTTCCCACTACCT
GCCTGCACGTGGTGCCGAGCGTCAAGGAACTGGGGCGGCCGGCGACGTCGGCATCG
ACCGTCACCCTCGCAGATGTCAGCGTCAGCACCACGGGGTGTGTGATTGTCGCTACC
GAGTATCACGGTGCGGTCCAGAACTATGCGATCAGGCTTGTGGCCATTGGCTGTTGAG
GTGACCCATGATTTTTTTCCATGCCGCCACTGGCGGTTTCTATTCGAAGGATGTCCACG
GCGACCGCATGCCCATCGACGCGCGGATGTATCCCCTCGAGGAAGCTGAATACCTGG
CGCTGTTGGTAGCCCAGAGCGAGGGCAAGCAGATCGTCGCCGACGCCGCCGGCCGT
CCGTTCTGTATCGACCCGCCGGCCCCGGCAGAAGAGGTCCTGGCCCACCGTGAGCGC
ATCTGGCGCGATCGCCAGTTGACGCTTACTGATGGCCCCATCGCCCGTCACCGGGATG
AGCTCGACCTGGGCAAGATTACGACCCTGAACCAGGCGCAACTGCTCGAACTCACGC
TGTACCGCGCCAGCCTTCGAGACTGGCCGGCATCCGCGGCGTTTCCTGATTTAGGCGC
AAGACCCGAGCCGCCGCTGTGGCTCGAACCGCTCATCACCCCCTGAACCCCGCCCCG
TGCGGGGTTTTTCATTAATGGAGATCTACCTATGAGTTTCTTCCACGGCGTTACGGTAA
CCAACGTCGATATCGGTGCGCGCACCATCGCGCTGCCGGCCAGCTCCGTCATCGGCCT
CTGCGATGTGTTCACGCCGGGGGCGCAGGCAAGCGCCAAGCCCAACGTTCCGGTGC
TGCTCACCAGCAAGAAGGACGCCGCCGCGGCGTTCGGCATCGGCTCGTCTATCTACC
TGGCCTGCGAGGCCATCTATAACCGTGCCCAGGCCGTGATCGTGGCGGTGGGCGTGG
AAGCCGCGGAGACTCCCGAGGCCCAGGCCAGCGCCGTCATAGGTGGTGTCAGCGCC
GCCGGCGAGCGCACCGGACTTCAGGCTCTGCTAGACGGCAAGAGCCGCTTCAATGCT
CAGCCGCGTCTACTAGTTGCGCCGGGTCATTCGGCCCAGCAAGCGGTGGCCACCGCC
ATGGACGGGCTGGCCGAGAAACTGCGGGCCATCGCCATTCTCGATGGCCCCAATAGC
ACCGACGAGGCGGCCGTCGCCTACGCCAAGAACTTCGGCAGCAAGCGCCTGTTCATG
GTCGACCCGGGCGTGCAGGTTTGGGACAGCGCCACCAATGCCGCACGCAAGGCCCC
GGCTTCGGCCTACGCCGCCGGCCTGTTCGCCTGGACCGACGCCGAGTACGGCTTCTG
GTCGTCGCCGTCGAACAAGGAGATCAAGGGCATCACCGGCACCAGCCGTCCGGTGG
AGTTCCTCGACGGTGATGAGACCTGTCGCGCCAACCTGCTCAACAACGCCAATATCG
CCACGATCATTCGCGACGATGGCTATCGCCTGTGGGGCAATCGCACCTTGTCCAGCGA
CAGCAAGTGGGCTTTCGTCACCCGCGTTCGGACCATGGACCTGGTGATGGATGCGAT
CCTCGCCGGGCACAAGTGGGCGGTGGACCGCGGCATCACCAAGACCTACGTGAAGG
ATGTCACCGAGGGCCTGCGCGCCTTCATGCGCGATCTGAAGAACCAGGGCGCGGTGA
TCAACTTCGAGGTCTATGCCGACCCAGACCTGAACAGCGCCAGCCAGCTGGCCCAGG
GCAAGGTGTACTGGAACATCCGCTTCACCGATGTGCCGCCTGCCGAGAACCCCAATT
TCCGTGTCGAGGTGACCGATCAGTGGCTCACCGAAGTTCTGGATGTCGCCTAAGGAG
CGCCCGTGATGATTCCGCAAACCCTGACCAATACCAACCTGTTCATCGACGGCGTGA
GCTTCGCCGGTGACGTGCCATCCCTGACCCTGCCCAAGCTGGCTGTGAAGACCGAGC
AATACCGTGCCGGCGGCATGGATGCGCCGGTATCCATCGACATGGGCCTGGAGGCGAT
GGAGGCCAAGTTCTCCACCAACGGTGCCCGCCGAGAAGCACTGAATTTCTTCGGCCT
GGCCGACCAGAGCGCTTTCAATGGCGTGTTCCGCGGCTCCTTCAAGGGCCAGAAGG
GCGCCAGCGTGCCAGTGGTGGCTACCCTGCGCGGCCTGCTCAAGGAAGTCGACCCG
GGCGACTGGAAGGCCGGCGAGAAAGCCGAGTTCAAGTACGCCGTTGCGGTCAGCTA
CTACAAGCTGGAAGTCGATGGCCGCGAGGTCTACGAGATCGATCCGGTCAACGGTGT
CCGCGCGATCAACGGTGTCGACCAGTTGGCCGGCATGCGCAACGACCTCGGCCTGTA
AGAGGAGCTCCGGACATGACCCAAGAGAATCGACTGCCGGGCTGGCTGACGCTGGA
TGCTGACGCCGCCCTCGTTCGTCTCTCGCGTCCGGCACAGTGCAACGGGGTCAGCGT
CGACACGCTGACCCTGCGTGCACCCACCGTGCGTGATATCCGCCTGGCCGGCAAGGT
GGCCGGCGACGATGCCGAGGAGCGCGAACTGCAACTATTCGCCTCGCTGGCGCAGGT
CAGCCGCCAGGACCTGGAGGGGCTGAAGCTGAGCGACTACCAGCGGCTGCAAGGCG
CCTACTTTCGCCTGGTGCAAGACGACACGGACGACACCTTCGCGTATGCGTCAACTG
GCGAGGCGCCTGGCCATTGAGCTGGGCTTCCAGGCCAGCGAACTGGAGCGCATGAC
CCTGGGCGACCTGCTCTGGTGGCTCGCCGAGGGCGAGGAGTGAGCGACGGCGCCGT
CCGCCAGGCGAGCGGCGCCCGCGCTACTTCAGTAGGTCCGCGGAGGCGGACATCGAT
AACAGACGAGACGAACCGTCATGAGTAAAGACATGGACCTGGTGGTCTCCATCGGCG
GCATCGCCGACCCCTCCTTGGGCAAGGCATTCGAGACCGTCAAGGCACGGCTCGACA
GTCTCCAGGAACGTTCCCGCCAGGCGTCCAGCCTGAGGGATGTGCTGGGAGACGCC
ATACGCCTGGAGCGAGAACTCGCCGATATGCGCAAGGTCGGGGACCGCGGCGTTGCT
GAGCATGCCCGGCAGCTTGGCGAACGCCAGGAGCAACTGAAGCGACTCGGCATCGA
GGCCAGGGCCGCGGGCGATGCCTACGCTCGACTGGGCGAGATGCAGCGTGGCCTGG
ATATGCAGGTCCGCGGCCTGCAACGGCTGGAGCAGGCCAGCCAGGCAATGCCATTGG
CTAGCGCATTTTCCGGACTGGTCGTGGAAGCCAGCAAGACGGCTGCCGGTTATCAAG
CGCGGTTGCGCGACCTGGCGATCCGCAACGGCCTGGACGTCGGCCGGGAGCCAGCC
TTGGCATCCCTGATCCAGGACAGTGCCAGCCAGAGCGGCCTGGGACGCACGGCGAC
GCTGGACATGCTGGAGCACTTGAACGCCACCGGCATGGGGTTCGCCGCCGCGCAAAT
GAATCTGGGACTGGCGGGCCGCTTCGGCTTTGGCCAAGGGATTGCTTCAGCCGAGGT
TGCGGGGCTGGTTCGAGCGTTGCAACTGGCCCAGGGTTCGGACTCGCCAGAGCAATT
GTCCGCCTCCCTCGACCGCCTGGTCGTCCTGGGTAAAGGCAGAGTCGGCAGTGAGGC
CCTGGCGCGTCGCCTGCCTGCCTTGTTGTCAGCGCTGGGCAATGCGGGAGAGGCCAC
GGCCGGCGATGTCGGTGCACTGGGGGCCTTGCTGGAGATTCAGGCAAAGAACACCA
CGCCAGACAAAGCGGACGTGCGGATGAAGGCCTGGCTGGAGTTCGTCGGCAGCGGC
AGCTTGAAACGCGCTTATGGCCAGGACTACGACCGGGACCTGGAAGCGCTGCGCAA
GGACGGAGCGAGTCTGCTGGAGGCCAACCTGGAGCTGGCTGCGCGCTATCGGGATA
AGGGCGGCAAGCTCAGCGCCGGCGTGGCGTCGCCGGCGCTTGAAGCCTATCGAGCG
TCGCGCGGCGAGTTCCAGGGCTTGCTCGAATCCCAGCAGTCTTCCGTAGGCTCTTCG
GAGCGCGATGCGCAGCGTCGCAAGGGGATGTCCCAGGAGCTGTGGAAGGCTTCCAG
CGACAGTTGGGAGAGGGCGCAGACCGCTTTGGGCAGCGCCCTGAATCCATATCTGGA
CAACCTGGCCAAGGGGAGCGCGGTACTCGGCGAGTCGACTGCGGAGCTGCTCGAAG
CCTATCCGCGGACGACGGCCGGTCTTACCGCCGCCGCAGGTGCGGTGTTATCCGGGT
ATCTCGCCTACAAGGGAGGGCGCGGCGCTATCGACGTGCTGCGTGGCGGTCGGCTCG
GTCGGCGAGGGACCGCTGCCGTCGGCGACCTGATCGAACGGGGTGCAGGCCGGGTA
TCGGGTGGTAGCGAAATACAGCGCGTGTTCGTTACCAACTGGCCGGTGCCGGGAGGC
GACTCTACGCTGGAGTCCGCGAGGAGGCCAGCACAACGAAAGCGCGGTCAAACACC
ACGCAGGAAGAGAGGCAAGGGCGGTGGGCTGAAGGCCCGCTCCCTTCCATCCCTTG
GCTTCTCGGCTGGAGGAGGCTTGGGGGCGATGGCAGGAAAGCTGCCGCGCCTGTCG
CGTTTGCCCATACGCAACGCGCCGCTGCAAGTGGCCTCGTCGTTGATCGATGTTGCTG
AGGTCTACTCCAGCGACCTGTCGGAGAGCGAAAAGACCGTTGCCTATGGCGAGGCA
GGTGGCTCCTTGGCTGGTTCCCTGGCTGGCGCCGCTCTGGGAGCGAGCATCGGCTCG
GTGGTGCCGGTGGTCGGTACGCTGATCGGTGGATTGGTTGGCGGCGCTATCGGCGCC
TGGGGCGGTAGCGAACTGGGGGGGCGCCTGGGGCGCAGTCTGGCTGGCGATCCGCC
GGCGGCCTCGGACAACAAGCCGGCGGTGGCCGTACCACAGGCCGGACCCGTCGCGG
CTGCGCCCAACTGGACCTTCGCGCCGCAGATCAACCTGACGGTGCAAGGCAACGTG
CACGAGCCGCAGCGCCTGGCCGACGAGTTGCTGCCCTACCTGCAACGCATGCTTGTC
GACTTCGCCGACGAGCGGCAGCGGCGCAGCCTCTACGACCCGGCGATGGTTTAAGG
AGTCCCCATGGCCTATCTGGAACAATTGCAGGCCGGCCTGAGGTACCTGGGTCGCGC
CGGCGAGTCCGGACGCAAGAGTCTGGACAAGGTGGTCGCTCCGGTGAACGGCGCGA
TCAGCGAGATCCGCGGCGCAGCCGCGGAGCTGGAGAACCTGCCCGGCGTATCGCCG
GAAATGGCTGCCCGGCTGCAGCGTGCCATGCGCGGCATCGGCCAGGCGCAGGGCAA
GGTGAACCGCGTGGTCTCCACCTATGACCGGGCGAGCCGGGCGTTGCTTGGTATCGA
CGAACGCCTGGATGCGCTGAAGGTGCAGGTGAACAGTGCCGCGCAGGCGGTCGGCA
AGGTCGCTGGCGACATCAGTCCGACGCTGGCGGGGGTGCTGCCGTCGTGGCTGCTGG
CACCCTCGGCGACGCCCCCGAGCGAGGCCGCAGCGCCCTTGCCGCACCTGCTGGTAC
TGCAGCCGCTGACCGCCAATGCCCAACCGTTCTACTTCAACCTGAATACTGCCGCCTT
CGACGCCCTGCAGCGCAACAGTGCCTACAACTGGAGCGGGCAGGTGCGCCTGGGTC
GGCGGCCGGCGCTGCAGAGCGTCGGCATGGGCGAGGAGAGCATCCTGCTCAAGGGT
GCGGTATTCCCGCTGCGTCGACAGGTAGGTAACCAGGAAAAGGTCGTCGGTCTGGAG
CAGCTCGAAACGCTGCGCCGGTTGGCGGAGCGGCGTGAGCCGCTGATCCTGAGCAG
CGGCTACGGCGAGGTGCAGATGGGCCTCTGGTGTCTGGTGCGGATCAGCGAGAACCA
GAGCGCCCTACTGGGCAACGGCGCTCCCCGCAAACAAACCTTCGACCTGGAGTTCA
AGCGCTATGGCGACGACCTGCCGAACCGCTGACGGCGACATGCTGGACAGCCTCTGC
TACCACGTCTATGGCCATCTGTTGGGCTGCGTCGAGGCGACCCTCGACGCCAATCCCG
GGCTGGCCGATGAGCAGCAGCCATTCCGCGCCGGCTTGCTGATCAGTTTCCCTGACAT
GCCGGTGGTCAATGTCGAACAGGTGCGCCTGTGGGATTGATCGACCGCTCACCCGCA
ACCCCGCCTTGGCGGGGTTTTTCTTTTCTGGAGAAACCAGGTGCAACCGAGTTTCCG
TATCGTTGCCGACGGCACCGACGTCACCCAGCGGCTGAATGATCGCCTGCTCAAGCT
GACCCTGCTGGACAAGCCGGGCATGGAGTCCGACAGCCTGACCTTGAGGATCGACG
ATCGCGATGGACAGGTGGCCTTACCCAGGCGCGGTGCGGTGCTGGAGGTTCATCTCG
GCTATGCCGGCGAGCCACTGATGCGCATGGGACGCTTTACCGTGGACACCTTGCAGT
GGGCTGGTCCGCCGGACTGCCTGACCGTCACTGCCAAGGCCGGCGACATGCGCGGC
AGTGGCAAGACGATACGCAGCGGAGGTTGGGAGGGCACTACCCTGGCTCAGGTCTG
CCGCGATGTTGGCGCACGCAACGGCTGGCGCGTGGAGTGTCCGTTGCAGGTGGCGAT
CGCCCGGGTCGACCAGGTCAATGAGTCCGACTACCACTTCGTCACCCGTCTGGCGCG
CCAGTACGACTGCACCGCCAAGCTGGCCGAGGGCATGCTCATGGTGCTGCCGCGACA
GAGCGGGCAGAGCGCCACGGGGCGTCGGATCGAACCTTTGGTGTTGGGACGTGCTG
ACGTCGGTAGCTTCGACGTTACCTTCGACGACCGCAGCCTGATGAGAACGGTGAAGA
CCCGCTACCAATTGCCCGGCAGCGGCGAGGTCAAGAGCGTCGAGTTGAAGAACCCG
AAGGCACCGGCTACGGCTATGGGCGAGCATGTCGACCGGCACCTCTATACCAGCCGT
GGAGAGGCCGAGCAGGCGGCGAAGGCTCGCCTGGCGAGCTTCAGCCGCTCCAGCGC
CAGTGTGCGCCTGGGACTGCCAGGGCGTGGCGACCTGTTCGCCGAGCGCAGCCTGC
TGCTCCAAGGCTTCAAGGCGGGAATCGACGGCGAGTTCCTGATCGACTCGGTGGAGC
ACACCTACAGCTCCAGCGGATGGACCACTGTCGTGCAATGCAACGGCGGCCGAGGC
GGCAAGGGGTGA
R5_tail
SEQ ID NO: 12
ATGACGACCAATACTCCGAAATACGGTGGCCTGCTCACCGACATAGGTGCCGCTGCG
CTGGCTGCGGCCAGCGCAGCAGGCAAGAAATGGCAGCCGACTCATATGCTGATCGGC
GATGCTGGCGGCGCACCGGGTGCTACTCCGGATCCGATACCCGCAGCCACGCAGACG
AAACTGATCAACCAGCGCTACCGGGCGCAGCTCAATCGCTTGTTCGTGTCGGACAAG
AACATCAATACTCTGGTTGCCGAGGTGGTGCTACCAGTTGAGGTAGGTGGCTTCTGG
ATCCGCGAGATCGGCCTGCAGGATGCCGACGGCAAGTTCGTCGCGGTATCCAACTGC
CCGCCCAGCTACAAGGCAGCAATGGAAAGTGGCAGTGCGCGGACCCAGACCATTCG
GGTGAACATCGCGCTCTCCGGCCTGGAGAATGTCCAGTTGCTGATCGACAACGGCAT
CATCTACGCCACTCAGGACTGGGTGAAGGAAAAGGTCGCTGCCGATTTCAAGGGCCG
CAAGATCCTGGCTGGCAATGGCCTGGTCGGTGGGGGCGATCTTTCTGCCGACCGCAG
CATTGGCCTGGCGCCTTCCGGCGTGACGGCGGGCAGCTATCGCTCGGTCACGGTGAA
CGCCAACGGGGTGGTCACCCAGGGCAGCAATCCGAGCACCCTGGCCGGCTATGCGAT
CGGAGATGCCTATACCAAGGCCGATACCGACGGAAAACTGGCGCAGAAAGCGAACA
AGGCCACCACCCTGGCCGGCTATGGCATCACCGATGCGCTGCGAGTCGATGGCAACG
CCGTGTCATCCAGCAGGCTGGCCGCACCGCGTAGCCTGGCAGCCAGTGGCGATGCCT
CCTGGTCGGTGACCTTCGACGGCAGTGCCAATGTTTCTGCGCCGCTGAGTCTTTCCGC
TACCGGTGTGGCAGCGGGCAGCTATCCGAAGGTGACCGTGGATACGAAGGGAAGGG
TGACTGCTGGAATGGCGCTGGCGGCGACGGACATTCCCGGGCTGGATGCTTCGAAGT
TGGTCAGCGGGGTGCTGGCCGAGCAGCGTTTGCCGGTATTCGCGCGCGGGTTGGCTA
CTGCTGTCTCGACCACTAGCGATCCGAACACCGCGACCGTGCCGTTGATGCTGACCA
ATCATGCGAACGGACCTGTTGCCGGACGGTACTTCTACATCCAGTCGATGTTCTATCC
GGATCAGAACGGCAATGCTTCGCAGATTGCAACGAGCTACAACGCTACATCCGAGAT
GTATGTACGGGTGTCCTACGCGGCCAACCCTAGCGCCCGGGACTGGCTGCCCTGGAA
GCGCTGCGACATAGGTGGTTCGTTCAGCAAGGAGGCGGACGGGGCCTTGGGCGGTG
CGGTCAATCTCAACTCGCTGATCACGTCGGGATGGTGGTACCAGACGGCCAATGCAC
AGGCCGAAAGTGGGGCGAACTACCCGGTCCCCCGGGCCGGCTTGCTTCAAGTGCATA
ATGCAGGCACCAATTTCATCTACCAGACATACCAGGTTTATGACGGTGAAGGGTTCTA
TTTTCGTTGCCGCTACACCAACACCTGGTATCCATGGCGGCGTGTATGGCATGGAGCG
GACTTCAATCCGAACGACTATCTGCTGAAAAGTGGCTTTACGTGGGCCGCCCTGCCA
GGAAAGCCAGCGACCTTTCCGCCTACTGGCCACAACCACGACGCCGCCCAGATCACG
TCGGGCATCCTGCCTCTGGCGCGCGGCGGTCTTGGTTCGAATACGGCAGCGGGGGCG
CGCAACAATATCGGGGCTGGGGTTCCCGCGACGGCGAACCGATCGCTGAATGGCTGG
TGGAAGGATAACGATACAGGACTCATCGTGCAGTGGATGACAGTGAGTGTCGGCGAT
CATCCGGGTGGAATCGTCAACCGCTCGCTGACCTTTCCGATCGCGTTTCCCACTACCT
GCCTGCACGTGGTGCCGAGCGTCAAGGAACTGGGGCGGCCGGCGACGTCGGCATCG
ACCGTCACCCTCGCAGATGTCAGCGTCAGCACCACGGGGTGTGTGATTGTCGCTACC
GAGTATCACGGTGCGGTCCAGAACTATGCGATCAGGCTTGTGGCCATTGGCTGTTGA
R5_pyocin_tail_fiber
SEQ ID NO: 13
MTTNTPKYGGLLTDIGAAALAAASAAGKKWQPTHMLIGDAGGAPGATPDPIPAATQTKL
INQRYRAQLNRLFVSDKNINTLVAEVVLPVEVGGFWIREIGLQDADGKFVAVSNCPPSYK
AAMESGSARTQTIRVNIALSGLENVQLLIDNGIIYATQDWVKEKVAADFKGRKILAGNGL
VGGGDLSADRSIGLAPSGVTAGSYRSVTVNANGVVTQGSNPSTLAGYAIGDAYTKADTD
GKLAQKANKATTLAGYGITDALRVDGNAVSSSRLAAPRSLAASGDASWSVTFDGSANV
SAPLSLSATGVAAGSYPKVTVDTKGRVTAGMALAATDIPGLDASKLVSGVLAEQRLPVF
ARGLATAVSTTSDPNTATVPLMLTNHANGPVAGRYFYIQSMFYPDQNGNASQIATSYNAT
SEMYVRVSYAANPSARDWLPWKRCDIGGSFSKEADGALGGAVNLNSLITSGWWYQTAN
AQAESGANYPVPRAGLLQVHNAGTNFIYQTYQVYDGEGFYFRCRYTNTWYPWRRVWH
GADFNPNDYLLKSGFTWAALPGKPATFPPTGHNHDAAQITSGILPLARGGLGSNTAAGA
RNNIGAGVPATANRSLNGWWKDNDTGLIVQWMTVSVGDHPGGIVNRSLTFPIAFPTTCL
HVVPSVKELGRPATSASTVTLADVSVSTTGCVIVATEYHGAVQNYAIRLVAIGC
galU
SEQ ID NO: 14
ATGATCAAGAAATGTCTTTTCCCGGCCGCCGGTTACGGCACCCGTTTCCTCCCCGCCA
CCAAGGCCATGCCCAAGGAAATGCTGCCGGTGGTGAACAAGCCGCTGATCCAGTACG
CGGTGGAGGAGGCGCTGGAAGCCGGCCTTTCCGAGATCGGCATCGTCACCGGCCGC
GGCAAGCGTTCGCTGGAAGACCACTTCGACATCAGCTACGAGCTGGAACACCAGATC
CGCAACACCGACAAGGAAAAGTACCTGGTCGGCATCCGTCGGCTGATCGACGAGTG
CACCTTCGCCTACACCCGCCAGGTGGAGATGAAGGGCCTCGGCCACGCCATCCTCAC
CGGTCGTCCGCTGATCGGCGACGAGCCGTTCGCCGTGGTCCTGGCCGACGACCTGTG
CCTGAACCTCGAAGGCGACAGCGTGCTGAAGCAGATGGTCAAGCTGTACAACCAGT
TCCGCTGCTCCATCGTGGCGATCCAGGAAGTGCCGCCGGAAGAGACCAACAAGTACG
GCGTGATCGCCGGCGAGATGATCCGCGACGATATCTTCCGGGTGAACACCATGGTCG
AGAAGCCGAAGCCGGAAGAGGCGCCGTCGAACCTGGCGATCATCGGCCGCTACATC
CTGACCCCGGACATCTTCGACCTGATCGAGCAGACTGAACCGGGCAAGGGCGGCGA
GATCCAGATCACCGATGCCCTGATGAAGCAGGCCCAGGACGGCTGCGTGCTGGCCTA
CAAGTTCAAGGGCAAGCGTTTCGACTGCGGCAGCGCCGAGGGTTACATCGAGGCGA
CCAACTTCTGCTACGAAAACCTCTACAAGACCGGCAAGGCTCACTGA
hmgA
SEQ ID NO: 15
ATGAACCTCGACTCCACTGCCCTCGCCTATCAATCGGGCTTCGGCAACGAATTCAGCA
GCGAAGCGCTCCCCGGCGCCCTGCCGGTCGGCCAGAACTCCCCGCAGAAAGCGCCC
TACGGCCTGTACGCCGAACTGCTCTCCGGCACCGCCTTCACCATGGCTCGCAGCGAG
GCCCGGCGCACCTGGCTGTACCGCATCACGCCGTCGGCCAAGCATCCGCCGTTCCGC
CGCCTGGAACGACAGATCGCCGGTGCCGAACTGGATGCGCCGACTCCCAACCGCCTG
CGCTGGGACCCGCTGGCACTGCCCGAGCAGCCCACCGACTTCCTCGACGGCCTGCTG
CGCATGGCCGCCAACGCGCCCGGCGACAAGCCCGCCGGCGTGAGCATCTACCAGTAC
CTGGCCAACCGCTCGATGGAGCGTTGCTTCTACGACGCCGATGGCGAACTGCTGCTG
GTCCCGCAATTGGGCCGTCTGCGCCTGTGCACCGAACTCGGCGCGCTGCAAGTCGAA
CCGCTGGAGATCGCGGTGATCCCGCGCGGGATGAAGTTCCGCGTCGAGCTGCTCGAC
GGCGAGGCACGCGGCTATATCGCCGAGAACCACGGCGCGCCGCTGCGCCTGCCCGAC
CTCGGCCCGATCGGCAGCAATGGCCTGGCCAATCCGCGCGACTTCCTGACCCCGGTG
GCGCGCTACGAGGACAGCCGCCAGCCGCTGCAACTGGTGCAGAAATACCTCGGCGA
GCTGTGGGCCTGCGAGCTTGACCACTCGCCGCTGGACGTGGTCGCCTGGCACGGCA
ACAACGTGCCCTACAAGTACGACCTGCGCCGCTTCAACACCATCGGCACGGTCAGCT
TCGACCACCCGGACCCGTCGATCTTCACCGTGCTGACCTCCCCCACCAGCGTCCCCG
GCCTGGCCAACATCGACTTCGTGATCTTCCCGCCGCGCTGGATGGTGGCCGAGAACA
CCTTCCGTCCGCCATGGTTCCACCGCAACCTGATGAACGAATTCATGGGCCTGATCCA
GGGCGCCTATGACGCCAAGGCCGGCGGCTTCGTGCCCGGCGGCGCCTCGCTGCACAG
TTGCATGAGCGCCCACGGCCCGGACGCGGAAAGCTGCGACAAGGCCATCGCCGCCG
ACCTCAAGCCGCACAGGATCGACCAGACCATGGCCTTCATGTTCGAGACCAGCCAGG
TCCTCCGGCCGAGCCGTGCCGCCCTCGAGACGCCGGCCCTGCAGAATGACTACGATG
CCTGCTGGGCGTCGCTCGTATCCACCTTCAACCCGCAACGGAGATAA
MexX
SEQ ID NO: 16
ATGCACATCCAATGGACCGGCTCGCTGCGCGGGCTGCTGGCGGCCCTGGTCGCCCTAT
TCCTGCTGGGCTGCGAAGAAGCAGCGGACGCCGGGAAGACTGCGGAGGCCCCCGCC
GAGGTCGGCGTGATCGTCGCCAGGCCGGCGCCTATCGGCATCACCAGCGAGCTGCCC
GGACGCCTGGAAGCGTACCGCCAGGCTGAAGTGCGGGCGCGCGTCGCCGGCATCGT
CACCCGTCGCCTGTACGAGGAAGGCCAGGACGTCCGCGCCGGCACCGTGCTGTTCCA
GATCGACCCTGCGCCCTTGAAGGCGGCCCTGGACATCAGCCGCGGCGCCCTGGCCCG
GGCCGAGGCCAGCCACGCGGCGGCGGCCGACAAGCTCAAGCGCTACGCCGACCTGA
TCAAGGACCGCGCCATCAGCGAACGCGAGTACACCGAAGCGCAGACCGACGCGCGC
CAGGCCCTGGCGCAGATCGCCTCGGCCAAGGCCGAACTGGAGCAGGCCCGCCTGCG
CCTGGGCTACGCCACGGTCACCGCGCCGATCGACGGCCGCGCGCGGCGTGCGCTGGT
CACCGAAGGCGCGCTGGTCGGCGAGGACTCGCCGACACCGCTGACCCGCGTCGAGC
AGATCGATCCGATCTACGTGAACTTCTCCCAGCCGGCCGGCGAAGTCGCCGCCATGC
AGCGGGCGATCCGCGAAGGCCAGGTGAAGGGTGTCGCCGACAAGGACATCGCCGTG
CGCCTGGTCCTGGCCGACGGCAGCGAGTACCCGCTGGCCGGCGAGCTGCTGTTCTCC
GACCTGGCGGTCGACCCCGGCACCGACACCATCGCCATGCGTGCCCTGTTCCGCAAT
CCGCATCGCGAATTGCTGCCCGGCGGCTACGTGCAGGTGCGCCTGCAGCGCGCGGTG
AACCCGCAGGCGATCACCGTCCCGCGCGACGCGCTGATCCGTACCGCCCAGTCCGCC
GTGGTCAAGGTGGTCAACCCAAAGGGCTTGGTGGAAGACGTGGAGGTCCGCGCCGA
CACCCTGCAGGGCCGCGACTGGATCATCAGCCGCGGGCTCAAGGGCGGCGAGTGGG
TGATCGTCGAGAACGCCGCCCAGCATGCCGCCGGCTCCAGCGTCCAGGCGGTGGTCC
GCCAGCCGGCCAGCGCCGACGCCCCCTCACCGCTGGCCGCCTCGCCGGCGGGCCAG
TGA
MexY
SEQ ID NO: 17
ATGGCTCGTTTCTTCATTGACCGGCCGGTCTTCGCCTGGGTGATCTCCCTGCTGATCGT
GCTCGCCGGGGTCCTGGCGATCCGCTTCCTGCCGGTCGCCCAGTACCCGGACATCGC
GCCGCCGGTGGTCAACGTCAGCGCCAGCTATCCCGGCGCCTCGGCCAAGGTGGTCGA
GGAAGCGGTGACCGCGATCATCGAGCGCGAGATGAACGGCGCGCCCGGCCTGCTCTA
CACCAAGGCCACCAGCAGCACCGGCCAGGCCTCGCTGACCCTGACCTTCCGCCAGG
GCGTGAACGCCGACCTCGCCGCGGTGGAAGTGCAGAACCGCCTGAAGATCGTCGAG
TCGCGCCTGCCCGAATCGGTGCGGCGCGACGGCATCTACGTGGAGAAGGCGGCGGA
CAGCATCCAGCTGATCGTTACCCTTACCTCCTCCAGCGGCCGCTACGACGCCATGGAG
CTGGGCGAGATCGCCTCGTCCAACGTGTTGCAGGCGCTGCGCCGGGTGGAGGGCGT
GGGCAAGGTCGAGACCTGGGGCGCCGAGTACGCCATGCGCATCTGGCCCGACCCGG
CCAAGCTGACCTCGATGAACCTCAGCGCCAGCGACCTGGTCAACGCCGTGCGCCGG
CACAACGCCCGCCTCACCGTGGGCGACATCGGCAACCTCGGGGTCCCCGACTCGGC
GCCGATCAGCGCCACGGTGAAGGTCGACGACACCCTGGTGACGCCCGAGCAGTTCG
GCGAAATTCCGCTGCGCATCCGCGCCGACGGCGGCGCGATCCGCCTGCGCGACGTGG
CCCGCGTCGAGTTCGGCCAGAGCGAGTACGGCTTCGTCTCGCGGGTCAACCAAATGA
CCGCCACCGGCCTGGCGGTGAAGATGGCGCCCGGCTCCAACGCGGTGGCCACCGCC
AAGCGCATCCGCGCCACCCTCGACGAGCTGTCGCGCTACTTCCCGGAGGGCGTGAGC
TACAACATCCCCTATGACACCTCGGCGTTCGTCGAGATCTCGATCAGGAAGGTGGTCA
GCACCCTGCTCGAGGCGATGCTGCTGGTGTTCGCCGTGATGTACCTGTTCATGCAGAA
CTTCCGCGCCACCCTGATCCCGACACTGGTGGTGCCGGTGGCCCTGCTGGGCACCTT
CACGGTGATGCTCGGCCTGGGCTTCTCGATCAACGTGCTGACCATGTTCGGCATGGTC
CTGGCGATCGGCATCCTGGTGGACGACGCGATCATCGTGGTGGAGAACGTCGAGCGG
CTGATGGCCGAGGAAGGCCTGTCGCCGCACGACGCCACGGTCAAGGCGATGCGCCA
GATCAGCGGGGCCATCGTCGGCATCACCGTAGTGCTGGTCTCGGTGTTCGTGCCGATG
GCGTTCTTCAGCGGCGCGGTGGGCAACATCTACCGCCAGTTCGCGGTGACCCTGGCG
GTCTCCATCGGCTTCTCGGCGTTCCTCGCGCTGTCGCTGACCCCGGCCCTGTGCGCCA
CCCTGCTGCGCCCGATCGACGCCGACCACCACGAGAAGCGCGGCTTCTTCGGCTGGT
TCAACCGCGCCTTCCTGCGCCTGACCGGACGCTACCGCAACGCGGTGGCCGGCATCC
TCGCCCGGCCGATCCGCTGGATGCTGGTCTACACCCTGGTCATCGGCGTGGTCGCCCT
GCTCTTCGTGCGCCTGCCGCAGGCGTTCCTGCCGGAAGAGGACCAGGGCGACTTCAT
GATCATGGTGATGCAGCCCGAAGGCACGCCGATGGCGGAGACCATGGCCAACGTCGG
CGACGTCGAGCGCTACCTGGCGGAGCACGAACCGGTGGCCTACGCCTATGCGGTCGG
CGGCTTCAGCCTGTACGGCGACGGCACCAGCTCGGCGATGATCTTCGCCACCCTGAA
GGACTGGTCGGAACGCCGGGAGGCCAGCCAGCACGTCGGCGCCATCGTCGAGCGCA
TCAACCAGCGCTTCGCCGGCCTGCCCAACCGTACGGTGTATGCGATGAACTCGCCGC
CGCTGCCGGACCTGGGTTCCACCAGCGGCTTCGACTTCCGCCTGCAGGACCGTGGCG
GGGTTGGCTACGAGGCCCTGGTCAAGGCCCGCGACCAGTTGCTGGCGCGCGCCGCC
GAGGACCCGCGCCTGGCCAACGTGATGTTCGCCGGCCAGGGCGAGGCGCCGCAGAT
CCGCCTGGACATCGACCGGCGCAAGGCGGAGACCCTTGGCGTGAGCATGGACGAGA
TCAACACCACCCTGGCGGTGATGTTCGGCTCGGACTACATCGGCGACTTCATGCACG
GCAGCCAGGTGCGCAAGGTGGTGGTCCAGGCCGACGGCGCCAAGCGCCTGGGCATC
GACGACATCGGCCGGCTTCACGTGCGCAACGAGCAGGGCGAGATGGTGCCGCTGGC
GACGTTCGCCAAGGCCGCCTGGACCCTCGGCCCGCCGCAACTGACCCGCTACAACG
GCTATCCCTCGTTCAACCTCGAGGGCCAGGCCGCGCCGGGCTACAGCAGCGGCGAAG
CCATGCAGGCGATGGAGCAATTGATGCAGGGACTGCCCGAGGGCATCGCCCACGAGT
GGTCCGGCCAGTCCTTCGAAGAACGCCTGTCCGGCGCCCAGGCGCCGGCGCTGTTCG
CCCTCTCGGTGTTGATCGTGTTCCTCGCCCTGGCCGCCCTCTACGAAAGCTGGTCGAT
CCCGCTGGCGGTGATCCTGGTGGTGCCGCTGGGCGTACTCGGCGCACTGCTCGGGGT
GAGCCTGCGCGGTCTGCCCAACGACATCTACTTCAAGGTCGGCCTGATCACCATCATC
GGCCTCTCGGCGAAGAACGCCATCCTCATCATCGAGGTGGCCAAGGACCATTACCAG
GAAGGCATGAGCCTGCTGCAGGCGACCCTGGAGGCCGCGCGCCTGCGCCTGCGACC
GATCGTCATGACCTCGCTGGCGTTCGGTTTCGGCGTGGTCCCGCTGGCGCTCTCCAGC
GGCGCCGGTTCCGGCGCCCAGGTCGCCATCGGCACCGGGGTGCTCGGCGGGATCGTC
ACCGCCACGGTACTCGCGGTGTTCCTGGTACCGCTGTTCTTCCTGGTGGTCGGGCGCC
TGTTCCGGTTGCGCAAGGCGCCGCGCACCGGCAACTCGCCCCAGATCCCCACGGAGC
AAGCCTGA
MRSN2101_R1_pyocin
SEQ ID NO: 18
TGAAGCACCGGAACCCGGCCCTGGCGCCGGGTTTTTTATTGCCCGGGAAAACGTTCG
TGCGGACGTGCCCGGCGCTGCCTGCCAAGGAGGGACCCATGGGCAACGAACCGCAG
ACACTGACGGAAATGCCGCTCTGGGTACTGATCCTGCTCGCCGCGCTGGGCGGCGTC
AGCGGCGAGATGTGGCGTGCCGACAAGGCCGGTCTCGGCGGCTGGGCCTTGTTGCG
GCGCCTGGCGCTGCGCTCCGGCGCGTCGATTGTCTGCGGCGTGGCGGTGATGCTGCT
GGCATTGGCTTGCGGCGCCGCGCTGCTGTTCGCCGCGGCGCTGGGCAGCCTGACCGC
CGCGGCCGGCGCGGAGATCGCAGTCGGTCTCTACGAACGCTGGGCCGCCCGGCGCCT
GGGGGTTTGCGAGCTGCCCGAGGAACAGTCGGGCGACCGTGGCCCGCATTGAAACT
GATCGAAGGAGTCAACCATGCCTGAACAGGCTGTCACGCTCGAGGCTCTGTACGCGG
CCATCGAGCAGGTACTGCGTGAGCGTCTGCCGGAGGCGCAGTTGATCGGTTTCTGGC
CAGGCGTGCCGGAAAATACCCCGGCGGTTTCCCTGGAAATAGCCGAGCTGCTGCCCG
AGCGCGATCCCGGTACCGGCGAGAGCGCCCTGCTGTGCCGCCTGCAGGCGCGGATAA
TGGTGCCGCTTGGTGGCGATCGCCAGGCGGTATCCATTGCTTGCGGAATCGTTCGGAC
ATTGCGCGAGCAGACCTGGAACCTGTCTCTGGAACCGGCGCGCTTCGTACGCTCGGC
CGTCGACGGCAGTCGCGAGGAGCTGAAGAGCCTGCGTGTCTGGCTGGTCGAGTGGA
CGCAGTCGCTGCGTCTCGGAGACCCGGAGTGGGCCTGGGAGGACCAGCCGCCGGGC
AGCCTGATGCTGGGCTTCGACCCGCAGACCGGCCCCGGCCATGAGCCGGACTACTTT
GCTCCGGAGGCGTTGGCATGAGCTATGTCAGTGCGGAGCATGACCGCATGCTCGCCG
CGATGATCCTGCCCTGCGTGGTGGTCGCCGTGGACCTGGCAGCGGCGCGGGTACGGG
TGCGCTCCGGCGACTGGACCAGCGGCTGGCTGCGCTGGCACTCCCTGGCAGCCGGC
AAGGTTCGCCACTGGCGTGCGCCGAGCATAGGCGAACAGGGGGTACTGCTCAGTCC
GTCGGGCGGAGTGTCAATGGGTACCTTTATTCCCGGTCTGTACGGCGATGCGGGCACG
GCGCCGGACAACAGCGCCAGCAGTGAGACCTGGCGTTTCGACGACGGCGCCTCGTT
GAGTTACGACTGGGCTGCGCATCGCTACCGCGTCGAGCTGCCCAGCGGCACCGTGGA
AGTGAGGGTCGGCGCCAGCGAGGTGCGGGTCAGCGACGGGGCGGTCAGTCTCAAGG
CGCCGAAGATCAGCCTGGAAGGACCGGTGGAGATCGCCGGGACACTGACGGTCAGC
GGAGACATCCTCGGCGGCGGCTCGATCATCGACACCGCCGGCAACAGCAACCACCA
CACCCATTGAACATACGACGGGGCTGCCGAAGGGCGGTCCGTCGAACATTCAACCGG
CCCGCGCGAGCGGGCCTTTTCGTTTGCGGAGTTCGCCATGGGCAACACTCACAGCCA
TTCGGAGCCAGGCGCGGCCTGTCTCTGCCGGAGCGGGAGGGCAGGGCGATGATCGG
GATGGATCGCCGTAGCGGGCTACCCCTGTCCGGCCTGGCTCATCTTAAACAGTCCGTC
GAGGACATCCTGACCACCCCGTTGGGCAGCAGGCGCATGCGCCCCGAGTACGGCAG
CAAGCTGCGGCGGATGGTCGACATGCCGGTGAGCGAAGGCTGGAAAAGCGCCGTGC
AGGCCGAGGTAGCCCGTTCCCTGGGGCGCTGGGAACCGCGCATCAGATTGTCTGCCG
TGCGAGTCGTCGCGGTCGTCGATGGCCGCGTGGATCTGCTCCTGAGCGGCGTGTTCG
AGGGCGAGAACATCAATATGGAGGTCTCGGCGTGATCATCGATCTTTCCCAGTTGCCG
GAGCCAGAGGTTATCGAAAACCTCGATTTCGAGACGATTTACCAAGAGCTGTTGGGC
GACTTCCGCGAAGCCATGGCTGGCGAATGGACAGCGGAGGTGGAGTCCGATCCGGTT
CTCAAGCTTCTGCAACTGGCGGCCTATCGAGAACTGCTGCTGCGGGCGCGGATCAAC
GATGCGGCGCGGGCGGTGATGCTGGCATACGCCAGCGGTGCCGATCTCGACCAGATC
GGTGCCGGCTTCAATGTGCAGCGTTTGCTGATCAGGCCCGCTCAGCCCGAGGCGGTA
CCGCCGGTGGAGGCGCAATACGAGAGCGACAAGTCGCTGCGCAATCGCATCCAGCTC
GCGTTCGAGCAGCTATCCGTCGCAGGACCGCGGAACGCCTATATAGCCCATGCGCTGG
GCGCGGATGGAAGGGTGGCGGATGCCTCTGCGACCAGTCCGGCGCCCTGCGAAGTG
CTGATCAGCGTGCTCGGGGTGGAAGGCAACGGGCAGGCACCGGAAGCGGTGTTGCA
GGCAGTGCGCCTGGCGCTGAACGCGGAGGACGTGCGTCCTGTCGCGGATCGGGTAA
CGGTGCGCTCGGCAGGCATCGTTCCCTATCAGGTCAAGGCGCAGCTCTACCTGTTTCC
CGGTCCCGAGGCCGAACTGATCCGTGCCGCCGCCGAGGCTTCGCTGCGCGACTACAT
TTCCGCCCAGCGCCGCCTGGGCCGCGACATCCGGCGTTCGGCCCTGTTCGCCACCCT
GCATGTCGAAGGCGTGCAGCGCGTCGAACTGCAGGAGCCTGCGGCCGACGTGGTCC
TGGATGAAACCCAGGCGGCCTATTGCACGGGGTACGCGATCACCTTGGGAGGCGTCG
ATGAGTAGCCGCCTGCTGCCGCCAAACAGGAGTTCTCTGGAACGCTCTCTGGGTGAT
GTATTGCCTGCCGAACTGCCGGTGCCGCTTCGTGAGCTTCACGATCCGGCACGCTGTG
AGGCGGCCTTGTTGCCCTACCTGGCCTGGACGCGCTCGGTGGACCGCTGGGACCCGG
ACTGGAGCGACGAGGCCAAGCGCAATGCGGTAGCGACGTCCTTCGTCCTGCACCAG
CGCAAAGGCACGCTGACCGCGTTGCGCCAAGTGGTCGAGCCGATCGGTGCGCTGAG
CGAGGTCACCGAATGGTGGCAGCGAAGCCCGACCGGCGTGCCGGGGACCTTCGAGA
TCACCGTGGACGTCAGCGACCGTGGCATCGACGAAGGCACCGTACTGGAGCTGGAG
CGCTTGCTCGATGACGTCCGCCCGGTGAGCAGACACCTGACCCGGCTGGACCTGCGC
ATTACCCCGGTAATCCGGTCCCGTCACGGACTGGCCGTGACCGACGGCGACACCCTG
GAAATCTTCCCTTGGAAACAGTGACATGACGACCAATACTCCGAAATACGGTGGCCT
GCTCACCGACATAGGTGCCGCTGCGCTGGCTGCGGCCAGTGCAGCAGGCAAGAAAT
GGCAGCCGACTCATATGCTGATCGGCGATGCCGGCGGTGCGCCGGGCGACACGCCGG
ATCCATTGCCTTCTGCGGCGCAGAAGAGCCTGATCAACCAACGCCATCGGGCTCAGC
TGAATCGGCTGTTCGTTTCCGACAAGAACGCCAATACCTTGGTTGCCGAGGTGGTGC
TGCCAGTTGAGGTAGGTGGCTTCTGGATCCGCGAGATCGGCCTGCAGGATGCCGACG
GCAAGTTCGTCGCGGTATCCAACTGCCCGCCCAGCTACAAGGCTGCAATGGAAAGTG
GCAGTGCGCGGACCCAGACCATTCGGGTGAACATCGCGCTCTCCGGCCTGGAGAATG
TCCAGCTGCTGATCGACAACGGCATCATCTACGCCACTCAGGACTGGGTGAAGGAAA
AGGTCGCTGCCGATTTCAAGGGCCGCAAGATTCTGGCTGGCAATGGCTTGGTCGGTG
GGGGCGATCTTTCTGCCGACCGCAGCATTGGTCTGGCGCCTTCCGGCGTGACGGCGG
GCAGCTATCGTTCGGTCACGGTGAACGCCAACGGGGTGGTCACCCAGGGCAGCAATC
CGACCACCCTGGCCGGCTATGCGATCGGAGATGCCTATACCAAGGCCGATACCGATGG
AAAACTGGCGCAGAAAGCGAACAAGGCCACCACCCTGGCCGGCTATGGCATTACCG
ATGCGCTGCGAGTCGATGGCAACGCCGTGTCATCCAGCAGGCTGGCCGCACCGCGTA
GCCTGGCAGCCAGTGGCGATGCCTCCTGGTCGGTGACCTTCGACGGCAGTGCCAATG
TTTCTGCGCCGCTGAGTCTTTCCGCTACCGGTGTGGCGGCGGGCAGCTATCCGAAGG
TGACCGTGGATACGAAGGGAAGGGTGACTGCTGGAATGGCGCTGGCGGCGACGGAC
ATTCCCGGGCTGGATGCTTCGAAGTTGGTCAGCGGGGTGCTGGCCGAGCAGCGTTTG
CCGGTATTCGCGCGCGGGTTGGCTACTGCTGTCTCGAACAGTAGCGATCCGAACACC
GCGACCGTGCCGTTGATGCTGACCAATCATGCGAACGGACCTGTTGCCGGACGATAC
TTCTACATCCAGTCGATGTTCTATCCGGATCAGAACGGCAATGCTTCGCAGATTGCAA
CGAGCTACAACGCTACATCCGAGATGTATGTACGGGTGTCCTACGCGGCCAACCCTAG
CATCCGGGAGTGGTTGCCCTGGCAGCGTTGCGACATTGGAGGCTCCTTCACGAAGAC
GACTGACGGATCCATTGGAAATGGCGTCAATATAAACAGCTTCGTCAATTCCGGATGG
TGGTTGCAATCGACATCGGAATGGGCGGCGGGTGGAGCTAACTATCCCGTGGGGCTG
GCCGGTTTGCTGATTGTCTACCGCGCACATGCAGACCATATCTATCAGACCTACGTAAC
ACTCAACGGAAGCACATATTCGCGCTGCTGCTATGCGGGCTCTTGGCGTCCGTGGCG
GCAGAACTGGGACGATGGAAACTTCGATCCGGCCAGCTACCTGCCAAAGGCGGGATT
TACCTGGGCGGCTTTGCCGGGTAAGCCGGCAACTTTCCCGCCCTCAGGGCATAACCA
CGATACCAGCCAGATCACCTCCGGCATCTTGCCTCTCGCTCGTGGTGGCCTTGGCGCC
AATACAGCGGCCGGAGCACGCAACAACATTGGTGCCGGAGTGCCGGCCACGGCGAG
CCGGGCGCTCAATGGTTGGTGGAAGGACAACGATACCGGTCTGATCGTCCAGTGGAT
GCAGGTGAACGTAGGAGATCATCCCGGTGGAATAATCGATCGTACCTTGACGTTCCCG
ATCGCGTTCCCTGGCGCCTGTCTGCATGTCGTACCGACTGTCAAGGAGGTGGGGCGA
CCAGCGACGTCCGCGTCGACCGTTACGGTCGCCGATGTCAGCGTCAGCAACACGGGA
TGTGTGATCGTTTCCTCCGAGTACTACGGACTGGCTCAAAACTATGGCATCAGAGTGA
TGGCCATCGGCTATTGAGGTGAAAATGATTTTTTTCCATGCCGCCACGGGCGGCTTTT
ATTCGAAGGAAATTCATGGGTCCCGCATGCCTCTGGAGGATGAAATGCATCCTCTCGA
AGACGCCGAGTACCAGGCTCTTCTTCGTGCGCAGAGCGAAGGGAAACGAATTGTCA
CGGACCACACCGGTCGCCCTATCTGCGTCGATCCACCGGCTCCGGCCAAGGACATTC
TGGTCCAACGGGAACGCATCTGGCGCGACCGGCAGTTACAGCTCACCGACGGGCCTC
TCGCTCGGCATCGTGACGAGCAGGACCTGGGAAAAACTACGACTCTGAGCCAGGAG
CAGCTTCGTGAGCTAACTCTCTATCGCGCCGTTCTTCGCGACTGGCCTATTGCCGCGG
AGTTTCCCGACCTGAACGCAAGGCCCGAGCCGCCTGCCTGGCTCCAATCGCTCATCA
CCCCCTGAACCCCGCCTTGTGCGGGGTTTTTCATTAATGGAGATCTACCTATGAGTTTC
TTCCACGGCGTTACGGTAACCAACGTCGATATCGGTGCGCGCACCATCGCGCTGCCGG
CCAGTTCCGTCATCGGCCTCTGCGATGTGTTCACGCCGGGGGCGCAGGCAAGCGCCA
AGCCCAACGTCCCGGTGCTGCTCACCAGCAAGAAGGACGCCGCCGCGGCGTTCGGC
ATCGGCTCGTCGATCTACCTGGCCTGCGAGGCCATCTATAACCGTGCCCAGGCCGTGA
TCGTGGCGGTGGGCGTGGAGGCCGCGGAGACTCCCGAGGCCCAGGCCAGCGCCGTC
ATAGGTGGTGTCAGTGCCGCCGGCGAGCGCACCGGGCTGCAGGCTTTGCTAGACGGC
AAGAGCCGCTTCAATGCCCAGCCACGTCTACTAGTTGCGCCGGGTCATTCGGCCCAG
CAAGCGGTGGCCACCGCCATGGACGGGCTGGCCGAGAAACTGCGGGCCATCGCCAT
TCTCGATGGTCCCAATAGCACCGACGAGGCGGCCGTCGCCTACGCCAAGAACTTCGG
CAGCAAGCGCCTGTTCATGGTCGACCCGGGCGTTCAGGTTTGGGACAGCGCCACCAA
TGCCGCGCGCAACGCCCCGGCTTCGGCCTACGCCGCCGGCCTGTTCGCCTGGACCGA
CGCCGAGTACGGCTTCTGGTCCTCGCCGTCGAACAAGGAGATCAAGGGCGTCACCGG
CACCAGCCGTCCGGTGGAGTTCCTCGACGGCGATGAGACCTGTCGCGCCAACCTGCT
CAACAACGCCAATATCGCGACGATCATTCGCGACGATGGCTATCGCCTGTGGGGCAAC
CGCACCTTGTCCAGCGACAGCAAGTGGGCTTTCGTCACCCGTGTTCGGACCATGGAC
CTGGTAATGGATGCGATCCTCGCCGGGCACAAGTGGGCGGTGGACCGCGGTATCACC
AAGACCTACGTGAAGGATGTCACCGAGGGCCTGCGCGCCTTCATGCGCGATCTGAAG
AACCAGGGAGCGGTGATCAACTTCGAGGTCTATGCCGACCCGGACCTGAACAGCGCC
AGCCAGCTGGCCCAGGGCAAGGTGTACTGGAACATCCGCTTCACCGATGTGCCGCCT
GCCGAGAACCCCAATTTCCGTGTCGAGGTGACCGATCAGTGGCTCACCGAAGTTCTG
GATGTCGCCTAAGGAGCGCCCGTGATGATTCCGCAAACCCTGACCAATACCAATCTGT
TCATCGACGGCGTGAGCTTCGCCGGTGACGTGCCATCCCTGACCCTGCCCAAGCTGG
CTGTGAAGACCGAGCAATACCGTGCCGGCGGCATGGATGCGCCGGTATCCATCGACA
TGGGCCTGGAGGCGATGGAAGCCAAGTTCTCCACCAACGGTGCCCGCCGAGAAGCG
CTGAATTTCTTCGGCCTGGCCGACCAGAGCGCTTTCAATGGCGTGTTCCGTGGCTCCT
TCAAGGGCCAGAAGGGCGCCAGCGTGCCAGTGGTGGCTACCCTGCGCGGCCTGCTC
AAGGAGGTCGACCCGGGCGACTGGAAAGCCGGCGAGAAAGCCGAGTTCAAGTACG
CCGTTGCGGTCAGCTACTACAAGCTGGAAGTCGATGGCCGCGAGGTCTACGAGATCG
ATCCGGTCAACGGTGTCCGCGCGATCAACGGTGTCGACCAGTTGGCCGGCATGCGCA
ACGACCTCGGCCTGTAAGAGGAGCTCCGGACATGACCCAAGAGAATCGACTGCCGG
GCTGGCTGACGCTGGATGCCGACGCCGCCCTCGTTCGTCTCTCGCGTCCGGCACAGT
GCAACGGGGTCAGCGTCGACACGCTGACCCTGCGTGCACCCACCGTGCGTGATATCC
GCCTGGCCGGCAAGGTGGCCGGCGACGATGCCGAGGAGCGCGAACTGCAACTGTTC
GCCTCGCTGGCGCAGGTCAGCCGCCAGGACCTGGAGGGGCTGAAGCTGAGCGACTA
CCAGCGGCTGCAAGGCGCCTACTTTCGCCTGGTGCAAGACGACACGGACGACACCTT
CGCGTATGCGTCAACTGGCGAGGCGCCTGGCCATTGAGCTGGGCTTCCAGGCCAGCG
AACTGGAGCGCATGACCCTGGGCGACCTGCTCTGGTGGCTCGCCGAGGGCGAGGAG
TGAGCGACGGCGCCGTCCGCCAGGCGAGCGGCGCCCGCGCTACTTCAGTAGGTCCG
CGGAGGCGGACATCGATAACAGACGAGACGAACCGTCATGAGTAAAGACATGGACC
TGGTGGTCTCCATCGGCGGCATCGCCGACCCCTCCTTGGGCAAGGCATTCGAGACCG
TCAAGGCACGGCTCGACAGTCTCCAGGAACGTTCCCGCCAGGCTTCCAGCCTGAGG
GATGTGCTGGGAGACGCCATACGCCTGGAGCGAGAGCTCGCCGATATGCGCAAGGTC
GGGGACCGCGGCGTTGCTGAGCATGCCCGGCAGCTTGGCGAACGCCAGGAGCAACT
GAAGCGACTCGGCATCGAGGCCAGGGCCGCGGGCGATGCCTACGCTCGACTGGGCG
AGATGCAGCGTGGCCTGGATATGCAGGTCCGCGGCCTGCAACGGCTGGAGCAGGCCA
GCCAGGCAATGCCATTGGCTAGCGCATTTTCCGGACTGGTCGTGGAAGCCAGCAAGA
CGGCTGCCGGTTATCAAGCGCGGTTGCGCGACCTGGCGATCCGCAACGGCCTGGACG
TCGGCCGGGAGCCAGCCTTGGCATCCCTGATCCAGGACAGCGCCAACCAGAGCGGC
CTGGGACGCACGGCGACGCTGGACATGCTGGAGCACTTGAACGCCACCGGCATGGG
GTTCGCCGCCGCGCAAATGAATCTGGGACTGGCGGGCCGCTTCGGCTTTGGCCAAGG
GATTGCTTCAGCCGAGGTTGCGGGGCTGGTTCGAGCGTTGCAACTGGCCCAGGGTTC
GGACTCGCCAGAGCAATTGTCCGCCTCCCTCGACCGTCTGGTCGTCCTGGGTAAAGG
CAGAGTCGGCAGTGAGGCCCTGGCGCGTCGCCTGCCTGCCTTGTTGTCAGCGCTGGG
CAATGCGGGAGAGGCCACGGCCGGCGATGTCGGTGCACTGGGTGCCTTGCTGGAGAT
TCAGGCAAAGAACACCACGCCAGACAAAGCGGACGTGCGGATGAAGGCCTGGCTGG
AGTTCGTCGGCAGCGGCAGCTTGAAACGCGCTTATGGCCAGGACTACGACCGGGACC
TGGAAGCGCTGCGCAAGGACGGAGCGAGTCTGCTGGAGGCCAACCTGGAGCTGGCT
GCGCGCTATCGGGATAAGGGCGGCAAGCTCAGCGCCGGCGTGGCGTCGCCGGCGCTT
GAAGCCTATCGAGCGTCGCGCGGCGAGTTCCAGGGCTTGCTCGAATCCCAGCAGTCT
TCCGTAGGCTCTTCGGAGCGCGATGCGCAGCGTCGCAAGGGGATGTCCCAGGAGCTG
TGGAAGGCTTCCAGCGACAGTTGGGAGAGGGCGCAGACCGCTTTGGGCAGCGCCCT
GAATCCATATCTGGACAACCTGGCCAAGGGGAGCGCGGTACTCGGCGAGTCGACTGC
GGAGCTGCTCGAAGCCTATCCGCGGACGACGGCCGGTCTTACCGCCGCCGCAGGTGC
GGTGTTATCCGGGTATCTCGCCTACAAGGGAGGGCGCGGCGCTATCGACGTGCTGCGT
GGCGGTCGGCTCGGTCGGCGAGGGACCGCTGCCGTCGGCGACCTGATCGAACGGGG
TGCAGGCCGGGTATCGGGTGGTAGCGAAATACAGCGCGTGTTCGTTACCAACTGGCC
GGTGCCGGGAGGCGACTCTACGCTGGAGTCCGCGAGGAGGCCAGCACAACGAAAGC
GCGGTCAAACACCACGCAGGAAGAGAGGCAAGGGCGGTGGGCTGAAGGCCCGCTC
CCTTCCATCCCTTGGCTTCTCGGCTGGAGGAGGCTTGGGGGCGATGGCAGGAAAGCT
GCCGCGCCTGTCGCGTTTGCCCATACGCAACGCGCCGCTGCAAGTGGCCTCGTCGTT
GATCGATGTTGCTGAGGTCTACTCCAGCGACCTGTCGGAGAGCGAAAAGACCGTTGC
CTATGGCGAGGCAGGTGGCTCCCTGGCTGGTTCCCTGGCTGGCGCCGCTCTGGGAGC
GAGCATCGGCTCGGTGGTGCCGGTGGTCGGTACGCTGATCGGTGGATTGGTTGGCGG
CGCTATCGGCGCCTGGGGCGGTAGCGAACTGGGGGGGCGCCTGGGGCGCAGTCTGG
CTGGCGATCCGCCGGCGGCCTCGGACAACAAGCCGGCGGTGGCCGTACCACAGGCC
GGACCCGTCGCGGCTGCGCCCAACTGGACCTTCGCGCCGCAGATCAACCTGACGGTG
CAAGGCAACGTGCACGAGCCGCAGCGCCTGGCCGACGAGTTGCTGCCCTACCTGCA
ACGCATGCTTGTCGACTTCGCCGACGAGCGGCAGCGGCGCAGCCTCTACGACCCGGC
GATGGTTTAAGGAGTCCCCATGGCATATCTGGAACAATTGCAGGCCGGCCTGAGGTAC
CTGGGTCGCGCCGGCGAGTCCGGACGCAAGAGTCTGGACAAGGTGGTCGCTCCGGT
GAACGGCGCGATCAGCGAGATCCGCGGCGCAGCCGCGGAGCTGGAGAACCTGCCCG
GCGTATCGCCGGAAATGGCTGCCCGGCTGCAGCGTGCCATGCGCGGCATCGGCCAGG
CGCAGGGCAAGGTGAACCGCGTGGTCTCCACCTATGACCGGGCGAGCCGGGCGTTG
CTTGGTATCGACGAACGCCTGGATGCGCTGAAGGTGCAGGTGAACAGTGCCGCGCAG
GCGGTCGGCAAGGTCGCTGGCGACATCAGTCCGACGCTGGCGGGGGTGCTGCCGTC
GTGGCTGCTGGCACCCTCGGCGACGCCCCCGAGCGAGGCCGCAGCGCCCTTGCCGC
ACCTGCTGGTACTGCAGCCGCTGACCGCCAATGCCCAACCGTTCTACTTCAACCTGA
ATACTGCCGCCTTCGACGCCCTGCAGCGCAACAGTGCCTACAACTGGAGCGGGCAGG
TGCGCCTGGGTCGGCGGCCGGCGCTGCAGAGCGTCGGCATGGGCGAGGAGAGCATC
CTGCTCAAGGGTGCGGTATTCCCGCTGCGTCGACAGGTAGGTAACCAGGAAAAGGTC
GTCGGTCTGGAGCAGCTCGAAGCGCTGCGCCGGTTGGCGGAGCGGCGTGAGCCGCT
GATCCTGAGCAGCGGCTACGGCGAGGTGCAGATGGGCCTCTGGTGTCTGGTGCGGAT
CAGCGAGAACCAGAGCGCCCTACTGGGCAACGGCGCTCCCCGCAAACAAACCTTCG
ACCTGGAGTTCAAGCGCTATGGCGACGACCTGCCGAACCGCTGACGGCGAGATGCTG
GACAGCCTCTGCTACCACGTCTATGGCCATCTGTTGGGCTGCGTCGAGGCGACCCTCG
ACGCCAATCCCGGGCTGGCCGATGAGCAGCAGCCATTCCGCGCCGGCTTGCTGATCA
GTTTCCCTGACATGCCGGTGGTCAATGTCGAACAGGTGCGCCTGTGGGATTGATCGAC
CGCTCACCCGCAACCCCGCCTTGGCGGGGTTTTTCTTTTCTGGAGAAACCAGGTGCA
ACCGAGTTTCCGTATCGTTGCCGACGGCACCGACGTCACCCAGCGGCTGAATGACCG
CCTGCTCAAGCTGACCCTGCTGGACAAGCCGGGCATGGAGTCCGACAGCCTGACCTT
GAGGATCGACGATCGCGATGGACAGGTGGCCTTACCCAGGCGCGGTGCGGTGCTGGA
GGTTCATCTCGGCTATGCCGGCGAGCCACTGATGCGCATGGGACGCTTTACCGTGGAC
ACCTTGCAGTGGGCTGGTCCGCCGGACTGCCTGACCGTCACTGCCAAGGCCGGCGA
CATGCGCGGCAGTGGCAAGACGATACGCAGCGGAGGTTGGGAGGGCACTACCCTGG
CTCAGGTCTGCCGCGATGTTGGCGCACGCAACGGCTGGCGCGTGGAGTGTCCGTTGC
AGGTGGCGATCGCCCGGGTCGACCAGGTCAACGAGTCCGACTACCACTTCGTCACCC
GTCTGGCGCGCCAATACGACTGCACCGCCAAGCTGGCCGAGGGCATGCTCATGGTGC
TGCCGCGACAGAGCGGGCAGAGCGCCACGGGGCGTCGGATCGAACCTTTGGTGTTG
GGACGTGCTGACGTCGGCAGCTTCGACGTTACCTACGACGACCGCAGCCTGATGAGA
ACGGTGAAGACCCGCTACCAATTGCCCGGCAGCGGCGAGGTCAAGAGCGTCGAGCT
GAAGAACCCGAAGGCACCGGCTACGGCTACGGGCGAGCATGTCGACCGGCATCTCTA
TGCCAGCCGTGGAGAGGCCGAGCAGGCGGCGAAGGCTCGCCTGGCGAGCTTCAGCC
GCTCCAGTGCCAGTGTGCGCCTGGAGCTGCCAGGGCGTGGCGACCTGTTCGCCGAG
CGCAGCCTGCTGCTCCAAGGCTTCAAGGCGGGAATCGACGGCGAGTTCCTGATCGAC
TCGGTGGAGCACACCTACAGCTCCAGCGGATGGACCACTGTCGTGCAATGCAACGGC
GGCCGAGGCGGCAAGGGGTGACGCCTGAAACCCACCGGAGTGCAGGAGGATCGATG
AAACTGACCGAGCAGCAATTGCTGCGCATTTTTCCCAACGCCCGCCTCGTCGCGGGC
GTTTTCGTTGCGGCGTTGCAACGGGCCATGGACGAGAGGGAGATCGACACACCGGC
GCGGCGTACCGCGTTTCTCGCCCAGGTCGGCCACGAAAGCAGCCAGTTGACCCGGCT
GGTGGAGAACCTCAATTACAGCGCCCAAGGCTTGGCGGCGACCTGGCCGGGTCGCTA
TCTCGGCCCCGACGGGCAGCCCAACGCGTTGGCCTTGCGGCTGGCGCGCAATCCGCA
GGCGATTGCCGACAACACCTACGCCACGCGCAACGGCAATGGCGACGAAGCGTCCG
GCGATGGCTGGCGCTTCCGTGGGCGTGGCTTGCTACAGATCACCGGGCGTGCCAACT
ACCGGTTGGTCGGCGAGGCCCTCGGCGAGCCGCTGGAAGCCGAGCCCTGGCGCCTG
GAGCAGCCCGTGCGGGCGGCCCGCAGCGCCGCCTGGTGGTGGGCCGGTCACGGGCT
CAACGAGCTGGCCGACCGCGGCGAGTTCGCTGCCATCACCCGCCGCATCAACGGCG
GCCTGAATGGCCAGGCGGAGCGCCTGGCGTTGTGGCAGCGGGCCAGGGCGGTGCTG
TCATGAGCCGGCTTGCTCTGCTCCTGCCGGCCGTGTTGCTGGTCTTGCTGGCCGGCGC
CTTGCTCGGCGGCGGCCTGGTTGCCCGCCATTATCGTCCGCAACTGGAGGAGGCCCT
GGGCCAACTCACTGCCAGCCGCGTCGCCAGCGGCCAGCTCGAGGCTTTGCTCGATGA
GCAGCAACGAGCGCTGGCGGCGGTGCGGGCGAGCGCCGAGAGGCGCGCGAAGGAT
GCCGAGCAGGCACTCGGCGAGGCCAGGGCGCAAGCCGCGGAGCAGTATGCCGCGGC
CGTGCGTCTGCTCCAGGAACCCGACTTTGGCACGGACTGCCAGGCGGCAGGTGCGG
CGATCGACCGGGAGCTGGGACTATGACTCGTCTCCTGCTGGGGCTTTGTCTGCTTTTC
GCGGGCTGCGCAGCCTCACCGACGACACCTCGCCCAGTGCGCGTCGAGGTTCCCCTG
GCAGTGCCCTGCCGTGTACCTGACGTGCGCCCGCCGAGCTGGGCCGGCGCCACGCTG
AAGGCCGGCGATTCGCTGCAGGCCAAGGTTCGCGCATTGCTCGCCGAGCGCCGCCAG
CGGCAGGGCTACGAACTCGAATTGCAGGCGGCATTGCGTGCCTGCCGCTGAGACACT
GGGCGCATATGGACGTCGATGGACGTGTGTCCGGCGCATATTGCCGTGTATTGATTCC
GCAGCGTTGTCGAGCCGGGAATCGGTCGGTACAACGTAGTCATGCTTGTACAGGTGT
GTCCCCCCAGGGATGTCACCTGCAACCTCAGAGCCCGGCCAGTGTGCCGGGCTTTTT
CGTTTGCATCCGACAACGGCTCGGGACGTGGAGGCTCCTCGCCGACCGCGTACCGCG
CCACGGCTGACCGTCCTGGAGGCGGGGCCAGACGACCCGCCTGGTGCGGGTCTTTTC
ATGTGCATAAAGGAGAGTTTTCCATGTCCATCCTGACTCAAGGTACCCAGATCTACGC
CCTGGTTCCGCCGGTATCCGGTACCGGTGCCGCTACCGTCCTGGAGATCGAAGGCGT
GACTTCGTTCAACCCGGGCGGCAATCCGGCCGACCAGATCGAAGACCCGTGCCTGAG
CGACACTTCGCGCAAGTACAAGAAAGGCCTGCGCACTCCTGGCCAGGCGACCCTCG
GCATCAACGCCGATCCACGCCTGGCCAGTCACGTTCGGTTGTTCCAGCTGTCCGAGA
AAGATGGCGAGACGAGCGTCAAGTGGGCCATCGGCTGGTCCGACGGAATCGACGTA
AAGCCGACCGTCAGCACCGAAGGCGACGATTTCGTGCTGCCGCCGGCGCGCACCTG
GTTCACCTTCGAAGGCTACGTCAGCGACTTCCCCTTCGATTTCGCCAGCAACACGCTG
GTCGCTACCCAGGCCACCATCCAGCGTTCCGGCGCCGGCAAGTGGACGCCGAAGTCG
GCTTGAGGAGCGCGTAGATGAATCTCAACGAACTTCGTGCGGCGGGCGGTTTCATCG
AGTCGGCGCTGGTGCGCAAGGAGATTACCTGGACCCGCGTTCCCGCCGGCAGGAAG
AAGGCGGTCAGCGACACCTTCCAGGTGTTCGTCCGACGCAACAGCTTTGGCGCGGT
GGAGCGCCTGTTCTCCGCCGAAGGCGACCAGCAGAGCCGCAACGCGCGCTACCTCG
CCGAATGCATTCGCCTGGGCGAGACGGGAGAGGAAAGCCTGACCTACGAACAGGCC
TACGACCTCGACCCGGCGCTGGGCTTCCTGCTGCTGCAGGCGGTCGGCGAGGTCAAC
CGGGTCGAGGACGCGGAAAAAAACTGACCCCCGCCGACGAGGTTTGGCACGAACTG
GTGCTGAACGGCGTCGGCGGGTCGACCATTGCCGAGGCCAAGGAGCGGCTCAGCTA
CGCCGAGTACCGCGCCTGGGTCGCCTATCTGAACAAGCGCGGCTCGCTCCATCCGGG
ACACCGGCTGGAGCTTGCGCTGGCTCGGATCGCTGCGTTGCTCGGGCATGCGCTGGG
AGCGGACGCCGATCCCGACGCGTTCCGTCCGCATATGGCTCTCCAGCCCCTTTCGCTG
CATCAGGCGATGGATCAATGGGCATGACAGGCCACCCCGCTGCGGCGGGGTCTTTTT
CTGGAAGACATGAATCATGGCCACGAATACCGACGGCAGCCTGACGCTCGACCTGGT
CCTTCGCAGCGAAGGGTACAGGGCCGGGATGGACAAGGTTGGCAGGATCAACGATC
AGAAAATGCGTGCCATGGAGGCGCGCGCGGAAAAGGCTGGCAAGGCTATCGGCAAA
AGCCTGGACAGTTCGGCACTGATTGCCAGCAGCGTGCTGGACCAGGCGCTGGACATG
CTGGGCAGGACCAGTCGCCAGGCGGGTCAGGCCAAGAAGCCTGTGCAGAGCGCCCA
GGACAAGGTACTGGCCGAGTGGAAGACCCGGCAGAAGGAGCTGGGCGAAGCCTGG
AAGAGCTATCGCGAACCACTCCAGGATCTGTCCAAGCTCAACGAAGCACTACTGAAG
AACTCTTCCGACAAGCTCGACAAGGCGCTGCTCAATCTCAGCGAGACCGGCAAGCT
GTCGCTTGCCAACGTGGGCAAGGCCGCCTACGCCGATGCCGCGCGCCTCGCCTCGCG
GCAGATGACGCTGATGCTGCTGGACGGGCTGTTTGGCTGGGTCGCCAGCGTCGGTAC
CGAGAAGCCCAAGGTCGACGACAAGACGGGCAAGGGACAGGCGAAGGCCGGCGAC
GACGAGAAGGAACAGCCGTCGCTCCAGTCGCAGGTCTTCAAGCAGTGGCTGTTGCA
GATGAACAGTGTCTGGGGCGCCTACCGCGCGCCGCTGCAGGATATCTCCGGGATGAC
CGACGAGCTGTTCAGGAATGCGTCGGAGAAGCTCGAGAAGTCGCTGTTCAATTTCGC
CACTACCGGAAAGCTGTCCTTGAGCAACTTCGCCAAGACGGTGATCGACGATGTCGC
CCGGATCGCCGCGCGGCAGCTTTCAATGCTCGCCCTGGACGGATTGTTCGGCTGGAT
GAATGGCAAGGCCGGCATCACCGAGGCGCAACTGGCCAGCCAGAAGCCCTATACCTC
GCTACTGGAAAAGGCCCGCGCAGCTGCGGGACAAGCGGCAGCGGGCGCTCCCGCGG
CCCAGGGTGCCGCGCCAATGCCGGCAGCGGCGATGGATGTCGGCGCGATGGTGGCCA
CTGCTTCCGGGCAGACCGGGGACGGTTCCAAGGTATCGGCTGGAGGGGCTTCGGCG
AGCGCTGGCAAGCCGGTGGGCAGTTGGGTCGAACAGATGGACGCCTCCTGGGCGAG
CTTGCGCGACCAGGCGCAGGACGTCTCGGGAATGATGGACATGCTGTTTACCAACGC
CTTCACCAATATGGAGAACGCCCTGTTCACCTTTGCCACCACGGGCAAGCTGTCGTTC
AAGGATTTCGCCGACTCGGTGATCCAGGATATGGCGCGGATCGCCGCGCGGCAGGCG
ACGCTGCAGATCATCGGCGGCATCGTCGGTGCGGTCAGCGGGTTCTTCGGTAGCGGC
GCAACGGCGGGCTCGCGGATTTCCGACTACACCGGCTCGGACATGGCCAATTGGGTC
AGCAAGCAACGCGCCGGAGGCATGCCTGGGTTCGCCAGGGGCGGTGCTTTCAACGA
TGGCATCCAGAGCGCGCCGGCGCTGTTCAGCATGGCCGGCGGTCGTCCGGCGCTGAT
CGGCGAGCGTGGGCCGGAAGCCATCATGCCGCTGAGTCGCGGTTCCGATGGCGTGCT
CGGCGTGCGCGCGCTCGGCGGCGGCGAGGGTGGCAACGTCTTCAATTTCTCCACCAG
CGTCAGCCTGGGCGGCGGCCGCGAGGGCGCGGCGACGGCCAGCGGCGACGACGGTA
CGGGACAGCAGCTGGCGGGAATGATCAACGATGCCGCGCGCAACGTGGTGGCGCAG
GAGCTGCGCCCCGGCGGCCTGGTATGGAGGATGGTGAATGGCTGATCTGGAACGCTT
TACCTGGGACATCTCGATCGATTCCGCCGGCCAGGCGAACCAACTGGTGCGCCAGGT
GCAGTACGGCGGCGGCTACAGCCAGGCGCTCGGCGACGGGCTGAACAACCTCAGCG
AGACCTGGCAGGTTTCGCGTACCGGCGATCTCGCGCTGATCGGCCCGATCCGCGATTT
CCTCAAGCGCCACGGCGGCTACCGCTCGTTCCTCTGGACCTTGCCCACTGGCGAACC
GGTACGGGTGCGCGCCCAGGGCTGGCAATTGCGACCGCGCGGCAACGGCGTGTTCA
CCCTGAACACCACCTTCCAGCAAGTCTTCAATCCGTGAGGTAAGCATGACCATCACA
GCCGATGACCAGGCCCTCGAGCCTGGGGCGCTGGTGCGCCTGTTCGACCTGGATTGC
ACCGGGTTCGGCGGCGAGATGCTGCGCTTCCACGGCCACCTGCAGCAGGGGCCGATC
CACTGGCAGGGCAACGTCTACCAAGCTTGGCCGCTGGAGGCGCGCGGCTTCGAGCA
GCGCGGCGACGGCCGGGCCAGTTCGCCGACCCTTAGCGTGGGCAACATTGACGGCA
GCATCAGCGCGCTCTGCCTGTTCTTCGATGGCCTGGTAGGCGCGCGCCTGACCGTGC
GCGAGACCTATGCGCACTACCTGGATGCGGCCAACTTCGCCGAAGGCAACCCGCAGG
CCGACCCCTCCCAGGAGCGCCTGAACATCTGGTTCCTCGAGCAGAAGACCGCCGAG
AACAGCGTCCAGGTGACCTGGGAGCTGTCCGCTCCGCCGGACTTCCAGGGCCAGCA
GATCCCGGCGCGCCAGATCACCTCGCTGTGCCACTGGTGCATCACCAACGAGTACCG
CGGGCAGGACTGCAACTACACCGGCACGGCGATGTTCGATGCCGACGGCAATCCGGT
GGACGATCCGGCGCTGGACCGCTGCGGCGGCCGGGTCAGCGATTGCAAGCTGCGCTT
CGGCGCGGACAACCCGCTGTCCCACGGCGGCTTCGCCGGCGCCGGCCTGGTCAGGA
TGTGAGCATGGAACTGAGCCGCAGCCTGCAGCGGGCCATCGCCGCACACGCCGCCC
GCGAGCATCCGCGCGAATGCTGCGGGCTGATCGTTCGCGGTGTGCGTCAACGCCGCT
ACGTGGCCTGTCGCAACGCAGCCGGATCGCCCAGCGAGCACTTCGTGATCGATCACC
AGGACTGGTGCGCTGCCGAGGACCAGGGCGAGGTCCTGGCCATCGTCCACAGCCAC
CCGGACGTTCCGGCCACGCCGAGCATGGCCGATCGGGTCAGTTGCGAACTGCATGGT
CTGCCCTGGGTGATCCTGTCCTGGCCTGAAGGCGATGTCGCGCATCTAGCGCCGGAG
GGCTATCGGGCGCCGCTGCTCGGCCGCGAGTTCGCCCACGGCGTGCTCGACTGCTGG
AGCCTCTGTCGCGACTGGTACCGCCGCGAGGCAGGTTTGGAGCTTCCGGACTATCCG
CGCCGCGACGGTTGGTGGGAAACCGGCGAGAGCCTCTACGAGCAGCACTATGCGGC
GGCCGGATTCCGGCCGGTGCCGCTGGCCGGAATCCGCCGCGGCGACATGCTGGTGAT
GCAGGTCGGGAGGGCGCTGCACCCTAACCACGCGGGCATCTACCTGGGCAATGACTG
GCGTCTGGACAGCGAGCCGGTCCAGGCGCTCGGCGGCGACGGACCGTTCCTGCTGC
ACCACCTGTACGGACGGCTGTCGACCCGCGACGTGTTCGGCGGACCCTGGATCGAAC
GCACGCGCCTGGTCTTGCGGCACACGCAGATGCCGCAGTGAACGACATATTCAAGCG
AGCCGTCGGAATCGGCTCTTCACGAGAGGAACAGGTCCATGAGTGACACCCTGAGTC
AGGGCCTCACCACCATCCGTCTATACGGGGTTCTGGGCAAGCGCTTCGGCCGCATGC
ACGGCCGGTTGTTGGAAAGCGGCACGGTACGCGAGGCGATGAGTGCCCTGAAGCAC
ACCATGGAGGGATTCGAGACGTTCATGCGCGAGGCGGAGTCGAAAGGGCTGACCTT
CGCCGTGTTCCGCGGGCGTACCAACCTGTCCGGCGAGCAACTGGACATGCGCGGACG
CGAGGATATCCGCATCGTGCCGTTGGTGATCGGGAGCAAGCAGGCGGGGCTTTTCCA
GACGATTCTGGGGGCTGCCTTGATTGTCGTGGGTGGATTCACGACGTTCTTCTCCGGT
GGAACCAGTTCGTTCCTAGTAACCGTTGGAGTCAGCATGTTGGCGGGCGGCGTCATG
CAGATGCTCAGCCCCCAACCCAAGGGCCTGAAGGGCCGAGAGGCCCCCGAGAACGC
CCCCAGCTATGCCTTCGGCGGCCCGGTCAACACCATCGCCCAGGGCCATCCGGTCGG
CGTGCTCTATGGCAAGCGCCGCATCGGCGGCGCGGTGATCAGCGCCGGCATCTATGCC
GAGGACCGGCTGTAGCCGGCAACGCCGTAACAGGCCCGCCATGCGCGGGCGTTTTTT
TGCCTGAAGGAACGTCATGAACAAGACCATCACGGGCCACAAGGGTGGCAGCAAGA
AGCCGCGCCAGCCGGTGGAGATGCCGGACTCGGTGCGCTCGATCGCGCGGGCGAAG
ATTCTCCTGGCACTGGGCGAAGGCGAGTTCGACGGTGGCGTCGACGGCCGTTCGATC
TACCTGGACGATACGCCGCTGCTGGCGGCGGACGGCTCGGTGAACTTCCCCGGAGTG
ACCTGGGAGTTCCGTCCGGGCTCGGTGGACCAGGAACACATTGCCGGTGTGCCCGCC
GTGGAAAACGAACTGGCGGTCGGCGTCGAACTCAAGAGTGACGCGCCCTGGGTCCG
CGCGGTGAACAACACCCAGCTCTCGGCGGTGCGCCTGCGCCTGTCCTGGCCGGCCAT
CCAGCGCCAGCAGGAAAACGGTGACGTGGTCGGCTACCGCATCGACTACGCGATCG
ACATCGCCGTCGACGGCGGTGCCTGGCAGGAAGCGCTGAAGGCTTCGCTGGACGAC
AAGTCCACCAGCCGCTACGAGCGCTCCCACCGTGTCGACCTGCCGGAGGCGCGGAG
CGGCTGGCAGGTGCGCGTGCGCCGCCTGACGCCGAACCAGAACAACAACCGCATCG
CCGACACCATGCGGGTCGAGGCGATCACCGAGGTGATCGACGCCAAGCTGCGCTACC
CGAACACCGCGCTGCTGTTCGTCGAGTTCGATGCCAGCCAGTTCCAGAGCATTCCGC
AGATATCGGTGGAAGCGCGCGGCCGGCGGGTGCGGGTGCCGAGCAACTACGATCCG
CAGACCCGTAGCTACAGCGGCACCTGGGACGGCTCGTTCAAGTCGGCCTGGACCAG
CAACCCGGCCTGGCACTGGTACGACATCGTGTTGCACAAGCGCTTCGGCCTCGGTCG
GCGGATCGACGCGAGCATGGTCGACAAGTGGTCGCTGTACCGCATCGCCCAGTACTG
CGACCAGTCGGTGCCCGACGGCAAGGGCGGCCAGGAGCCGCGCTTCAGCTGCAACC
TGTACCTGCAGAGTCGCGCCGAAGCCTGGACCGTGCTGCGCGACCTGGCAGCGATCT
TCCGCGGCATGTCCTACTGGTCCGGCGCGGAAATGGTGGCGGTATCCGACATGCCGG
AGGACGAGGCCTACACCTTCTCGCCGTCGAACACCGTGCGTGGCGACGACGGCAGC
CACTTCAACTACAGCAGCAGCCGCCAGCGCGATCGCCACACCCTGGCCCTGGTCAAC
TACGACAATCCGGGCAACGGTTACCAGAGCCAACCGGTAGCGGTGAACAATGACCGC
GCGCAGCGCCGCTACGGCATCAGCCAGTTGGAGATCACCGCGATCGGCTGCACCTCC
GAGGGCGAGGCGCAGCGTCGTGGCCAGTGGGCGCTGCTGACCGAGGAGCTGGAGC
AGGACGCGGTGACCTTCCGCACCGGCATGGATGGCCGTGGGCTGGCGCCGGGGAAG
ATCATCGCCGTAGCCGACCCGGTCAAGTCCGGCAAGCAGATCGGCGGACGCCTGAGC
GCGGTGGATGGCCGCGCGCTGACCCTCGACCGCGACGTCGAGGCCCGACCCGGCGA
TCGCCTGCTGGTCAACCTGCCGAACGGCAAGGCCGAGGCGCGCAGCGTCCAGTCGG
TGGTAGGCCGCGTGCTGAGCGTGACCGCCGCCTATTCGGAGACGCCTCGGCCCCAGG
GGCAGTGGGCGCTGCAGAGCAACAGCCTGACCACCCAGCGCTTCCGCATCATGAGC
ATCACCCGGCCGGAGGACAATCTTTTCGAGATCACCGCGCTGCAACACAACGCGAGC
AAGTTCGACGCCATCGACAACGGTGCGCGCATCGAGCTGCCGCCGGTCACCAGCATT
CCGCCGGGCGTGCAGGCGCCGCCGCAGAACGTGCGGATCAAGGCTTTCACCAAGGT
CGACCAGGGGTTGGCGGTGACCAGCCTGTCGGCCTCCTGGGATGCCGCGCCGAACG
CGGTGGCCTACGAGGCCGAATGGCGCAAGGACTCGGGCAACTGGGTGCGGGTGCCG
CGAACCTCGGCGCTCGGTTTCGACGTGCCGGGCATCTATGCCGGTCGCTATCTGGTGC
GGGTACGCGCCTTGAACGTGATGGAGGTCGGTTCGGTCTACGCCAGCAGTGTGGAGA
CCGCTCTTGAGGGCAAGACCACGCCGCCGCCGGCGCTGGCCTACCTGCGCTGCGTGG
CCGGCCCCTGGCGCATCGGCCTGGAGTGGGGGTTCCCGACCAGCGGCGCAGCGGAC
ACCGCCTACACCGAGATCCAGCAGTCCGCCACGCCCGGCGGCAGCGAGGAGACCGC
ACGGGCGCTGGGCCTGTTCGCCTACCCAGGCAATACCCACCTGGTATCGCCGATACCG
GCCGGCGAACGGCTGGCGTTCCGCGGTCGCTTGATCGACCGTAGCGGCAACGTCGGC
GCCTGGTCGAACTGGGTCACCGGCACCAGCTCCAGCGACGCCAGCGAATACAACCA
GTTGATCACCCAGGAGTACGTCGAGTCGGCGCTGGGCCAGCAGTTCTTCTCCGATATC
GAACGGATGCAGGTGGATATCGGGGGCTTGCAGAAGCAGGTCGGCGACCTCGCCGA
CGTTCTGCTGTACGACCCGGCCAAGGTCTACGCGAAGAACGACATGGTGCGACAGG
GGCAGCGGTTGTACCAGGCACTGAAGGCTGTGCCGGCGAAGACGGCGCCGCCGAAC
GCGGCCTACTGGTCCGATATCGGCCAGTCGCTGGAAACCGCCAACGGGCTGGCGCAG
CAGGTGGCGAGCCATACCGCTGAAATCAGCGAACTCGACGGCAGCCTTACCGCCCAG
GCATCGCGCCTTGGCGTACTGCAGGCGGCGACCCGTGACGACGCGGATGACGGCAAT
GGCGCCATGGCCGATGCCCTGCGCGGCTGGAAGACCGTTGCCCGGGCAGCCCAGGA
GGAAACCGTACGGGCCACCGAAAACGAGGCCCAGGCCACTCGCACGACGCTACTGG
AGGCGCGCACCGCCGATGCCGAAGGGCGAATCGCCACGGTGGAACGGGTCGCGACC
AGCGATCGCCAGGCCACCGCGCAACGTCTGGACCAGCTCTCGGCCTCGATCGGTGGC
ACCGCCGCCAGCCTACAGAGCGAACAGACCGCCCGCGCTAACGCCGACAGCGCCCT
CGCACAGCGGATCGACACCGTGCAGGCGCGTACCGACACCAACAGCGCGGCGATCC
AGACCACCTCCCAGGCGGTCACCTCGCTGGATGGCAACGTCAAGGCGATGTACAGCG
TGAAGCTCCAGGCGCATGCCAACGGACAAAAGTATGCGGCGGGGTGGCAGCTGGGG
TTCGACAGTGGGACCAGTGTGTCAACCATGGCGTTTCAGGCGGATCGGTTTATCTGGT
TCGACAGTTCGAGTGGGGCAACGGTAGCGCCGGTTTCAATAGTTAACGGGCAGATGT
TTATCAAGAGCGCCCTGATTCAGGACGGAGCGATAGATAACGCGAAAATTGGAAATG
TCATTCAGTCAAATGCAATATCAAATAATGGTATGCCGATATGGAGGCTGGATAAATCA
GGTACTTATACAGTGCGAGACAGTGCAGGCCAAGTCAGAGTTGAGATGGGGCTTCTA
CAGTCATGAGTATCTATGGGTTACGTATCTATCGAGAGAATGGGGATGTCGCTGTCGAT
ATAACTGACAGGGCGTTGCGGGTTGTATATTCCAGGCGGGTCGATGCAGTTTCGAAAG
GAGAAGCTGCCACACCCGGCTTTGGCCCGAACAATGCTTCAGTCTATGTGATTGGTG
ATCAGTACAATAAGCGTCCGGTCTGGGCCCGAATGGGTGATGGCGTTGTTCAATGGG
GCTACGAGGATTGGTGGCCCTCACTATTTCACACTTCTGGAACCTTATATGTGGTGGC
CAAGGTATGAGTGAGTATGGGGTCTTGGTAAGGAATGGCCTGGGGCAGACTGTAATA
GATGGAGAGTTTCATAATCTTTCTCTTCTGTTGGAAAGAAATATTGAAGTCGAGACAA
GTCAGTGGTTCTCACTGGATTTTCCATATGCGGTTACCTCAGCTGCACCTCCTGTTCTA
GCAGTTCAAGCTTGGAAGAATAAATTTCTTTACTTCGACTCAGTACAGTATCGAGGCG
GGCCAGGAAACTGGACAGGCGCCAGTCTGAGTTTTTCGGGGTATGGAGCACATACAT
CCGGCTCCGTAAGAGTTCGAGTCTATGCCTATGCATTGCCTTTGCTGAGAGGATATGG
GCTTCGTGTTCGAAACTCGGCGGGAAGTGTCGTTTTCGATTCTCTAAGACTTCCCTTG
GTTTTTAGTGCGGAACTGGGCGGGGCGCCAGAAGATTGGGTTAGGGTATCTGGGGAA
CCGATTATAGGAGCTGGCCGCATAGACATATATCGCCCAGCGACATGGGGGCAGCCTG
CCGATAGCTACATTGCAGTAGGCATGGCGCTTGACGTGGAATTTGGCAGAGTGGCGG
TATCTGGTGGCACGGCCAACGCAATTATTTATATACGCTGGGGTTTCACTGAGGAGGG
AGTGCCAAGGCTTATGCAGCATGCATATACCGGGCTTCCAAGTTCTTATCCTGGAATTC
CAGCTGCTGTTTATTATGCTATGCCTTCAATTCCAATAATAAAGGCTTGAGTTGATTTTT
GGGAGATAATGCTATGGCTTGGCATTCGAAAGGTTCGGTTTCCGTCACGCTGAATTCC
GAAGCGGTGTTAGGAAGCGCTACTGATTTTATCGCCAATGTGCGAACCGGCGATGCGT
TCCGTGGCCCTGACGGCCGGTGGTACGAAATCACCAATGTGACCAGTGCAACGGTTA
TTTCTATCAAGCCCAATTATCAGGGCGCGACCGCTAGCGGCCAAGTTTATGCGGTTGT
CCCAGTACATGGTTATTCAAAAAACTTGGCAGACCAATTCCGCGATATCAACAACCAG
TGGGGAGCCACCCTGGCAGGGATCAAACCTTGGGCGGTGTCCTCGACGGGCCAGCA
GGCGCAGGCCGACATGGGAATCTCGGCTGTTGGTCGGGCCCTGAACAATGCCTCGAC
GCCGGCCAACGCCTTGAGTTACCTGGGGGGCGTCGCGCCCAATCAGATGGGCTGGGC
TGGCAACGCGATGAATACGGCGGACCTCGATTTGCTGACCGTCTCGGGGCTATACGCT
CATGGCACGGCGGTGCCTTCGCCGGTGAACAATGCCCAGGGCTATGTCTTGCATATGC
AGCACGGCAACCCGGACTTCGCCGTCCAGCAGTGGTACCAGTTGAATTCCGCCACTG
GCCAGTACATGCGGATCAAGGCCGGGGGTAACTGGTCGCGCTGGGTGTTGCAGTACA
GCCAGTTCAACCTTGTCGGACTGGCGAGCTTCGATGCATCGAACAATCCATCCGGCG
CCATCATCCAGCGCGGCGGGACTGTCGGTTTCAACGAGTATGTACGCTACGCCGACG
GAACCCAGATCTGCTGGGGCAACACCACTACCAATGTGGGAGCCACCATGGCCTACC
AGCCGGCTGGTACGTTGTCGTTCTATATCACTCCGGTTGCTTATTCGTGGGGGTTCCCG
GTCTCGTTCTCCCGGCCTCCGTCTGTCATGGTCAACCCGATGAGAGCAGCAGGAAAC
AACGCTTCCCGTCCCTGGGGTTCGACGATGTCGGTCACCGAAACGCTGTTTTCCTGG
TACGGCTACGACACCGCCAGCGTGGCCAGCGGGATGGCCGCCAGCTATGTCGCCATG
GGGAGGTGGAATTGATGAAGCTGTTGTTGAAACCTTTGTTGCAGCAGGGCATCACGC
CTGACAGGGAACGTCTCAGCGATGTCCAGGTGCGAGGGTCGGTGTTTATCCTGGATG
GCGTCGAATACGATTTCGGAAGAATGCAGCCTGGCGGGTACCTGCCTCCGGAGGCAT
ACCATGGTACGCCATTCATTGATATTCGCTTCGTGGACGGCGACCGCCTGTACCTGCA
CTACATCCACCAGGTGACCTCTGAAGTGATGATGCAGTTTCCCAGGGAGGTCGAGTC
AATTCCGGTCGACCAGGATGGAAAAGTCGAGGTAGCGTTCAGCTATGAACATCCAAT
GGGATAAGTACGTCAGCCCTGCAAAGGCCGCGGCAGATGTGCGCGATCAGGCCTTGG
CCAGTGCTCAGGCCAAGCGCCTTCTGGCCTATCGGGAAGAAAGCGATCCGCTCAAGA
CCGAAGCCGAGTTCGATGCGATCAAGGCCGGCGTCGAGCCGGACTATGGCGCCTGGA
TCGCCAAGGTGGAAGAGATCAAGTCACGATACCCGATGCCTGAATAGCCGAACTCCA
GGCTCGCCTGGGCGTCAGCCCTGTGGTTCCGAACTAACCCTATCAAGCCCGCCGACT
GCGGGTTTTTCATTATCTGGAGAACGTCATGCCTTGGTATTCCACAGGCACGGTTTCC
GTCGTCCTCAATTCGGACACGGTGACCGGCAGCGGCACCGCCTTCAGCGCCAATGCG
CGCGCCGGCGATGCCTTCAGAGGGCCGGATGGCCGTTGGTACGAGATCGGCAACGTC
ACCAGCGCCACCGTGTTGACCATCAAGCCCGCATATCAAGGAGCTACCGCCAACGGG
CAGGCATATTCGATCACCCCGGTGCAGGGGTACTCGAAGACACTGGCGGATCAGTTC
CGTGACCTCAGCAATCAATGGGGTTCCCTCCTGGCGGCAGTGAAGCCTTGGGCGATA
GCGTCTACCGGTTCGCAGGCGCAGGCTGACATGGGGATCACCGAAGTTGGCCGTGCT
ATCAATGGAGCCTCTACCGTAGGCAATGCATTGGGGTTTTTGGGAGGTGTTTCCAAGA
CCCAAGCCCCCATGGCCCTGGATATGGACACGGTGAACGAAAGCGGATGGTTCTCGA
TAACCCCCAATACTTACAACGTGCCTCTCGGAAACAACAATATCAGCGGTGTGAACG
GCCATGTCGCTCTGTCGATGGTATTCGACGCCAGTACCCGCTATCAGCTGTTCTTCGTG
AGAAATACCAACCTCCCAGAGGTTTGGTACAGGAGCTGTACCAATGGAACCTGGAAG
GAATGGGTCAGGTTTTATACGACAGACAATATCGTGGGGACCGTAACGAGGCGACTT
GTTACGGGTAAGCCCACCGGGGCTGTGATGGAGAGCGGGACAACTTCCAACGGCTG
GTACGTTCGCTTTGCGGATGGTACCCAGATGGCAGCGGCAAGATCCGAACCGGGCCT
TTCGTTTGGAGCTAACGTGATACAGCTACCAGCTGCCTTCGTTACGGGCTTCAATACC
GGGGTGACCTGCAATTGGATTCCCTCCAGCGGATGGCCTGCCACTGCAGGGCAGGGG
GTTCGTGGAGCCTATCTCAACGGTAGCAGCTCTGTTTCTTTCGCGACGGCGCAGGCA
CTGGGGGCTAACGACACCATTACGGTGATGGCTGTGGGGAGGTGGTACTGA
MRSN2101_R1_pyocin_tail_fiber
SEQ ID NO: 19
ATGACGACCAATACTCCGAAATACGGTGGCCTGCTCACCGACATAGGTGCCGCTGCG
CTGGCTGCGGCCAGTGCAGCAGGCAAGAAATGGCAGCCGACTCATATGCTGATCGGC
GATGCCGGCGGTGCGCCGGGCGACACGCCGGATCCATTGCCTTCTGCGGCGCAGAAG
AGCCTGATCAACCAACGCCATCGGGCTCAGCTGAATCGGCTGTTCGTTTCCGACAAG
AACGCCAATACCTTGGTTGCCGAGGTGGTGCTGCCAGTTGAGGTAGGTGGCTTCTGG
ATCCGCGAGATCGGCCTGCAGGATGCCGACGGCAAGTTCGTCGCGGTATCCAACTGC
CCGCCCAGCTACAAGGCTGCAATGGAAAGTGGCAGTGCGCGGACCCAGACCATTCG
GGTGAACATCGCGCTCTCCGGCCTGGAGAATGTCCAGCTGCTGATCGACAACGGCAT
CATCTACGCCACTCAGGACTGGGTGAAGGAAAAGGTCGCTGCCGATTTCAAGGGCCG
CAAGATTCTGGCTGGCAATGGCTTGGTCGGTGGGGGCGATCTTTCTGCCGACCGCAG
CATTGGTCTGGCGCCTTCCGGCGTGACGGCGGGCAGCTATCGTTCGGTCACGGTGAA
CGCCAACGGGGTGGTCACCCAGGGCAGCAATCCGACCACCCTGGCCGGCTATGCGAT
CGGAGATGCCTATACCAAGGCCGATACCGATGGAAAACTGGCGCAGAAAGCGAACA
AGGCCACCACCCTGGCCGGCTATGGCATTACCGATGCGCTGCGAGTCGATGGCAACG
CCGTGTCATCCAGCAGGCTGGCCGCACCGCGTAGCCTGGCAGCCAGTGGCGATGCCT
CCTGGTCGGTGACCTTCGACGGCAGTGCCAATGTTTCTGCGCCGCTGAGTCTTTCCGC
TACCGGTGTGGCGGCGGGCAGCTATCCGAAGGTGACCGTGGATACGAAGGGAAGGG
TGACTGCTGGAATGGCGCTGGCGGCGACGGACATTCCCGGGCTGGATGCTTCGAAGT
TGGTCAGCGGGGTGCTGGCCGAGCAGCGTTTGCCGGTATTCGCGCGCGGGTTGGCTA
CTGCTGTCTCGAACAGTAGCGATCCGAACACCGCGACCGTGCCGTTGATGCTGACCA
ATCATGCGAACGGACCTGTTGCCGGACGATACTTCTACATCCAGTCGATGTTCTATCCG
GATCAGAACGGCAATGCTTCGCAGATTGCAACGAGCTACAACGCTACATCCGAGATG
TATGTACGGGTGTCCTACGCGGCCAACCCTAGCATCCGGGAGTGGTTGCCCTGGCAG
CGTTGCGACATTGGAGGCTCCTTCACGAAGACGACTGACGGATCCATTGGAAATGGC
GTCAATATAAACAGCTTCGTCAATTCCGGATGGTGGTTGCAATCGACATCGGAATGGG
CGGCGGGTGGAGCTAACTATCCCGTGGGGCTGGCCGGTTTGCTGATTGTCTACCGCGC
ACATGCAGACCATATCTATCAGACCTACGTAACACTCAACGGAAGCACATATTCGCGC
TGCTGCTATGCGGGCTCTTGGCGTCCGTGGCGGCAGAACTGGGACGATGGAAACTTC
GATCCGGCCAGCTACCTGCCAAAGGCGGGATTTACCTGGGCGGCTTTGCCGGGTAAG
CCGGCAACTTTCCCGCCCTCAGGGCATAACCACGATACCAGCCAGATCACCTCCGGC
ATCTTGCCTCTCGCTCGTGGTGGCCTTGGCGCCAATACAGCGGCCGGAGCACGCAAC
AACATTGGTGCCGGAGTGCCGGCCACGGCGAGCCGGGCGCTCAATGGTTGGTGGAA
GGACAACGATACCGGTCTGATCGTCCAGTGGATGCAGGTGAACGTAGGAGATCATCC
CGGTGGAATAATCGATCGTACCTTGACGTTCCCGATCGCGTTCCCTGGCGCCTGTCTG
CATGTCGTACCGACTGTCAAGGAGGTGGGGCGACCAGCGACGTCCGCGTCGACCGTT
ACGGTCGCCGATGTCAGCGTCAGCAACACGGGATGTGTGATCGTTTCCTCCGAGTAC
TACGGACTGGCTCAAAACTATGGCATCAGAGTGATGGCCATCGGCTATTGA
MRSN317_R2_pyocin
SEQ ID NO: 20
CACCCCTTGCCGCCTCGGCCGCCGTTGCATTGCACGACAGTGGTCCATCCGCTGGAG
CTGTAGGTGTGCTCCACCGAGTCGATCAGGAACTCGCCGTCGATTCCCGCCTTGAAG
CCTTGGAGCAGCAGGCTGCGCTCGGCGAACAGGTCACCACGCCCCGGCAACTCCAG
GCGCACACTGGCACTGGAGCGGCTGAAGCTCGCCAGGCGAGCCTTCGCCGCCTGCT
CGGCCTCTCCACGGCTGGCATAGAGATGCCGGTCGACATGCTCGCCCGTAGCCGTAG
CCGGTGCCTTCGGGTTCTTCAGCTCGACGCTCTTGACCTCGCCGCTGCCGGGCAATTG
GTAGCGGGTCTTCACCGTTCTCATCAGGCTGCGGTCGTCGTAGGTAACGTCGAAGCT
GCCGACGTCAGCACGTCCCAACACCAAAGGTTCGATCCGACGCCCCGTGGCGCTCTG
CCCGCTCTGTCGCGGCAGCACCATGAGCATGCCCTCGGCCAGCTTGGCGGTGCAGTC
GTATTGGCGCGCCAGACGGGTGACGAAGTGGTAGTCGGACTCATTGACCTGGTCGAC
CCGGGCGATCGCCACCTGCAACGGACACTCCACGCGCCAGCCGTTGCGTGCGCCAA
CATCGCGGCAGACCTGAGCCAGGGTAGTGCCCTCCCAACCTCCGCTGCGTATCGTCTT
GCCACTGCCGCGCATGTCGCCGGCCTTGGCAGTGACGGTCAGGCAGTCCGGCGGAC
CAGCCCACTGCAAGGTGTCCACGGTAAAGCGTCCCATGCGCATCAGTGGCTCGCCGG
CATAGCCGAGATGAACCTCCAGCACCGCACCGCGCCTGGGTAAGGCCACCTGTCCAT
CGCGATCGTCGATCCTCAAGGTCAGGCTGTCGGACTCCATGCCCGGCTTGTCCAGCA
GGGTCAGCTTGAGCAGGCGGTCATTCAGCCGCTGGGTGACGTCGGTGCCGTCGGCA
ACGATACGGAAACTCGGTTGCACCTGGTTTCTCCAGAAAAGAAAAACCCCGCCAAG
GCGGGGTTGCGGGTGAGCGGTCGATCAATCCCACAGGCGCACCTGTTCAACATTGAC
CACCGGCATGTCAGGGAAACTGATCAGCAAGCCGGCGCGGAATGGCTGCTGCTCATC
GGCCAGCCCGGGATTGGCGTCGAGGGTCGCCTCGACGCAGCCCAACAGATGGCCATA
GACGTGGTAGCAGAGGCTGTCCAGCATGTCGCCGTCAGCGGTTCGGCAGGTCGTCGC
CATAGCGCTTGAACTCCAGGTCGAAGGTTTGTTTGCGGGGAGCGCCGTTGCCCAGCA
GGGCGCTCTGGTTCTCGCTGATCCGCACCAGACACCAGAGGCCCATCTGCACCTCGC
CGTAGCCGCTGCTCAGGATCAGCGGCTCACGCCGCTCCGCCAACCGGCGCAGCGCTT
CGAGCTGCTCCAGACCGACGACCTTTTCCTGGTTGCCTACCTGTCGACGCAGCGGGA
ATACCGCCCCCTTGAGCAGGATGCTCTCCTCGCCCATGCCGACGCTCTGCAGCGCCGG
CCGCCGGCCCAGGCGCACCTGCCCGCTCCAGTTGTAGGCACTGTTGCGCTGCAGGGC
GTCGAAGGCGGCAGTATTCAGGTTGAAGTAGAACGGTTGGGCATTGGCGGTCAGCGG
CTGCAGTACCAGCAGGTGCGGCAAGGGCGCTGCGGCCTCGCTCGGGGGCGTCGCCG
AGGGTGCCAGCAGCCACGACGGCAGCACCCCCGCCAGCGTCGGACTGATGTCGCCA
GCGACCTTGCCGACCGCCTGCGCGGCACTGTTCACCTGCACCTTCAGCGCATCCAGG
CGTTCGTCGATACCAAGCAACGCCCGGCTCGCCCGGTCATAGGTGGAGACCACGCGG
TTCACCTTGCCCTGCGCCTGGCCGATGCCGCGCATGGCACGCTGCAGCCGGGCAGCC
ATTTCCGGCGATACGCCGGGCAGGTTCTCCAGCTCCGCGGCTGCGCCGCGGATCTCG
CTGATCGCACCGTTCACCGGAGCGACCACCTTGTCCAGACTCTTGCGTCCGGACTCG
CCGGCGCGACCCAGGTACCTCAGGCCGGCCTGCAATTGTTCCAGATATGCCATGGGG
ACTCCTTAGACCATCGCCGGGTCGTAGAGGCTGCGCCGCTGCCGCTCGTCGGCGAAG
TCGACAAGCATGCGTTGCAGGTAGGGCAGCAACTCGTCGGCCAGGCGCTGCGGCTC
GTGTACGTTGCCTTGCACCGTCAGGTTGATCTGCGGCGCGAAGGTCCAGTTGGGCGC
AGCCGCGACGGGCTCGGCCTGTGGTGCGGGCACCGCCGGCTTGTTGTCCGAGGCCG
CCGGCGGATCGCCAGCCAGACTGCGTCCCAGACGCCCCCCCAGTTCGCTACCGCCCC
AGGCGCCGATAGCGCCGCCAACCAATCCACCGATCAGCGTACCGACCACCGGCACCA
CCGAGCCGATGCTCGCTCCCAGAGCGGCGCCAGCCAGGGAACCAGCCAGGGAGCCA
CCGGCCTCGCCATAGGCGATGGTCTTTTCGCTCTCCGACAGGTCGCTGGAGTAAACCT
CAGCAACATCGATCAACGACGAGGCCACTTGCAGCGGCGCGTTGCGTATGGGCAAAC
GCGACAGGCGCGGCAGCTTTCCTGCCATCGCCCCCAAGCCTCCTCCAGCCGAGAAGC
CAAGGGATGGAAGGGAGCGGGCCTTCAGCCCACCGCCCTTGCCTCTCTTCCTGCGTG
GTGTTTGACCGCGCTTTCGTTGTGCCGGCCTCCTCGCGGACTCCAGCGTAGAGTCGCT
TCCCGGCACCGGCCAGTTGGTAACGAACACGCGCTGTATTTCGCTACCACCCGATACC
CGGCCTGCGCCCTGTTCGATCGGATCGCCGACGGCAGCGCTCCCTCGCCGACCGAGC
CGACCGCCACGCAACACGTCGATAGCGCCGCGCCCTCCCTTGTAGGCGAGATACCCG
GATAACACCGCACCTGCGGCGGCGGTAAGACCGGCCGTCGTCCGCGGATAGGCTTCG
AGCAGTTCCGCAGTCGACTCGCCGAGTACCGCGCTCCCCTTGGCCAGGTTGTCCAGA
TATGGATTCAGGGCGCTGCCCAAAGCGGTCTGCGCCCTCTCCCAACTGTCGCTGGAA
GCCTTCCACAGCTCCTGGGACATCCCCTTGCGACGCCGCGCATCGCGCTCCGAAGAG
CCTACGGAAGACTGCTGGGATTCGAGCAAGCCCTGGAACTCGCCGCGCGACGCTCG
ATAGGCTTCAAGCGCCGGCGACGCCGCACCGGCGCTGAGCTTGCCGCCCTTATCCCG
ATAGCGCGCAGCCAGCTCCAGGTTGGCCTCCAGCAGACTCGCTCCGTCCTTGCGCAG
CGCTTCCAGGTCCCGGTCGTAGTCCTGGCCATAAGCGCGTTTCAAGCTGCCGCTGCTG
ACGAACTCTAGCCAGGCCTTCATCCGCACGTCCGCTTTGTCTGGCGTGGTGTTCTTTG
CCTGAATCTCCAGCAAGGCCCCCAGTGCACCGACATCGCCGGCCGTGGCCTCTCCCG
CATTGCCCAGCGCTGACAACAAGGCAGGCAGGCGACGCGCCAGGGCCTCACTGCCG
ACTCTGCCTTTACCCAGGACGACCAGGCGGTCGAGGGAGGCGGACAATTGCTCTGGC
GAGTCCGAACCCTGGGCCAGTTGCAACGCTCGAACCAGCCCCGCAGCCTCGGCTGA
AGCAATCCCTTGGCCAAAGCCGAAGCGGCCCGCCAGTCCCAGATTCATTTGCGCGGC
GGCGAACCCCATGCCGGTGGCGTTCAAGTGCTCCAGCATGTCCAGCGTCGCCGTGCG
TCCCAGGCCGCTCTGGCTGGCACTGTCCTGGATCAGGGATGCCAAGGCTGGCTCCCG
GCCGACGTCCAGGCCGTTGCGGATCGCCAGGTCGCGCAACCGCGCTTGATAACCGGC
AGCCGTCTTGCTGGCTTCCACGACCAGTCCGGAAAATGCGCTAGCCAATGGCATTGC
CTGGCTGGCCTGCTCCAGCCGTTGCAGGCCGCGGACCTGCATATCCAGGCCACGCTG
CATCTCGCCCAGTCGAGCGTAGGCATCACCCGCAGCCCTGGCCTCGATGCCGAGTCG
CTTCAGTTGCTCCTGGCGTTCGCCAAGCTGCCGGGCATGCTCAGCAACGCCGCGGTC
CCCGAGCTTGCGCATATCGGCGAGTTCTCGCTCCAGGCGTATGGCGTCTCCCAGCACA
TCCCTTAGGCTGGACGCCTGGCGGGAACGTTCCTGGAGACTGTCGAGCCGTGCCTTG
ACGGTCTCGAATGCCTTGCCCAAGGAGGGGTCGGCGATGCCGCCGATGGAGACCACC
AGGTCCATGTCTTTACTCATGACGGTTCGTCTCGTCTGTTATCGATGTCCGCCTCCGCG
GACCTACTGAAGTAGCGCGGGCGCCGCTCGCCTGGCGGACGGCGCCGTCGCTCACTC
CTCGCCCTCGGCGAGCCACCAGAGCAGGTCGCCCAGGGTCATGCGCTCCAGTTCGCT
GGCCTGGAAGCCCAGCTCAATGGCCAGGCGCCTCGCCAGTTGACGCATACGCGAAG
GTGTCGTCCGTGTCGTCTTGCACCAGGCGAAAGTAGGCGCCTTGCAGCCGCTGGTAG
TCGCTCAGCTTCAGCCCCTCCAGGTCCTGGCGGCTGACCTGCGCCAGCGAGGCGAAC
AGTTGCAGTTCGCGCTCCTCGGCATCGTCGCCGGCCACCTTGCCGGCCAGGCGGATAT
CACGCACGGTGGGTGCACGCAGGGTCAGCGTGTCGACGCTGACCCCGTTGCACTGT
GCCGGACGCGAGAGACGAACGAGGGCGGCGTCGGCATCCAGCGTCAGCCAGCCCGG
CAGTCGATTCTCTTGGGTCATGTCCGGAGCTCCTCTTACAGGCCGAGGTCGTTGCGCA
TGCCGGCCAACTGGTCGACACCGTTGATCGCGCGGACACCGTTGACCGGATCGATCT
CGTAGACCTCGCGGCCATCGACTTCCAGCTTGTAGTAGCTGACCGCAACGGCGTACTT
GAACTCGGCTTTCTCGCCGGCCTTCCAGTCGCCCGGGTCGACCTCCTTGAGCAGGCC
GCGCAGGGTAGCCACCACTGGCACGCTGGCGCCCTTCTGGCCCTTGAAGGAGCCGC
GGAACACGCCATTGAAAGCGCTCTGGTCGGCCAGGCCGAAGAAATTCAGTGCTTCTC
GGCGGGCACCGTTGGTGGAGAACTTGGCCTCCATCGCCTCCAGGCCCATGTCGATGG
ATACCGGCGCATCCATGCCGCCGGCACGGTATTGCTCGGTCTTCACAGCCAGCTTGGG
CAGGGTCAGGGATGGCACGTCACCGGCGAAGCTCACGCCGTCGATGAACAGGTTGG
TATTGGTCAGGGTTTGCGGAATCATCACGGGCGCTCCTTAGGCGACATCCAGAACTTC
GGTGAGCCACTGATCGGTCACCTCGACACGGAAATTGGGGTTCTCGGCAGGCGGCAC
ATCGGTGAAGCGGATGTTCCAGTACACCTTGCCCTGGGCCAGCTGGCTGGCGCTGTT
CAGGTCCGGGTCGGCATAGACCTCGAAGTTGATCACCGCGCCCTGGTTCTTCAGATC
GCGCATGAAGGCGCGCAGGCCCTCGGTGACATCCTTCACGTAGGTCTTGGTGATGCC
GCGGTCCACCGCCCACTTGTGCCCGGCGAGGATCGCATCCATCACCAGGTCCATGGT
CCGAACGCGGGTGACGAAAGCCCACTTGCTGTCGCTGGACAAGGTGCGGTTGCCCC
ACAGGCGATAGCCATCGTCGCGAATGATCGTGGCGATATTGGCGTTGTTGAGCAGGTT
GGCGCGACAGGTCTCATCGCCGTCGAGGAACTCCACCGGACGGCTGGTGCCGGTGAT
GCCCTTGATCTCCTTGTTCGACGGCGAGGACCAGAAGCCGTACTCGGCGTCGGTCCA
GGCGAACAGGCCGGCGGCGTAGGCCGAAGCCGGGGCCTTGCGTGCGGCATTGGTGG
CGCTGTCCCAAACCTGCACGCCCGGGTCGACCATGAACAGGCGCTTGCTGCCGAAGT
TCTTGGCGTAGGCGACGGCCGCCTCGTCGGTGCTATTGGGACCATCGAGAATGGCGA
TGGCCCGCAGTTTCTCGGCCAGCCCGTCCATGGCGGTGGCCACCGCTTGCTGGGCCG
AATGACCCGGCGCAACTAGTAGACGTGGCTGGGCATTGAAGCGGCTCTTGCCGTCTA
GCAGAGCCTGAAGTCCGGTGCGCTCGCCGGCGGCGCTGACACCACCTATGACGGCG
CTGGCCTGGGCCTCGGGAGTCTCCGCGGCTTCCACGCCCACCGCCACGATCACGGCC
TGGGCACGGTTATAGATGGCCTCGCAGGCCAGGTAGATAGACGAGCCGATGCCGAAC
GCCGCGGCGGCGTCCTTCTTGCTGGTGAGCAGCACCGGAACGTTGGGCTTGGCGCTT
GCCTGCGCCCCCGGCGTGAACACATCGCAGAGGCCGATGACGGAGCTGGCCGGCAG
CGCGATGGTGCGCGCACCGATATCGACGTTGGTTACCGTAACGCCGTGGAAGAAACT
CATAGGTAGATCTCCATTAATGAAAAACCCCGCACGAGGCGGGGTTCAGGGGGTGAC
ATAACGTCTGAGCCAATCTGGTACGGACGGACGTCCGCCACTGTCGGGAAATAAAGG
CTCTTCCGGCCAATCCCGTAAGCTGGCTCTGTACGACATGAGTTCATGGTATTGGACA
GGGAGTAAGGTCGTTTCCTTTCCGGTTTCCAATTGATCTCGATGGCGAACAACTAGGC
CGTCGGTCGCCAACAGCTGACTATCCCGCCATCTCCGCTCATGGGCGCTACGTTGCTG
ACGGGAAAGCGGAGGGCGCTCGATCGCTACTGGGTTCCCCTTCACGTCGGAACTGAT
TGCTCGGCCTGCCGCCTGTTCGTTCATCAGTTGTTCATGTAGCTCTGCGCTGACCCAC
TCGCCATCGACTGGCCAGCAGCCTGCGTATTCATAAACCTCTCGCAAGGAGGCCGGA
TAGAATGCCACCTGGCTTGGAGAGAAATAATATTCGCCCTTCATCGTCCAAATGCCTC
CCATGAAAGAACCGCCTGAGCAAAGTAGCCATTGCGAATCACCGCGGTAGTGGTAGT
CGCTCCACGGAAACCTGTACTGGCATCCGTTCCCGGATGGAAAGCACTCGTCTGATTC
GCATATCCGCCGAGGCATAGCGTAGGGAAAGGAATCGGGAACGTAATCGAAGCATCG
GCATCGGCGGGGCAAGTGACCTGCCCCCATTGCCTAATGAGGCCAGTGTCATTGTCCC
GCCACCATCCGCTCGCCCCAAGGGAAGCAGTCGCAGGAACCCCTGCTCCGATAGTGC
TACGTGCTCCCGCTGCCGTATTCGAACCGACGCCGCCACGTGCCAGTGGGAGAATGC
CCGAAGTAAGCTGTCCGACGTCATGGTTATGTGCGGATGGTGGAAAAGTGGCAGGTT
TTCCCGGTAACGCATTCCAATAGAACCCCGACTTCAACAGATAGTCACTGGGGTTGAA
GTCTCCGCCATGCCACATGCGACGCCAGGGAAACCAGGTATTTGAATGCCGGCACCG
GAAATAGAAACTCTCACCATCGTAGGCTTGATACGTCTGATAGATGAAATTGCTACTC
GCGGCGTACACATGAAGCAGGCCGGCCCGAACTATAGGGTAGTTGGCTCCACTGGCA
GCTTGGGCAGTAAAACTCTGGCTCCACCACCCTGAGGTCACCATCGAATCCAGGTTG
ACGCCTCCAGGCAGTTCACCATCGGCCTCTTTGGTGAAGGAACCTCCAATGTCGCAG
CGCTGCCAGGGCAACCACTCCCGGATGCTAGGGTTGGCCGCGTAGGACACCCGTACA
TACATCTCGGATGTAGCGTTGTAGCTCGTTGCAATCTGCGAAGCATTGCCGTTCTGATC
CGGATAGAATATCGACTGGATGTAGAAGTATCGTCCGGCAACAGGTCCGTTCGCATGA
TTGGTCAGCATCAACGGCACGGTCGCGGTGTTCGGATCGCTACTGTTCGAGACAGCA
GTAGCCAACCCGCGCGCGAATACCGGCAAACGCTGCTCGGCCAGCACCCCGCTGACC
AGCTTCGACGCATCCAGCCCGGGAATGTCCGTCGCCGCCAGCGCCATTCCAGCAGTC
ACCCTTCCCTTCGTATCCACGGTCACCTTCGGATAGCTGCCCGCCGCCACACCGGTAG
CGGAAAGACTCAGCGGCGCAGAAACATTGGCACTGCCGTCGAAGGTCACCGACCAG
GAGGCATCGCCACTGGCTGCCAGGCTGCGCGGTGCGGCCAGCCTGCTGGATGACAC
GGCGTTGCCATCGACTCGCAGCGCATCGGTGATGCCATAGCCGGCCAGGGTGGTGGC
CTTGTTCGCTTTCTGCGCCAGTTTTCCGTCGGTATCGGCCTTGGTATAGGCATCTCCGA
TCGCATAGCCGGCCAGGGTGGTCGGATTGCTGCCCTGGGTGACCACCCCGTTGGCGT
TCACCGTGACCGAACGATAGCTGCCCGCCGTCACGCCGGAAGGCGCCAGGCCAATGC
TGCGGTCGGCAGAAAGATCGCCCCCACCGAGCAGGCCATTGCCAGCCAGGATCTTGC
GGCCCTTGAAATCGGCAGCGACCTTTTCCTTCACCCAGTCCTGAGTGGCGTAGATGAT
GCCGTTGTCGATCAGCAGCTGGACATTCTCCAGGCCGGAGAGCGCGATGTTCACCCG
AATGGTCTGGGTCCGCGCACTGCCACTTTCCATCGCAGCCTTGTAGCTGGGCGGGCA
GTTGGATACCGCGACGAACTTGCCGTCGGCATCCTGCAGGCCGATCTCGCGGATCCA
GAAGCCACCTACCTCAACTGGCAGCACCACCTCGGCAACCAAGGTATTGGCGTTCTT
GTCGGAAACGAACAGCCGATTCAGCTGGGCCCGATGGCGTTGGTTGATCAGGCTCTT
CTGCGCCGCCGAAGGCAATGGATCCGGCGTGTCGCCCGGCGCACCGCCAGCATCGCC
GATCAGCATATGAGTCGGCTGCCATTTCTTGCCTGCTGCGCTGGCCGCAGCCAGCGCA
GCGGCACCTATGTCGGTGAGCAGGCCACCGTATTTCGGAGTATTGGTCGTCATGTCAC
TGTTTCCAGGGGAAGATTTCCAGGGTGTCGCCGTCGGTCACGGCCAGTCCGTGACGG
GACCGGATTACCGGGGTGATGCGCAGGTCCAGGCGGGTCAGGTGTCGGCTCACCGG
GCGGACGTCATCGAGCAAGCGCTCCAGCTCCAGTACGGTGCCTTCGTCGATGCCACG
GTCGCTGACGTCCACGGTGATCTCGAAGGTCCCCGGCACGCCGGTCGGGCTTCGCTG
CCACCATTCGGTGACCTCGCTCAGCGCACCGATCGGCTCGACCACTTGGCGCAACGC
GGTCAGCGTGCCTTTGCGCTGGTGCAGGACGAAGGACGTCGCTACCGCATTGCGCTT
GGCCTCGTCGCTCCAGTCCGGGTCCCAGCGGTCCACCGAGCGCGTCCAGGCCAGGTA
GGGCAACAAGGCCGCCTCACAGCGTGCCGGATCGTGAAGCTCACGAAGCGGCACCG
GCAGTTCGGCAGGCAATACATCACCCAGAGAGCGTTCCAGAGAACTCCTGTTTGGCG
GCAGCAGTCGGCTACTCATCGACGCCTCCCAAGGTGATCGCGTACCCCGTGCAATAG
GCCGCCTGGGTTTCATCCAGGACCACGTCGGCCGCAGGCTCCTGCAGTTCGACGCGC
TGCACGCCTTCGACATGCAGGGTGGCGAACAGGGCCGAACGCCGGATGTCGCGGCC
CAGGCGGCGCTGGGCGGAAATGTAGTCGCGCAGCGAAGCCTCGGCGGCGGCACGGA
TCAGCTCGGCCTCGGGACCGGGAAACAGGTAGAGCTGCGCCTTGACCTGATAGGGA
ACGATGCCTGCCGAGCGCACCGTTACCCGATCCGCGACAGGACGCACGTCCTCCGCG
TTCAGCGCCAGGCGCACTGCCTGCAACACCGCTTCCGGTGCCTGCCCGTTGCCTTCC
ACCCCGAGCACGCTGATCAGCACTTCGCAGGGCGCCGGACTGGTCGCAGAGGCATC
CGCCACCCTTCCATCCGCGCCCAGCGCATGGGCTATATAGGCGTTCCGCGGTCCTGCG
ACGGATAGCTGCTCGAACGCGAGCTGGATGCGATTGCGCAGCGACTTGTCGCTCTCG
TATTGCGCCTCCACCGGCGGTACCGCCTCGGGCTGAGCGGGCCTGATCAGCAAACGC
TGCACATTGAAGCCGGCACCGATCTGGTCGAGATCGGCACCGCTGGCGTATGCCAGC
ATCACCGCCCGCGCCGCATCGTTGATCCGCGCCCGCAGCAGCAGTTCTCGATAGGCC
GCCAGTTGCAGAAGCTTGAGAACCGGATCGGACTCCACCTCCGCTGTCCATTCGCCA
GCCATGGCTTCGCGGAAGTCGCCCAACAGCTCTTGGTAAATCGTCTCGAAATCGAGG
TTTTCGATAACCTCTGGCTCCGGCAACTGGGAAAGATCGATGATCACGCCGAGACCTC
CATATTGATGTTCTCGCCCTCGAACACGCCGCTCAGGAGCAGATCCACGCGGCCATCG
ACGACCGCGACGACTCGCACGGCAGACAATGCGATGCGCGGTTCCCAGCGCCCCAG
GGAACGGGCTACCTCGGCCTGCACGGCGCTTTTCCAGCCTTCGCTCACCGGCATGTC
GACCATCCGCCGCAGCTTGCTGCCGTACTCGGGGCGCATGCGCCTGCTGCCCAACGG
GGTGGTCAGGATGTCCTCGACGGACTGTTTAAGATGAGCCAGGCCGGACAGGGGCA
GCCCGCTACGGCGATCCATCCCGATCATCGCCCCGTCCTCCCGCTCCGGCAGAGACAG
ACCGCGCCTGGCTCCGAATGGCTGTGAGTGTTGCCCATGGCGAACTCCGCAAACGAA
AAGGCCCGCTCGCGCGGGCCGGTTGAATGTTCGACGGACCGCCCTTCGGCAGCCCCG
TCGTATGTTCAATGGGTGTGGTGGTTGCTGTTGCCGGCGGTGTCGATGATCGAGCCGC
CGCCGAGGATGTCTCCGCTGACCGTCAGTGTCCCGGCGATCTCCACCGGTCCTTCCA
GGCTGATCTTCGGCGCCTTGAGACTGACCGCCCCGTCGCTGACCCGCACCTCGCTGG
CGCCGACCCTCACTTCCACGGTGCCGCTGGGCAGCTCGACGCGGTAGCGATGCGCAG
CCCAGTCGTAACTCAACGAGGCGCCGTCGTCGAAACGCCAGGTCTCACTGCTGGCGC
TGTTGTCCGGCGCCGTGCCCGCATCGCCGTACAAACCGGGAATAAAGGTACCCATTG
ACACTCCGCCCGACGGACTGAGCAGTACCCCCTGTTCGCCTATGCTCGGCGCACGCC
AGTGGCGAACCTTGCCGGCTGCCAGGGAGTGCCAGCGCAGCCAGCCGCTGGTCCAG
TCGCCGGAGCGCACCCGTACCCGCGCCGCTGCCAGGTCCACGGCGACCACCACGCA
GGGCAGGATCATCGCGGCGAGCATGCGGTCATGCTCCGCACTGACATAGCTCATGCC
AACGCCTCCGGAGCGAAGTAGTCCGGCTCATAGCCGGGGCCGGTCTGCGGGTCGAA
GCCCAGCATCAGGCTGCCCGGCGGCTGGTCCTCCCAGGCCCACTCCGGGTCTCCGAG
ACGCAGCGACTGCGTCCACTCGACCAGCCAGACACGCAGGCTCTTCAGCTCCTCGCG
ACTGCCGTCGACGGCCGAGCGTACGAAGCGCGCCGGTTCCAGAGACAGGTTCCAGG
TCTGCTCGCGCAATGTCCGAACGATTCCGCAAGCAATGGATACCGCCTGGCGATCGGC
ACCAGGCGGCACCATTATCCGCGCCTGCAGGCGGCACAGCAGGGCACTCTCGCCGGT
ACCGGGATCGCGCTCGGGCAGTAGCTCGGCTATTTCCAGGGAAACCGCCGGGGTATT
TTCCGGCACGCCTGGCCAGAAGCCGATCAACTGCGCCTCCGGCAGACGCTCACGCA
GTACCTGCTCGATGGCCGCGTACAGAGCCTCGAGCGTAACAGCCTGTTCAGGCATGG
TTGACTCCTTCGATCAGTTTCAATGCGGGCCACGGTCGCCCGACTGTTCCTCGGGCAG
CTCGCAAACCCCCAGGCGCCGGGCGGCCCAGCGTTCGTAGAGACCGACGGCGATCT
CCGCGCCGGCCGCGGCGGTCAGACTGCCCAGCGCCGCGGCGAACAGCAGCGCGGCA
CCGCAAGCCAACGCCAGCAGCATCACCGCCACGCCGCAGACGATCGACGCGCCGGA
GCGCAGCGCCAGGCGCCGCAACAAGGCCCAGCCGCCGAGACCGGCCTTGTCGGCAC
GCCACATCTCGCCGCTGACGCCGCCCAGCGCGGCGAGCAGGATCAGTACCCAGAGC
GGCATTTCCGTCAGTGTCTGCGGTTCGTTGCCCATGGGTCCCTCCTTGGCAGGCAGCG
CCGGGCACGTCCGCACGAACGTTTTCCCGGGCAATAAAAAACCCGGCGCCAGGGCC
GGGTTCCGGTGCTTCACGAGTGCCTCCCTGGGGACGCACCTTTACAAGAATGACTAC
TTTTTACCGCCCGATTCCCAGCCCGGCAATGCCTTCTCGACAATCCACTGCAATAGGA
GGGAGACGCACCACGATATGAGGGTGATATACCGCCTTGGGAACCTTCGCCAGGCAT
CCGGGCAAGTGCGTACCCACTTCCGATATGGAAAAGCCCGGCATCGACTGACGAAAA
GGACGGCTCCACCAGCCCGAACCCGCCTCTACCGTGCAGGGTCCGCTGCTGTGCATG
GGATTCGCCGCCTCCGTGGGCGACAGGCATAAAAAAAACCGGCCCGAAGGCCGGTT
GGAAAATTCCAGGGGGGACCGGGATCCGGTCCAGGAACTCAGGCCGCCCGTCGCAG
CAGGCCGTCCTGGATACCCTGGTGCGCCACATGCAGGCGCAGATAGAAGGTGTTGCG
GCTGCAACCGCAGTGACGGTACTTCTGCTCGGGGAAGCTGTCGCGATTGAGATAGTG
CTCCACCACCACGGTCTTCAGTTCCTCGGGTAGGCGATTTACTAGGCGATCGACCTCC
GCCACACGGTCGAGGATCACCCGGCTGCCACGGTGGCCGCGGACCACCTCGCCCTTG
CTGGCGATCAGCATGGCGATCAGGTTGCCGCCGGCGTAGCCGCCTCCGTTGCTGCCT
GGCGCGTGCATTTCCTCGGCCCAGAGCTTGAGCATTTCATCGATGGCTTTGATCACGG
TCGTGTCTCCTTTCACTGCCCGCGAGGGCTCGGCATGAGGTCGGTTCAACGGCGCTC
GTGGCGGTCCTGGCAGTCGATGCAGCGACTGCAACCCGGTGCCGCCCGGCGGCGCG
CCTGGGGAATGGGCTCGCCGCAGTCCTCGCAGTCTTCCGCGGACTCGCGGATGGCCA
GCGCCGGGCGCGCCGCCAGGAGGCCGTCGAGGCGAGCCAGGACCAGTTCGTTGGCG
TGATCGGCAAGGTCAGCCATGGAAAGCCTCCCTGGCGTGCCCGACGCCCGACGGCC
GAATGGATGGAACATGACAGATGCGCGGCGCCGGTTCGGCGACCTGCGTTCCTCTGC
GCGGAAATGGCGAGTGCGTATCTCGCGACATGGCAATGACTCCCTGTACGACGCCGC
CGTACCTGAAGCAGGTCGGCGACAGAATGAATGAGCGGGACGGAGCGTCTGCATGG
ACGGCGCTCCGGCCCTCCAGGTGCCGGAGCGCTTTCCCGTGGCCCCGGCACCCAAGC
AGTCGGCACCTGCATCGTCGGTCACCGACAACCGGGTGCGTCCCTGCACCCATGTGA
AATCTCTGAAAGGCGGGTGGGAAGTTGTGGAACTCACCCCCTTCCGCTTTCTGACGG
AAGAAAAATTACAACCAAAGCTATTAATCGTCAACACCTAGGCTCTTTACAGAGAATC
CATCGGTCTGTAGATTGCCGAACATGGACAAGAGCACCCAGATCCCGCCCGACAGCT
TCGCCGCTCGCCTCAAGCAGGCCATGGCGATGCGCAACCTGAAGCAGGAAACCCTC
GCCGAAGCGGCAGGGGTTTCGCAGAACACCATTCACAAGCTGACCTCGGGCAAGGC
CCAGAGCACCCGCAAGCTGATCGAGATCGCGGCGGCCCTGGGCGTCTCGCCGGTCTG
GCTGCAGACCGGCGAAGGCGCTCCAGCCGCGCGCAGTGCCGTGTCCGTGGCCGATG
GCAGCCCATTGGTGCTGGAACCGCTGCATCCGTGGGACAGCGACACACCGCTGGAC
GAAGACGAAGTGGAACTGCCGCTGTACAAGGAAGTGGAGATGTCCGCCGGCGCCGG
ACGCACTGCGGTACGCGAGATAGAGGGGCGCAAGCTGCGTTTTTCCTATGCCACGCT
GCGAGCCTCGGGCGTCGATCCGTCGGCGGCGATCTGCGCCCAACTCACCGGCAACAG
CATGGAACCGCTGATCATGGATGGCTCCACCATCGGCGTGGACACCGCCACCACCCA
TATCACCGATGGCGAGATCTACGCCCTCGAACATGACGGCATGCTGCGGGTGAAGTTC
GTCTATCGCCTGCCCGGCGGCGGCATTCGGCTGCGCAGCTTCAACCGCGAGGAATAC
CCGGACGAGGAGTACTCGCCGGAGGACATGCGCAGCCGCCAGATCAGCATGATCGGC
TGGGTCTTCTGGTGGTCCACCGTACGCCACCGGCGCGCCCCGTCCCTGGTGCGGTGA
MRSN317_R2_pyocin_tail_fiber
SEQ ID NO: 21
ATGACGACCAATACTCCGAAATACGGTGGCCTGCTCACCGACATAGGTGCCGCTGCG
CTGGCTGCGGCCAGCGCAGCAGGCAAGAAATGGCAGCCGACTCATATGCTGATCGGC
GATGCTGGCGGTGCGCCGGGCGACACGCCGGATCCATTGCCTTCGGCGGCGCAGAAG
AGCCTGATCAACCAACGCCATCGGGCCCAGCTGAATCGGCTGTTCGTTTCCGACAAG
AACGCCAATACCTTGGTTGCCGAGGTGGTGCTGCCAGTTGAGGTAGGTGGCTTCTGG
ATCCGCGAGATCGGCCTGCAGGATGCCGACGGCAAGTTCGTCGCGGTATCCAACTGC
CCGCCCAGCTACAAGGCTGCGATGGAAAGTGGCAGTGCGCGGACCCAGACCATTCG
GGTGAACATCGCGCTCTCCGGCCTGGAGAATGTCCAGCTGCTGATCGACAACGGCAT
CATCTACGCCACTCAGGACTGGGTGAAGGAAAAGGTCGCTGCCGATTTCAAGGGCCG
CAAGATCCTGGCTGGCAATGGCCTGCTCGGTGGGGGCGATCTTTCTGCCGACCGCAG
CATTGGCCTGGCGCCTTCCGGCGTGACGGCGGGCAGCTATCGTTCGGTCACGGTGAA
CGCCAACGGGGTGGTCACCCAGGGCAGCAATCCGACCACCCTGGCCGGCTATGCGAT
CGGAGATGCCTATACCAAGGCCGATACCGACGGAAAACTGGCGCAGAAAGCGAACA
AGGCCACCACCCTGGCCGGCTATGGCATCACCGATGCGCTGCGAGTCGATGGCAACG
CCGTGTCATCCAGCAGGCTGGCCGCACCGCGCAGCCTGGCAGCCAGTGGCGATGCCT
CCTGGTCGGTGACCTTCGACGGCAGTGCCAATGTTTCTGCGCCGCTGAGTCTTTCCGC
TACCGGTGTGGCGGCGGGCAGCTATCCGAAGGTGACCGTGGATACGAAGGGAAGGG
TGACTGCTGGAATGGCGCTGGCGGCGACGGACATTCCCGGGCTGGATGCGTCGAAGC
TGGTCAGCGGGGTGCTGGCCGAGCAGCGTTTGCCGGTATTCGCGCGCGGGTTGGCTA
CTGCTGTCTCGAACAGTAGCGATCCGAACACCGCGACCGTGCCGTTGATGCTGACCA
ATCATGCGAACGGACCTGTTGCCGGACGATACTTCTACATCCAGTCGATATTCTATCCG
GATCAGAACGGCAATGCTTCGCAGATTGCAACGAGCTACAACGCTACATCCGAGATG
TATGTACGGGTGTCCTACGCGGCCAACCCTAGCATCCGGGAGTGGTTGCCCTGGCAG
CGCTGCGACATTGGAGGTTCCTTCACCAAAGAGGCCGATGGTGAACTGCCTGGAGGC
GTCAACCTGGATTCGATGGTGACCTCAGGGTGGTGGAGCCAGAGTTTTACTGCCCAA
GCTGCCAGTGGAGCCAACTACCCTATAGTTCGGGCCGGCCTGCTTCATGTGTACGCCG
CGAGTAGCAATTTCATCTATCAGACGTATCAAGCCTACGATGGTGAGAGTTTCTATTTC
CGGTGCCGGCATTCAAATACCTGGTTTCCCTGGCGTCGCATGTGGCATGGCGGAGACT
TCAACCCCAGTGACTATCTGTTGAAGTCGGGGTTCTATTGGAATGCGTTACCGGGAAA
ACCTGCCACTTTTCCACCATCCGCACATAACCATGACGTCGGACAGCTTACTTCGGGC
ATTCTCCCACTGGCACGTGGCGGCGTCGGTTCGAATACGGCAGCGGGAGCACGTAGC
ACTATCGGAGCAGGGGTTCCTGCGACTGCTTCCCTTGGGGCGAGCGGATGGTGGCGG
GACAATGACACTGGCCTCATTAGGCAATGGGGGCAGGTCACTTGCCCCGCCGATGCC
GATGCTTCGATTACGTTCCCGATTCCTTTCCCTACGCTATGCCTCGGCGGATATGCGAA
TCAGACGAGTGCTTTCCATCCGGGAACGGATGCCAGTACAGGTTTCCGTGGAGCGAC
TACCACTACCGCGGTGATTCGCAATGGCTACTTTGCTCAGGCGGTTCTTTCATGGGAG
GCATTTGGACGATGA
TuD199_R5_pyocin_tail_fiber
SEQ ID NO: 22
ATGACGACCAATACTCCGAAATACGGTGGCCTGCTCACCGACATAGGTGCCGCTGCG
CTGGCTGCGGCCAGCGCAGCAGGCAAGAAATGGCAGCCGACTCATATGCTGATCGGC
GATGCTGGCGGCGCACCGGGTGCTACTCCGGATCCGATACCCGCAGCCACGCAGACG
AAACTGATCAACCAGCGCTACCGGGCGCAGCTCAATCGCTTGTTCGTGTCGGACAAG
AACATCAATACTCTGGTTGCCGAGGTGGTGCTACCAGTTGAGGTAGGTGGCTTCTGG
ATCCGCGAGATCGGCCTGCAGGATGCCGACGGCAAGTTCGTCGCGGTATCCAACTGC
CCGCCCAGCTACAAGGCAGCAATGGAAAGTGGCAGTGCGCGGACCCAGACCATTCG
GGTGAACATCGCGCTCTCCGGCCTGGAGAATGTCCAGTTGCTGATCGACAACGGCAT
CATCTACGCCACTCAGGACTGGGTGAAGGAAAAGGTCGCTGCCGATTTCAAGGGCCG
CAAGATCCTGGCTGGCAATGGCCTGGTCGGTGGGGGCGATCTTTCTGCCGACCGCAG
CATTGGCCTGGCGCCTTCCGGCGTGACGGCGGGCAGCTATCGCTCGGTCACGGTGAA
CGCCAACGGGGTGGTCACCCAGGGCAGCAATCCGAGCACCCTGGCCGGCTATGCGAT
CGGAGATGCCTATACCAAGGCCGATACCGACGGAAAACTGGCGCAGAAAGCGAACA
AGGCCACCACCCTGGCCGGCTATGGCATCACCGATGCGCTGCGAGTCGATGGCAACG
CCGTGTCATCCAGCAGGCTGGCCGCACCGCGCAGCCTGGCAGCCAGTGGCGATGCCT
CCTGGTCGGTGACCTTCGACGGCAGTGCCAATGTTTCTGCGCCGCTGAGTCTTTCCGC
TACCGGTGTGGCAGCGGGCAGCTATCCGAAGGTGACCGTGGATACGAAGGGAAGGG
TGACTGCTGGAATGGCGCTGGCGGCGACGGACATTCCCGGGCTGGATGCTTCGAAGT
TGGTCAGCGGGGTGCTGGCCGAGCAGCGTTTGCCGGTATTCGCGCGCGGGTTGGCTA
CTGCTGTCTCGACCACTAGCGATCCGAACACCGCGACCGTGCCGTTGATGCTGACCA
ATCATGCGAACGGACCTGTTGCCGGACGGTACTTCTACATCCAGTCGATGTTCTATCC
GGATCAGAACGGCAATGCTTCGCAGATTGCAACGAGCTACAACGCTACATCCGAGAT
GTATGTACGGGTGTCCTACGCGGCCAACCCTAGCGCCCGGGACTGGCTGCCCTGGAA
GCGCTGCGACATAGGTGGTTCGTTCAGCAAGGAGGCGGACGGGGCCTTGGGCGGTG
CGGTCAATCTCAACTCGCTCATCACGTCGGGATGGTGGTACCAGACGGCCAATGCAC
AGGCCGAAAGTGGGGCGAACTACCCGGTCCCCCGGGCCGGCTTGCTTCAAGTGCATA
ATGCAGGCACCAATTTCATCTACCAGACATACCAGGTTTATGACGGTGAAGGGTTCTA
TTTTCGTTGCCGCTACACCAACACCTGGTATCCATGGCGGCGTGTATGGCATGGAGCG
GACTTCAATCCGAACGACTATCTGCTGAAAAGTGGCTTTACGTGGGCCGCCCTGCCA
GGAAAGCCCGCGACCTTTCCGCCTACTGGCCACAACCACGACGCCGCCCAGATCACG
TCGGGCATCCTGCCTCTGGCGCGCGGCGGTCTTGGTTCGAATACGGCAGCGGGGGCG
CGCAACAATATCGGGGCTGGGGTTCCCGCGACGGCGAACCGATCGCTGAATGGCTGG
TGGAAGGATAACGATACAGGACTCATCGTGCAGTGGATGACAGTGAGTGTCGGCGAT
CATCCGGGTGGAATCGTCAACCGCTCGCTGACCTTTCCGATCGCGTTTCCCACTACCT
GCCTGCACGTGGTGCCGAGCGTCAAGGAACTGGGGCGGCCGGCGACGTCGGCATCG
ACCGTCACCCTCGCAGATGTCAGCGTCAGCACCACGGGGTGTGTGATTGTCGCTACC
GAGTATCACGGTGCGGTCCAGAACTATGCGATCAGGCTTGTGGCCATTGGCTGTTGA
MRSN2101_R1_tail_fiber
SEQ ID NO: 23
MTTNTPKYGGLLTDIGAAALAAASAAGKKWQPTHMLIGDAGGAPGDTPDPLPSAAQKS
LINQRHRAQLNRLFVSDKNANTLVAEVVLPVEVGGFWIREIGLQDADGKFVAVSNCPPS
YKAAMESGSARTQTIRVNIALSGLENVQLLIDNGIIYATQDWVKEKVAADFKGRKILAGN
GLVGGGDLSADRSIGLAPSGVTAGSYRSVTVNANGVVTQGSNPTTLAGYAIGDAYTKAD
TDGKLAQKANKATTLAGYGITDALRVDGNAVSSSRLAAPRSLAASGDASWSVTFDGSA
NVSAPLSLSATGVAAGSYPKVTVDTKGRVTAGMALAATDIPGLDASKLVSGVLAEQRLP
VFARGLATAVSNSSDPNTATVPLMLTNHANGPVAGRYFYIQSMFYPDQNGNASQIATSYN
ATSEMYVRVSYAANPSIREWLPWQRCDIGGSFTKTTDGSIGNGVNINSFVNSGWWLQST
SEWAAGGANYPVGLAGLLIVYRAHADHIYQTYVTLNGSTYSRCCYAGSWRPWRQNWD
DGNFDPASYLPKAGFTWAALPGKPATFPPSGHNHDTSQITSGILPLARGGLGANTAAGAR
NNIGAGVPATASRALNGWWKDNDTGLIVQWMQVNVGDHPGGIIDRTLTFPIAFPGACLH
VVPTVKEVGRPATSASTVTVADVSVSNTGCVIVSSEYYGLAQNYGIRVMAIGY
MRSN317_R2_pyocin_tail_fiber
SEQ ID NO: 24
MTTNTPKYGGLLTDIGAAALAAASAAGKKWQPTHMLIGDAGGAPGDTPDPLPSAAQKS
LINQRHRAQLNRLFVSDKNANTLVAEVVLPVEVGGFWIREIGLQDADGKFVAVSNCPPS
YKAAMESGSARTQTIRVNIALSGLENVQLLIDNGIIYATQDWVKEKVAADFKGRKILAGN
GLLGGGDLSADRSIGLAPSGVTAGSYRSVTVNANGVVTQGSNPTTLAGYAIGDAYTKAD
TDGKLAQKANKATTLAGYGITDALRVDGNAVSSSRLAAPRSLAASGDASWSVTFDGSA
NVSAPLSLSATGVAAGSYPKVTVDTKGRVTAGMALAATDIPGLDASKLVSGVLAEQRLP
VFARGLATAVSNSSDPNTATVPLMLTNHANGPVAGRYFYIQSIFYPDQNGNASQIATSYNA
TSEMYVRVSYAANPSIREWLPWQRCDIGGSFTKEADGELPGGVNLDSMVTSGWWSQSF
TAQAASGANYPIVRAGLLHVYAASSNFIYQTYQAYDGESFYFRCRHSNTWFPWRRMWH
GGDFNPSDYLLKSGFYWNALPGKPATFPPSAHNHDVGQLTSGILPLARGGVGSNTAAGA
RSTIGAGVPATASLGASGWWRDNDTGLIRQWGQVTCPADADASITFPIPFPTLCLGGYAN
QTSAFHPGTDASTGFRGATTTTAVIRNGYFAQAVLSWEAFGR
TuD199_R5_pyocin_tail_fiber
SEQ ID NO: 25
MTTNTPKYGGLLTDIGAAALAAASAAGKKWQPTHMLIGDAGGAPGATPDPIPAATQTKL
INQRYRAQLNRLFVSDKNINTLVAEVVLPVEVGGFWIREIGLQDADGKFVAVSNCPPSYK
AAMESGSARTQTIRVNIALSGLENVQLLIDNGIIYATQDWVKEKVAADFKGRKILAGNGL
VGGGDLSADRSIGLAPSGVTAGSYRSVTVNANGVVTQGSNPSTLAGYAIGDAYTKADTD
GKLAQKANKATTLAGYGITDALRVDGNAVSSSRLAAPRSLAASGDASWSVTFDGSANV
SAPLSLSATGVAAGSYPKVTVDTKGRVTAGMALAATDIPGLDASKLVSGVLAEQRLPVF
ARGLATAVSTTSDPNTATVPLMLTNHANGPVAGRYFYIQSMFYPDQNGNASQIATSYNAT
SEMYVRVSYAANPSARDWLPWKRCDIGGSFSKEADGALGGAVNLNSLITSGWWYQTAN
AQAESGANYPVPRAGLLQVHNAGTNFIYQTYQVYDGEGFYFRCRYTNTWYPWRRVWH
GADFNPNDYLLKSGFTWAALPGKPATFPPTGHNHDAAQITSGILPLARGGLGSNTAAGA
RNNIGAGVPATANRSLNGWWKDNDTGLIVQWMTVSVGDHPGGIVNRSLTFPIAFPTTCL
HVVPSVKELGRPATSASTVTLADVSVSTTGCVIVATEYHGAVQNYAIRLVAIGC

What is claimed is:

1. A therapeutic composition comprising an R-pyocin and a pharmaceutically acceptable carrier, wherein the R-pyocin is isolated from an engineered strain of Pseudomonas aeruginosa.

2. The therapeutic composition of claim 1, wherein the engineered strain of Pseudomonas aeruginosa is selected from PAK, PAO1, Pa-Tud-199, PA-14, PA7, UCBPP-PA14 or RP73.

3. The therapeutic composition of claim 2, wherein the R-pyocin comprises SEQ ID NO: 1, SEQ ID NO: 4, SEQ ID NO: 7, SEQ ID NO: 9, SEQ ID NO:11, SEQ ID NO: 18, SEQ ID NO: 20, or a sequence at least 70% identical to SEQ ID NO: 1, SEQ ID NO: 4, SEQ ID NO: 7, SEQ ID NO: 9, SEQ ID NO:11, SEQ ID NO: 18, or SEQ ID NO: 20.

4. A method of treating a multidrug-resistant bacterial infection in a subject in need thereof, the method comprising administering to the subject a therapeutic composition comprising an R-pyocin and a pharmaceutically acceptable carrier, wherein the R-pyocin is isolated from an engineered strain of Pseudomonas aeruginosa or from a sample obtained from the subject.

5. The method of claim 4, wherein the engineered strain of Pseudomonas aeruginosa is selected from PAK, PAO1, Pa-Tud-199, PA-14, PA7, UCBPP-PA14, or RP73.

6. The method of claim 4, wherein the R-pyocin comprises SEQ ID NO: 1, SEQ ID NO: 4, SEQ ID NO: 7, SEQ ID NO: 9, SEQ ID NO:11, SEQ ID NO: 18, SEQ ID NO: 20, or a sequence at least 70% identical to SEQ ID NO: 1, SEQ ID NO: 4, SEQ ID NO: 7, SEQ ID NO: 9, SEQ ID NO:11, SEQ ID NO: 18, or SEQ ID NO: 20.

7. The method of claim 4, wherein the sample is selected from the group consisting of blood, cerebral spinal fluid (CSF), serum, tissue, wound exudate, abscess exudate, burn exudate, sputum, eye sample, and ear sample.

8. The method of claim 4, wherein the method further comprises administering at least one antibiotic.

9. The method of claim 8, wherein the at least one antibiotic comprises an aminoglycoside or fluoroquinolone.

10. The method of claim 9, wherein the aminoglycoside comprises amikacin, gentamicin, and/or tobramycin.

11. A method of re-sensitizing a multidrug-resistant bacteria to an antibiotic, comprising,
a. isolating an R-pyocin from an engineered strain of Pseudomonas aeruginosa;
b. contacting the multidrug-resistant bacteria with the R-pyocin;
c. contacting the multidrug-resistant bacteria with at least one antibiotic; and
d. measuring colony forming unit (CFU) ability of the multidrug-resistant bacteria, wherein a decrease in CFU ability of the multidrug-resistant bacteria denotes increased antibiotic re-sensitization.

12. The method of claim 11, wherein the antibiotic comprises an aminoglycoside or a fluoroquinolone.

13. The method of claim 12, wherein the aminoglycoside comprises amikacin, gentamicin, and/or tobramycin.

14. The method of claim 11, wherein the engineered strain of Pseudomonas aeruginosa is selected from PAK, PAO1, Pa-Tud-199, PA-14, PA7, UCBPP-PA14, or RP73.

15. The method of claim 11, wherein the R-pyocin comprises SEQ ID NO: 1, SEQ ID NO: 4, SEQ ID NO: 7, SEQ ID NO: 9, SEQ ID NO:11, SEQ ID NO: 18, SEQ ID NO: 20, or a sequence at least 70% identical to SEQ ID NO: 1, SEQ ID NO: 4, SEQ ID NO: 7, SEQ ID NO: 9, SEQ ID NO:11, SEQ ID NO:18, SEQ ID NO: 20.

16. The method of claim 11, wherein the multidrug-resistant bacteria comprise a deletion comprising SEQ ID NO: 14, SEQ ID NO: 15, SEQ ID NO: 16, SEQ ID NO: 17, or a sequence at least 70% identical to SEQ ID NO: 14, SEQ ID NO: 15, SEQ ID NO: 16, or SEQ ID NO: 17.
